(19) United States
(12) Patent Application Publication
(10) Pub. No.: US 2009/0186900 A1
(43) Pub. Date: Jul. 23, 2009

(54) COMPOUND

(76) Inventors: Nigel VICKER, Slough (GB); Gillian Margaret ALLAN, Slough (GB); Harshani Rithma Ruchiranani LAWRENCE, Slough (GB); Joanna Mary DAY, Slough (GB); Atul PUROHIT, Slough (GB); Michael John REED, Slough (GB); Barry Victor Lloyd POTTER, Slough (GB)

Correspondence Address: FROMMER LAWRENCE & HAUG, 745 FIFTH AVENUE- 10TH FL., NEW YORK, NY, 10151, US

(21) Appl. No.: 12/199,364

(22) Filed: Aug. 27, 2008

Related U.S. Application Data
(63) Continuation-in-part of application No. PCT/GB2007/000655, filed on Feb. 26, 2007.

(30) Foreign Application Priority Data
Feb. 27, 2006 (GB) 0603894.7
Aug. 3, 2006 (GB) 0615464.5

(51) Int. Cl. A61K 31/4965; C07C 49/577; A61K 31/122; C07D 213/56; A61K 31/4406; C07D 241/12

(52) U.S. Cl. 514/252.1; 568/327; 514/681; 546/337; 514/357; 544/336

(57) ABSTRACT

There is provided a compound of Formula I

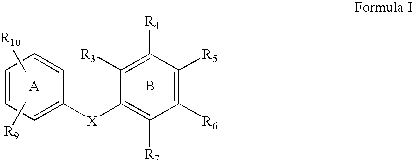

wherein the various symbols are as defined in the description, and a method of manufacturing a medicament for use in the therapy of a condition or disease associated with 17β-hydroxysteroid dehydrogenase (17β-HSD), comprising a compound of Formula I.

### INCORPORATION BY REFERENCE

This application is a continuation-in-part of International Patent Application PCT/GB2007/000655 filed Feb. 26, 2007 and published as WO 2007/096647 on Aug. 30, 2007, which claims priority from Great Britain Patent Application Nos. 0603894.7 filed Feb. 27, 2006 and 0615464.5 filed Aug. 3, 2006.

Each of the above referenced applications, and each document cited in this text (“application cited documents”) and each document cited or referenced in each of the application cited documents, and any manufacturer's specifications or instructions for any products mentioned in this text and in any document incorporated into this text, are hereby incorporated herein by reference; and, technology in each of the documents incorporated herein by reference can be used in the practice of this invention.

It is noted that in this disclosure, terms such as “comprises”, “comprised”, “comprising”, “contains”, “containing” and the like can have the meaning attributed to them in U.S. patent law; e.g., they can mean “includes”, “included”, “including” and the like. Terms such as “consisting essentially of” and “consists essentially of” have the meaning attributed to them in U.S. patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. patent law; namely, that these terms are closed ended.

These and other embodiments are disclosed or are obvious from and encompassed by, the following Detailed Description.

### FIELD OF INVENTION

The present invention relates to a compound. In particular the present invention provides compounds capable of inhibiting 17β-hydroxysteroid dehydrogenase (17β-HSD).

### BACKGROUND TO THE INVENTION

Breast cancer is a devastating disease which remains to be a major cause of death for women in most Western countries. It is estimated to affect approximately 1 million women per year across the globe.¹

Britain has one of the highest mortality rate for breast cancer in the world with over 35,000 women diagnosed each year accounting for nearly one in five of all cancer cases. It is estimated that 1 in 10 women living to the age of 85 in Britain will develop breast cancer during the course of her life. Although modern methods of treatment as well as an earlier detection of the disease have greatly improved survival rates, breast cancer remains the leading cause of death for women aged between 35-54.²

All women are at risk of breast cancer although a number of risk factors have been identified, most of them being related to women's hormonal and reproductive history as well as their family background of the disease. Women at higher risk are generally those with a strong family history of the disease, early onset of menarche, late onset of menopause or a first full-term pregnancy after the age of 30.²

In the earliest stages of a breast cancer, surgery appears to be the treatment of choice. In most of the cases, breast conserving surgical techniques, such as local incision of lump(s) in the breast(s), are involved rather than mastectomy. To prevent any recurrence of the disease, radiotherapy is often prescribed, particularly if breast conserving techniques have been involved.³ It is also used to reduce large tumours to an operable size so that conservational surgery can be carried out.⁴

For advanced breast cancers, when the tumour has spread or recurred, the aim in the treatment is no longer to cure but to reach a palliative control. This is the case when metastases of the tumour have reached locations such as bones, skin, lymph, node or brain. The treatment varies depending on the hormonal status of the patient (whether it is a pre- or post-menopausal woman to be treated) and depending on the type of tumour. Certain tumours have indeed been proven to rely on estrogens for their growth and development, leading to what is called a Hormone Dependent Breast Cancer (HDBC, see I-1). While non HDBC are treated with chemotherapy, where the aim is to kill differentially tumour cells using a combination of cytotoxic agents,⁵ HDBC are expected to respond to endocrine therapy.

The concept of hormone dependent tumours appeared in the early 1960s, when the model of estrogens action was first introduced.⁶ In order for estrogens to regulate cell growth and function in humans, a specific protein, called the human Oestrogen Receptor (hER), must be present.⁷ This protein, localised in the nucleus, interacts with estrogens resulting in the formation of a binding complex. This acts as a transcription factor by activating production of m-RNA from specific genes, one or more of which are probably essential for efficient tumour cell growth.

Patients with a measurable level of receptor protein are classified as oestrogen-receptor-positive (ER+) with opposition to oestrogen-receptor-negative (ER−). About 50% of pre-menopausal women and 75% of post-menopausal women fall into the ER+ group⁸ where the development of breast cancers can be directly linked to the presence of estrogens. Endocrine therapy, where the use of drugs results in a deprivation of estrogenic stimulation to cells, has proven to be an effective approach to the treatment of HDBC. Originally, two classes of drugs, responding to different strategies, were developed: anti-oestrogens and aromatase inhibitors.

Anti-oestrogens, as antagonists of the oestrogen receptor, have been one of the first treatments considered for HDBC. Their action relies on their ability to bind competitively to the specific receptor protein hER, thus preventing access of endogenous estrogens to their specific binding site. Consequently, the natural hormone is unable to maintain tumour growth.

Of the anti-oestrogens commonly used in breast cancer therapy, tamoxifen (below) is the most widely used because of the very low toxicity profile of the molecule. Despite its non-steroidal skeleton, tamoxifen possesses a mixed agonist-antagonist activity that limits its therapeutic potential.⁹ In addition, some form of drug resistance has been reported in patients after long-term tamoxifen treatment.¹⁰

Novel pure anti-oestrogenic drugs, such as ICI 164384 (below), have since been discovered but the loss of potency compared with that of tamoxifen suggested the need to design more highly potent targets.¹¹

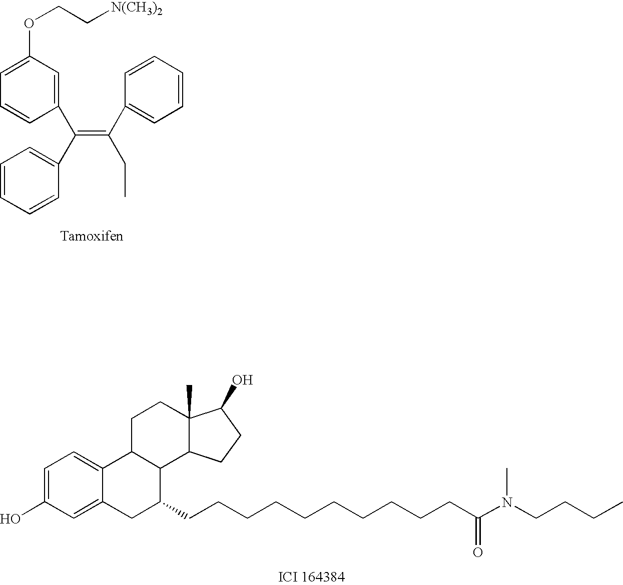

For some years now, a new type of anti-oestrogen has emerged, combining oestrogen agonism on target tissues such as bone or liver and antagonism and/or minimal agonism in reproductive tissues such as breasts or uterus.¹² These compounds, designed as Selective Oestrogen Receptor Modulators (SERMs), are not only potentially effective in reducing a patient's risk of breast carcinoma but they have also been shown to increase bone mineral density and prevent osteoporosis in post-menopausal women. Raloxifen is the first of this class of compounds to be used clinically.¹³ More SERMs are currently in clinical trials and these molecules might one day replace tamoxifen as the first line treatment for women with HDBC.

The use of therapeutic agents that inhibit one or several enzyme of the steroid biosynthesis pathway represents another important strategy to control of the development of oestrogen-dependent tumours.¹⁴ The enzyme aromatase, which converts androgenic C19 steroids to estrogenic C18 steroids, has been the prime target for reducing oestrogen levels. This enzyme complex, which contains a cytochrome P450 haemoprotein, catalyses the aromatisation of the androgen A-ring with the subsequent loss of the C19 methyl group to yield estrogens.

Aminoglutethimide (below) was the first aromatase inhibitor used for the treatment of breast cancer. It however showed a number of undesirable side effects given its wide spectrum of inhibitory effects on other P450-dependant enzymes, and attempts to improve on the original structure have led to a number of non-steroidal compounds entering clinical trials.¹⁵ The last generation developed compounds such as letrozole, which combine high potency and high selectivity for the enzyme, and are also better tolerated.

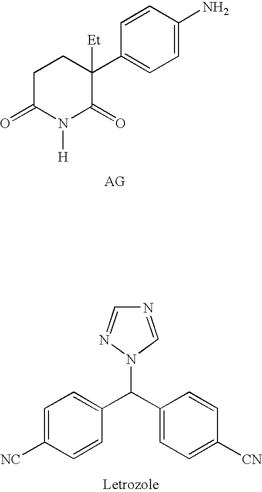

Structure of different types of aromatase inhibitors. Generation I: aminoglutethimide, AG; generation III, letrozole.

Traditionally, aromatase inhibitors are reserved as second line treatment for advanced HDBC patients whose diseases are no longer controlled by tamoxifen. However, because of the extreme good toxicity profile of some of the latest aromatase inhibitors, recent clinical trials have been conducted to assess their suitability as first line treatment for HDBC.

Strong evidence has emerged over the past decade, both biochemically and clinically, that the sole inhibition of the enzyme aromatase cannot afford an effective reduction of estrogenic stimulation to HDBC, the reason being that other pathways are involved in oestrogen biosynthesis. The sulphatase pathway is now considered to be the major route for breast tumour oestrogen synthesis since sulphatase activity was found to provide 10 fold more oestrone than the aromatase activity.¹⁶

In the sulphatase pathway, estrogens are synthesised from the highly available precursor oestrone-sulphate, via two enzymes (scheme below): steroid sulphatase (STS) which hydrolyses oestrone-sulphate into oestrone, and 17β-hydroxysteroid dehydrogenase (17β-HSD) which reduces oestrone into oestradiol. These two enzymes represent the latest targets for oestrogen deprivation strategies.

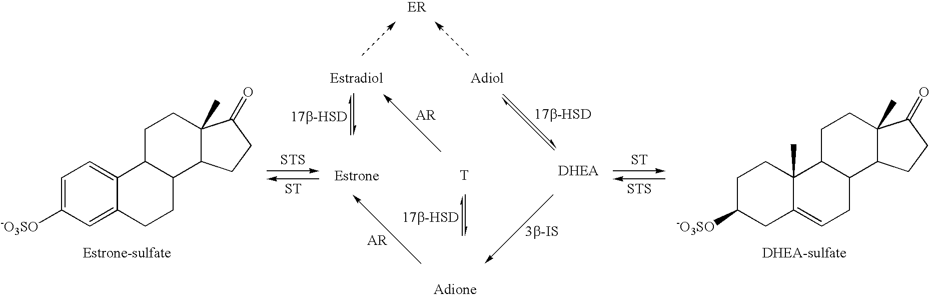

Origin of estrogens in normal and tumoral breast cells. AR, aromatase; ST: steroid sulfotransferase; STS, steroid sulphatase; 17β-HSD, 17β-hydroxysteroid dehydrogenase; 3β-IS, 3β-hydroxysteroid dehydrogenase Δ⁵,Δ⁴-isomerase; ER, oestrogen receptor.

Several potent inhibitors have been identified for steroid sulphatase. They all share the common structural feature of an aromatic ring bearing a substituent that mimics the phenolic A-ring of the enzyme substrate, oestrone-sulphate. On the development of steroidal inhibitors, a wide variety of chemical groups have been introduced at C3, of which the 3-O-sulfamate was found to be the most potent for the oestrone molecule. The resulting compound, estrone-3-O-sulfamate (below) led to the identification of the aryl-O-sulphamate structure as an active pharmacophore required for potent inhibition of STS. EMATE was shown to inhibit steroid sulphatase activity in a time- and concentration-dependent manner¹⁷ and was active in vivo on oral administration.¹⁸ It was however revealed to be highly estrogenic which raised the need to design STS inhibitors devoid of agonist activity on hER.

To avoid the problems linked to an active steroid nucleus, non steroid-based inhibitors have been synthesised. Coumarin sulphamate such as 4-methylcoumarin-7-O-sulfamate (COUMATE, below), where the active pharmacophore is conserved, have been among the first inhibitors of that type to be identified.¹⁹ Although COUMATE is less potent than EMATE, it has the advantage of being non estrogenic.²⁰ Some tricyclic coumarin-based sulphamates have also been developed and turned out to be much more potent than COUMATE, while retaining its non estrogenic characteristic.²¹ 667COUMATE, which is some 3 times more potent than EMATE in vitro is now in pre-clinical development for clinical trials.²²

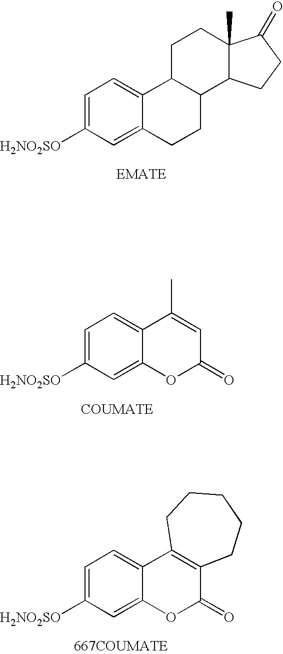

### Structures of the Steroid Sulphatase Inhibitors EMATE, COUMATE and 667COUMATE.

PCT/GB92/01587 teaches novel steroid sulphatase inhibitors and pharmaceutical compositions containing them for use in the treatment of oestrone dependent tumours, especially breast cancer. These steroid sulphatase inhibitors are sulphamate esters, such as N,N-dimethyl oestrone-3-sulphamate and, preferably, oestrone-3-sulphamate (EMATE). It is known that EMATE is a potent E1-STS inhibitor as it displays more than 99% inhibition of E1-STS activity in intact MCF-7 cells at 0.1 mM. EMATE also inhibits the E1-STS enzyme in a time- and concentration-dependent manner, indicating that it acts as an active site-directed inactivator. Although EMATE was originally designed for the inhibition of E1-STS, it also inhibits dehydroepiandrosterone sulphatase (DHA-STS), which is an enzyme that is believed to have a pivotal role in regulating the biosynthesis of the oestrogenic steroid androstenediol. Also, there is now evidence to suggest that androstenediol may be of even greater importance as a promoter of breast tumour growth. EMATE is also active in vivo as almost complete inhibition of rat liver E1-STS (99%) and DHA-STS (99%) activities resulted when it is administered either orally or subcutaneously. In addition, EMATE has been shown to have a memory enhancing effect in rats. Studies in mice have suggested an association between DHA-STS activity and the regulation of part of the immune response. It is thought that this may also occur in humans. The bridging O-atom of the sulphamate moiety in EMATE is important for inhibitory activity. Thus, when the 3-O-atom is replaced by other heteroatoms as in oestrone-3-N-sulphamate and oestrone-3-S-sulphamate, these analogues are weaker non-time-dependent inactivators.

Although optimal potency for inhibition of E1-STS may have been attained in EMATE, it is possible that oestrone may be released during sulphatase inhibition and that EMATE and its oestradiol congener may possess oestrogenic activity.

17β-HSD, which catalyses the final step in estrogens and androgens biosynthesis, also appeared as a target for oestrogen deprivation strategies. This enzyme is responsible for the interconversion of the oxidised form (less active) and the reduced form (more active) of steroids. Its activity directly supports the growth and development of oestrogen dependent tumours since it preferably reduces oestrone into estradiol²⁵ and in a minor extend, via the conversion of the androgen DHEA into androstenediol (Adiol), which has recently been proven to have estrogenic properties and to be able to bind to the oestrogen receptor.²⁶

17β-HSD belongs to a family of isoenzymes, 13 of which have been so far identified and cloned.²⁷ Each type has a selective substrate affinity and directional activity which means that selectivity of drug action has to be achieved. 17β-HSD type 1 is the isotype that catalyses the interconversion of oestrone and oestradiol.

Unlike STS inhibitors, only few 17β-HSD inhibitors have been reported. Most of the steroidal inhibitors for 17β-HSD type 1 have in common a D-ring modified structure. Oestradiol derivatives which contain a side-chain with a good leaving group at the 16α-position have been shown to be a potent class of inhibitors. In particular, 16α-(bromoalkyl)-estradiol²⁸ where the side-chains exhibit high reactivity towards nucleophilic amino-acids residues in the active site of the enzyme were found to be promising irreversible inhibitors. Analogues containing short bromoalkyl moieties at position 16 exhibited the highest activity with 16α-(Bromopropyl)-oestradiol, followed by 16α-(Bromobutyl)-oestradiol, the most potent of the series (3 and 4). They, however, turned out to be pure agonists of the oestrogen receptor.

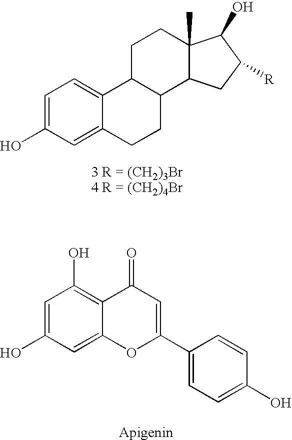

17β-HSD type 1 inhibitors: 16α-(bromopropyl)-oestradiol, 3; 16α-(bromopropyl)-oestradiol, 4 and a flavone derivative, apigenin.

In an attempt to eliminate the intrinsic oestrogenicity of potent inhibitors and possibly at the same time engineer anti-oestrogenic properties into the molecule, several 16α-(broadly)-oestradiol derivatives bearing the C7α-alkylamide side chain of the known anti-oestrogen ICI 164384 were synthesised.²⁹ However, rather poor inhibition of 170-HSD type 1 was obtained, with estrogenic and anti-oestrogenic properties not completely abolished or introduced respectively.

In parallel, non-steroidal inhibitors of 17β-HSD type 1 have been designed. Flavonoids, which are structurally similar to estrogens, are able to bind to the oestrogen receptor with estrogenic or anti-estrogenic activities.³⁰ Their action on aromatase activity is well documented and in recent studies, they were found to reduce the conversion of oestrone into oestradiol catalysed by 17β-HSD type 1.³¹ Flavone derivatives, such as apigenin emerged from a SAR study as a promising compounds with some inhibitory activity on 170-HSD type 1 without being estrogenic at the inhibitory concentration.³²

Ahmed et al (Biochem Biophys Res Commun 1999 Jan. 27; 254(3): 811-5) report on a structure-activity relationship study of steroidal and nonsteroidal inhibitors of STS.

Steroid dehydrogenases (DH) such as oestradiol 17β-hydroxysteroid dehydrogenases (E2HSD) have pivotal roles in regulating the availability of ligands to interact with the oestrogen receptor. E2HSD Type I reduces oestrone (E1) to the biologically active oestrogen, oestradiol (E2), while E2HSD Type II inactivates E2 by catalysing its oxidation to E1. Thus the identification of compounds having DH inhibitory activity, in particular, inhibitors of E2HSD Type I, could be of therapeutic value in inhibiting the formation of E2.

### SUMMARY ASPECTS OF THE PRESENT INVENTION

The present invention provides novel compounds which are capable of acting as effective 17β-hydroxysteroid dehydrogenase (17β-HSD) inhibitors. The present invention identifies that the compounds of the present application are effective 17β-hydroxysteroid dehydrogenase (17β-HSD) inhibitors.

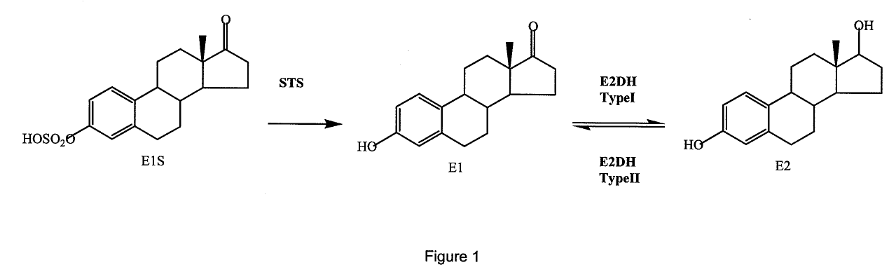

FIG. 1 shows some of the enzymes involved in the in situ synthesis of oestrone from oestrone sulphate, and oestradiol. “STS” denotes Steroid Sulphatase, “E2DH Type I” denotes Oestradiol 17β-hydroxysteroid dehydrogenase Type I or Oestradiol 17β-hydroxysteroid dehydrogenase Type 1, 3, 5 and/or 7 and “E2DH Type II” denotes Oestradiol 17β-hydroxysteroid dehydrogenase Type II or Oestradiol 17β-hydroxysteroid dehydrogenase Type 2 and/or 8.

As can be seen, two enzymes that are involved in the peripheral synthesis of oestrogens are the enzyme Oestradiol 17β-hydroxysteroid dehydrogenase and the enzyme steroid sulphatase.

In situ synthesis of oestrogen is thought to make an important contribution to the high levels of oestrogens in tumours and therefore specific inhibitors of oestrogen biosynthesis are of potential value for the treatment of endocrine-dependent tumours.

Moreover, even though oestrogen formation in malignant breast and endometrial tissues via the sulphatase pathway makes a major contribution to the high concentration of oestrogens, there are still other enzymatic pathways that contribute to in vivo synthesis of oestrogen.

Thus, there is an urgent need to develop new therapies for the treatment of these cancers.

The present invention therefore seeks to overcome one or more of the problems associated with the prior art methods of treating breast and endometrial cancers.

In one aspect, therefore, the present invention provides a use of a compound for the preparation of a medicament that can or affect, such as substantially inhibit, the steroid dehydrogenase pathway—which pathway converts oestrone to and from oestradiol.

This aspect of the present invention is advantageous because by the administration of one type of compound it is possible to block the synthesis of oestradiol from oestrone. Hence, the present invention provides compounds that have considerable therapeutic advantages, particularly for treating breast and endometrial cancers.

The compounds of the present invention may comprise other substituents. These other substituents may, for example, further increase the activity of the compounds of the present invention and/or increase stability (ex vivo and/or in vivo).

### DETAILED ASPECTS OF THE PRESENT INVENTION

Aspects of the invention are described in the claims of the present application.

In one aspect the present invention provides a compound of Formula I

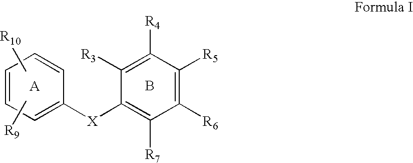

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted
wherein X is a bond or a linker group
wherein
(A) (i) R₉ is selected from alkyl and halogen groups; and
(ii) R₁₀ is selected from —OH, oxyhydrocarbyl and —OSO₂NR₁R₂; wherein R₁ and
   R₂ are independently selected from H and hydrocarbyl,
   wherein when R₉ is a halogen group and R₁₀ is —OH, at least one of R₃, R₄, R₅, R₆ and R₇ are as defined in (B), (C), (D) or (E);
   or
   (B) at least one of R₃, R₄, R₅, R₆ and R₇ is the group —C(═O)—CR₁₁R₁₂-R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
   wherein R₁, and R₁₂ are independently selected from H and hydrocarbyl;
   or
   (C) at least one of R₃, R₄, R₅, R₆ and R₇ together with another of R₃, R₄, R₅, R₆ and R₇ forms a ring containing —C(═O)—;
   or
   (D) at least one of R₃, R₄, R₅, R₆ and R₇ is selected from alkylheterocycle group, alkenylheterocycle group, alkylheteroaryl group, alkenylheteroaryl group, and heteroaryl groups
   or
   (E) at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
   wherein R₁₃ and R₁₄ are independently selected from H and hydrocarbyl.

According to one aspect of the present invention, there is provided a compound for use in medicine, wherein the compound is of Formula I

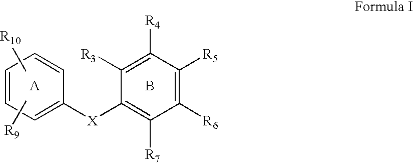

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted and/or optionally contains one or more hetero atoms (such as N)
wherein X is a bond or a linker group
wherein
(A) (i) R₉ is selected from alkyl and halogen groups; or (ii) R₁₀ is selected from —OH, oxyhydrocarbyl and —OSO₂NR₁R₂; wherein R₁ and R₂ are independently selected from H and hydrocarbyl; or (iii) ring A contains one or more hetero atoms
or
(B) at least one of R₃, R₄, R₅, R₆ and R₇ is the group —C(═O)—CR₁₁R₁₂—R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
   wherein R₁₁ and R₁₂ are independently selected from H and hydrocarbyl;
   or
   (C) at least one of R₃, R₄, R₅, R₆ and R₇ together with another of R₃, R₄, R₅, R₆ and R₇ forms a ring containing —C(═O)—;
   or
   (D) at least one of R₃, R₄, R₅, R₆ and R₇ is selected from alkylheterocycle group, alkenylheterocycle group, alkylheteroaryl group, alkenylheteroaryl group, and heteroaryl groups
   or
   (E) at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
   wherein R₁₃ and R₁₄ are independently selected from H and hydrocarbyl.

According to one aspect of the present invention, there is provided a compound according to the present invention for use in medicine.

According to one aspect of the present invention, there is provided a pharmaceutical composition comprising a compound of Formula I

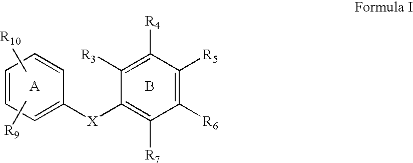

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted and/or optionally contains one or more hetero atoms (such as N)
wherein X is a bond or a linker group
wherein
(A) (i) R₉ is selected from alkyl and halogen groups; or (ii) R₁₀ is selected from —OH, oxyhydrocarbyl and —OSO₂NR₁R₂; wherein R₁ and R₂ are independently selected from H and hydrocarbyl; or (iii) ring A contains one or more hetero atoms
or
(B) at least one of R₃, R₄, R₅, R₆ and R₇ is the group —C(═O)—CR₁₁R₁₂-R₃ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
   wherein R₁₁ and R₁₂ are independently selected from H and hydrocarbyl;
   or
   (C) at least one of R₃, R₄, R₅, R₆ and R₇ together with another of R₃, R₄, R₅, R₆ and R₇ forms a ring containing —C(═O)—;
   or
   (D) at least one of R₃, R₄, R₅, R₆ and R₇ is selected from alkylheterocycle group, alkenylheterocycle group, alkylheteroaryl group, alkenylheteroaryl group, and heteroaryl groups
   or
   (E) at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
   wherein R₁₃ and R₁₄ are independently selected from H and hydrocarbyl.
   optionally admixed with a pharmaceutically acceptable carrier, diluent, excipient or adjuvant.

In one aspect the present invention provides use of a compound in the manufacture of a medicament for use in the therapy of a condition or disease associated with 17β-hydroxysteroid dehydrogenase (17β-HSD), wherein the compound is of Formula I

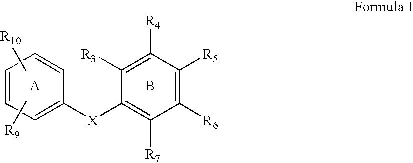

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted and/or optionally contains one or more hetero atoms (such as N)
wherein X is a bond or a linker group
wherein
(A) (i) R₉ is selected from alkyl and halogen groups; or (ii) R₁₀ is selected from —OH, oxyhydrocarbyl and —OSO₂NR₁R₂; wherein R₁ and R₂ are independently selected from H and hydrocarbyl; or (iii) ring A contains one or more hetero atoms
or
(B) at least one of R₃, R₄, R₅, R₆ and R₇ is the group —C(═O)—CR₁₁R₁₂-R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
   wherein R₁₁ and R₁₂ are independently selected from H and hydrocarbyl;
   or
   (C) at least one of R₃, R₄, R₅, R₆ and R₇ together with another of R₃, R₄, R₅, R₆ and R₇ forms a ring containing —C(═O)—;
   or
   (D) at least one of R₃, R₄, R₅, R₆ and R₇ is selected from alkylheterocycle group, alkenylheterocycle group, alkylheteroaryl group, alkenylheteroaryl group, and heteroaryl groups
   or
   (E) at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
   wherein R₁₃ and R₁₄ are independently selected from H and hydrocarbyl.

According to one aspect of the present invention, there is provided the use of a compound according to the present invention in the manufacture of a medicament for use in the therapy of a condition or disease associated with steroid dehydrogenase.

According to one aspect of the present invention, there is provided the use of a compound in the manufacture of a medicament for use in the therapy of a condition or disease associated with adverse steroid dehydrogenase levels, wherein the compound is of Formula I

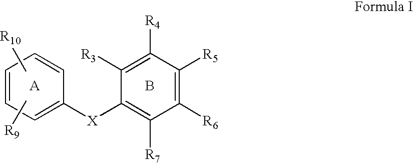

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted and/or optionally contains one or more hetero atoms (such as N)
wherein X is a bond or a linker group
wherein
(A) (i) R₉ is selected from alkyl and halogen groups; or (ii) R₁₀ is selected from —OH, oxyhydrocarbyl and —OSO₂NR₁R₂; wherein R₁ and R₂ are independently selected from H and hydrocarbyl; or (iii) ring A contains one or more hetero atoms
or
(B) at least one of R₃, R₄, R₅, R₆ and R₇ is the group —C(═O)—CR₁₁R₁₂-R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
   wherein R₁, and R₁₂ are independently selected from H and hydrocarbyl;
   or
   (C) at least one of R₃, R₄, R₅, R₆ and R₇ together with another of R₃, R₄, R₅, R₆ and R₇ forms a ring containing —C(═O)—;
   or
   (D) at least one of R₃, R₄, R₅, R₆ and R₇ is selected from alkylheterocycle group, alkenylheterocycle group, alkylheteroaryl group, alkenylheteroaryl group, and heteroaryl groups
   or
   (E) at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
   wherein R₁₃ and R₁₄ are independently selected from H and hydrocarbyl.

According to one aspect of the present invention, there is provided the use of a compound according to the present invention in the manufacture of a medicament for use in the therapy of a condition or disease associated with adverse steroid dehydrogenase levels.

### Some Advantages

One key advantage of the present invention is that the compounds of the present invention can act as 17β-HSD inhibitors.

Another advantage of the compounds of the present invention is that they may be potent in vivo.

Some of the compounds of the present invention may be non-oestrogenic compounds. Here, the term “non-oestrogenic” means exhibiting no or substantially no oestrogenic activity.

Another advantage is that some of the compounds may not be capable of being metabolised to compounds which display or induce hormonal activity.

Some of the compounds of the present invention are also advantageous in that they may be orally active.

Some of the compounds of the present invention may useful for the treatment of cancer, such as breast cancer, as well as (or in the alternative) non-malignant conditions, such as the prevention of auto-immune diseases, particularly when pharmaceuticals may need to be administered from an early age.

Thus, some of the compounds of the present invention are also believed to have therapeutic uses other than for the treatment of endocrine-dependent cancers, such as the treatment of autoimmune diseases.

For ease of reference, these and further aspects of the present invention are now discussed under appropriate section headings. However, the teachings under each section are not necessarily limited to each particular section.

### Further/Preferable Aspects

As discussed herein, in some aspects of the present invention the compound is of Formula I

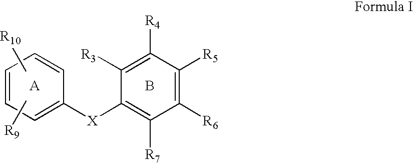

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted and/or optionally contains one or more hetero atoms (such as N)
wherein X is a bond or a linker group
wherein
(A) (i) R₉ is selected from alkyl and halogen groups; or (ii) R₁₀ is selected from —OH, oxyhydrocarbyl and —OSO₂NR₁R₂; wherein R₁ and R₂ are independently selected from H and hydrocarbyl; or (iii) ring A contains one or more hetero atoms
or
(B) at least one of R₃, R₄, R₅, R₆ and R₇ is the group —C(═O)—CR¹¹R₁₂-R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
   wherein R₁₁ and R₁₂ are independently selected from H and hydrocarbyl;
   or
   (C) at least one of R₃, R₄, R₅, R₆ and R₇ together with another of R₃, R₄, R₅, R₆ and R₇ forms a ring containing —C(═O)—;
   or
   (D) at least one of R₃, R₄, R₅, R₆ and R₇ is selected from alkylheterocycle group, alkenylheterocycle group, alkylheteroaryl group, alkenylheteroaryl group, and heteroaryl groups
   or
   (E) at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
   wherein R₁₃ and R₁₄ are independently selected from H and hydrocarbyl.

In one aspect (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl.

In a further aspect (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl, wherein when R₉ is a halogen group and R₁₀ is —OH, at least one of R₃, R₄, R₅, R₆ and R₇ are as defined in (B), (C), (D) or (E);

As discussed herein, in some aspects of the present invention the compound is of Formula I

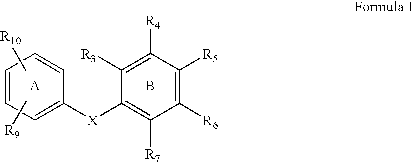

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted
wherein X is a bond or a linker group
wherein
(A) (i) R₉ is selected from alkyl and halogen groups; and
(ii) R₁₀ is selected from —OH, oxyhydrocarbyl and —OSO₂NR₁R₂; wherein R₁ and R₂ are independently selected from H and hydrocarbyl,
   wherein when R₉ is a halogen group and R₁₀ is —OH, at least one of R₃, R₄, R₅, R₆ and R₇ are as defined in (B), (C), (D) or (E);
   or
   (B) at least one of R₃, R₄, R₅, R₆ and R₇ is the group —C(═O)—CR₁₁R₁₂-R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
   wherein R₁₁ and R₁₂ are independently selected from H and hydrocarbyl;
   or
   (C) at least one of R₃, R₄, R₅, R₆ and R₇ together with another of R₃, R₄, R₅, R₆ and R₇ forms a ring containing —C(═O)—;
   or
   (D) at least one of R₃, R₄, R₅, R₆ and R₇ is selected from alkylheterocycle group, alkenylheterocycle group, alkylheteroaryl group, alkenylheteroaryl group, and heteroaryl groups
   or
   (E) at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
   wherein R₁₃ and R₁₄ are independently selected from H and hydrocarbyl.

In one preferred aspect of the present invention the compound is of Formula II

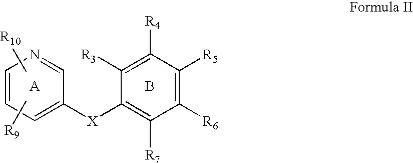

In one preferred aspect of the present invention the compound is of Formula III

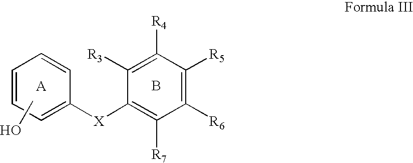

In one preferred aspect of the present invention the compound is of Formula IV

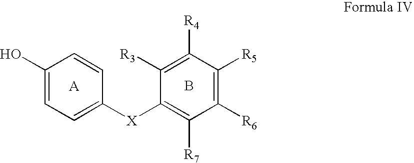

In one preferred aspect of the present invention the compound is of Formula V

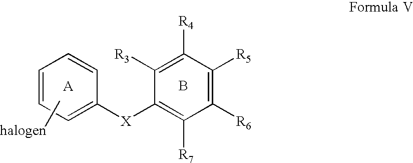

wherein halogen is preferably F.

In one preferred aspect of the present invention the compound is of Formula VI

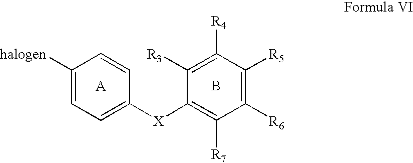

wherein halogen is preferably F.

In one preferred aspect of the present invention the compound is of Formula VII

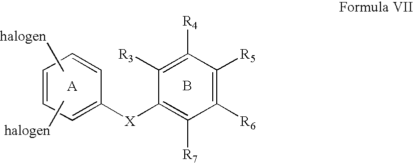

wherein halogen is preferably F.

In one preferred aspect of the present invention the compound is of Formula VII

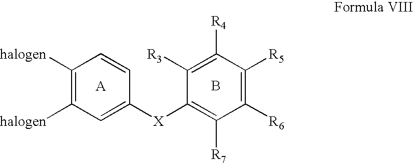

wherein halogen is preferably F.

In one preferred aspect of the present invention at least one of R₃, R₄, R₅, R₆ and R₇ is selected from alkylheterocycle group, alkenylheterocycle group, alkylheteroaryl group, alkenylheteroaryl group, and heteroaryl groups.

In one preferred aspect of the present invention at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;

wherein R₁₃ and R₁₄ are independently selected from H and hydrocarbyl.

In one preferred aspect of the present invention R₉ is selected from or R₉ of (A) is selected from ethyl and fluoro groups.

In one preferred aspect of the present invention R₁₀ is selected from or R₁₀ of (A) is selected from is selected from —OH and methoxy.

### A Ring

The compound of or for use in the present invention comprises ring A. It will be understood by one skilled in the art that the ring may contain any suitable atoms. For example ring A may contain C and optionally one or more hetero atoms. Typical hetero atoms include O, N and S, in particular N. In one aspect ring A contains C and optionally one or more N atoms. In one aspect ring A contains only C atoms i.e. it is carbocyclic.

As discussed herein ring A is optionally further substituted. In one aspect ring A is substituted only by groups R₉ and R₁₀. It will be understood that the remaining valencies of the atoms of the ring are occupied by H.

Typically ring A will contain from 4 to 10 members. Preferably ring A will contain 5, 6 or 7 members.

In one preferred aspect of the present invention ring A contains a nitrogen.

As discussed herein group X is a bond or a linker group. In one aspect X is a bond. In one aspect X is a linker group.

### X

When X is a bond Formula I may be denoted as follows:

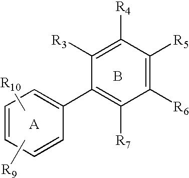

The linker group may be any suitable linker group. In preferred aspects the linker is selected from a bond and (R₂₂)₁₋₃ wherein each R₂₂ is independently selected from O, S, S═O, NR₂₃, S(═O)₂ and C═O, wherein R₂₃ is selected from H and hydrocarbyl. Thus X may be selected from a bond and (R₂₂)₁₋₃ wherein each R₂₂ is independently selected from O, S, S═O, NR₂₃, S(═O)₂ and C═O, wherein R₂₃ is selected from H and hydrocarbyl.

In one preferred aspect X is selected from a bond and (R₂₂)₃ wherein each R₂₂ is independently selected from O, S, S═O, NR₂₃, S(═O)₂ and C═O, wherein R₂₃ is selected from H and hydrocarbyl.

In one preferred aspect X is selected from a bond and (R₂₂)₂ wherein each R₂₂ is independently selected from O, S, S═O, NR₂₃, S(═O)₂ and C═O, wherein R₂₃ is selected from H and hydrocarbyl.

In one preferred aspect X is selected from a bond and R₂₂ wherein R₂₂ is independently selected from O, S, S═O, NR₂₃, S(═O)₂ and C═O, wherein R₂₃ is selected from H and hydrocarbyl.

In one preferred aspect X is selected from a bond, NR₂₃S(═O)₂, NR₂₃C═O, S═O, O, and S, wherein R₂₃ is selected from H and hydrocarbyl.

Preferably R₂₃ is an alkyl group. In this aspect, the R₂₃ alkyl group preferably has from 1 to 20 carbons, preferably from 1 to 10 carbons, preferably from 1 to 5 carbons, preferably 1, 2 or 3 carbons.

The R₂₃ alkyl group may be branched or straight chain. Preferably the alkyl group is straight chained.

In one preferred aspect R₂₃ is selected from H and alkyl groups having from 1 to 20 carbons, preferably selected from H and alkyl groups having from 1 to 10 carbons, preferably selected from H and alkyl groups having from 1 to 5 carbons, preferably selected from H and alkyl groups having from 1, 2 or 3 carbons, most preferably H or methyl.

In one preferred aspect X is selected from CH₂, O, S and a bond.

Preferably X is selected from O, S and a bond.

### B Ring

The compound of or for use in the present invention comprises ring B. It will be understood by one skilled in the art that the ring may contain any suitable atoms. For example ring B may contain C and optionally one or more hetero atoms. Typical hetero atoms include O, N and S, in particular N. In one aspect ring A contains C and optionally one or more N atoms. In one aspect ring B contains only C atoms i.e. it is carbocyclic.

Typically ring B will contain from 4 to 10 members. Preferably ring B will contain 5, 6 or 7 members.

### R₃, R₃, R₅, R₆, R₇, R₉, and R₁₀

As discussed herein R₃, R₄, R₅, R₆, R₇, R₉, and R₁₀ are independently selected from —H, —OH, hydrocarbyl groups, oxyhydrocarbyl groups, cyano (—CN), nitro (—NO₂), and halogens.

The term “hydrocarbyl group” as used herein means a group comprising at least C and H and may optionally comprise one or more other suitable substituents. Examples of such substituents may include hydroxyl, ═O (to provide a carbonyl group), halo, alkoxy, nitro, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group.

A typical hydrocarbyl group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch.

In some aspects of the present invention, the hydrocarbyl group is selected from optionally substituted alkyl group, optionally substituted haloalkyl group, aryl group, alkylaryl group, alkylarylakyl group, and an alkene group.

In some aspects of the present invention, the hydrocarbyl group is an optionally substituted alkyl group.

In some aspects of the present invention, the hydrocarbyl group is selected from C₁-C₁₀ alkyl group, such as C₁-C₆ alkyl group, and C₁-C₃ alkyl group. Typical alkyl groups include C₁ alkyl, C₂ alkyl, C₃ alkyl, C₄ alkyl, C₅ alkyl, C₇ alkyl, and C₈ alkyl.

In some aspects of the present invention, the hydrocarbyl group is selected from C₁-C₁₀ haloalkyl group, C₁-C₆ haloalkyl group, C₁-C₃ haloalkyl group, C₁-C₁₀ bromoalkyl group, C₁-C₆ bromoalkyl group, and C₁-C₃ bromoalkyl group. Typical haloalkyl groups include C₁ haloalkyl, C₂ haloalkyl, C₃ haloalkyl, C₄ haloalkyl, C₅ haloalkyl, C₇ haloalkyl, C₈ haloalkyl, C₁ bromoalkyl, C₂ bromoalkyl, C₃ bromoalkyl, C₄ bromoalkyl, C₅ bromoalkyl, C₇ bromoalkyl, and C₈ bromoalkyl.

In some aspects of the present invention, the hydrocarbyl group is selected from aryl groups, alkylaryl groups, alkylarylakyl groups, —(CH₂)₁₋₁₀-aryl, —(CH₂)₁₋₁₀-Ph, (CH₂)₁₋₁₀-Ph-C₁₋₁₀ alkyl, —(CH₂)₁₋₅-Ph, (CH₂)₁₋₅-Ph-C₁₋₅ alkyl, —(CH₂)₁₋₃-Ph, (CH₂)₁₋₃-Ph-C₁₋₃ alkyl, —CH₂-Ph, and —CH₂-Ph-C(CH₃)₃.

When the hydrocarbyl, group is or contains an aryl group, the aryl group or one or more of the aryl groups may contain a hetero atom. Thus the aryl group or one or more of the aryl groups may be carbocyclic or more may heterocyclic. Typical hetero atoms include O, N and S, in particular N.

In some aspects of the present invention, the hydrocarbyl group is selected from —(CH₂)₁₋₁₀-cycloalkyl, —(CH₂)₁₋₁₀—C₃₋₁₀cycloalkyl, —(CH₂)₁₋₇—C₃₋₇cycloalkyl, —(CH₂)₁₋₅—C₃₋₅cycloalkyl, —(CH₂)₁₋₃—C₃₋₅cycloalkyl, and —CH₂—C₃cycloalkyl.

In some aspects of the present invention, the hydrocarbyl group is an alkene group.

Typical alkene groups include C₁-C₁₀ alkene group, C₁-C₆ alkene group, C₁-C₃ alkene group, such as C₁, C₂, C₃, C₄, C₅, C₆, or C₇ alkene group. In a preferred aspect the alkene group contains 1, 2 or 3 C═C bonds. In a preferred aspect the alkene group contains 1 C═C bond. In some preferred aspect at least one C═C bond or the only C═C bond is to the terminal C of the alkene chain, that is the bond is at the distal end of the chain to the ring system.

The term “oxyhydrocarbyl” group as used herein means a group comprising at least C, H and O and may optionally comprise one or more other suitable substituents. Examples of such substituents may include, but not limited to halo-, alkoxy-, nitro-, an alkyl group, a cyclic group etc. In addition to the possibility of the substituents being a cyclic group, a combination of substituents may form a cyclic group. If the oxyhydrocarbyl group comprises more than one C then those carbons need not necessarily be linked to each other. For example, at least two of the carbons may be linked via a suitable element or group. Thus, the oxyhydrocarbyl group may contain hetero atoms. Suitable hetero atoms will be apparent to those skilled in the art and include, for instance, sulphur and nitrogen.

In one embodiment of the present invention, the oxyhydrocarbyl group is a oxyhydrocarbon group.

Here the term “oxyhydrocarbon” means any one of an alkoxy group, an oxyalkenyl group, an oxyalkynyl group, which groups may be linear, branched or cyclic, or an oxyaryl group. The term oxyhydrocarbon also includes those groups but wherein they have been optionally substituted. If the oxyhydrocarbon is a branched structure having substituent(s) thereon, then the substitution may be on either the hydrocarbon backbone or on the branch; alternatively the substitutions may be on the hydrocarbon backbone and on the branch.

In one embodiment of the present invention, the oxyhydrocarbyl group is an alkoxy group.

Typically, the oxyhydrocarbyl group is of the formula C₁₋₆O (such as a C₁₋₃O).

For some compounds of the present invention, it is highly preferred that the oxyhydrocarbyl group is and in particular the A ring of the ring system is substituted with, an alkoxy group.

Preferably the alkoxy group is methoxy.

It is of course understood that halogens refer to F, Cl, Br and I. In one aspect the halogen, each halogen or at least one halogen is F. In one aspect the halogen, each halogen or at least one halogen is Cl. In one aspect the halogen, each halogen or at least one halogen is Br. In one aspect the halogen, each halogen or at least one halogen is I.

In one aspect at least one of R₃, R₄, R₅, R₆, R₇, R₉, and R₁₀ is —OH.

In one aspect at least one of R₃, R₄, R₅, R₆, R₇, R₉, and R₁₀ is a hydrocarbyl group.

In one aspect at least one of R₃, R₄, R₅, R₆, R₇, R₉, and R₁₀ is a oxyhydrocarbyl groups.

In one aspect at least one of R₃, R₄, R₅, R₆, R₇, R₉, and R₁₀ is cyano (—CN).

In one aspect at least one of R₃, R₄, R₅, R₆, R₇, R₉, and R₁₀ is nitro (—NO₂).

In one aspect at least one of R₃, R₄, R₅, R₆, R₇, R₉, and R₁₀ is a halogen.

### A

In one aspect of the present invention (i) R₉ is selected from alkyl and halogen groups; or (ii) R₁₀ is selected from —OH, oxyhydrocarbyl and —OSO₂NR₁R₂; wherein R₁ and R₂ are independently selected from H and hydrocarbyl; or (iii) ring A contains one or more hetero atoms

In one aspect of the present invention (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl.

In one aspect of the present invention (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl,

wherein when R₉ is a halogen group and R₁₀ is —OH, at least one of R₃, R₄, R₅, R₆ and R₇ are as defined in (B), (C), (D) or (E);

In one aspect R₉ is alkyl and R₁₀ is —OH.

In one aspect R₉ is alkyl and R₁₀ is oxyhydrocarbyl.

In one aspect R₉ is alkyl and R₁₀ is —OSO₂NR₁R₂.

In one aspect R₉ is a halogen and R₁₀ is —OH.

In one aspect R₉ is a halogen and R₁₀ is oxyhydrocarbyl/

In one aspect R₉ is a halogen and R₁₀ is —OSO₂NR₁R₂.

In one aspect (a) R₉ is alkyl and R₁₀ is —OH or (b) R₉ is alkyl and R₁₀ is oxyhydrocarbyl or (c) R₉ is alkyl and R₁₀ is —OSO₂NR₁R₂ or (d) R₉ is a halogen and R₁₀ is oxyhydrocarbyl or (e) R₉ is a halogen and R₁₀ is —OSO₂NR₁R₂.

### B

In one aspect of the present invention at least one of R₃, R₄, R₅, R₆ and R₇ is the group —C(═O)—CR₁₁R₁₂-R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
   wherein R₁₁ and R₁₂ are independently selected from H and hydrocarbyl.

In one preferred aspect of the present invention at least one of R₃, R₄, R₅, R₆ and R₇ is the group —C(═O)—CH₂—R₈

### R₈

### Alkyloxyalkyl Group

In one preferred aspect of the present invention R₈ is an alkyloxyalkyl group.

The alkyloxyalkyl group is a -alkyl-O-alkyl group.

Each alkyl of the group is preferably independently has from 1 to 20 carbons, preferably from 1 to 10 carbons, preferably from 1 to 5 carbons, preferably 1, 2 or 3 carbons

Each alkyl of the group may be independently branched or straight chain. Preferably one or each alkyl is straight chain

Particularly preferred alkyloxyalkyl groups are -EtOEt, -EtOMe, -MeOEt, and -MeOMe.

### Nitrile Group

In one preferred aspect of the present invention R₈ may be a nitrile group or comprise a nitrile group. Nitrile group is understood to mean a —C═N group.

In one preferred aspect of the present invention R₈ is a nitrile group.

In one preferred aspect when R₈ is a nitrile group, R² is —OH.

In one preferred aspect R₈ is a nitrile group and R² is —OH.

Preferably when R₈ is a nitrile group, herein R₂ is capable of forming a hydrogen bond.

The term “capable of forming a hydrogen bond” as used herein means a group having a region of negative charge capable of forming one part of a hydrogen bond.

### Alkylaryl Group

In one preferred aspect of the present invention R₈ is an alkylaryl group.

It will be understood that by alkylaryl group it is meant a group denoted by -alkyl-aryl.

The alkyl group preferably has from 1 to 20 carbons, preferably from 1 to 10 carbons, preferably from 1 to 5 carbons, preferably 1, 2 or 3 carbons.

The alkyl group may be branched or straight chain. Preferably the alkyl group is straight chained.

The aryl group typically has a six membered ring

The aryl group is substituted. In preferred aspects the aryl group of the alkylaryl group is substituted by a group selected from N,N-dialkyl, alkoxy, nitro, nitrile, and azaalkyl groups.

Preferably the N,N-dialkyl is —N(C₁₋₁₀ alkyl)₂, —N(C₁₋₅ alkyl)₂, or —N(C₁₋₃ alkyl)₂. Particularly preferred is NMe₂.

Preferably the alkoxy group is C₁₋₁₀ alkoxy, C₁₋₅ alkoxy, and C₁₋₃ alkoxy. Particularly preferred is methoxy.

In a highly preferred aspect the alkylaryl group is —CH₂-Ph.

### Alkenylaryl Group

In one preferred aspect of the present invention R₈ is an alkenylaryl group.

It will be understood that by alkylaryl group it is meant a group denoted by -alkenyl-aryl.

In one preferred aspect of the present invention, such as in the present use or method the aryl group of the alkenylaryl group is substituted.

The alkenyl group preferably has from 1 to 20 carbons, preferably from 1 to 10 carbons, preferably from 1 to 5 carbons, preferably 1, 2 or 3 carbons.

The alkenyl group may be branched or straight chain. Preferably the alkenyl group is straight chained.

The aryl group typically has a six membered ring

The aryl group is substituted. In preferred aspects the aryl group of the alkylaryl group is substituted by a group selected from N,N-dialkyl, alkoxy, nitro, nitrile, and azaalkyl groups.

Preferably the N,N-dialkyl is —N(C₁₋₁₀ alkyl)₂, —N(C₁₋₅ alkyl)₂, or —N(C₁₋₃ alkyl)₂. Particularly preferred is NMe₂.

Preferably the alkoxy group is C₁₋₁₀ alkoxy, C₁₋₅ alkoxy, and C₁₋₃ alkoxy. Particularly preferred is methoxy.

In a highly preferred aspect the alkenylaryl group is ═CH-Ph.

### Alkylheteroaryl Group

In one preferred aspect of the present invention R₈ is an alkylheteroaryl group.

The alkyl group preferably has from 1 to 20 carbons, preferably from 1 to 10 carbons, preferably from 1 to 5 carbons, preferably 1, 2 or 3 carbons.

The alkyl group may be branched or straight chain. Preferably the alkyl group is straight chained.

The aryl group typically has a six membered ring

The heteroaryl group contain carbon and hetero atoms. Typical hetero atoms include O, N and S, in particular N.

The aryl group may be substituted or unsubstituted. Preferably the aryl group is substituted. In preferred aspects the aryl group of the alkylaryl group is substituted by a group selected from N,N-dialkyl, alkoxy, nitro, nitrile, and azaalkyl groups.

Preferably the N,N-dialkyl is —N(C₁₋₁₀ alkyl)₂, —N(C₁₋₅ alkyl)₂, or —N(C₁₋₃ alkyl)₂. Particularly preferred is NMe₂

Preferably the alkoxy group is C₁₋₁₀ alkoxy, C₁₋₅ alkoxy, and C₁₋₃ alkoxy. Particularly preferred is methoxy.

### Alkenylheteroaryl Group

In one preferred aspect of the present invention R₈ is an alkenylheteroaryl group. In one preferred aspect, R₈ is an alkenylheteroaryl group, wherein the aryl group is substituted. Preferably R₈ is an alkenylheteroaryl group wherein the aryl group is unsubstituted.

In one preferred aspect of the present invention R₈ is an alkylheteroaryl group.

The alkenyl group preferably has from 1 to 20 carbons, preferably from 1 to 10 carbons, preferably from 1 to 5 carbons, preferably 1, 2 or 3 carbons.

The alkenyl group may be branched or straight chain. Preferably the alkenyl group is straight chained.

The aryl group typically has a six membered ring

The heteroaryl group contain carbon and hetero atoms. Typical hetero atoms include O, N and S, in particular N.

The aryl group may be substituted or unsubstituted. Preferably the aryl group is substituted. In preferred aspects the aryl group of the alkylaryl group is substituted by a group selected from N,N-dialkyl, alkoxy, nitro, nitrile, and azaalkyl groups.

Preferably the N,N-dialkyl is —N(C₁₋₁₀ alkyl)₂, —N(C₁₋₅ alkyl)₂, or —N(C₁₋₃ alkyl)₂. Particularly preferred is NMe₂

Preferably the alkoxy group is C₁₋₁₀ alkoxy, C₁₋₅ alkoxy, and C₁₋₃ alkoxy. Particularly preferred is methoxy.

In a highly preferred aspect the alkenylheteroaryl group is selected from

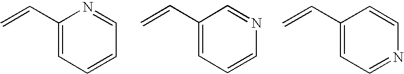

### Oxime Group (═N—O-Alkyl or ═N—O—H Group)

In one preferred aspect of the present invention R₈ is an oxime group, namely ═N—O-alkyl or ═N—O—H group.

Preferably the oxime group is a ═N—O-alkyl group.

In one preferred aspect when R₈ is selected from ═N—O-alkyl and ═N—O—H groups and R³ is a ═N—O-alkyl group or ═N—O—H group.

The alkyl group preferably has from 1 to 20 carbons, preferably from 1 to 10 carbons, preferably from 1 to 5 carbons, preferably 1, 2 or 3 carbons.

The alkyl group may be branched or straight chain. Preferably the alkyl group is straight chained.

### Branched Alkenyl

In one preferred aspect of the present invention R₈ is a branched alkenyl group.

The branched alkenyl group preferably has from 1 to 20 carbons, preferably from 1 to 10 carbons, preferably from 1 to 5 carbons, preferably 1, 2, 3 or 4 carbons.

### Alkyl-Alcohol Group Or Alkenyl-Alcohol Group

In one preferred aspect of the present invention R₈ is an alkyl-alcohol group or alkenyl-alcohol group.

In one preferred aspect of the present invention R₈ is an alkyl-alcohol group.

The alkyl group may be branched or straight chain. Preferably the alkyl group is straight chained.

By alkyl-alcohol group or alkenyl-alcohol group it is meant a group of the formula CₓH_{2x-2n-y}(OH)_{y} wherein x is an integer, y is an integer and n is the degree of unsaturation.

Typically x is from 1 to 20, preferably from 1 to 10, preferably from 1 to 5, preferably 1, 2, 3 or 4. Typically y is 1, 2 or 3. Typically n is 0, 1, 2 or 3.

Preferably the alkenyl-alcohol has the following structural formula:

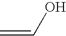

In one preferred aspect the alkenyl alcohol is substituted. In this aspect preferably a substituent replaces Hₐ and/or H_{b} in the following structural formula:

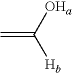

Suitable substituents include ester groups, haloalkyl groups, aryl groups such as heteroaryl groups, and alkyl groups.

Preferred substituted alkenyl-alcohol groups include

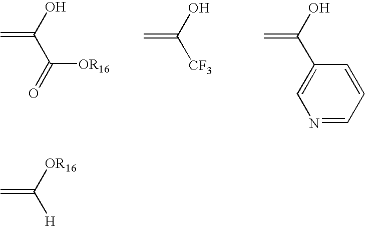

wherein R₁₆ is a hydrocarbyl group, preferably a hydrocarbon, more preferably an alkyl group, preferably having from 1 to 20 carbons, preferably from 1 to 10 carbons, preferably from 1 to 5 carbons, preferably 1, 2, or 3 carbons. In a highly preferred aspect R₁₆ is an ethyl group.

### Amide or Alkylamide

In one preferred aspect of the present invention R₈ is an amide or alkylamide group.

In one aspect, R₈ is an amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, alkylamine group, alkyloxyalkyl group, alkylaryl group, straight or branched alkyl group,

Preferably, in one aspect, R₈ is an amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,

In one preferred aspect the amide is of the formula —C(═O)NR₁₇R₁₈ or —N(CO—R₁₉)R₂₀, wherein R₁₇, R₁₈, R₁₉ and R₂₀ are independently selected from H and hydrocarbyl groups.

Preferred amide groups include NHCO—C₁₋₁₀alkyl, CONH C₁₋₁₀alkyl, NHCO(CH₂)₁₋₁₀CH₃, CONH(CH₂)₁₋₁₀CH₃, NHCO(CH₂)₃₋₇CH₃, CONH(CH₂)₃₋₇CH₃, NHCO(CH₂)₆CH₃, CONH(CH₂)₆CH₃.

In one preferred aspect the amide or alkylamide is an alkylamide.

Preferred alkylamide groups include C₁₋₁₀alkyl-NHCO—C₁₋₁₀alkyl, C₁₋₁₀alkyl-CONH—C₁₋₁₀alkyl, C₁₋₁₀alkyl-NHCO(CH₂)₁₋₁₀CH₃, C₁₋₁₀alkyl-CONH(CH₂)₁₋₁₀CH₃, C₁₋₁₀alkyl-NHCO(CH₂)₃₋₇CH₃, C₁₋₁₀alkyl-CONH(CH₂)₃₋₇CH₃, C₁₋₁₀alkyl-NHCO(CH₂)₆CH₃, C₁₋₁₀alkyl-CONH(CH₂)₆CH₃.

In one preferred aspect the substituents of the di-substituted amide together form a cyclic structure.

In one preferred aspect the substituents of the di-substituted amide together form an aryl ring.

In one preferred aspect the substituents of the di-substituted amide together form a heterocyclic ring.

### —CHO (Enol Tautomer)

In one preferred aspect of the present invention R₈ is —CHO, or together with another of R₃, R₄, R₅, R₆ and R₇ the enol tautomer thereof.

In one preferred aspect of the present invention R₈ is —CHO.

In one preferred aspect of the present invention R₈ or together with another of R₃, R₄, R₅, R₆ and R₇ the enol tautomer of a —CHO group.

In one preferred aspect of the present invention R₈ is selected from

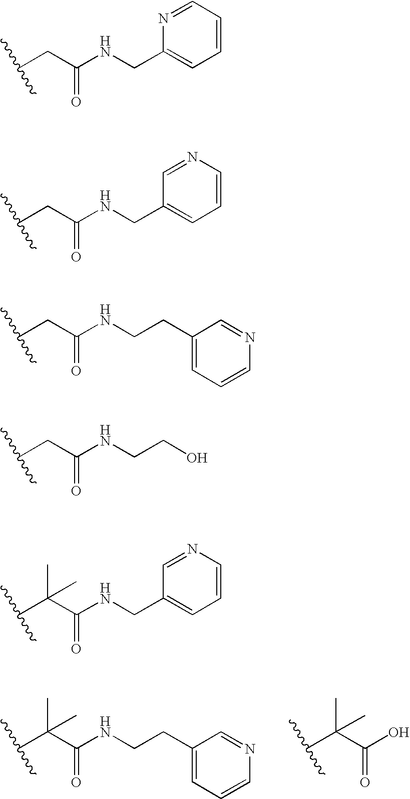

### R₁₁ and R₁₂

As discussed herein R₁₁ and R₁₂ are independently selected from H and hydrocarbyl.

In one aspect at least on one of R₁₁ and R₁₂ is H.

In one aspect both R₁₁ and R₁₂ are H.

In one aspect at least on one of R₁₁ and R₁₂ is hydrocarbyl.

In one aspect both R₁₁ and R₁₂ are hydrocarbyl.

The meaning of the term “hydrocarbyl” is as discussed herein.

In some aspects of the present invention, the hydrocarbyl group is selected from optionally substituted alkyl group, optionally substituted haloalkyl group, aryl group, alkylaryl group, alkylarylakyl group, and an alkene group.

In some aspects of the present invention, the hydrocarbyl group is an optionally substituted alkyl group.

In some aspects of the present invention, the hydrocarbyl group is selected from C₁-C₁₀ alkyl group, such as C₁-C₆ alkyl group, and C₁-C₃ alkyl group. Typical alkyl groups include C₁ alkyl, C₂ alkyl, C₃ alkyl, C₄ alkyl, C₅ alkyl, C₇ alkyl, and C₈ alkyl.

### C

In one aspect of the present invention at least one of R₃, R₄, R₅, R₆ and R₇ together with another of R₃, R₄, R₅, R₆ and R₇ forms a ring containing —C(═O)—;

It will be understood by one skilled in the art that the ring may contain any suitable atoms. For example ring A may contain C and optionally one or more hetero atoms. Typical hetero atoms include O, N and S, in particular N. In one aspect ring A contains C and optionally one or more N atoms. In one aspect ring A contains only C atoms i.e. it is carbocyclic.

Typically the ring will contain from 4 to 10 members. Preferably the ring will contain 5, 6 or 7 members. Preferably in these aspects the compound of one of the following formulae

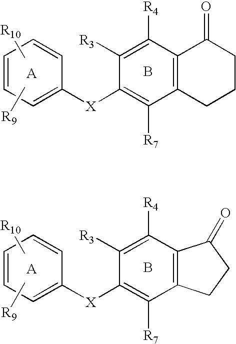

The ring containing —C(═O)—, formed from at least one of R₃, R₄, R₅, R₆ and R₇ together with another of R₃, R₄, R₅, R₆ and R₇, may be optionally substituted. In one aspect the ring containing —C(═O)—, formed from at least one of R₃, R₄, R₅, R₆ and R₇ together with another of R₃, R₄, R₅, R₆ and R₇, is unsubstituted. In one aspect the ring containing —C(═O)—, formed from at least one of R₃, R₄, R₅, R₆ and R₇ together with another of R₃, R₄, R₅, R₆ and R₇, is substituted.

When the ring containing —C(═O)—, formed from at least one of R₃, R₄, R₅, R₆ and R₇ together with another of R₃, R₄, R₅, R₆ and R₇, is (optionally) substituted, the substituents may be selected from any suitable substituents. Suitable substituents may be selected from:

(i) an alkyloxyalkyl group
(ii) a nitrile group
(iii) alkylaryl group, wherein the aryl group is substituted by other than a methyl group
(iv) alkenylaryl group
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) carboxylic acid esters
(ix) CO₂H or alkyl-CO₂H group
(x) branched alkenyl
(xi) alkyl-alcohol group or alkenyl-alcohol group, and
(xii) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group.

Each of the suitable substituents (i) to (xii) are preferably as defined herein in respect of R₈.

When the suitable substituent is a carboxylic acid ester the ester may be selected from —CO₂R₂₁, —CH₂CO₂R₂₁, and —CH₂CH₂CO₂R₂₁, wherein R₂₁ is a hydrocarbyl group.

Preferably R₂₁ is a hydrocarbon group. More preferably R₂₁ is an alkyl group such as an alkyl group preferably having from 1 to 20 carbons, preferably from 1 to 10 carbons, preferably from 1 to 5 carbons, preferably 1, 2 or 3 carbons.

The alkyl group may be branched or straight chain. Preferably the alkyl group is straight chained.

When the suitable substituent is —CO₂H or alkyl-CO₂H group (a carboxylic acid or alkyl-carboxylic acid), the alkyl group may be branched or straight chain. Preferably the alkyl group is straight chained. The alkyl group preferably has from 1 to 20 carbons, preferably from 1 to 10 carbons, preferably from 1 to 5 carbons, preferably 1, 2 or 3 carbons.

When the suitable substituent is —CO₂H or alkyl-CO₂H group (a carboxylic acid or alkyl-carboxylic acid, the alkyl may be a CH₂ or CH₂CH₂ group.

In one preferred aspect of the present invention one of R₃, R₄, R₅, R₆ and R₇ together with another of R₃, R₄, R₅, R₆ and R₇ forms the ring

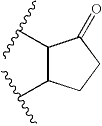

In one preferred aspect of the present invention the compound is of Formula IX

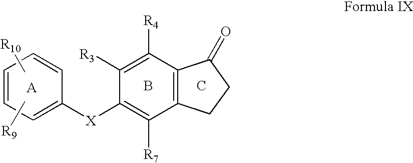

wherein ring C is optionally substituted, preferably wherein ring C is optionally substituted with a group selected from

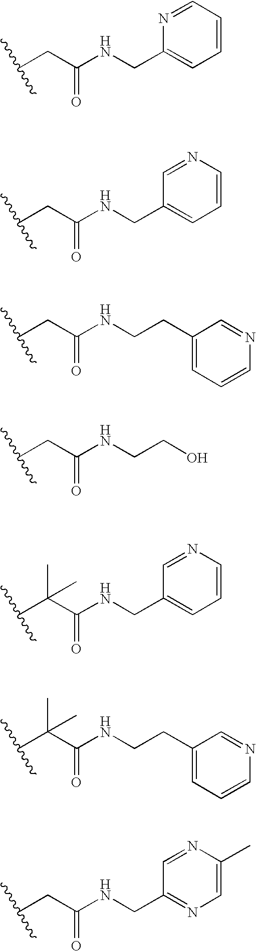

### D

In one aspect of the present invention at least one of R₃, R₄, R₅, R₆ and R₇ is selected from alkylheterocycle group, alkenylheterocycle group, alkylheteroaryl group, alkenylheteroaryl group, and heteroaryl groups.

In one aspect of the present invention at least one of R₃, R₄, R₅, R₆ and R₇ is selected from alkylheterocycle groups

In one aspect of the present invention at least one of R₃, R₄, R₅, R₆ and R₇ is selected from alkenylheterocycle groups

In one aspect of the present invention at least one of R₃, R₄, R₅, R₆ and R₇ is selected from alkylheteroaryl groups.

In one aspect of the present invention at least one of R₃, R₄, R₅, R₆ and R₇ is selected from alkenylheteroaryl groups

In one aspect of the present invention at least one of R₃, R₄, R₅, R₆ and R₇ is selected from heteroaryl groups.

Each of the alkylheterocycle group, alkenylheterocycle group, alkylheteroaryl group, alkenylheteroaryl group, and heteroaryl groups are preferably as defined herein in respect of R₈.

### E

In one aspect of the present invention at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;

wherein R₁₃ and R₁₄ are independently selected from H and hydrocarbyl.

In one aspect of the present invention at least one of R₃, R₄, R₅, R₆ and R₇ is —CN.

In one aspect of the present invention at least one of R₃, R₄, R₅, R₆ and R₇ is selected from —C(R₁₃)═N—O-alkyl groups, wherein R₁₃ is selected from H and hydrocarbyl.

In one aspect of the present invention at least one of R₃, R₄, R₅, R₆ and R₇ is selected from —C(R₁₄)═N—O—H groups, R₁₄ is selected from H and hydrocarbyl.

In one aspect of the present invention at least one of R₃, R₄, R₅, R₆ and R₇ is selected from optionally substituted pyrazole.

The optional substituents may be selected from —OH, hydrocarbyl groups, —CN, nitro, and halogens. In one preferred aspect the optional substituents are selected from —(R₃₁)₀₋₁—OH, —(R₃₁)₀₋₁—CN, —(R₃₁)₀₋₁halogen, —(R₃₁)₀₋₁-NR₂₄R₂₈, —(R₃₁)₀₋₁—NR₂₅—C(═O)—R₂₈, —(R₃₁)₀₋₁—C(═O)—NR₂₆-R₂₈, —(R₃₁)₀₋₁—CO₂—R₂₉, —(R₃₁)₀₋₁—CO₂—H, —(R₃₁)₀₋₁—NR₂₇—S(═O)₂—R₃₂, —(R₃₁)₀₋₁—O—R₃₀; wherein each of R₂₄, R₂₅, R₂₆, and R₂₇ is independently selected from H and hydrocarbyl, wherein each R₂₈ is independently selected from H and hydrocarbyl; and wherein each of R₂₉, R₃₀, R₃₂ are independently selected from alkyl groups.

In one aspect of the present invention at least one of R₃, R₄, R₅, R₆ and R₇ is selected from optionally substituted thiazole.

The optional substituents may be selected from —OH, hydrocarbyl groups, —CN, nitro, and halogens. In one preferred aspect the optional substituents are selected from —(R₃₁)₀₋₁—OH, —(R₃₁)₀₋₁—CN, —(R₃₁)₀₋₁halogen, —(R₃₁)₀₋₁—NR₂₄R₂₈, —(R₃₁)₀₋₁—NR₂₅—C(═O)—R₂₈, —(R₃₁)₀₋₁—C(═O)—NR₂₆—R₂₈, —(R₃₁)₀₋₁—CO₂—R₂₉, —(R₃₁)₀₋₁—CO₂—H, —(R₃₁)₀₋₁—NR₂₇—S(═O)₂—R₃₂, —(R₃₁)₀₋₁—O—R₃₀; wherein each of R₂₄, R₂₅, R₂₆, and R₂₇ is independently selected from H and hydrocarbyl, wherein each R₂₈ is independently selected from H and hydrocarbyl; and wherein each of R₂₉, R₃₀, R₃₂ are independently selected from alkyl groups.

In one aspect of the present invention at least one of R₃, R₄, R₅, R₆ and R₇ is selected from optionally substituted oxazole.

The optional substituents may be selected from —OH, hydrocarbyl groups, —CN, nitro, and halogens. In one preferred aspect the optional substituents are selected from —(R₃₁)₀₋₁—OH, —(R₃₁)₀₋₁—CN, —(R₃₁)₀₋₁halogen, —(R₃₁)₀₋₁—NR₂₄R₂₈, —(R₃₁)₀₋₁—NR₂₅—C(═O)—R₂₈, —(R₃₁)₀₋₁—C(═O)—NR₂₆—R₂₈, —(R₃₁)₀₋₁—CO₂—R₂₉, —(R₃₁)₀₋₁—CO₂—H, —(R₃₁)₀₋₁—NR₂₇—S(═O)₂—R₃₂, —(R₃₁)₀₋₁—O—R₃₀; wherein each of R₂₄, R₂₅, R₂₆, and R₂₇ is independently selected from H and hydrocarbyl, wherein each R₂₈ is independently selected from H and hydrocarbyl; and wherein each of R₂₉, R₃₀, R₃₂ are independently selected from alkyl groups.

In one aspect of the present invention at least one of R₃, R₄, R₅, R₆ and R₇ is selected from optionally substituted isoxazole.

The optional substituents may be selected from —OH, hydrocarbyl groups, —CN, nitro, and halogens. In one preferred aspect the optional substituents are selected from —(R₃₁)₀₋₁—OH, —(R₃₁)₀₋₁—CN, —(R₃₁)₀₋₁halogen, —(R₃₁)₀₋₁—NR₂₄R₂₈, —(R₃₁)₀₋₁—NR₂₅—C(═O)—R₂₈, —(R₃₁)₀₋₁—C(═O)—NR₂₆—R₂₈, —(R₃₁)₀₋₁—CO₂—R₂₉, —(R₃₁)₀₋₁—CO₂—H, —(R₃₁)₀₋₁—NR₂₇—S(═O)₂—R₃₂, —(R₃₁)₀₋₁—O—R₃₀; wherein each of R₂₄, R₂₅, R₂₆, and R₂₇ is independently selected from H and hydrocarbyl, wherein each R₂₈ is independently selected from H and hydrocarbyl; and wherein each of R₂₉, R₃₀, R₃₂ are independently selected from alkyl groups.

In one aspect of the present invention at least one of R₃, R₄, R₅, R₆ and R₇ is selected from optionally substituted pyridine.

The optional substituents may be selected from —OH, hydrocarbyl groups, —CN, nitro, and halogens. In one preferred aspect the optional substituents are selected from —(R₃₁)₀₋₁—OH, —(R₃₁)₀₋₁—CN, —(R₃₁)₀₋₁halogen, —(R₃₁)₀₋₁, —NR₂₄R₂₈, —(R₃₁)₀₋₁—NR₂₅—C(═O)—R₂₈, —(R₃₁)₀₋₁—C(═O)—NR₂₆—R₂₈, —(R₃₁)₀₋₁—CO₂—R₂₉, —(R₃₁)₀₋₁—CO₂—H, —(R₃₁)₀₋₁—NR₂₇—S(═O)₂—R₃₂, —(R₃₁)₀₋₁—O—R₃₀; wherein each of R₂₄, R₂₅, R₂₆, and R₂₇ is independently selected from H and hydrocarbyl, wherein each R₂₈ is independently selected from H and hydrocarbyl; and wherein each of R₂₉, R₃₀, R₃₂ are independently selected from alkyl groups.

In one aspect of the present invention at least one of R₃, R₄, R₅, R₆ and R₇ is selected from optionally substituted pyrimidine.

The optional substituents may be selected from —OH, hydrocarbyl groups, —CN, nitro, and halogens. In one preferred aspect the optional substituents are selected from —(R₃₁)₀₋₁—OH, —(R₃₁)₀₋₁—CN, —(R₃₁)₀₋₁halogen, —(R₃₁)₀₋₁—NR₂₄R₂₈, —(R₃₁)₀₋₁—NR₂₅—C(═O)—R₂₈, —(R₃₁)₀₋₁—C(═O)—NR₂₆—R₂₈, —(R₃₁)₀₋₁—CO₂—R₂₉, —(R₃₁)₀₋₁—CO₂—H, —(R₃₁)₀₋₁—NR₂₇—S(═O)₂—R₃₂, —(R₃₁)₀₋₁—O—R₃₀; wherein each of R₂₄, R₂₅, R₂₆, and R₂₇ is independently selected from H and hydrocarbyl, wherein each R₂₈ is independently selected from H and hydrocarbyl; and wherein each of R₂₉, R₃₀, R₃₂ are independently selected from alkyl groups.

In one aspect of the present invention at least one of R₃, R₄, R₅, R₆ and R₇ together with another of R₃, R₄, R₅, R₆ and R₇ forms a nitrogen containing ring.

The optional substituents may be selected from —OH, hydrocarbyl groups, —CN, nitro, and halogens. In one preferred aspect the optional substituents are selected from —(R₃₁)₀₋₁—OH, —(R₃₁)₀₋₁—CN, —(R₃₁)₀₋₁halogen, —(R₃₁)₀₋₁—NR₂₄R₂₈, —(R₃₁)₀₋₁—NR₂₅—C(═O)—R₂₈, —(R₃₁)₀₋₁—C(═O)—NR₂₆—R₂₈, —(R₃₁)₀₋₁—CO₂—R₂₉, —(R₃₁)₀₋₁—CO₂—H, —(R₃₁)₀₋₁—NR₂₇—S(═O)₂—R₃₂, —(R₃₁)₀₋₁—O—R₃₀; wherein each of R₂₄, R₂₅, R₂₆, and R₂₇ is independently selected from H and hydrocarbyl, wherein each R₂₈ is independently selected from H and hydrocarbyl; and wherein each of R₂₉, R₃₀, R₃₂ are independently selected from alkyl groups.

The meaning of the term “hydrocarbyl” is as discussed herein.

In some aspects of the present invention, the hydrocarbyl group is selected from optionally substituted alkyl group, optionally substituted haloalkyl group, aryl group, alkylaryl group, alkylarylakyl group, and an alkene group.

In some aspects of the present invention, the hydrocarbyl group is an optionally substituted alkyl group.

In some aspects of the present invention, the hydrocarbyl group is selected from C₁-C₁₀ alkyl group, such as C₁-C₆ alkyl group, and C₁-C₃ alkyl group. Typical alkyl groups include C₁ alkyl, C₂ alkyl, C₃ alkyl, C₄ alkyl, C₅ alkyl, C₇ alkyl, and C₈ alkyl.

### Substituents

The compound of the present invention may have substituents other than those of the ring systems show herein. Furthermore the ring systems herein are given as general formulae and should be interpreted as such. The absence of any specifically shown substituents on a given ring member indicates that the ring member may substituted with any moiety of which H is only one example. The ring system may contain one or more degrees of unsaturation, for example is some aspects one or more rings of the ring system is aromatic. The ring system may be carbocyclic or may contain one or more hetero atoms.

The compound of the invention, in particular the ring system compound of the invention of the present invention may contain substituents other than those show herein. By way of example, these other substituents may be one or more of: one or more sulphamate group(s), one or more phosphonate group(s), one or more thiophosphonate group(s), one or more sulphonate group(s), one or more sulphonamide group(s), one or more halo groups, one or more O groups, one or more hydroxy groups, one or more amino groups, one or more sulphur containing group(s), one or more hydrocarbyl group(s)—such as an oxyhydrocarbyl group.

In general terms the ring system of the present compounds may contain a variety of non-interfering substituents. In particular, the ring system A′B′C′D′ may contain one or more hydroxy, alkyl especially lower (C₁-C₆) alkyl, e.g. methyl, ethyl, n-propyl, isopropyl, n-butyl, sec-butyl, tert-butyl, n-pentyl and other pentyl isomers, and n-hexyl and other hexyl isomers, alkoxy especially lower (C₁-C₆) alkoxy, e.g. methoxy, ethoxy, propoxy etc., alkinyl, e.g. ethinyl, or halogen, e.g. fluoro substituents.

For some compounds of the present invention, it is preferred that the ring system is substituted with a oxyhydrocarbyl group. More preferably the A′ ring of the ring system is substituted with a oxyhydrocarbyl group.

For some compounds of the present invention, it is preferred that the ring system is substituted with a hydrocarbylsulphanyl group. The term “hydrocarbylsulphanyl” means a group that comprises at least hydrocarbyl group (as herein defined) and sulphur, preferably —S-hydrocarbyl, more preferably —S-hydrocarbon. That sulphur group may be optionally oxidised.

Preferably the hydrocarbylsulphanyl group is —S—C₁₋₁₀ alkyl, more preferably —S—C₁₋₅ alkyl, more preferably —S—C₁₋₃ alkyl, more preferably —S—CH₂CH₂CH₃, —S—CH₂CH₃ or —SCH₃

For some compounds of the present invention, it is highly preferred that the ring system is substituted with an alkyl group.

Preferably the alkyl group is ethyl.

For some compounds of the present invention, it is highly preferred that the compound comprises at least two or more of sulphamate group, a phosphonate group, a thiophosphonate group, a sulphonate group or a sulphonamide group.

For some compounds of the present invention, it is highly preferred that the compound comprises at least two sulphamate groups.

For some compounds of the present invention, it is highly preferred that the compound comprises at least two sulphamate groups, wherein said sulphamate groups are not on the same ring.

For some compounds of the present invention, it is highly preferred that the A′ ring of the ring system comprises at least one sulphamate group and wherein the D′ ring of the ring system comprises at least one sulphamate group.

### Further Aspects

According to a further aspect the present invention provides a compound of Formula I compound of Formula I

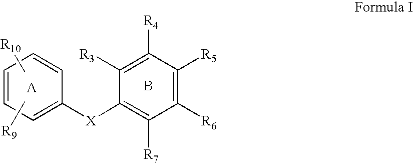

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted
wherein X is a bond or a linker group
wherein
(A) (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl

### OR

(B) at least one of R₃, R₄, R₅, R₆ and R₇ is the group —C(═O)—CR₁₁R₁₂—R₈ or together with another of R₃, R₄, R₅, R₆ and R₇ forms a ring containing —C(═O)—;
wherein R₈ is a selected from
(i) an alkyloxyalkyl group
(ii) a nitrile group, and wherein R² is capable of forming a hydrogen bond
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof

### OR

(C) at least one of R₃, R₄, R₅, R₆ and R₇ is selected from —CN, —C(R₁₁)═N—O-alkyl group, —C(R₁₁)═N—O—H group, optionally substituted pyrazole, optionally substituted thiazole, optionally substituted oxazole, optionally substituted pyridine, and optionally substituted pyrimidine, or together with another of R₃, R₄, R₅, R₆ and R₇ forms a nitrogen containing ring;
wherein R₁₁ and R₁₂ are independently selected from H and hydrocarbyl.

According to one aspect of the present invention, there is provided a compound for use in medicine, wherein the compound is of Formula I

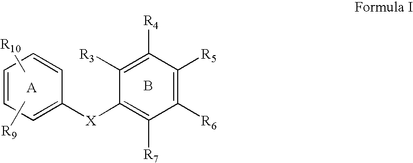

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted
wherein X is a bond or a linker group
wherein
(A) (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl

### OR

(B) at least one of R₃, R₄, R₅, R₆ and R₇ is the group —C(═O)—CR₁₁R₁₂—R₈ or together with another of R₃, R₄, R₅, R₆ and R₇ forms a ring containing —C(═O)—;
wherein R₈ is a selected from
(i) an alkyloxyalkyl group
(ii) a nitrile group, and wherein R² is capable of forming a hydrogen bond
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof

### OR

(C) at least one of R₃, R₄, R₅, R₆ and R₇ is selected from —CN, —C(R₁₁)═N—O-alkyl group, —C(R₁₁)═N—O—H group, optionally substituted pyrazole, optionally substituted thiazole, optionally substituted oxazole, optionally substituted pyridine, and optionally substituted pyrimidine, or together with another of R₃, R₄, R₅, R₆ and R₇ forms a nitrogen containing ring;
wherein R₁₁ and R₁₂ are independently selected from H and hydrocarbyl.

According to one aspect of the present invention, there is provided a pharmaceutical composition comprising a compound of Formula I

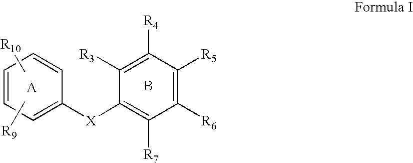

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted
wherein X is a bond or a linker group
wherein
(A) (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl

### OR

(B) at least one of R₃, R₄, R₅, R₆ and R₇ is the group —C(═O)—CR₁₁R₁₂-R₈ or together with another of R₃, R₄, R₅, R₆ and R₇ forms a ring containing —C(═O)—;
wherein R₈ is a selected from
(i) an alkyloxyalkyl group
(ii) a nitrile group, and wherein R² is capable of forming a hydrogen bond
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof

### OR

(C) at least one of R₃, R₄, R₅, R₆ and R₇ is selected from —CN, —C(R₁₁)═N—O-alkyl group, —C(R₁₁)═N—O—H group, optionally substituted pyrazole, optionally substituted thiazole, optionally substituted oxazole, optionally substituted pyridine, and optionally substituted pyrimidine, or together with another of R₃, R₄, R₅, R₆ and R₇ forms a nitrogen containing ring;
wherein R₁₁ and R₁₂ are independently selected from H and hydrocarbyl, optionally admixed with a pharmaceutically acceptable carrier, diluent, excipient or adjuvant.

In one aspect the present invention provides use of a compound in the manufacture of a medicament for use in the therapy of a condition or disease associated with 17β-hydroxysteroid dehydrogenase (17β-HSD), wherein the compound is of Formula I

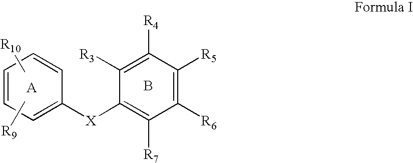

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted
wherein X is a bond or a linker group
wherein
(A) (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl

### OR

(B) at least one of R₃, R₄, R₅, R₆ and R₇ is the group —C(═O)—CR₁₁R₁₂—R₈ or together with another of R₃, R₄, R₅, R₆ and R₇ forms a ring containing —C(═O)—;
wherein R₈ is a selected from
(i) an alkyloxyalkyl group
(ii) a nitrile group, and wherein R² is capable of forming a hydrogen bond
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof

### OR

(C) at least one of R₃, R₄, R₅, R₆ and R₇ is selected from —CN, —C(R₁₁)═N—O-alkyl group, —C(R₁₁)═N—O—H group, optionally substituted pyrazole, optionally substituted thiazole, optionally substituted oxazole, optionally substituted pyridine, and optionally substituted pyrimidine, or together with another of R₃, R₄, R₅, R₆ and R₇ forms a nitrogen containing ring;
wherein R₁₁ and R₁₂ are independently selected from H and hydrocarbyl.

According to one aspect of the present invention, there is provided the use of a compound in the manufacture of a medicament for use in the therapy of a condition or disease associated with adverse steroid dehydrogenase levels, wherein the compound is of Formula I

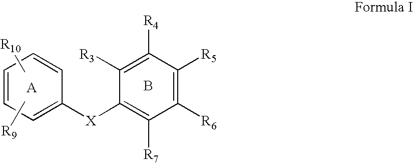

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted
wherein X is a bond or a linker group
wherein
(A) (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl

### OR

(B) at least one of R₃, R₄, R₅, R₆ and R₇ is the group —C(═O)—CR₁₁R₁₂—R₈ or together with another of R₃, R₄, R₅, R₆ and R₇ forms a ring containing —C(═O)—;
wherein R₈ is a selected from
(i) an alkyloxyalkyl group
(ii) a nitrile group, and wherein R² is capable of forming a hydrogen bond
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof

### OR

(C) at least one of R₃, R₄, R₅, R₆ and R₇ is selected from —CN, —C(R₁₁)═N—O-alkyl group, —C(R₁₁)═N—O—H group, optionally substituted pyrazole, optionally substituted thiazole, optionally substituted oxazole, optionally substituted pyridine, and optionally substituted pyrimidine, or together with another of R₃, R₄, R₅, R₆ and R₇ forms a nitrogen containing ring;
wherein R₁₁ and R₁₂ are independently selected from H and hydrocarbyl.

According to a further aspect of the present invention there is provided a method comprising (a) performing a steroid dehydrogenase assay with one or more candidate compounds having the formula as defined herein; (b) determining whether one or more of said candidate compounds is/are capable of modulating steroid dehydrogenase activity; and (c) selecting one or more of said candidate compounds that is/are capable of modulating steroid dehydrogenase activity.

According to a further aspect of the present invention there is provided a method comprising (a) performing a steroid dehydrogenase assay with one or more candidate compounds having the formula as defined herein; (b) determining whether one or more of said candidate compounds is/are capable of inhibiting steroid dehydrogenase activity; and (c) selecting one or more of said candidate compounds that is/are capable of inhibiting steroid dehydrogenase activity.

In any one of the methods of the present invention, one or more additional steps may be present. For example, the method may also include the step of modifying the identified candidate compound (such as by chemical and/or enzymatic techniques) and the optional additional step of testing that modified compound for steroid dehydrogenase inhibition effects (which may be to see if the effect is greater or different). By way of further example, the method may also include the step of determining the structure (such as by use of crystallographic techniques) of the identified candidate compound and then performing computer modelling studies—such as to further increase its steroid dehydrogenase inhibitory action. Thus, the present invention also encompasses a computer having a dataset (such as the crystallographic co-ordinates) for said identified candidate compound. The present invention also encompasses that identified candidate compound when presented on a computer screen for the analysis thereof—such as protein binding studies.

According to one aspect of the present invention, there is provided a compound identified by the method of the present invention.

For some applications, preferably the compounds have no, or a minimal, oestrogenic effect.

For some applications, preferably the compounds have an oestrogenic effect.

For some applications, preferably the compounds have a reversible action.

For some applications, preferably the compounds have an irreversible action.

In one embodiment, the compounds of the present invention are useful for the treatment of breast cancer.

The compounds of the present invention may be in the form of a salt.

In a highly preferred aspect the compound of the present invention or for use in the present invention is selected from the following compounds:

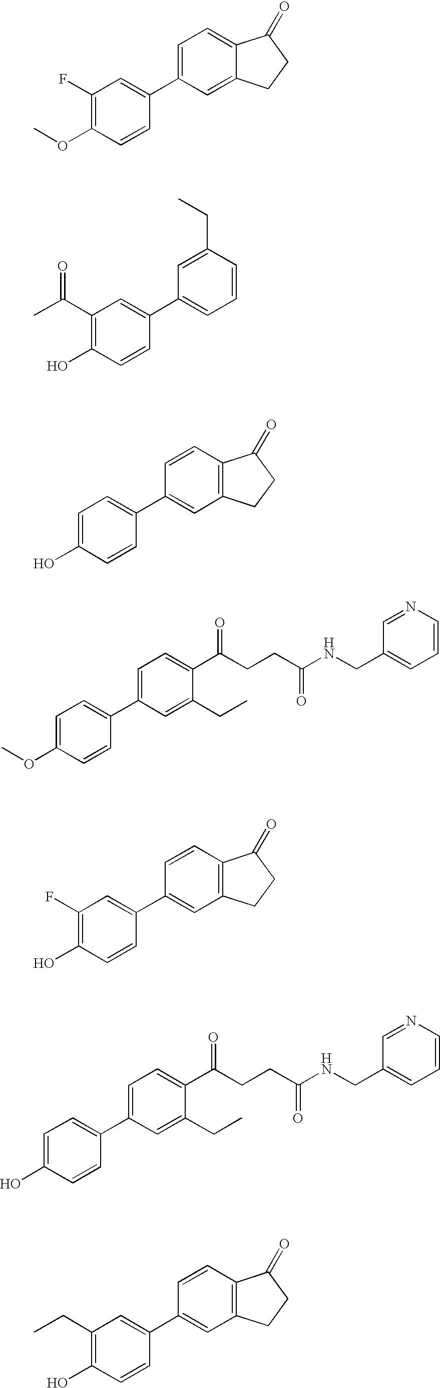

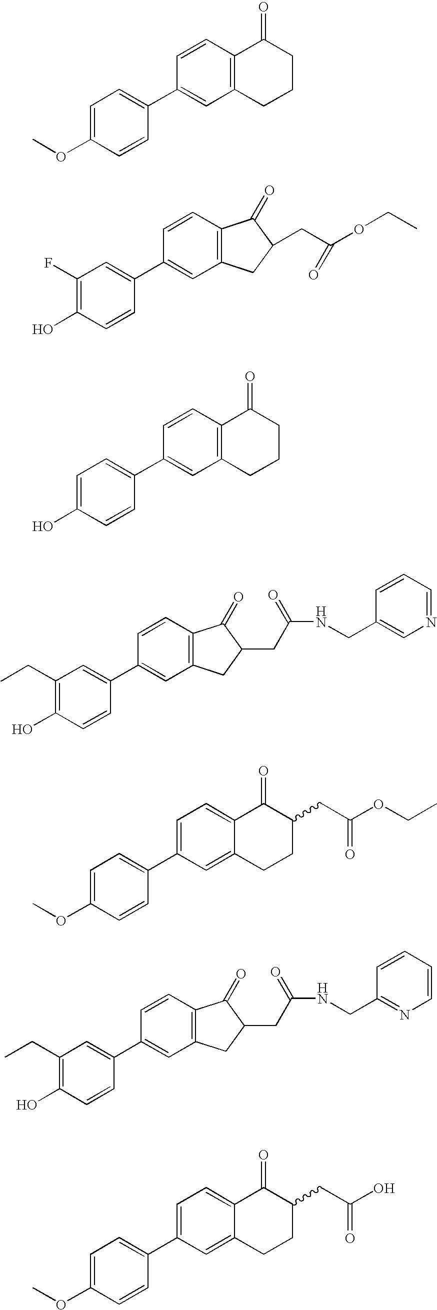

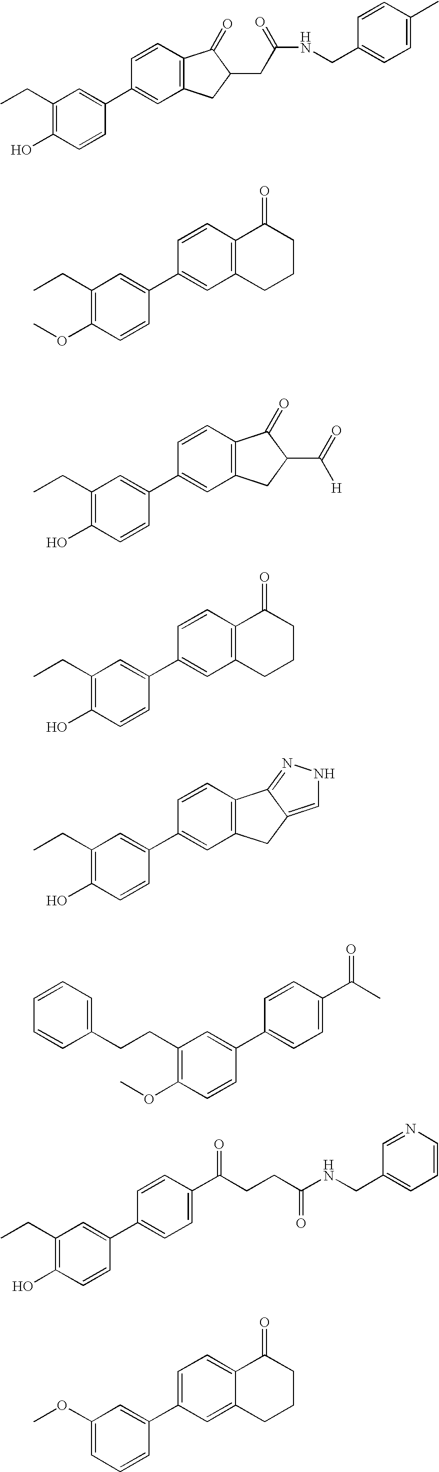

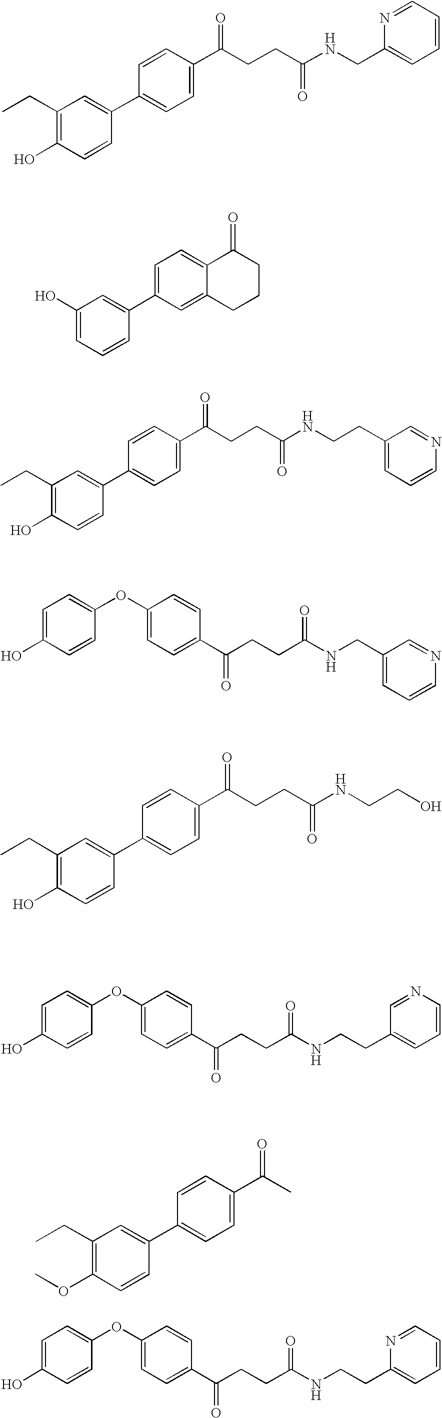

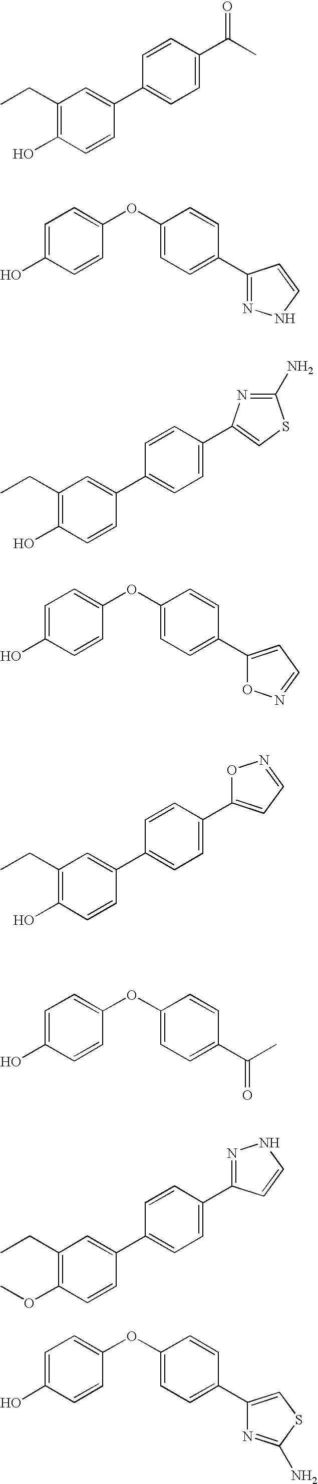

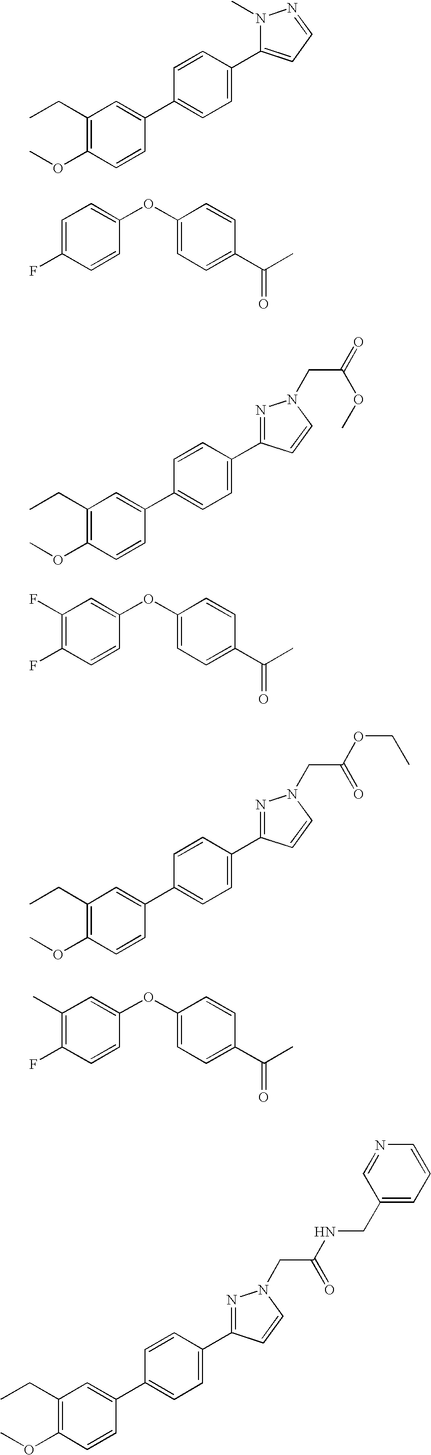

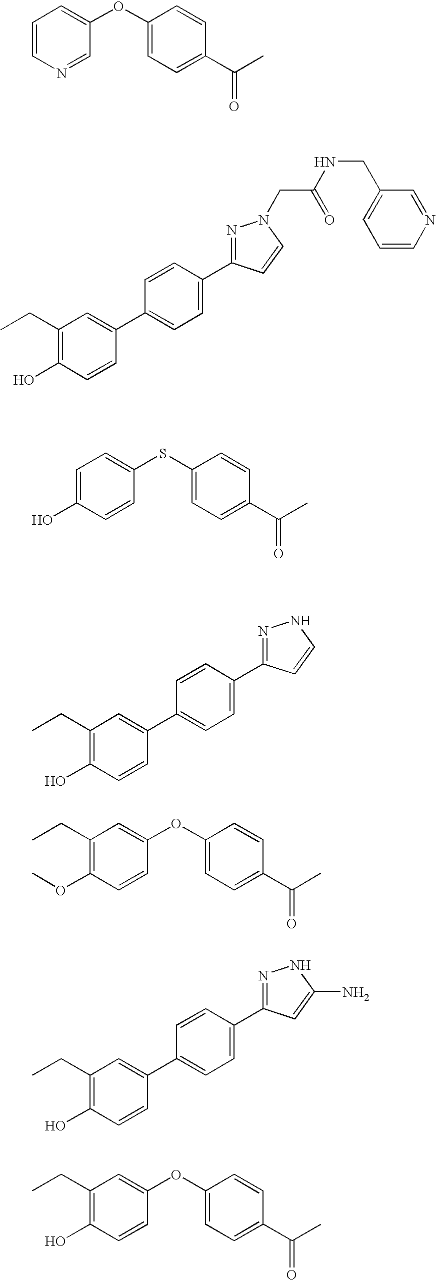

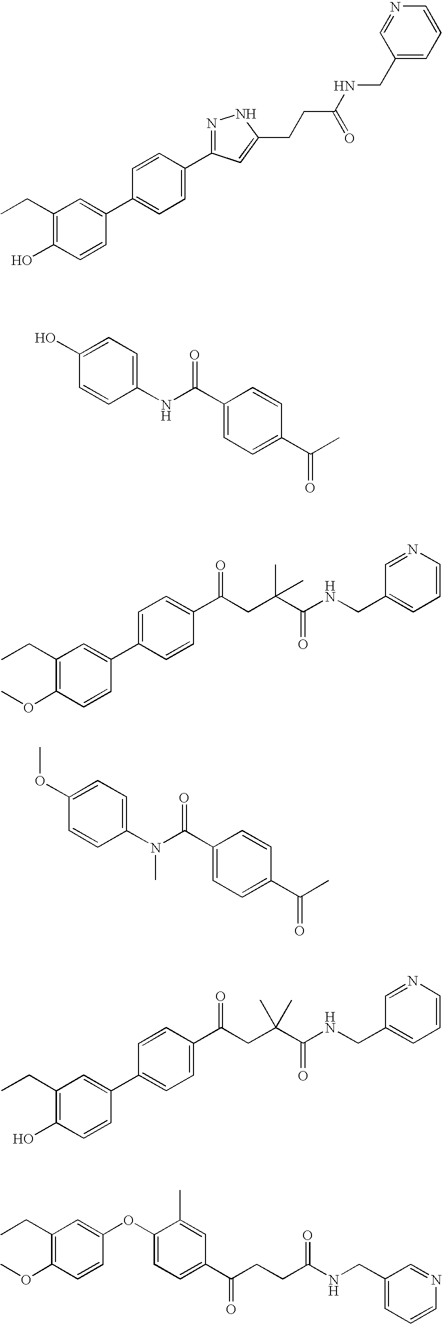

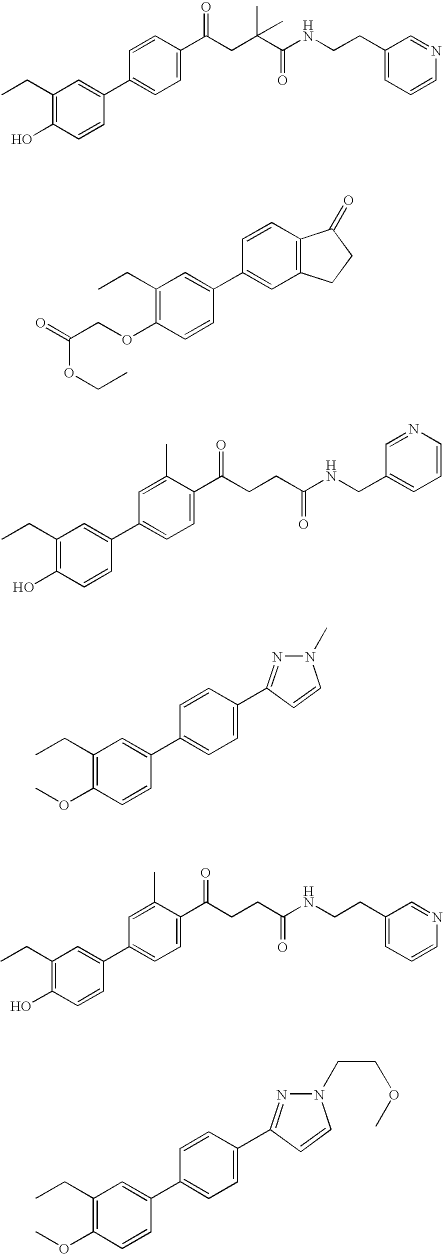

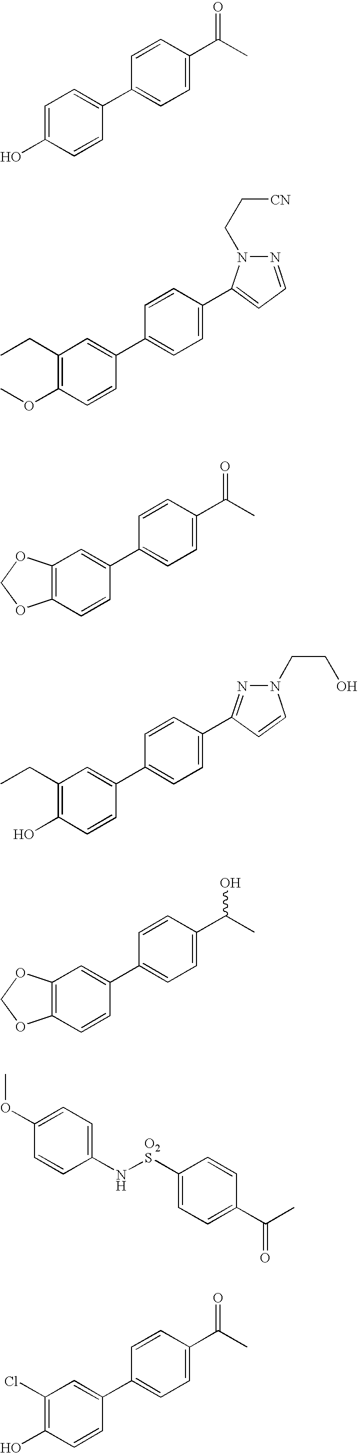

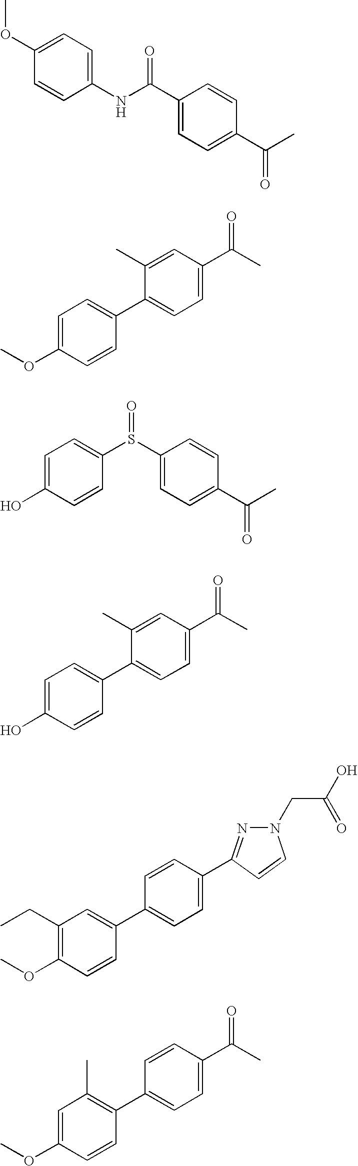

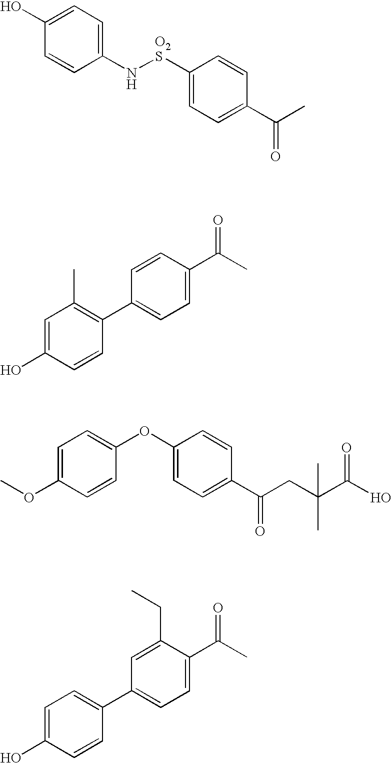

The present invention also covers novel intermediates that are useful to prepare the compounds of the present invention. For example, the present invention covers novel alcohol precursors for the compounds. By way of further example, the present invention covers bis protected precursors for the compounds. Examples of each of these precursors are presented herein. The present invention also encompasses a process comprising each or both of those precursors for the synthesis of the compounds of the present invention.

We have also identified that in some aspects of the present invention the present compounds may also inhibit the activity of steroid dehydrogenase (HSD).

By steroid dehydrogenase or HSD it is meant 17β hydroxy steroid dehydrogenase. In one aspect the 17β hydroxy steroid dehydrogenase is EC 1.1.1.62

Preferably the HSD is of Type 3, 5 and/or 7. Preferably the HSD converts androstenedione to testosterone.

Preferably the HSD is of Type 1, 3, 5 and/or 7. Preferably the HSD converts oestrone to oestradiol.

Preferably the HSD is of Type 2 and/or 8. Preferably the HSD converts oestradiol to oestrone.

In some aspects of the present invention, it is preferred that the steroid dehydrogenase is steroid dehydrogenase Type I.

In some aspects of the present invention, it is preferred that the steroid dehydrogenase is steroid dehydrogenase Type II

Preferably the HSD is of Type 1, 3, 5 and/or 7. Preferably the HSD converts oestrone to oestradiol.

Preferably the HSD is of Type 2 and/or 8. Preferably the HSD converts oestradiol to oestrone.

### Steroid Dehydrogenase

Steroid dehydrogenase or “DH” for short may be classified as consisting of two types—Type I and Type II. The two types of enzyme, such as oestradiol 17β-hydroxysteroid dehydrogenases (E2HSD), have pivotal roles in regulating the availability of ligands to interact with the oestrogen receptor. Type I reduces oestrone (E1) to the biologically active oestrogen, oestradiol (E2) while E2HSD Type II inactivates E2 by catalysing its oxidation to E1.

### DH Inhibition

It is believed that some disease conditions associated with DH activity are due to conversion of a nonactive, oestrone to an active, oestradiol. In disease conditions associated with DH activity, it would be desirable to inhibit DH activity.

Here, the term “inhibit” includes reduce and/or eliminate and/or mask and/or prevent the detrimental action of DH.

### DH Inhibitor

In accordance with the present invention, the compound of the present invention is capable of acting as a DH inhibitor.

Here, the term “inhibitor” as used herein with respect to the compound of the present invention means a compound that can inhibit DH activity—such as reduce and/or eliminate and/or mask and/or prevent the detrimental action of DH. The DH inhibitor may act as an antagonist.

The ability of compounds to inhibit steroid dehydrogenase activity can be assessed using either T47D breast cancer cells in which E2HSD Type I activity is abundant or MDA-MB-231 cells for Type II inhibitor studies. In both cell lines formation of products is linear with respect to time and cell numbers. Details on a suitable Assay Protocol are presented in the Examples section.

It is to be noted that the compound of the present invention may have other beneficial properties in addition to or in the alternative to its ability to inhibit DH activity.

### Steroid Sulphatase

In some aspects the compounds defined herein may also inhibit steroid sulphatase.

Steroid sulphatase—which is sometimes referred to as steroid sulphatase or steryl sulphatase or “STS” for short—hydrolyses several sulphated steroids, such as oestrone sulphate, dehydroepiandrosterone sulphate and cholesterol sulphate. STS has been allocated the enzyme number EC 3.1.6.2.

STS has been cloned and expressed. For example see Stein et al (J. Biol. Chem. 264:13865-13872 (1989)) and Yen et al (Cell 49:443-454 (1987)).

STS is an enzyme that has been implicated in a number of disease conditions.

By way of example, workers have found that a total deficiency in STS produces ichthyosis. According to some workers, STS deficiency is fairly prevalent in Japan. The same workers (Sakura et al, J Inherit Metab Dis 1997 November; 20(6):807-10) have also reported that allergic diseases—such as bronchial asthma, allergic rhinitis, or atopic dermatitis—may be associated with a steroid sulphatase deficiency.

In addition to disease states being brought on through a total lack of STS activity, an increased level of STS activity may also bring about disease conditions. By way of example, and as indicated above, there is strong evidence to support a role of STS in breast cancer growth and metastasis.

STS has also been implicated in other disease conditions. By way of example, Le Roy et al (Behav Genet 1999 March; 29(2):131-6) have determined that there may be a genetic correlation between steroid sulphatase concentration and initiation of attack behaviour in mice. The authors conclude that sulphatation of steroids may be the prime mover of a complex network, including genes shown to be implicated in aggression by mutagenesis.

### STS Inhibition

It is believed that some disease conditions associated with STS activity are due to conversion of a nonactive, sulphated oestrone to an active, nonsulphated oestrone. In disease conditions associated with STS activity, it would be desirable to inhibit STS activity.

Here, the term “inhibit” includes reduce and/or eliminate and/or mask and/or prevent the detrimental action of STS.

### STS Inhibitor

In accordance with the present invention, the compound of the present invention is capable of acting as an STS inhibitor.

Here, the term “inhibitor” as used herein with respect to the compound of the present invention means a compound that can inhibit STS activity—such as reduce and/or eliminate and/or mask and/or prevent the detrimental action of STS. The STS inhibitor may act as an antagonist.

The ability of compounds to inhibit steroid sulphatase activity can be assessed using either intact MCF-7 breast cancer cells or placental microsomes. In addition, an animal model may be used. Details on suitable Assay Protocols are presented in following sections. It is to be noted that other assays could be used to determine STS activity and thus STS inhibition. For example, reference may also be made to the teachings of WO-A-99/50453.

Preferably, for some applications, the compound is further characterised by the feature that if the sulphamate group were to be substituted by a sulphate group to form a sulphate derivative, then the sulphate derivative would be hydrolysable by an enzyme having steroid sulphatase (E.C. 3.1.6.2) activity—i.e. when incubated with steroid sulphatase EC 3.1.6.2 at pH 7.4 and 37° C.

In one preferred embodiment, if the sulphamate group of the compound were to be replaced with a sulphate group to form a sulphate compound then that sulphate compound would be hydrolysable by an enzyme having steroid sulphatase (E.C. 3.1.6.2) activity and would yield a Km value of less than 200 mmolar, preferably less than 150 mmolar, preferably less than 100 mmolar, preferably less than 75 mmolar, preferably less than 50 mmolar, when incubated with steroid sulphatase EC 3.1.6.2 at pH 7.4 and 37° C.

In one preferred embodiment, if the sulphamate group of the compound were to be replaced with a sulphate group to form a sulphate compound then that sulphate compound would be hydrolysable by an enzyme having steroid sulphatase (E.C. 3.1.6.2) activity and would yield a Km value of less than 200 μmolar, preferably less than 150 μmolar, preferably less than 100 μmolar, preferably less than 75 μmolar, preferably less than 50 μmolar, when incubated with steroid sulphatase EC 3.1.6.2 at pH 7.4 and 37° C.

In a preferred embodiment, the compound of the present invention is not hydrolysable by an enzyme having steroid sulphatase (E.C. 3.1.6.2) activity.

For some applications, preferably the compound of the present invention has at least about a 100 fold selectivity to a desired target (e.g. STS), preferably at least about a 150 fold selectivity to the desired target, preferably at least about a 200 fold selectivity to the desired target, preferably at least about a 250 fold selectivity to the desired target, preferably at least about a 300 fold selectivity to the desired target, preferably at least about a 350 fold selectivity to the desired target.

It is to be noted that the compound of the present invention may have other beneficial properties in addition to or in the alternative to its ability to inhibit HSD activity.

### Sulphamate Group

In one embodiment, the ring X has a sulphamate group as a substituent. The term “sulphamate” as used herein includes an ester of sulphamic acid, or an ester of an N-substituted derivative of sulphamic acid, or a salt thereof.

If R¹⁰ is a sulphamate group then the compound of the present invention is referred to as a sulphamate compound.

Typically, the sulphamate group has the formula:

(R₁)(R₂)N—S(O)(O)—O—

wherein preferably R₁ and R₂ are independently selected from H, alkyl, cycloalkyl, alkenyl and aryl, or combinations thereof, or together represent alkylene, wherein the or each alkyl or cycloalkyl or alkenyl or optionally contain one or more hetero atoms or groups.

When substituted, the N-substituted compounds of this invention may contain one or two N-alkyl, N-alkenyl, N-cycloalkyl or N-aryl substituents, preferably containing or each containing a maximum of 10 carbon atoms. When R₁ and/or R₂ is alkyl, the preferred values are those where R₁ and R₂ are each independently selected from lower alkyl groups containing from 1 to 6 carbon atoms, that is to say methyl, ethyl, propyl etc. R₁ and R₁ may both be methyl. When R₁ and/or R₂ is aryl, typical values are phenyl and tolyl (PhCH₃; o). Where R₁ and R₂ represent cycloalkyl, typical values are cyclopropyl, cyclopentyl, cyclohexyl etc. When joined together R₁ and R₂ typically represent an alkylene group providing a chain of 4 to 6 carbon atoms, optionally interrupted by one or more hetero atoms or groups, e.g. to provide a 5 membered heterocycle, e.g. morpholino, pyrrolidino or piperidino.

Within the values alkyl, cycloalkyl, alkenyl and aryl substituted groups are included containing as substituents therein one or more groups which do not interfere with the HSD inhibitory activity of the compound in question. Exemplary non-interfering substituents include hydroxy, amino, halo, alkoxy, alkyl and aryl.

In some embodiments, the sulphamate group may form a ring structure by being fused to (or associated with) one or more atoms in or on group X.

In some embodiments, there may be more than one sulphamate group. By way of example, there may be two sulphamates (i.e. bis-sulphamate compounds). If these compounds are based on a steroidal nucleus, preferably the second (or at least one of the additional) sulphamate group is located at position 17 of the steroidal nucleus. These groups need not be the same.

In some preferred embodiments, at least one of R₁ and R₂ is H.

In some further preferred embodiments, each of R₁ and R₂ is H.

### Assay for Determining Sts Activity Using Cancer Cells

### Protocol 1

Inhibition of Steroid Sulphatase Activity in MCF-7 cells

Steroid sulphatase activity is measured in vitro using intact MCF-7 human breast cancer cells. This hormone dependent cell line is widely used to study the control of human breast cancer cell growth. It possesses significant steroid sulphatase activity (MacIndoe et al. Endocrinology, 123, 1281-1287 (1988); Purohit & Reed, Int. J. Cancer, 50, 901-905 (1992)) and is available in the U.S.A. from the American Type Culture Collection (ATCC) and in the U.K. (e.g. from The Imperial Cancer Research Fund).

Cells are maintained in Minimal Essential Medium (MEM) (Flow Laboratories, Irvine, Scotland) containing 20 mM HEPES, 5% foetal bovine serum, 2 mM glutamine, non-essential amino acids and 0.075% sodium bicarbonate. Up to 30 replicate 25 cm2 tissue culture flasks are seeded with approximately 1×10⁵ cells/flask using the above medium. Cells are grown to 80% confluency and the medium is changed every third day.

Intact monolayers of MCF-7 cells in triplicate 25 cm² tissue culture flasks are washed with Earle's Balanced Salt Solution (EBSS from ICN Flow, High Wycombe, U.K.) and incubated for 3-4 hours at 37° C. with 5 μmol (7×10⁵ dpm) [6, 7-3H]oestrone-3-sulphate (specific activity 60 Ci/mmol from New England Nuclear, Boston, Mass., U.S.A.) in serum-free MEM (2.5 ml) together with oestrone-3-sulphamate (11 concentrations: 0; 1fM; 0.01 pM; 0.1 pM; 1 pM; 0.01 nM; 0.1 nM; 1 nM; 0.01 mM; 0.1mM; 1 mM). After incubation each flask is cooled and the medium (1 ml) is pipetted into separate tubes containing [14C]oestrone (7×103 dpm) (specific activity 97 Ci/mmol from Amersham International Radiochemical Centre, Amersham, U.K.). The mixture is shaken thoroughly for 30 seconds with toluene (5 ml). Experiments have shown that >90% [14C] oestrone and <0.1% [3H]oestrone-3-sulphate is removed from the aqueous phase by this treatment. A portion (2 ml) of the organic phase is removed, evaporated and the 3H and 14C content of the residue determined by scintillation spectrometry. The mass of oestrone-3-sulphate hydrolysed was calculated from the 3H counts obtained (corrected for the volumes of the medium and organic phase used and for recovery of [14C] oestrone added) and the specific activity of the substrate. Each batch of experiments includes incubations of microsomes prepared from a sulphatase-positive human placenta (positive control) and flasks without cells (to assess apparent non-enzymatic hydrolysis of the substrate). The number of cell nuclei per flask is determined using a Coulter Counter after treating the cell monolayers with Zaponin. One flask in each batch is used to assess cell membrane status and viability using the Trypan Blue exclusion method (Phillips, H. J. (1973) In: Tissue culture and applications, [eds: Kruse, D. F. & Patterson, M. K.]; pp. 406-408; Academic Press, New York).

Results for steroid sulphatase activity are expressed as the mean±1 S.D. of the total product (oestrone+oestradiol) formed during the incubation period (20 hours) calculated for 106 cells and, for values showing statistical significance, as a percentage reduction (inhibition) over incubations containing no oestrone-3-sulphamate. Unpaired Student's t-test was used to test the statistical significance of results.

### Assay for Determining Sts Activity Using Placental Microsomes

### Protocol 2

Inhibition of Steroid Sulphatase Activity in Placental Microsomes

Sulphatase-positive human placenta from normal term pregnancies are thoroughly minced with scissors and washed once with cold phosphate buffer (pH 7.4, 50 mM) then re-suspended in cold phosphate buffer (5 ml/g tissue). Homogenisation is accomplished with an Ultra-Turrax homogeniser, using three 10 second bursts separated by 2 minute cooling periods in ice. Nuclei and cell debris are removed by centrifuging (4° C.) at 2000 g for 30 minutes and portions (2 ml) of the supernatant are stored at 20° C. The protein concentration of the supernatants is determined by the method of Bradford (Anal. Biochem. 72, 248-254 (1976). Incubations (1 ml) are carried out using a protein concentration of 100 mg/ml, substrate concentration of 20 mM [6,7-3H]oestrone-3-sulphate (specific activity 60 Ci/mmol from New England Nuclear, Boston, Mass., U.S.A.) and an incubation time of 20 minutes at 37° C. If necessary eight concentrations of compounds are employed: 0 (i.e. control); 0.05 mM; 0.1 mM; 0.2 mM; 0.4 mM; 0.6 mM; 0.8 mM; 1.0 mM. After incubation each sample is cooled and the medium (1 ml) was pipetted into separate tubes containing [14C]oestrone (7×103 dpm) (specific activity 97 Ci/mmol from Amersham International Radiochemical Centre, Amersham, U.K.). The mixture is shaken thoroughly for 30 seconds with toluene (5 ml). Experiments have shown that >90% [14C]oestrone and <0.1% [3H]oestrone-3-sulphate is removed from the aqueous phase by this treatment. A portion (2 ml) of the organic phase was removed, evaporated and the 3H and 14C content of the residue determined by scintillation spectrometry. The mass of oestrone-3-sulphate hydrolysed is calculated from the 3H counts obtained (corrected for the volumes of the medium and organic phase used, and for recovery of [14C]oestrone added) and the specific activity of the substrate.

### Animal Assay Model for Determining Sts Activity

### Protocol 3

Inhibition of steroid sulphatase activity in vivo

The compounds of the present invention may be studied using an animal model, in particular in ovariectomised rats. In this model compounds which are oestrogenic stimulate uterine growth.

The compound (10 mg/Kg/day for five days) was administered orally to rats with another group of animals receiving vehicle only (propylene glycol). A further group received the compound EMATE subcutaneously in an amount of 10 μg/day for five days. At the end of the study samples of liver tissue were obtained and steroid sulphatase activity assayed using 3H oestrone sulphate as the substrate as previously described (see PCT/GB95/02638).

### Animal Assay Model for Determining Oestrogenic Activity

### Protocol 4

### Lack of In Vivo Oestrogenicity

The compounds of the present invention may be studied using an animal model, in particular in ovariectomised rats. In this model, compounds which are oestrogenic stimulate uterine growth.

The compound (10 mg/Kg/day for five days) was administered orally to rats with another group of animals receiving vehicle only (propylene glycol). A further group received the estrogenic compound EMATE subcutaneously in an amount of 10 μg/day for five days. At the end of the study uteri were obtained and weighed with the results being expressed as uterine weight/whole body weight×100.

Compounds having no significant effect on uterine growth are not oestrogenic.

### Therapy

The compounds of the present invention may be used as therapeutic agents—i.e. in therapy applications.

The term “therapy” includes curative effects, alleviation effects, and prophylactic effects.

The therapy may be on humans or animals, preferably female animals.

### Pharmaceutical Compositions

In one aspect, the present invention provides a pharmaceutical composition, which comprises a compound according to the present invention and optionally a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include, but are not limited to sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

### Combination Pharmaceutical

The compound of the present invention may be used in combination with one or more other active agents, such as one or more other pharmaceutically active agents.

By way of example, the compounds of the present invention may be used in combination with other STS inhibitors and/or other inhibitors such as an aromatase inhibitor (such as for example, 4hydroxyandrostenedione (4-OHA)) and/or steroids—such as the naturally occurring sterneurosteroids dehydroepiandrosterone sulfate (DHEAS) and pregnenolone sulfate (PS) and/or other structurally similar organic compounds. Examples of other STS inhibitors may be found in the above references. By way of example, STS inhibitors for use in the present invention include EMATE, and either or both of the 2-ethyl and 2-methoxy 17-deoxy compounds that are analogous to compound 5 presented herein.

In addition, or in the alternative, the compound of the present invention may be used in combination with a biological response modifier.

The term biological response modifier (“BRM”) includes, but not limited to cytokines, immune modulators, growth factors, haematopoiesis regulating factors, colony stimulating factors, chemotactic, haemolytic and thrombolytic factors, cell surface receptors, ligands, leukocyte adhesion molecules, monoclonal antibodies, preventative and therapeutic vaccines, hormones, extracellular matrix components, fibronectin, etc. For some applications, preferably, the biological response modifier is a cytokine. Examples of cytokines include: interleukins (IL)—such as IL-1, IL-2, IL-3, IL-4, IL-5, IL-6, IL-7, IL-8, IL-9, IL-10, IL-11, IL-12, IL-19; Tumour Necrosis Factor (TNF)— such as TNF-α; Interferon alpha, beta and gamma; TGF-β. For some applications, preferably the cytokine is tumour necrosis factor (TNF). For some applications, the TNF may be any type of TNF—such as TNF-α, TNF-β, including derivatives or mixtures thereof. More preferably the cytokine is TNF-α. Teachings on TNF may be found in the art—such as WO-A-98/08870 and WO-A-98/13348.

### Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration. Depending upon the need, the agent may be administered at a dose of from 0.01 to 30 mg/kg body weight, such as from 0.1 to 10 mg/kg, more preferably from 0.1 to 1 mg/kg body weight.

By way of further example, the agents of the present invention may be administered in accordance with a regimen of 1 to 4 times per day, preferably once or twice per day. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Aside from the typical modes of delivery—indicated above—the term “administered” also includes, but not limited to delivery by techniques such as lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular or subcutaneous route.

Thus, for pharmaceutical administration, the STS inhibitors of the present invention can be formulated in any suitable manner utilising conventional pharmaceutical formulating techniques and pharmaceutical carriers, adjuvants, excipients, diluents etc. and usually for parenteral administration. Approximate effective dose rates may be in the range from 1 to 1000 mg/day, such as from 10 to 900 mg/day or even from 100 to 800 mg/day depending on the individual activities of the compounds in question and for a patient of average (70 Kg) bodyweight. More usual dosage rates for the preferred and more active compounds will be in the range 200 to 800 mg/day, more preferably, 200 to 500 mg/day, most preferably from 200 to 250 mg/day. They may be given in single dose regimes, split dose regimes and/or in multiple dose regimes lasting over several days. For oral administration they may be formulated in tablets, capsules, solution or suspension containing from 100 to 500 mg of compound per unit dose. Alternatively and preferably the compounds will be formulated for parenteral administration in a suitable parenterally administrable carrier and providing single daily dosage rates in the range 200 to 800 mg, preferably 200 to 500, more preferably 200 to 250 mg. Such effective daily doses will, however, vary depending on inherent activity of the active ingredient and on the bodyweight of the patient, such variations being within the skill and judgement of the physician.

### Cell Cycling

The compounds of the present invention may be useful in the method of treatment of a cell cycling disorder.

As discussed in “Molecular Cell Biology” 3rd Ed. Lodish et al. pages 177-181 different eukaryotic cells can grow and divide at quite different rates. Yeast cells, for example, can divide every 120 min., and the first divisions of fertilised eggs in the embryonic cells of sea urchins and insects take only 1530 min. because one large pre-existing cell is subdivided. However, most growing plant and animal cells take 10-20 hours to double in number, and some duplicate at a much slower rate. Many cells in adults, such as nerve cells and striated muscle cells, do not divide at all; others, like the fibroblasts that assist in healing wounds, grow on demand but are otherwise quiescent.

Still, every eukaryotic cell that divides must be ready to donate equal genetic material to two daughter cells. DNA synthesis in eukaryotes does not occur throughout the cell division cycle but is restricted to a part of it before cell division.

The relationship between eukaryotic DNA synthesis and cell division has been thoroughly analysed in cultures of mammalian cells that were all capable of growth and division. In contrast to bacteria, it was found, eukaryotic cells spend only a part of their time in DNA synthesis, and it is completed hours before cell division (mitosis). Thus a gap of time occurs after DNA synthesis and before cell division; another gap was found to occur after division and before the next round of DNA synthesis. This analysis led to the conclusion that the eukaryotic cell cycle consists of an M (mitotic) phase, a G₁ phase (the first gap), the S (DNA synthesis) phase, a G₂ phase (the second gap), and back to M. The phases between mitoses (G₁, S, and G₂) are known collectively as the interphase.

Many nondividing cells in tissues (for example, all quiescent fibroblasts) suspend the cycle after mitosis and just prior to DNA synthesis; such “resting” cells are said to have exited from the cell cycle and to be in the G₀ state.

It is possible to identify cells when they are in one of the three interphase stages of the cell cycle, by using a fluorescence-activated cell sorter (FACS) to measure their relative DNA content: a cell that is in G₁ (before DNA synthesis) has a defined amount x of DNA; during S (DNA replication), it has between x and 2x; and when in G₂ (or M), it has 2x of DNA.

The stages of mitosis and cytokinesis in an animal cell are as follows:

(a) Interphase. The G₂ stage of interphase immediately precedes the beginning of mitosis. Chromosomal DNA has been replicated and bound to protein during the S phase, but chromosomes are not yet seen as distinct structures. The nucleolus is the only nuclear substructure that is visible under light microscope. In a diploid cell before DNA replication there are two morphologic chromosomes of each type, and the cell is said to be 2n. In G₂, after DNA replication, the cell is 4n. There are four copies of each chromosomal DNA. Since the sister chromosomes have not yet separated from each other, they are called sister chromatids.
b) Early prophase. Centrioles, each with a newly formed daughter centriole, begin moving toward opposite poles of the cell; the chromosomes can be seen as long threads. The nuclear membrane begins to disaggregate into small vesicles.
(c) Middle and late prophase. Chromosome condensation is completed; each visible chromosome structure is composed of two chromatids held together at their centromeres. Each chromatid contains one of the two newly replicated daughter DNA molecules. The microtubular spindle begins to radiate from the regions just adjacent to the centrioles, which are moving closer to their poles. Some spindle fibres reach from pole to pole; most go to chromatids and attach at kinetochores.
(d) Metaphase. The chromosomes move toward the equator of the cell, where they become aligned in the equatorial plane. The sister chromatids have not yet separated.
(e) Anaphase. The two sister chromatids separate into independent chromosomes. Each contains a centromere that is linked by a spindle fibre to one pole, to which it moves. Thus one copy of each chromosome is donated to each daughter cell. Simultaneously, the cell elongates, as do the pole-to-pole spindles. Cytokinesis begins as the cleavage furrow starts to form.
(f) Telophase. New membranes form around the daughter nuclei; the chromosomes uncoil and become less distinct, the nucleolus becomes visible again, and the nuclear membrane forms around each daughter nucleus. Cytokinesis is nearly complete, and the spindle disappears as the microtubules and other fibres depolymerise. Throughout mitosis the “daughter” centriole at each pole grows until it is full-length. At telophase the duplication of each of the original centrioles is completed, and new daughter centrioles will be generated during the next interphase.
(g) Interphase. Upon the completion of cytokinesis, the cell enters the G₁ phase of the cell cycle and proceeds again around the cycle.

It will be appreciated that cell cycling is an extremely important cell process. Deviations from normal cell cycling can result in a number of medical disorders. Increased and/or unrestricted cell cycling may result in cancer. Reduced cell cycling may result in degenerative conditions. Use of the compound of the present invention may provide a means to treat such disorders and conditions.

Thus, the compound of the present invention may be suitable for use in the treatment of cell cycling disorders such as cancers, including hormone dependent and hormone independent cancers.

In addition, the compound of the present invention may be suitable for the treatment of cancers such as breast cancer, ovarian cancer, endometrial cancer, sarcomas, melanomas, prostate cancer, pancreatic cancer etc. and other solid tumours.

For some applications, cell cycling is inhibited and/or prevented and/or arrested, preferably wherein cell cycling is prevented and/or arrested. In one aspect cell cycling may be inhibited and/or prevented and/or arrested in the G₂/M phase. In one aspect cell cycling may be irreversibly prevented and/or inhibited and/or arrested, preferably wherein cell cycling is irreversibly prevented and/or arrested.

By the term “irreversibly prevented and/or inhibited and/or arrested” it is meant after application of a compound of the present invention, on removal of the compound the effects of the compound, namely prevention and/or inhibition and/or arrest of cell cycling are still observable. More particularly by the term “irreversibly prevented and/or inhibited and/or arrested” it is meant that when assayed in accordance with the cell cycling assay protocol presented herein, cells treated with a compound of interest show less growth after Stage 2 of the protocol I than control cells. Details on this protocol are presented below.

Thus, the present invention provides compounds which: cause inhibition of growth of oestrogen receptor positive (ER+) and ER negative (ER−) breast cancer cells in vitro by preventing and/or inhibiting and/or arresting cell cycling; and/or cause regression of nitroso-methyl urea (NMU)-induced mammary tumours in intact animals (i.e. not ovariectomised), and/or prevent and/or inhibit and/or arrest cell cycling in cancer cells; and/or act in vivo by preventing and/or inhibiting and/or arresting cell cycling and/or act as a cell cycling agonist.

### Cell Cycling Assay

### Protocol 5

### Procedure

### Stage 1

MCF-7 breast cancer cells are seeded into multi-well culture plates at a density of 105 cells/well. Cells were allowed to attach and grown until about 30% confluent when they are treated as follows:

### Control—No Treatment

### Compound of Interest (COI) 20 μM

Cells are grown for 6 days in growth medium containing the COI with changes of medium/COI every 3 days. At the end of this period cell numbers were counted using a Coulter cell counter.

### Stage 2

After treatment of cells for a 6-day period with the COI cells are re-seeded at a density of 10⁴ cells/well. No further treatments are added. Cells are allowed to continue to grow for a further 6 days in the presence of growth medium. At the end of this period cell numbers are again counted.

### Assay for Determining Dh Activity Using Cancer Cells

### Protocol 6

Conversion of oestrone to oestradiol (E1→E2, E2DH Type I) and oestradiol to oestrone (E2→E1, E2DH Type II) was measured in intact cell monolayers of T47D and MDA-MB-231 breast cancer cells respectively. Cells were cultured in flasks until they were 80-90% confluent. ³H-E1 or ³H-E2 (6 μmol, ˜90 Ci/mmol) were added to each flask in the absence (control) or presence of various test compounds (10 μM) in 2.5 ml of medium. Substrate was also added to flasks without cells and incubated in parallel (blanks).

After incubation with T47D cells for 30 min or MDA cells for 3 h at 37° C., 2 ml of the medium was added to test tubes containing ¹⁴C-E2 or ¹⁴C-E1 (˜5000 cpm) and 50 μg E2 or E1 respectively. Steroids were extracted from the aqueous medium with diethyl ether (4 ml). The ether phase was decanted into separate tubes after freezing the aqueous phase in solid carbon dioxide-methanol mixture. The ether was evaporated to dryness under a stream of air at 40° C. The residue was dissolved in a small volume of diethyl ether and applied to TLC plates containing a fluorescent indicator. E1 and E2 were separated by TLC using DCM-Ethyl acetate (4:1 v/v). The position of the product from each incubation flask was marked on the TLC plate after visualisation under UV light. The marked regions were cut out and placed in scintillation vials containing methanol (0.5 ml) to elute the product. The amount of ³H-product formed and ¹⁴C-E1 or ¹⁴C-E2 recovered were calculated after scintillation spectrometry. The amount of product formed was corrected for procedural losses and for the number of cells in each flask.

### Cancer

As indicated, the compounds of the present invention may be useful in the treatment of a cell cycling disorder. A particular cell cycling disorder is cancer.

Cancer remains a major cause of mortality in most Western countries. Cancer therapies developed so far have included blocking the action or synthesis of hormones to inhibit the growth of hormone-dependent tumours. However, more aggressive chemotherapy is currently employed for the treatment of hormone-independent tumours.

Hence, the development of a pharmaceutical for anti-cancer treatment of hormone dependent and/or hormone independent tumours, yet lacking some or all of the side-effects associated with chemotherapy, would represent a major therapeutic advance.

It is known that oestrogens undergo a number of hydroxylation and conjugation reactions after their synthesis. Until recently it was thought that such reactions were part of a metabolic process that ultimately rendered oestrogens water soluble and enhanced their elimination from the body. It is now evident that some hydroxy metabolites (e.g. 2-hydroxy and 16alpha-hydroxy) and conjugates (e.g. oestrone sulphate, E1S) are important in determining some of the complex actions that oestrogens have in the body.

Workers have investigated the formation of 2- and 16-hydroxylated oestrogens in relation to conditions that alter the risk of breast cancer. There is now evidence that factors which increase 2-hydroxylase activity are associated with a reduced cancer risk, while those increasing 16alpha-hydroxylation may enhance the risk of breast cancer. Further interest in the biological role of oestrogen metabolites has been stimulated by the growing body of evidence that 2-methoxyoestradiol is an endogenous metabolite with anti-mitotic properties. 2-MeOE2 is formed from 2-hydroxy oestradiol (2-OHE2) by catechol oestrogen methyl transferase, an enzyme that is widely distributed throughout the body.

Workers have shown that in vivo 2-MeOE2 inhibits the growth of tumours arising from the subcutaneous injection of Meth A sarcoma, B16 melanoma or MDA-MB-435 oestrogen receptor negative (ER—) breast cancer cells. It also inhibits endothelial cell proliferation and migration, and in vitro angiogenesis. It was suggested that the ability of 2-MeOE2 to inhibit tumour growth in vivo may be due to its ability to inhibit tumour-induced angiogenesis rather than direct inhibition of the proliferation of tumour cells.

The mechanism by which 2-MeOE2 exerts its potent anti-mitogenic and anti-angiogenic effects is still being elucidated. There is evidence that at high concentrations it can inhibit microtubule polymerisation and act as a weak inhibitor of colchicine binding to tubulin. Recently, however, at concentrations that block mitosis, tubulin filaments in cells were not found to be depolymerised but to have an identical morphology to that seen after taxol treatment. It is possible, therefore, that like taxol, a drug that is used for breast and ovarian breast cancer therapy, 2-MeOE2 acts by stabilising microtubule dynamics.

While the identification of 2-MeOE2 as a new therapy for cancer represents an important advance, the bioavailability of orally administered oestrogens is poor. Furthermore, they can undergo extensive metabolism during their first pass through the liver. As part of a research programme to develop a steroid sulphatase inhibitor for breast cancer therapy, oestrone-3-O-sulphamate (EMATE) was identified as a potent active site-directed inhibitor. Unexpectedly, EMATE proved to possess potent oestrogenic properties with its oral uterotrophic activity in rats being 100-times higher than that of oestradiol. Its enhanced oestrogenicity is thought to result from its absorption by red blood cells (rbcs) which protects it from inactivation during its passage through the liver and which act as a reservoir for its slow release for a prolonged period of time. A number of A-ring modified analogues were synthesised and tested, including 2-methoxyoestrone-3-O-sulphamate.

While this compound was equipotent with EMATE as a steroid sulphatase inhibitor, it was devoid of oestrogenicity.

We believe that the compound of the present invention provides a means for the treatment of cancers and, especially, breast cancer.

In addition or in the alternative the compound of the present invention may be useful in the blocking the growth of cancers including leukaemias and solid tumours such as breast, endometrium, prostate, ovary and pancreatic tumours.

### Therapy Concerning Oestrogen

We believe that some of the compounds of the present invention may be useful in the control of oestrogen levels in the body—in particular in females. Thus, some of the compounds may be useful as providing a means of fertility control—such as an oral contraceptive tablet, pill, solution or lozenge. Alternatively, the compound could be in the form of an implant or as a patch.

Thus, the compounds of the present invention may be useful in treating hormonal conditions associated with oestrogen.

In addition or in the alternative the compound of the present invention may be useful in treating hormonal conditions in addition to those associated with oestrogen. Hence, the compound of the present invention may also be capable of affecting hormonal activity and may also be capable of affecting an immune response.

### Neurodegenerative Diseases

We believe that some of the compounds of the present invention may be useful in the treatment of neurodenerative diseases, and similar conditions.

By way of example, it is believed that STS inhibitors may be useful in the enhancing the memory function of patients suffering from illnesses such as amnesia, head injuries, Alzheimer's disease, epileptic dementia, presenile dementia, post traumatic dementia, senile dementia, vascular dementia and post-stroke dementia or individuals otherwise seeking memory enhancement.

### TH1

We believe that some of the compounds of the present invention may be useful in TH1 implications.

By way of example, it is believed that the presence of STS inhibitors within the macrophage or other antigen presenting cells may lead to a decreased ability of sensitised T cells to mount a TH1 (high IL-2, IFNγ low IL-4) response. The normal regulatory influence of other steroids such as glucocorticoids would therefore predominate.

### Inflammatory Conditions

We believe that some of the compounds of the present invention may be useful in treating inflammatory conditions—such as conditions associated with any one or more of: autoimmunity, including for example, rheumatoid arthritis, type I and II diabetes, systemic lupus erythematosus, multiple sclerosis, myasthenia gravis, thyroiditis, vasculitis, ulcerative colitis and Crohn's disease, skin disorders e.g. psoriasis and contact dermatitis; graft versus host disease; eczema; asthma and organ rejection following transplantation.

By way of example, it is believed that STS inhibitors may prevent the normal physiological effect of DHEA or related steroids on immune and/or inflammatory responses.

The compounds of the present invention may be useful in the manufacture of a medicament for revealing an endogenous glucocorticoid-like effect.

### Other Therapies

It is also to be understood that the compound/composition of the present invention may have other important medical implications.

For example, the compound or composition of the present invention may be useful in the treatment of the disorders listed in WO-A-99/52890-viz:

In addition, or in the alternative, the compound or composition of the present invention may be useful in the treatment of the disorders listed in WO-A-98/05635. For ease of reference, part of that list is now provided: cancer, inflammation or inflammatory disease, dermatological disorders, fever, cardiovascular effects, haemorrhage, coagulation and acute phase response, cachexia, anorexia, acute infection, HIV infection, shock states, graft-versus-host reactions, autoimmune disease, reperfusion injury, meningitis, migraine and aspirin-dependent anti-thrombosis; tumour growth, invasion and spread, angiogenesis, metastases, malignant, ascites and malignant pleural effusion; cerebral ischaemia, ischaemic heart disease, osteoarthritis, rheumatoid arthritis, osteoporosis, asthma, multiple sclerosis, neurodegeneration, Alzheimer's disease, atherosclerosis, stroke, vasculitis, Crohn's disease and ulcerative colitis; periodontitis, gingivitis; psoriasis, atopic dermatitis, chronic ulcers, epidermolysis bullosa; corneal ulceration, retinopathy and surgical wound healing; rhinitis, allergic conjunctivitis, eczema, anaphylaxis; restenosis, congestive heart failure, endometriosis, atherosclerosis or endosclerosis.

In addition, or in the alternative, the compound or composition of the present invention may be useful in the treatment of disorders listed in WO-A-98/07859. For ease of reference, part of that list is now provided: cytokine and cell proliferation/differentiation activity; immunosuppressant or immunostimulant activity (e.g. for treating immune deficiency, including, but not limited to infection with human immune deficiency virus; regulation of lymphocyte growth; treating cancer and many autoimmune diseases, and to prevent transplant rejection or induce tumour immunity); regulation of haematopoiesis, e.g. treatment of myeloid or lymphoid diseases; promoting growth of bone, cartilage, tendon, ligament and nerve tissue, e.g. for healing wounds, treatment of burns, ulcers and periodontal disease and neurodegeneration; inhibition or activation of follicle-stimulating hormone (modulation of fertility); chemotactic/chemokinetic activity (e.g. for mobilising specific cell types to sites of injury or infection); haemostatic and thrombolytic activity (e.g. for treating haemophilia and stroke); antiinflammatory activity (for treating e.g. septic shock or Crohn's disease); as antimicrobials; modulators of e.g. metabolism or behaviour; as analgesics; treating specific deficiency disorders; in treatment of e.g. psoriasis, in human or veterinary medicine.

In addition, or in the alternative, the composition of the present invention may be useful in the treatment of disorders listed in WO-A-98/09985. For ease of reference, part of that list is now provided: macrophage inhibitory and/or T cell inhibitory activity and thus, anti-inflammatory activity; anti-immune activity, i.e. inhibitory effects against a cellular and/or humoral immune response, including a response not associated with inflammation; inhibit the ability of macrophages and T cells to adhere to extracellular matrix components and fibronectin, as well as up-regulated fas receptor expression in T cells; inhibit unwanted immune reaction and inflammation including, but not limited to arthritis, rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery, bone marrow transplantation or other transplantation complications and/or side effects, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

### Sulphamate Compound Preparation

The sulphamate compounds of the present invention may be prepared by reacting an appropriate alcohol with a suitable chloride. By way of example, the sulphamate compounds of the present invention may be prepared by reacting an appropriate alcohol with a suitable sulfamoyl chloride, of the formula R¹R²NSO₂Cl.

Typical conditions for carrying out the reaction are as follows.

Sodium hydride and a sulfamoyl chloride are added to a stirred solution of the alcohol in anhydrous dimethyl formamide at 0° C. Subsequently, the reaction is allowed to warm to room temperature whereupon stirring is continued for a further 24 hours. The reaction mixture is poured onto a cold saturated solution of sodium bicarbonate and the resulting aqueous phase is extracted with dichloromethane. The combined organic extracts are dried over anhydrous MgSO₄. Filtration followed by solvent evaporation in vacuo and co-evaporated with toluene affords a crude residue which is further purified by flash chromatography.

Preferably, the alcohol is derivatised, as appropriate, prior to reaction with the sulfamoyl chloride. Where necessary, functional groups in the alcohol may be protected in known manner and the protecting group or groups removed at the end of the reaction.

Preferably, the sulphamate compounds are prepared according to the teachings of Page et al (1990 Tetrahedron 46; 2059-2068).

The phosphonate compounds may be prepared by suitably combining the teachings of Page et al (1990 Tetrahedron 46; 2059-2068) and PCT/GB92/01586.

The sulphonate compounds may be prepared by suitably adapting the teachings of Page et al (1990 Tetrahedron 46; 2059-2068) and PCT/GB92/01586. The thiophosphonate compounds may be prepared by suitably adapting the teachings of Page et al (1990 Tetrahedron 46; 2059-2068) and PCT/GB91/00270.

Preferred preparations are also presented in the following text.

### SUMMARY

In summation, the present invention provides compounds for use as steroid dehydrogenase inhibitors, and pharmaceutical compositions for the same.

### EXAMPLES

The present invention will now be further described by way of the following non-limiting examples.

### Experimental Details.

### 5-(−4-(Benzyloxy)-phenyl)-indan-1-one 1

A mixture of 5-bromoindanone (1.01 g, 4.48 mmol), 2M Na₂CO₃ (1.0 mL), EtOH (3.0 mL) and toluene (30 mL) was degassed by bubbling N₂ through the mixture for 35 minutes. 4-benzyloxyphenyl boronic acid (1.00 g, 4.40 mmol) was added followed by Pd(PPh₃)₄ (100 mg, 10% wt) and the mixture degassed for a further 5-10 minutes. The reaction mixture was heated at reflux for 24 h, cooled to r.t. and diluted with EtOAc (20 mL), washed with saturated brine (2×20 mL) and water (2×20 mL). The aqueous extracts were then extracted with EtOAc (2×20 mL) and DCM (2×20 mL). The combined organics were dried (Na₂SO₄) and concentrated and purified using SiO₂ chromatography (EtOAc/hexanes, gradient elution) to obtain the required product 1 (0.970 g, 75% yield) as white solid: ¹H NMR δ (CDCl₃, 270 MHz) 7.80-7.77 (d, J=7.9 Hz, 1H), 7.62-7.40 (m, 4H), 7.37-7.28 (m, 5H), 7.08-7.05 (d, J=8.9 Hz, 1H), 5.12 (s, CH₂, 2H); 3.19-3.15 (t, J=5.6 Hz, 2H, CH₂), 2.73-2.70 (t, J=4.2 Hz, 2H, CH₂). EtOAc:hexanes, 3:7, R_{f}=0.5.

### 5-(4-Methoxyphenyl)-indan-1-one 2

To a mixture of 5-bromo-1-indanone (0.105 g, 0.5 mmol), 4-methyoxyphenyl boronic acid (0.114 g, 0.75 mmol), K₂CO₃ (0.172 g, 1.25 mmol) and Bu₄NBr (0.161 g, 0.5 mmol) in EtOH (1.5 mL) and water (3.5 mL) was added Pd(OAc)₂ (catalytic) and the reaction was heated to 150° C. in the microwave for 20 min. The mixture was poured into water (15 mL) and the organics extracted into EtOAc (2×10 mL) The extracts were combined and concentrated and the product isolated by flash chromatography using an elution gradient of hexane to 20% EtOAc in hexane (R_{f} 0.5, 20% EtOAc in hexane). Yield 45 mg, 38%: ¹H NMR δ (270 MHz, CDCl₃) 2.70-2.75 (2H, m), 3.15-3.20 (2H, m), 3.86 (3H, s), 6.90 (2H, d, J=8.8 Hz), 7.54-7.62 (4H, m), 7.78 (1H, d, J=8.0 Hz); HPLC tr=4.49 min (>99%) 70% MeCN in H₂O; LC/MS (ES+) m/z 238.97 (M+H)⁺

### 5-(3-Fluoro-4-methoxyphenyl)-indan-1-one 3

Prepared as for 5-(4-methoxyphenyl)-indan-1-one 2 but using 3-fluoro-4-methoxyphenyl boronic acid. Purified by flash chromatography using an elution gradient of hexane to 25% EtOAc in hexane (R_{f} 0.3, 25% EtOAc in hexane). Yield of isolated product 25%: ¹H NMR δ (270 MHz, CDCl₃) 2.70-2.75 (2H, m), 3.15-3.20 (2H, m), 3.86 (3H, s), 6.90 (2H, d, J=8.8 Hz), 7.54-7.62 (4H, m), 7.78 (1H, d, J=8.0 Hz); HPLC tr=2.05 min (>95%) 90% MeCN in H₂O; HRMS (ES+) m/z 257.0981 (M+H)⁺, calcd 257.0972 for C₁₆H₁₄F₁O₂.

### 5-(4-(Benzyloxy-3-fluorophenyl)-indan-1-one 4

A mixture of 5-bromoindanone (0.470 g, 2.23 mmol), 2M Na₂CO₃ (1.0 mL), EtOH (1.5 mL) and toluene (15 mL) was degassed by bubbling N₂ through the mixture for 35 minutes. 3-Fluoro-4-O-benzyl boronic acid (0.500 g, 2.03 mmol) was added followed by Pd(PPh₃)₄ (50 mg) and degassed for a further 5-10 minutes. The reaction mixture was heated at reflux for 24 h, cooled to r.t. and diluted with EtOAc (20 mL), washed with saturated brine (2×20 mL) and water (2×20 mL). The aqueous extracts were then extracted with EtOAc (2×20 mL) and DCM (2×20 mL). The combined organics were dried (Na₂SO₄), concentrated and purified using SiO₂ chromatography (EtOAc/hexanes, gradient elution) to obtain the required product 4 (0.646 g, 95% yield) as a white solid: ¹H NMR δ (CDCl₃, 270 MHz) 7.80-7.77 (d, J=8.1 Hz, 1H), 7.59 (s, 1H), 7.53-7.50 (d, J=7.3 Hz, 1H), 7.47-7.28 (m, 7H) 7.10-7.04 (t, J=8.6 Hz, 1H), 5.19 (s, 2H), 3.19-3.15 (t, J=5.6 Hz, 2H), 2.75-2.70 (t, J=4.2 Hz, 2H). EtOAc:hexanes, 3:7, R_{f}=0.5.

### 4-Bromo-2-ethyl-1-methoxybenzene 5

To a stirred solution of 2-ethyl anisole (3.2 g, 23.5 mmol) in MeCN (100 mL) was added N-bromo succinimide (4.59 g, 25.8 mmol) and the reaction was stirred at rt for 18 h before being concentrated under reduced pressure. To the crude mix was added diethyl ether (100 mL) and the solution washed with water (2×100 mL). The ether layer was concentrated under reduced pressure and the product purified by flash chromatography using hexane to EtOAc as eluent (R_{f} 0.9, 30% EtOAc in hexane) to give colourless oil, 4.95 g, 98%: ¹H NMR δ (270 MHz, CDCl₃) 1.16 (t, J=7.5 Hz, 3H), 2.59 (q, J=7.5 Hz, 2H), 3.79 (s, 3H), 6.69 (d, J=9.4 Hz, 1H), 7.23-7.26 (m, 2H).

### 3-Ethyl-4-methoxyphenylboronic acid 6

To a stirred solution of 4-bromo-2-ethyl-1-methoxybenzene (4.59 g, 21 mmol) in dry THF (50 mL), cooled to −78° C., was added slowly drop-wise (over 45 min) n-BuLi (15 mL of a 1.6 M solution in hexanes, 24 mmol). The solution was stirred at −78° C. for 2 h before trimethylborate (3.6 mL, 31.5 mmol) was added drop-wise and the reaction was allowed to warm slowly to rt with stirring overnight. The reaction was quenched with 2M HCl (50 mL) and the products extracted with EtOAc (2×50 mL). These extracts were combined and concentrated under reduced pressure to give an oily substance. To this was added hexane followed by a small amount of DCM and the resulting white precipitate was collected by filtration and washed with hexane. Yield 1.55 g, 41%: ¹H NMR δ (270 MHz, CDCl₃) 1.27 (3H, t, J=7.5 Hz), 2.73 (2H, q, J=7.5 Hz), 3.90 (3H, s), 6.60 (˜1H, bs), 6.96 (1H, d, J=8.4 Hz), 7.98 (1H, d, J=1.5 Hz), 8.08 (1H, dd, J=8.2, 1.7 Hz); LC/MS (APCI) m/z 179.04 (M−H)⁻; HPLC tᵣ=2.71 min (>99%) 90% MeCN in H₂O.

### 5-(3-Ethyl-4-methoxyphenyl)-indan-1-one 7

A mixture of 5-bromoindanone (1.27 g, 6.05 mmol), 2M Na₂CO₃ (2.0 mL), EtOH (4.0 mL) and toluene (40 mL) was degassed bubbling N₂ through the mixture for 35 minutes. 3-Ethyl-4-methoxyphenyl boronic acid (1.00 g, 5.55 mmol) was added followed by Pd(PPh₃)₄ (100 mg) and the mixture degassed for a further 5-10 minutes. The reaction mixture was heated at reflux for 24 h, cooled to r.t. and diluted with EtOAc (20 mL), washed with saturated brine (2×20 mL) and water (2×20 mL). The aqueous extracts were then extracted with EtOAc (2×20 mL) and DCM (2×20 mL). The combined organics were dried (Na₂SO₄) and concentrated and purified using SiO₂ chromatography (EtOAc/hexanes, gradient elution) to obtain the required product 3 (0.970 g, 65% yield) as white solid: ¹H NMR δ (CDCl₃, 270 MHz) 7.79-7.76 (d, J=7.9 Hz, 1H), 7.63 (s, 1H), 7.58-7.55 (d, J=8.16 Hz, 1H), 7.46-7.27 (m, 2H), 6.93-6.90 (d, J=8.9 Hz, 1H), 3.87 (s, 3H), 3.19-3.15 (t, J=5.6 Hz, 2H), 2.74-2.68 (m, 4H), 1.28-1.20 (t, J=7.6 Hz, 3H). EtOAc:hexanes, 3:7, R_{f}=0.5.

### 5-(4-Hydroxyphenyl)-indan-1-one 8

5-(4-Benzyloxyphenyl)-indan-1-one 1 (0.100 g, 0.44 mmol) was suspended in DCM under inert atmosphere and cooled to −78° C. A solution of BBr₃ (1.0 M solution in DCM, 1.32 mL, 1.32 mmol) was added at −78° C. and the mixture stirred for 2 h. TLC analysis (EtOAc:hexanes, 3:7, R_{f}=0.19) indicated the completion of the reaction. The mixture was warmed to room temperature and water (20 mL) was added and the mixture extracted with EtOAc (2×20 mL) and DCM (2×20 mL). The combined organic extracts were dried (Na₂SO₄) and concentrated to obtain a pale yellow solid. The solid was purified by chromatography on SiO₂ (EtOAc/hexanes gradient elution) to afford the product 8 (0.49 g, 50%) as pale yellow solid: ¹H NMR δ (MeOD, 270 MHz) 7.70-7.72 (d {two doublets overlapped}, 2H), 7.63-7.53 (m, 3H), 6.90-6.87 (d {two doublets overlapped}, 2H), 3.21-3.17 (t, J=5.4 Hz, 2H), 2.71-2.69 (t, J=5.6 Hz, 2H); HPLC>95% (Rₜ=1.70, 90% MeCN in water); FAB-MS (M+H)⁺ 225 m/z.

### 5-(3-Fluoro-4-hydroxyphenyl)-indan-1-one 9

5-(4-(Benzyloxy-3-fluorophenyl)-indan-1-one 4 (0.150 g, 1.9 mmol) was dissolved in MeOH (10 mL) and degassed for 20-30 minutes by bubbling N₂ through the mixture. Then 10% Pd on C (50 mg) was added and the mixture was debenzylated using H₂ (balloon) for 24 h. TLC indicated 3 spots (R_{f}=0.09, 0.20 and 0.73; EtOAc:hexanes, 3:7, the R^{f} of the required product is 0.20). The crude mixture was then filtered through Celite, dried (Na₂SO₄), concentrated and purified (EtOAc/hexanes, gradient elution) to obtain pale yellow solid: ¹H NMR δ (MeOD, 270 MHz) 7.74-7.71 (d overlapped, 2H), 7.63-7.60 (d, J=8.41 Hz, 1H), 7.47-7.42 (dd, J=12.6, 2.2 Hz, 1H), 7.39-7.34 (dd, J=9.1, 2.9 Hz, 1H), 7.04-6.98 (t, J=8.6 Hz, 1H), 3.22-3.18 (t, 2H), 2.73-2.69 (t, J=3.9 Hz, 2H); HPLC>94% (Rₜ=1.66, 90% MeCN in water); FAB-MS (M)⁺ 242 m/z; FAB-HRMS calcd for C₁₅H₁₁FO₂ 243.08213 found (M+H)⁺ 243.08200.

### 5-(3-Ethyl-4-hydroxyphenyl)-indan-1-one 10

5-(3-Ethyl-4-methoxyphenyl)-indan-1-one 7 (0.178 g, 0.66 mmol) was suspended in DCM under an inert atmosphere and cooled to −78° C. A solution of BBr₃ (1.0 M solution in DCM, 0.730 ml, 0.73 mmol) was added dropwise at to −78° C. and stirred 2 h. TLC analysis (EtOAc/hexanes, 3:7, R_{f}=0.27 and 0.19 starting material and the product respectively) at this stage indicated the completion of the reaction. The mixture was warmed to room temperature and water (20 mL) was added and the mixture extracted with EtOAc (2×20 mL) and DCM (2×20 mL). The combined organic extracts were dried (Na₂SO₄) and concentrated to obtain pale yellow solid. The solid was purified by chromatography on SiO₂ (EtOAc/hexanes gradient elution) to afford the title compound (0.122 g, 72%) as pale yellow solid: ¹H NMR δ (MeOD, 270 MHz) 7.79-7.76 (d, J=7.9 Hz, 2H), 7.631 (s, 1H), 7.57-7.54 (d, J=7.9 Hz, 1H), 7.42-7.41 (appd, 1H), 7.37-7.34 (dd, J=8.1, 2.2 Hz, 1H), 6.87-6.84 (d, J=8.1 Hz, 1H), 3.19-3.15 (t, J=5.4 Hz, 2H), 2.75-2.66 (m, 4H), 1.31-1.26 (t, J=7.4 Hz, 3H); HPLC>96% (Rₜ=1.70, 80% MeCN in water); APCI-MS (M+H)⁺ 213 m/z.

### [5-(4-Benzyloxy-3-fluorophenyl)-1-oxo-indan-2-yl]-acetic Acid Ethyl Ester 11

To a solution of 5-(-4-(Benzyloxy-3-fluorophenyl)-indan-1-one 4 (0.420 g, 1.33 mmol) in dry THF (10 ml) under an inert atmosphere was cooled to −10° C. and stirred for 15 minutes. A solution of LDA was added (1.8 M solution in heptane/THF/ethyl benzene, 0.77 mL, 1.4 mmol) over 15 minutes at −10° C. The reaction mixture was cooled to −60° C., stirred at this temperature for 20 minutes and then ethyl bromoacetate (0.176 ml, 1.59 mmol) was added drop-wise and the mixture stirred at −60° C. for 2-3 h and allowed to warm to r.t. overnight. The overall reaction time was 18 h. The reaction mixture was quenched with sat. NH₄Cl and the organics extracted into DCM (3×20 ml), dried (MgSO₄) and concentrated to obtain a pale yellow solid. The solid was purified by chromatography on SiO₂ (EtOAc/hexanes, gradient elution) to obtain the title compound 11 (monoalkylated) as a mixture with the bis-alkylated product [2:1 ratio by ¹H NMR, 0.349 g] as colourless solid: ¹H NMR δ (MeOD, 270 MHz) 7.80-7.78 (m, 1H), 7.79-7.29 (m, 5H), 7.21-7.04 (t, J=8.65 Hz, 1H), 5.19-5.17 (s, 2H), 4.15-4.12 (q, J=7.7 Hz, 2H), 4.00-3.92 (q, J=7.7 Hz, 2H), 3.52-3.43 (dd, J=16.5, 7.4 Hz, 1H), 3.05-2.68 (m, 4H), 1.21-1.16 (t, J=7.1 Hz), 1.07-1.05 (t, J=7.17 Hz, 3H); APCI-MS (M−62)⁻ 356 m/z.

### [5-(3-Fluoro-4-hydroxyphenyl)-1-oxo-indan-2-yl]-acetic Acid Ethyl Ester 12

[5-(4-Benzyloxy-3-fluorophenyl)-1-oxo-indan-2-yl]-acetic acid ethyl ester 11 (0.070 g) was suspended in dry DCM at −78° C. under an inert atmosphere and BBr₃ (1.0 M solution in DCM, 0.52 mL, 0.52 mmol) was added drop-wise. The reaction was allowed to stir at −78° C. for 1.5 h and allowed to warm to r.t. overnight. The overall reaction time was 18 h. Water was added and the mixture stirred at r.t. for 15 min. before the organics were extracted with DCM (2×20 mL), EtOAc (2×20 mL), dried (MgSO₄), and concentrated to obtain a pale yellow solid. The solid was purified by chromatography on SiO₂ (EtOAc/hexanes, gradient elution) to obtain the title compound (0.058 g, 99%) as colourless solid: ¹H NMR δ (CDCl₃, 270 MHz) 7.82-7.71 (m, 2H), 7.64-7.60 (appd, J=9.4 Hz, 1H), 7.47-7.42 (dd, J=12.3, 2.3 Hz, 1H), 7.39-7.35 (dd, J=8.4, 2.2 Hz, 1H), 7.04-6.98 (t, J=8.6 Hz, 1H), 4.14-4.09 (q, J=7.17 Hz, 2H), 4.00-3.92 (small q, indicate the bis-alkylated product approximately 5%), 3.52-3.43 (dd, J=16.5, 7.4 Hz, 1H), 3.20-2.68 (m, 4H), 1.23-1.20 (t, J=3.9 Hz, 3H), 1.10-1.05 (a small t, indicate the presence of bis-alkylated compound about 5%); HPLC>83% (Rₜ=1.81, 90% MeCN in water); FAB-MS (M)⁺ 328 m/z; FAB-HRMS calcd for C₁₉H₁₇FO₄ 329.1189 found (M+H)⁺ 329.1198.

### [5-(3-Ethyl-4-methoxyphenyl)-1-oxo-indan-2-yl]-acetic Acid Ethyl Ester 13

A solution of 5-(3-ethyl-4-methoxyphenyl)-indan-1-one 7 (0.312 g, 2.08 mmol) in dry THF (12 mL) under an inert atmosphere was cooled to −10° C. and stirred for 15 minutes. A solution of LDA (1.8 M solution in heptane/THF/ethyl benzene, 0.711 mL, 1.28 mmol) was added over 15 minutes at −10° C. The mixture was cooled to −60° C. and stirred at this temperature for 20 minutes before ethyl bromoacetate (0.155 mL, 1.40 mmol) was added drop-wise and the mixture stirred at −60° C. for 2-3 h and allowed to warm to r.t. overnight. The overall reaction time was 18 h. The reaction mixture was quenched with sat. NH₄Cl and organics extracted with DCM (3×20 ml), dried (MgSO₄) and concentrated to obtain a pale yellow solid. The solid was purified by chromatography on SiO₂ (EtOAc/hexanes, gradient elution) to obtain the title compound (0.340 g, 68%) as colourless solid: ¹H NMR δ (CDCl₃, 270 MHz) 7.80-7.78 (m, 1H), 7.81-7.77 (m, 1H), 7.62-7.55 (m, 1H), 7.46-7.42 (m, 1H), 6.93-6.90 (d, J=8.41 Hz, 1H); 4.17-4.12 (q, J=7.17 Hz, 2H, CH₂), 3.52-3.43 (dd, J=17.0, 7.9 Hz, 1H, CH), 3.33 (s, 2H), 3.33-2.60 (m, 6H), 1.25-1.19 (m, 6H, 2×CH₃); HPLC>99% (Rₜ=2.93, 90% MeCN in water); APCI-MS (M+H)⁺ 350 m/z.

### [5-(3-Ethyl-4-methoxyphenyl)-1-oxo-indan-2-yl]-acetic Acid 14

[5-(3-Ethyl-4-methoxyphenyl)-1-oxo-indan-2-yl]-acetic acid ethyl ester 13 (0.530 g, 1.56 mmol) was suspended in THF:water (1:1, 5 mL) and NaOH (0.124 g, 3.12 mmol) was added. The mixture was stirred at r.t. for 2 days. Analysis by tlc (DCM:MeOH, 95:5, R_{f}=0.02 and 0.39 showed 2 major products. The resultant pale orange mixture was acidified to pH=2 with 2M HCl and extracted with DCM (2×20 mL) and EtOAc (2×20 ml). The combined organics were dried (Na₂SO₄) and concentrated. The crude product was purified by chromatography on SiO₂ (DCM:MeOH gradient elution). The compound with R_{f} 0.39 was separated from the compound with R_{f}=0.02, and it was shown the compound with R_{f} =0.39 was 14 (0.158 g, 31% yield) mono-alkylated product isolated as a pale yellow solid: ¹H NMR δ (CDCl₃, 270 MHz) 7.80-7.78 (d, J=7.9 Hz, 1H), 7.63-7.56 (m, 2H), 7.45-7.42 (m, 2H), 6.93-6.90 (d, J=8.4 Hz, 1H), 3.87 (s, 3H), 3.55-3.46 (dd, J=17.3, 7.6 Hz, 1H), 3.17-2.89 (m, 3H), 2.73-2.63 (m, 3H), 1.26-1.20 (t, J=7.6 Hz, 3H); HPLC>92% (Rₜ=2.01, 80% MeCN in water); FAB-MS (M+H)⁺ 325 m/z.

### 2-[5-(3-Ethyl-4-methoxyphenyl)-1-oxo-indan-2-yl]-N-pyridin-3-ylmethyl-acetamide 15

[5-(3-Ethyl-4-methoxyphenyl)-1-oxo-indan-2-yl]-acetic acid 14 (0.070 g, 0.21 mmol) was suspended in anhydrous DCM (12 mL) under an inert atmosphere. EDCI (0.123 g, 0.64 mmol) was added followed by DMAP (10 mg, cat. amount) and Et₃N (0.070 mL) and the mixture was stirred at r.t. for 20-30 minutes. 2-Aminomethylpyridine (0.046 g, 0.43 mmol) was added and the resultant mixture was stirred for 25 h. The reaction was quenched with sat. Na₂CO₃ and organics extracted into DCM (2×20 ml), dried (MgSO₄) and concentrated. The crude product (pre-absorbed on 1 g of silica gel) was purified by chromatography on SiO₂ (DCM:MeOH, gradient elution and the product was eluted with 10% MeOH in DCM) to give the title compound (20 mg, 23%) as pale yellow solid: ¹H NMR δ (CDCl₃, 270 MHz) 8.51-8.48 (m, 2H), 7.77-7.74 (d, J=7.6 Hz, 1H), 7.60-7.56 (m, 3H), 7.46-7.45 (d, J=2.2 Hz, 1H), 7.42 (s, 1H), 7.22-7.19 (m, 1H), 6.93-6.90 (d, J=8.4 Hz, 1H), 6.55 (broad t), 4.46-4.43 (apparent dd, J=5.9, 2.2 Hz, 2H), 3.87 (s, 3H), 3.51-3.45 (dd, J=16.8, 8.6 Hz, 1H), 3.20-2.59 (m, 6H), 1.26-1.20 (t, J=7.6 Hz, 3H); APCI-MS (M+H)⁺ 415 m/z.

### 2-[5-(3-Ethyl-4-methoxyphenyl)-1-oxo-indan-2-yl]-N-pyridin-2-ylmethyl-acetamide 16

[5-(3-Ethyl-4-methoxyphenyl)-1-oxo-indan-2-yl]-acetic acid 14 (0.050 g, 0.15 mmol) was suspended in anhydrous DCM (10 mL) under an inert atmosphere. EDCI (0.087 g, 0.46 mmol) was added followed by DMAP (10 mg, cat. amount) and Et₃N (0.050 mL) and the mixture stirred at r.t. for 20-30 minutes. 2-Aminomethylpyridine (0.031 mL, 0.30 mmol) was added and the resultant mixture stirred for 25 h. The reaction was quenched with sat. Na₂CO₃ and organics extracted into DCM (2×20 mL), dried (MgSO₄) and concentrated. The crude product was purified by chromatography on SiO₂ (DCM:MeOH, gradient elution and the product was eluted with 10% MeOH in DCM) to give the title compound (40 mg, 63%) as pale yellow solid: ¹H NMR δ (CDCl₃, 270 MHz) 8.51-8.50 (m, 1H), 7.79-7.76 (d, J=7.91 Hz, 1H), 7.66-7.55 (m, 3H), 7.46-7.41 (m, 3H), 7.20-7.17 (m, 1H), 6.93-6.90 (d, J=8.4 Hz, 1H), 4.57-4.55 (d, J=4.9 Hz, 2H), 3.87 (s, 3H), 3.47-3.04 (dd, J=17.8, 7.9 Hz, 1H, CH), 3.20-2.59 (m, 6H), 1.25-1.20 (t, J=7.4 Hz, 3H); APCI-MS (M+H)⁺ 415 m/z.

### 2-[5-(3-Ethyl-4-methoxyphenyl)-1-oxo-indan-2-yl]-N-(5-methyl-pyrazin-2-ylmethyl)-acetamide 17

[5-(3-Ethyl-4-methoxyphenyl)-1-oxo-indan-2-yl]-acetic acid 14 (0.050 g, 0.15 mmol) was suspended in anhydrous DCM (10 mL) under an inert atmosphere. EDCI (0.087 g, 0.46 mmol) was added follwed by DMAP (10 mg, catalytic amount) and Et₃N (0.050 mL) and the mixture stirred at r.t. for 20-30 minutes. 2-Aminomethyl-5-methylpyrazine (0.037 g, 0.30 mmol), was added and the resultant mixture stirred for 25 h. The reaction was quenched with sat. Na₂CO₃ and the organics extracted into DCM (2×20 mL), dried (MgSO₄) and concentrated. The crude product was purified by chromatography on SiO₂ (DCM:MeOH, gradient elution and the product was eluted with 10% MeOH in DCM) to give the title compound (28 mg, 42%) as pale yellow solid: ¹H NMR δ (CDCl₃, 270 MHz) 8.44 and 8.32 (2×s, 1H each), 8.08-8.07 (m, 1H), 7.78-7.75 (d, J=7.9 Hz, 1H), 7.59-7.55 (m, 2H), 7.45-7.37 (m, 2H), 6.93-6.90 (d, J=7.8 Hz, 1H), 6.84 (broad t, 1H), 4.56-4.54 (d, J=5.4 Hz, 2H), 3.87 (s, 3H), 3.14-2.47 (m, 6H), 1.25-1.20 (t, J=4.7 Hz); APCI-MS (M+H)⁺ 430 m/z.

### 2-[5-(3-Ethyl-4-hydroxyphenyl)-1-oxo-indan-2-yl]-N-pyridin-3-ylmethyl-acetamide 18

2-[5-(3-Ethyl-4-methoxyphenyl)-1-oxo-indan-2-yl]-N-pyridin-3-ylmethyl-acetamide 15 (0.020 g, 0.048 mmol) was suspended in DCM (5.0 mL) under an inert atmosphere and cooled to −78° C. A solution of BBr₃ (1.0 M solution in DCM, 0.24 ml, 0.24 mmol) was added at −78° C. and the mixture stirred for 2 h and allowed to warm to r.t. overnight. TLC analysis (EtOAc:hexanes, 3:7, R_{f}=0.4) indicated the completion of the reaction. Water was added to quench the reaction and the organics extracted into EtOAc (2×20 mL) and DCM (2×20 mL). The combined organic extracts were dried (Na₂SO₄) and concentrated to obtain a pale yellow solid. The solid was purified by chromatography on SiO₂ (MeOH/DCM gradient elution) to afford the title compound (6 mg, 31%) as pale yellow solid: ¹H NMR δ (MeOD, 270 MHz) 8.51 (m, 2H), 8.07 (s, 1H), 7.75-7.72 (d, J=7.9 Hz, 1H), 7.57-7.52 (m, 3H), 7.39 (appd, 1H), 7.30-7.27 (m, 1H), 6.83-6.80 (d, J=8.16 Hz, 1H), 6.60-6.50 (broad t), 4.66 (broad s, 1H), 4.47-4.44 (apparent q, J=5.6 Hz, 2H) 3.41-3.35 (dd, J=17.1, 8.1 Hz, 1H, CH), 3.06-2.65 (m, 6H), 1.21-1.23 (t, J=7.4 Hz, 2H), HPLC>96% (Rₜ=2.45, 50% MeCN in water); APCI-MS (M+H)⁺ 401 m/z.

### 2-[5-(3-Ethyl-4-hydroxyphenyl)-1-oxo-indan-2-yl]-N-pyridin-2-ylmethyl-acetamide 19

2-[5-(3-Ethyl-4-methoxyphenyl)-1-oxo-indan-2-yl]-N-pyridin-2-ylmethyl-acetamide 16 (0.030 g, 0.072 mmol) was suspended in DCM (6.0 mL) under an inert atmosphere and cooled to −78° C. A solution of BBr₃ (1.0 M solution in DCM, 0.36 ml, 0.36 mmol) was added at to −78° C. and stirred for 2 h and then allowed to warm to r.t. overnight. TLC analysis (EtOAc:hexanes, 3:7, R_{f}=0.4) indicated the completion of the reaction. Water was added to quench the reaction and the organics extracted into EtOAc (2×20 mL) and DCM (2×20 mL). The combined extracts were dried (Na₂SO₄) and concentrated to obtain pale yellow solid. The solid was purified by chromatography on SiO₂ (MeOH/DCM gradient elution) to afford the title compound (15 mg, 37%) as pale yellow solid: ¹H NMR δ (MeOD, 270 MHz) 8.47-8.46 (d, J=4.29, 1H), 8.02 (s, 1H), 7.71-7.69 (d, J=8.58 Hz, 1H), 7.61-7.57 (td, J=7.8, 1.56 Hz, 1H), 7.48-7.46 (2×s, 2H), 7.32 (appd, J=2 Hz, 1H), 7.24-7.21 (dd, J=8.19, 2.34 Hz, 1H), 7.18-7.12 (m, 1H), 6.94-6.93 (broad t, 1H), 6.74-6.72 (d, J=8.19 Hz, 1H), 4.61 (s, 1H), 4.53-4.51 (appq, J=2.7 Hz, 2H), 3.41-3.35 (dd, J=17.1, 8.1 Hz, 1H), 3.06-3.01 (m, 1H), 2.93-2.99 (t, J=5.4 Hz, 1H), 2.86-2.87 (t, J=3.9 Hz, 1H), 2.63-2.51 (m, 2H), 1.20-1.18 (t, J=7.4 Hz, 2H); HPLC>99% (Rₜ=2.39, 50% MeCN in water); APCI-MS (M+H)⁺ 401 m/z.

### 2-[5-(3-Ethyl-4-hydroxyphenyl)-1-oxo-indan-2-yl]-N-(5-methyl-pyrazin-2-ylmethyl)-acetamide 20

2-[5-(3-Ethyl-4-methoxyphenyl)-1-oxo-indan-2-yl]-N-(5-methyl-pyrazin-2-ylmethyl)-acetamide 17 (0.040 g, 0.093 mmol) was suspended in DCM (6.0 mL) under an inert atmosphere and cooled to −78° C. A solution of BBr₃ (1.0 M solution in DCM, 0.46 mL, 0.46 mmol) was added at to −78° C. and stirred for 2 h and allowed to warmed to r.t. overnight. TLC analysis (EtOAc:hexanes, 3:7, R_{f}=0.4) indicated the completion of the reaction. Water was added to quench the reaction and the organics extracted into EtOAc (2×20 mL) and DCM (2×20 mL). The combined extracts were dried (Na₂SO₄) and concentrated to obtain pale yellow solid. This was purified by chromatography on SiO₂ (MeOH/DCM gradient elution) to afford the title compound (15 mg, 39%) as pale yellow solid: According to ¹H NMR this compound exists as a mixture of rotamers. The analysis of the major rotamer: ¹H NMR δ (CDCl₃, 270 MHz) 8.50 (s, 1H), 8.39 (s, 1H), 8.12 (appd, J=1.1 Hz, 1H), 7.83-7.80 (d, J=8.1 Hz, 1H), 7.64-7.60 (m, 1H), 7.50-7.49 (appd, J=3.51 Hz, 1H), 7.47-7.46 (appd, J=2.3 Hz, 1H), 6.97-6.95 (d, J=8.1 Hz, 1H), 6.93-6.87 (broad t, 1H), 4.71 (s, 1H), 4.61-4.60 (d, J=5.4 Hz, 2H), 3.92 (s, 3H), 3.56-3.50 (dd, J=17.1, 7.8 Hz, 1H), 3.16-3.13 (m, 1H), 3.05-2.94 (m, 2H), 2.76-2.71 (q, J=7.4 Hz, 2H), 1.28-1.26 (t, J=7.41 Hz, 3H); HPLC>84% (Rₜ=2.43, 80% MeCN in water); APCI-MS (M+H)⁺ 416 m/z.

### 5-(3-Ethyl-4-methoxyphenyl)-1-oxo-indan-2-carbaldehyde 21

To a stirred solution of 5-(3-Ethyl-4-methoxyphenyl)-indan-1-one 7 (0.284 g, 1.06 mmol) in toluene (10 mL) was added ethyl formate (0.552 g, 7.46 mmol) followed by KO^{t}Bu (0.358 g, 3.20 mmol. The reaction was stirred at r.t. for 24 h before being acidified with AcOH. The crude mixture was diluted with EtOAc (20 mL), washed with water (20 mL), and saturated brine (20 mL). The organic phase was dried (Na₂SO₄) and concentrated to obtain the title compound (0.313 g, 99%) as yellow powder: ¹H NMR δ (CDCl₃, 270 MHz) 9.20 (s, 1H), 7.16-7.14 (d, 1H), 6.96-6.93 (m, 4H), 6.54 (s, 1H), 6.23-6.20 (appd, 1H), 3.17 (s, 3H), 2.00-1.98 (m, 2H), 0.55-0.53 (t, 3H); APCI-MS (M+H)⁺ 295 m/z.

### 5-(3-Ethyl-4-hydroxyphenyl)-1-oxo-indan-2-carbaldehyde 22

5-(3-Ethyl-4-methoxyphenyl)-1-oxo-indan-2-carbaldehyde 21 (0.060 g, 0.20 mmol), was suspended in anhydrous DCM (10 mL), under an inert atmosphere, and cooled to −78° C. BBr₃ (1.0 M solution in DCM, 0.61 mL, 0.61 mmol) was added at −78° C. and the mixture allowed to warm to r.t. overnight. The reaction was quenched with sat. Na₂CO₃ (20 mL) and organics extracted into DCM (2×20 mL) and EtOAc (2×20 mL). The extracts were combined, dried (Na₂SO₄) and concentrated under reduced pressure to obtain a dark brown solid. The solid was pre-absorbed to SiO₂ and purified by chromatography with DCM:MeOH gradient elution to obtain the title compound (0.050 g, 87%) as pale yellow solid: ¹H NMR δ (DMSO-d₆, 270 MHz) 9.59 (s, 1H), 7.72 (s, 1H), 7.68-7.64 (m, 2H), 7.48-7.47 (appd, J=1.97 Hz, 1H), 7.43-7.40 (dd, J=8.1, 2.7 Hz, 1H), 6.89-6.86 (d, J=8.41 Hz, 1H), 2.65-2.56 (q, J=7.9 Hz, 2H), 1.20-1.15 (t, J=7.4 Hz, 3H); HPLC>98% (Rₜ=2.22, 80% MeCN in water); APCI-MS (M+H⁺) 279 m/z.

### 6-(3-Ethyl-4-methoxyphenyl)-2,4-dihydro-indeno[1,2-c]pyrazole 23

To a solution of 5-(3-Ethyl-4-methoxyphenyl)-1-oxo-indan-2-carbaldehyde 21 (0.263 g, 0.89 mmol) in EtOH (10 mL) was added hydrazine monohydrate (0.067 g, 1.34 mmol) and the reaction mixture was heated for 2 h. After 2 h, the mixture was cooled to r.t., acidified with glacial acetic acid (pH=²) and concentrated under reduced pressure. The mixture was diluted with DCM (30 mL), washed with water (4×20 mL) and saturated Na₂CO₃ (2×20 mL). The organics were dried (Na₂SO₄) and concentrated under vacuum to obtain a brown solid. The solid was purified by chromatography with 5% MeOH in DCM (TLC: 5% MeOH/DCM, R_{f}=0.54) to obtain the title compound (0.240 g, 92%) as pale yellow solid: ¹H NMR δ (CDCl₃, 270 MHz) 7.82-7.79 (d, J=7.9 Hz, 1H), 7.68 (s, 1H), 7.57 (appd, 1H), 7.46-7.42 (m, 3H), 6.93-6.89 (d, J=9.1 Hz, 1H), 3.87 (s, 3H), 3.71 (s, 2H), 2.71-2.68 (q, J=7.6 Hz, 2H), 1.27-1.21 (t, J=7.42 Hz, 3H); HPLC>84% (Rₜ=3.04, 70% MeCN in water); APCI-MS (M+H⁺) 291 m/z.

### 4-(2,4-Dihydro-indeno[1,2-c]pyrazol-6-yl)-2-ethyl-phenol 24

6-(3-Ethyl-4-methoxyphenyl)-2,4-dihydro-indeno[1,2-c]pyrazole 23 (0.600 g, 2.06 mmol), was suspended in anhydrous DCM (20 mL), under an inert atmosphere, and cooled to −78° C. BBr₃ (1.0 M solution in DCM, 6.20 mL, 6.20 mmol) was added at −78° C. and the mixture was allowed to warm to r.t. overnight. The reaction was quenched with sat. Na₂CO₃ (20 mL) and the organics extracted into DCM (2×20 mL) and EtOAc (2×20 mL). The extracts were combined, dried (Na₂SO₄) and concentrated under reduced pressure to obtain a dark brown solid. The solid was pre-absorbed to SiO₂ and purified by chromatography with DCM:MeOH gradient elution to obtain the title compound (0.410 g, 71%) pale yellow solid: ¹H NMR δ (DMSO-d₆, 270 MHz) 7.72 (s, 1H), 7.64-7.61 (m, 2H), 7.55 (s, 1H), 7.41 (appd, 1H), 7.36-7.33 (appdd, J=8 Hz, 1H), 6.87-6.84 (d, J=8.41 Hz, 1H), 3.65 (s, 2H, CH₂), 2.61-2.59 (q, J=7.4 Hz, 2H), 1.20-1.15 (t, J=7.1 Hz, 3H); HPLC>99% (Rₜ=2.00, 80% MeCN in water); APCI-MS (M+H⁺) 277 m/z.

### 4-(4-Bromophenyl)-4-oxo-butanoic Acid 25

To a stirred mixture of powdered succinic anhydride (1.00 g, 10 mmol) in bromobenzene (6.5 mL, 61.7 mmol), cooled to −5° C. under N₂, was added anhydrous AlCl₃ (2.67 g, 20 mmol). The reaction temperature was maintained at −5° C. for 4 h before being allowed to warm to r.t. Stirring at r.t. was continued for a further 96 h before the mixture was poured into a cooled (ice bath), stirred solution of HCl (aq) (25 mL of 18%). The mixture was stirred for a further 30 min while being allowed to warm to r.t. The light cream precipitate was collected by filtration and washed with water. Recrystallisation from PhMe gave shiny white plates, mp 148-150° C.; ¹HNMR (270 MHz, DMSO-d₆) δ 2.57 (2H, t, J=6.3 Hz), 3.23 (2H, t, J=6.3 Hz), 7.75 (2H, d, J=8.7 Hz), 7.92 (2H, d, J=8.7 Hz), 12.17 (1H, bs)

### 4-(3′-Ethyl-4′-methoxy-biphenyl)-4-oxo-butanoic Acid 26

To a stirred solution of 3-ethyl-4-methoxyphenylboronic acid 6 (1.2 g, 6.7 mmol) in PhMe (36 mL), EtOH (4 mL) and 2M aq. Na₂CO₃ (4 mL) was added 4-(4-bromophenyl)-4-oxobutanoic acid 25 (1.56 g, 6.1 mmol) and the solution was degassed by bubbling N₂ through for 1 h. To this was then added Pd(PPh₃)₄ (catalytic) and the reaction was refluxed under N₂ for 20 h. The mixture was cooled, water (100 mL) was added and the organics extracted into EtOAc (2×100 mL). The extracts were combined and concentrated under reduced pressure to give a cream coloured powder. Flash chromatography using gradient elution of DCM to 10% MeOH in DCM, followed by recrystallisation from MeOH/H₂O yielded 1.2 g, 64% of title compound as pale pink powder: ¹H NMR δ (270 MHz, DMSO-d₆) 1.19 (t, J=7.4 Hz, 3H), 2.60-2.68 (4H), 3.27-3.30 (2H), 3.37 (bs, 1H), 3.86 (s, 3H), 7.08 (d, J=8.6 Hz, 1H), 7.57 (d, J=2.3 Hz, 1H), 7.60 (dd, J=8.2, 2.3 Hz, 2H), 7.80 (d, J=8.6 Hz, 2H), 8.04 (d, J=8.6 Hz, 2H); ¹³C NMR δ (100 MHz, DMSO-d₆) 14.8, 23.4, 28.4, 33.5, 55.9, 111.6, 126.2, 126.7, 128.0, 129.0, 131.3, 132.7, 134.9, 145.0. 157.9, 174.4, 198.4; HPLC>96%, (Rₜ 2.75, 80% MeCN in H₂O); APCI (M−H)⁻ 311.24 m/z; FAB-HRMS calcd for C₁₉H₂₀O₄ 312.1361 found (M⁺) 312.1353 m/z.

### General Procedure 1: Amide Coupling using a Greenhouse™ Synthesiser

To a stirred solution of 4-(3′-ethyl-4′-methoxy-biphenyl)-4-oxo-butanoic acid 26 (0.100 g, 0.32 mmol) and NEt₃ (60 μL) in dry DCM (2 mL) under N₂ was added a solution of EDCI (0.192 g, 1 mmol) and DMAP (catalytic) in dry DCM (2 mL) and the mixture was stirred for 40 min before addition of amine (0.06 mL). The reaction was stirred at rt for 24 h before quenching with sat. aq. Na₂CO₃. The organic layer was separated and concentrated under reduced pressure and the product purified by flash chromatography using gradient elution of DCM to 5% MeOH in DCM, followed by recrystallisation from DCM/hexane.

### 4-(3′-Ethyl-4′-methoxy-biphenyl)-4-oxo-N-pyridin-3-ylmethyl-butyramide 27

Prepared by general procedure 1 using 3-aminomethylpyridine. Yield 31%; ¹H NMR δ (270 MHz, CD₃OD) 1.21 (t, J=7.5 Hz, 3H), 2.65-2.73 (4H), 3.41-3.46 (2H), 3.87 (s, 3H), 4.62 (s, 2H), 7.02 (d, J=8.7 Hz, 1H), 7.45-7.55 (2H), 7.72 (d, J=8.7 Hz, 2H), 8.04-8.12 (3H), 8.64 (d, J=8.2 Hz, 1H), 8.76 (d, J=5.7 Hz, 1H), 8.89 (s, 1H); HPLC>99% (Rₜ 2.29, 90% MeCN in H₂O); APCI (M+H)⁺ 403.25 m/z.

### 4-(3′-Ethyl-4′-methoxy-biphenyl)-4-oxo-N-pyridin-2-ylmethyl-butyramide 28

Prepared by general procedure 1 using 2-aminomethylpyridine. Yield 31%; ¹H NMR δ (270 MHz, CDCl₃) 1.23 (t, J=7.5 Hz, 3H), 2.65-2.76 (4H), 3.39-3.45 (2H), 3.87 (s, 3H), 4.57 and 4.59 (2s, 2H), 6.90-6.93 (2H), 7.16-7.21 (m, 1H), 7.25-7.27 (1H), 7.42-7.47 (2H), 7.62-7.68 (3H), 8.03 (d, J=8.4 Hz, 2H), 8.53 (d, J=4.5 Hz, 1H); HPLC>96% (Rₜ 2.34, 90% MeCN in H₂O); APCI (M+H)⁺ 403.25 m/z.

### 4-(3′-Ethyl-4′-methoxy-biphenyl)-4-oxo-N-pyridin-3-ylethyl-butyramide 29

Prepared by general procedure 1 using 2-(pyridin-3-yl)ethanamine. Yield 30%; ¹H NMR δ (270 MHz, CDCl₃) 1.23 (t, J=7.5 Hz, 3H), 2.59 (t, J=6.4 Hz, 2H), 2.69 (q, J=7.5 Hz, 2H), 2.80-2.86 (m, 2H), 3.36 (t, J=6.4 Hz, 2H), 3.48-3.56 (m, 2H), 3.87 (s, 3H), 5.90 (bs, 1H, NH), 6.92 (d, J=8.7 Hz, 1H), 7.16-7.25 (m, 1H), 7.42-7.47 (2H), 7.52-7.56 (m, 1H), 7.65 (d, J=8.4 Hz, 2H), 8.01 (d, J=8.7 Hz, 2H), 8.45-8.48 (2H); HPLC>95% (Rₜ 2.33, 90% MeCN in H₂O); APCI (M+H)⁺ 417.30 m/z.

### 4-(3′-Ethyl-4′-methoxy-biphenyl)-4-oxo-N-pyridin-2-ylethyl-butyramide 30

Prepared by general procedure 1 using 2-(pyridin-2-yl)ethanamine. Yield 30%; ¹H NMR δ (270 MHz, CDCl₃) 1.23 (t, J=7.5 Hz, 3H), 2.58-2.73 (4H), 2.96-3.01 (m, 2H), 3.33-3.38 (m, 2H), 3.65-3.71 (m, 2H), 3.87 (s, 3H), 6.62 (bs, 1H, NH), 6.91 (d, J=8.4 Hz, 1H), 7.11-7.25 (2H), 7.39-7.46 (2H), 7.56-7.65 (3H), 8.00 (d, J=8.3 Hz, 2H), 8.52 (d, J=4.9 Hz, 1H); HPLC>99% (Rₜ 2.36, 90% MeCN in H₂O); APCI (M+H)⁺ 417.30 m/z.

### 4-(3′-Ethyl-4′-methoxy-biphenyl)-4-oxo-N-furan-2-ylmethyl-butyramide 31

Prepared by general procedure 1 using (furan-2-yl)methanamine. Yield 24%; ¹H NMR δ (270 MHz, CDCl₃) 1.23 (t, J=7.5 Hz, 3H), 2.63-2.73 (4H), 3.37-3.42 (2H), 3.87 (s, 3H), 4.44 and 4.46 (2s, 2H), 6.05 (bs, 1H, NH), 6.23 (d, J=3.2 Hz, 1H), 6.29-6.32 (m, 1H), 6.92 (d, J=7.9 Hz, 1H), 7.34-7.35 (m, 1H), 7.43-7.46 (2H), 7.64 (d, J=8.7 Hz, 2H), 8.01 (d, J=8.7 Hz, 2H); HPLC>99% (Rₜ 2.41, 90% MeCN in H₂O); APCI (M+H)⁺ 392.23 m/z.

### 4-(3′-Ethyl-4′-methoxy-biphenyl)-4-oxo-N-(2-methoxyethyl)-butyramide 32

Prepared by general procedure 1 using 2-methoxyethanamine. Yield 25%; ¹H NMR δ (270 MHz, CDCl₃) 1.23 (t, J=7.5 Hz, 3H), 2.61-2.73 (4H), 3.30-3.56 (9H), 3.86 (s, 3H), 6.10 (bs, 1H), 6.91 (d, J=8.7 Hz, 1H), 7.42-7.46 (2H), 7.63 (d, J=8.7 Hz, 2H), 8.01 (d, J=8.4 Hz, 2H); HPLC>99% (Rₜ 2.34, 90% MeCN in H₂O); APCI (M+H)⁺ 370.30 m/z.

### General Procedure 2: O-Demethylation using a Carousel™ Reaction Station

To a stirred solution of the requisite butyramide in dry DCM (5 mL), cooled to −78° C. under N₂, was added dropwise BBr₃ (0.3 mL of a 1M solution in DCM, 0.3 mmol) and the reaction was allowed to warn slowly to r.t. overnight. The reactions were quenched with water (5 mL) and if the product precipitated it was collected by filtration and washed with water and DCM before drying in vacuo. In cases where the product did not precipitate, the organic layer was separated and concentrated under reduced pressure.

### 4-(3′-Ethyl-4′-hydroxy-biphenyl)-4-oxo-N-pyridin-3-ylmethyl-butyramide 33

Prepared by general procedure 2 from 4-(3′-Ethyl-4′-methoxy-biphenyl)-4-oxo-N-pyridin-3-ylmethyl-butyramide 27. Yield 41%; ¹H NMR δ (270 MHz, CD₃OD) 1.23 (t, J=7.5 Hz, 3H), 2.65-2.72 (4H), 3.37-3.42 (2H), 4.44 (s, 2H), 6.83 (d, J=8.2 Hz, 1H), 7.35-7.43 (3H), 7.69 (d, J=8.7 Hz, 2H), 7.82 (d, J=7.4 Hz, 1H), 8.03 (3d, J=8.4 Hz, 2H), 8.41-8.44 (m, 1H), 8.52 (s, 1H); HPLC>96% (Rₜ 1.83, 90% MeCN in H₂O); APCI (M+H)⁺ 389.20 m/z; FAB-HRMS calcd for C₂₄H₂₅N₂O₃ 389.1865 found (M+H)⁺ 389.1867 m/z.

### 4-(3′-Ethyl-4′-hydroxy-biphenyl)-4-oxo-N-pyridin-2-ylmethyl-butyramide 34

Prepared by general procedure 2 from 4-(3′-Ethyl-4′-methoxy-biphenyl)-4-oxo-N-pyridin-2-ylmethyl-butyramide 28. Yield 5%; ¹H NMR δ (270 MHz, CD₃OD) 1.23 (t, J=7.5 Hz, 3H), 2.64-2.75 (4H), 3.40-3.46 (m, 2H), 4.51 (s, 2H), 6.84 (d, J=8.2 Hz, 1H), 7.28-7.50 (4H), 7.69 (d, J=8.7 Hz, 2H), 7.79-7.86 (m, 1H), 8.05 (d, J=8.7 Hz, 2H), 8.47 (d, J=4.7 Hz, 1H); HPLC>90% (Rₜ, 1.89, 90% MeCN in H₂O); APCI (M+H)⁺ 389.14 m/z; FAB-HRMS calcd for C₂₄H₂₅N₂O₃ 389.1865 found (M+H)⁺ 389.1871 m/z.

### 4-(3′-Ethyl-4′-hydroxy-biphenyl)-4-oxo-N-pyridin-3-ylethyl-butyramide 35

Prepared by general procedure 2 from 4-(3′-Ethyl-4′-methoxy-biphenyl)-4-oxo-N-pyridin-3-ylethyl-butyramide 29. Yield 72%; ¹H NMR δ (270 MHz, CD₃OD) 1.22 (t, J=7.5 Hz, 3H), 2.55 (t, J=6.2 Hz, 2H), 2.67 (q, J=7.5 Hz, 2H), 3.08 (t, J=6.2 Hz, 2H), 3.27-3.32 (m, 2H), 3.55-3.60 (m, 2H), 6.84 (d, J=8.2 Hz, 1H), 7.35 (dd, J=8.4 ,2.2 Hz, 1H), 7.41 (d, J=2.2 Hz, 1H), 7.67 (d, J=8.4 Hz, 2H), 7.98-8.04 (3H), 8.60 (d, J=8.2 Hz, 1H), 8.73 (d, J=5.7 Hz, 1H), 8.88 (s, 1H); HPLC>99% (Rₜ 1.85, 90% MeCN in H₂O); APCI (M+H)⁺ 403.19 m/z; FAB-HRMS calcd for C₂₅H₂₇N₂O₃ 403.2021 found (M+H)⁺ 403.2026 m/z.

### 4-(3′-Ethyl-4′-hydroxy-biphenyl)-4-oxo-N-pyridin-2-ylethyl-butyramide 36

Prepared by general procedure 2 from 4-(3′-Ethyl-4′-methoxy-biphenyl)-4-oxo-N-pyridin-2-ylethyl-butyramide 30. ¹H NMR δ (270 MHz, CD₃OD) 1.22 (t, J=7.5 Hz, 3H), 2.52 (t, J=6.2 Hz, 2H), 2.67 (q, J=7.5 Hz, 2H), 3.21-3.29 (4H), 3.62-3.66 (m, 2H), 6.83 (d, J=8.4 Hz, 1H), 7.35 (dd, J=8.4 ,2.2 Hz, 1H), 7.41 (d, J=2.2 Hz, 1H), 7.68 (d, J=8.4 Hz, 2H), 7.85-7.90 (m, 1H), 7.98-8.03 (3H), 8.45-8.50 (m, 1H), 8.72 (d, J=5.7 Hz, 1H); HPLC>99% (Rₜ 1.86, 90% MeCN in H₂O); APCI (M+H)⁺ 403.32 m/z.

### 4-(3′-Ethyl-4′-hydroxy-biphenyl)-4-oxo-N-(2-hydroxyethyl)-butyramide 37

Prepared by general procedure 2 from 4-(3′-Ethyl-4′-methoxy-biphenyl)-4-oxo-N-(2-methoxyethyl)-butyramide 32. Yield 41%; mp>110° C. (dec); ¹H NMR δ (270 MHz, CD₃OD) 1.22 (t, J=7.5 Hz, 3H), 2.61-2.71 (4H), 3.29-3.38 (4H), 3.59-3.63 (2H), 6.84 (d, J=8.4 Hz, 1H), 7.35 (dd, J=8.2 ,2.5 Hz, 1H), 7.42 (d, J=2.2 Hz, 1H), 7.68 (d, J=8.4 Hz, 2H), 8.02 (d, J=8.4 Hz, 1H); HPLC>93% (Rₜ 1.76, 90% MeCN in H₂O); APCI (M+H)⁺ 342.13 m/z; FAB-HRMS calcd for C₂₀H₂₄NO₄ 342.1700 found (M+H)⁺ 342.1700 m/z.

### 1-(3′-Ethyl-4′-methoxy-biphenyl-4-yl)-ethanone 38

A solution of 3-ethyl-4-methoxyphenylboronic acid 6 (0.500 g, 2.8 mmol) and 4-bromoacetophenone (0.507 g, 2.5 mmol) in PhMe (18 mL), EtOH (2 mL) and 2M aq. Na₂CO₃ (2 mL) was degassed by bubbling N₂ through for 40 min. To this was added Pd(PPh₃)₄ (catalytic) and the reaction was heated to reflux under N₂ for 20 h. The reaction was allowed to cool to rt before water (100 mL) was added and the products were extracted into EtOAc (2×100 mL). The organic layers were combined and concentrated under reduced pressure and the product purified by flash chromatography (20 g column, Flashmaster II) using gradient elution of 100% hexane to 100% EtOAc. Yield 88%: mp 69-72° C.; ¹H NMR δ (270 MHz, CDCl₃) 1.23 (t, J=7.5 Hz, 3H), 2.62 (s, 3H), 2.69 (q, J=7.5 Hz, 2H), 3.87 (s, 3H), 6.92 (d, J=8.2 Hz, 1H), 7.43-7.47 (2H), 7.64 (d, J=8.4 Hz, 2H), 7.99 (d, J=8.4 Hz, 2H); ¹³C NMR δ (100 MHz, CDCl₃) 14.2, 23.5, 26.7, 55.5, 110.5, 125.7, 126.7, 127.9, 128.9, 131.9, 132.0, 133.2, 135.1, 145.8, 157.8, 197.9; HPLC>99% (Rₜ 5.05, 80% MeCN in H₂O); APCI (M+H)⁺ 254.09 m/z; FAB-HRMS calcd for C₁₇H₁₉O₂ 255.1380 found (M+H)⁺ 255.1384 m/z.

### 1-(3′-Ethyl-4′-hydroxy-biphenyl-4-yl)-ethanone 39

To a stirred solution of 1-(3′-ethyl-4′-methoxy-biphenyl-4-yl)-ethanone 38 (0.051 g, 0.2 mmol) in dry DCM (2 mL) under N₂, cooled to −78° C. (dry ice/acetone bath) was added drop-wise BBr₃ (0.6 mL of a 1M solution in DCM, 0.6 mmol) and the reaction was allowed to warm slowly to r.t. overnight. Water (20 mL) was added and the products extracted with EtOAc (2×30 mL). The organic layers were combined and concentrated under reduced pressure and the product purified by flash chromatography (20 g column, Flashmaster II) using an elution gradient of 100% hexane to 100% EtOAc. Yield 42%: ¹H NMR δ (400 MHz, CDCl₃) 1.29 (t, J=7.5 Hz, 3H), 2.65 (s, 3H), 2.73 (q, J=7.5 Hz, 2H), 5.95 (s, 1H), 6.91 (d, J=8.1 Hz, 1H), 7.34-7.37 (m, 1H), 7.43 (d, J=2.1 Hz, 1H), 7.64 (d, J=8.4 Hz, 2H), 8.00 (d, J=8.4 Hz, 2H); ¹³C NMR δ (100 MHz, CDCl₃) 14.0, 23.2, 26.6, 115.7, 125.8, 126.6, 128.3, 129.0, 130.7, 132.1, 134.9, 145.9, 154.2, 198.6; HPLC>98% (Rₜ 2.24, 90% MeCN in H₂O); APCI (M−H)⁻ 239.03 m/z; FAB-HRMS calcd for C₁₆H₁₇O₂ 241.1229 found (M⁺) 241.1223 m/z.

### 4-(3′-Ethyl-4′-hydroxy-biphenyl-4-yl)-thiazol-2-ylamine 40

To a stirred solution of 1-(3′-Ethyl-4′-hydroxy-biphenyl-4-yl)-ethanone 39 (0.06 g, 0.25 mmol) in EtOH (1 mL) was added thiourea (0.06 g, 0.79 mmol) and iodine (0.063 g, 0.25 mmol) and the mixture was heated in an open flask at 180° C. for 2 h (EtOH evaporated). The crude residue was washed with ether and recrystallised from MeOH/H₂O to give a pale yellow shiny solid. This was further purified by flash chromatography (20 g column, Flashmaster II) using an elution gradient of DCM to 5% MeOH in DCM. Yield 55%: ¹H NMR δ (400 MHz, DMSO-d₆) 1.19 (t, J=7.5 Hz, 3H), 2.61 (q, J=7.5 Hz, 2H), 6.87 (d, J=8.3 Hz, 1H), 7.03 (s, 1H), 7.08 (s, 2H), 7.34-7.36 (m, 1H), 7.42 (d, J=2.4 Hz, 1H), 7.59 (d, J=8.6 Hz, 2H), 7.83 (d, J=8.3 Hz, 2H), 9.45 (s, 1H); ¹³C NMR δ (101 MHz, DMSO-d₆) 168.62, 155.29, 139.57, 133.38, 130.84, 127.66, 126.44, 126.36, 125.20, 115.73, 101.58, 23.43, 14.83; HPLC>97%, (Rₜ 2.09, 70% MeCN in H₂O); FAB-HRMS calcd for C₁₇H₁₇N₂OS 297.1062 found (M⁺) 297.1054 m/z.

### 1-(3′-Ethyl-4′-methoxy-biphenyl-4-yl)-3-hydroxy-propenone 41

To a stirred solution of 1-(3′-ethyl-4′-methoxy-biphenyl-4-yl)-ethanone 38 (0.254 g, 1 mmol) in PhMe (12 mL) was added ethyl formate (0.6 mL) and KO^{t}Bu (0.336 g, 3 mmol) and the reaction was stirred at r.t. for 19 h. During this time the reaction became pink in colour and a white precipitate formed. The suspension was acidified with glacial acetic acid (ppt dissolved), diluted with water (10 mL) and the products extracted with ethyl acetate (2×10 mL). The organic layers were combined, washed with water (20 mL) and brine (20 mL) before drying (MgSO₄) and concentration under reduced pressure. The resulting solution in acetic acid/toluene was diluted with water then a small amount of EtOH until the layers mixed and a white powder formed. Further concentration under reduced pressure resulted in further precipitation of the product as a pale yellow powder. Yield 70%: ¹H NMR δ (270 MHz, CDCl₃) 1.23 (t, J=7.4 Hz, 3H), 2.69 (q, J=7.6 Hz, 2H), 3.87 (s, 3H), 6.24 (d, J=4.2 Hz, 1H), 6.92 (d, J=8.2 hz, 1H), 7.43-7.46 (2H), 7.65 (d, J=8.4 Hz, 2H), 7.94 (d, J=8.4 Hz, 2H), 8.28 (d, J=4.2 Hz, 1H); HPLC>92% (Rₜ 3.00, 90% MeCN in H₂O); APCI (M−H)⁻ 281.19 m/z.

### 5-(3′-Ethyl-4′-methoxy-biphenyl-4-yl)-isoxazole 42

To a stirred solution of 1-(3′-ethyl-4′-methoxy-biphenyl-4-yl)-3-hydroxy-propenone 41 (0.076 g, 0.27 mol) in EtOH (5 mL) was added hydroxylamine hydrochloride (0.03 g, 0.43 mmol) and the reaction was heated to reflux for 1 h. The mixture was cooled, acidified with glacial acetic acid and concentrated under reduced pressure until a precipitate began to form. This precipitate was collected by filtration and washed with water. Yield 56%: ¹H NMR δ (270 MHz, CD₃OD) 1.22 (t, J=7.5 Hz, 3H), 2.69 (q, J=7.5 Hz, 2H), 3.87 (s, 3H), 6.78 (d, J=2.0 Hz, 1H), 7.01 (d, J=8.4 Hz, 1H), 7.45-7.52 (2H), 7.70-7.74 (2H), 7.86-7.90 (2H), 8.43 (d, J=1.7 Hz, 1H); HPLC>93% (Rₜ 2.84, 90% MeCN in H₂O); APCI (MH⁺) 280.12 m/z.

### 5-(3′-Ethyl-4′-hydroxy-biphenyl-4-yl)-isoxazole 43

To a stirred solution of 5-(3′-ethyl-4′-methoxy-biphenyl-4-yl)-isoxazole 42 (0.039 g, 0.14 mmol) in dry DCM (5 mL) under N₂, cooled to −78° C. (dry ice/acetone bath) was added slowly drop-wise BBr₃ (0.7 mL of a 1M solution in DCM, 0.7 mmol) and the reaction was allowed to warm slowly to r.t. with stirring overnight. The reaction was quenched with water (5 mL), the organic layer was separated and the aqueous layer washed with DCM (5 mL). The organic layers were combined and concentrated under reduced pressure and the product was purified by flash chromatography using a gradient elution of DCM to 10% MeOH in DCM. Yield 94%: ¹H NMR δ (400 MHz, CD₃OD) 1.27 (t, J=7.6 Hz, 3H), 2.72 (q, J=7.6 Hz, 2H), 6.80 (d, J=2.0 Hz, 1H), 6.87 (d, J=8.3 Hz, 1H), 7.38 (dd, J=8.3, 2.5 Hz, 1H), 7.44 (d, J=2.3 Hz, 1H), 7.73 (d, J=8.8 Hz, 2H), 7.90 (d, J=8.8 Hz, 2H), 8.46 (d, J=2.0 Hz, 1H); HPLC>92% (Rₜ 3.15, 70% MeCN in H₂O); APCI (M−H)-264.11 m/z; FAB-HRMS calcd for C₁₇H₁₅NO₂ 265.1103 found (M⁺) 265.1105 m/z.

### 3-(3′-Ethyl-4′-methoxy-biphenyl-4-yl)-1H-pyrazole 44

To a suspension of 1-(3′-ethyl-4′-methoxy-biphenyl-4-yl)-3-hydroxy-propenone 41 (1.9 mmol) in EtOH (20 mL) was added hydrazine monohydrate (160 μL) and the reaction was heated to reflux for 2 h before being cooled and acidified with glacial acetic acid. The mixture was concentrated under reduced pressure until a precipitate began to form before water was added and the resulting precipitate was collected by filtration, washed with water and dried in vacuo. Yield 1.8 mmol, 95%: mp=164-168° C.; ¹H NMR δ (400 MHz, CDCl₃) 1.25 (t, J=7.4 Hz, 3H), 2.71 (q, J=7.4 Hz, 2H), 3.88 (3H, s), 6.90-6.94 (m, 1H), 7.43-7.47 (m, 2H), 7.63 (d, J=8.2 Hz, 2H), 7.97 (d, J=8.2 Hz, 2H); ¹³C NMR δ (100 MHz, CDCl₃) 14.2, 15.0, 23.4, 55.4, 110.4, 125.3, 126.0, 126.5, 127.0, 127.7, 132.7, 133.0, 136.6, 142.3, 157.3, 157.6; HPLC>99% (Rₜ 2.77, 90% MeCN in H₂O); APCI (M+H)⁺ 279.40 m/z; FAB-HRMS calcd for C₁₈H₁₉N₂O 279.1492 found (M+H)⁺ 279.1488 m/z.

### General Procedure 3: Alkylation of 3-(3′-Ethyl-4′-methoxy-biphenyl-4-yl)-1H-pyrazole

To a stirred solution of 3-(3′-ethyl-4′-methoxy-biphenyl-4-yl)-1H-pyrazole 44 (0.100 g, 0.36 mmol) in dry DMF (4 mL), cooled to 0° C., was added NaH (0.022 g of a 60% dispersion, 0.54 mmol) and the mixture was stirred at 0° C. for 20 min. To this was then added the required alkyl halide (0.72 mmol) and the reaction was allowed to warm slowly to rt with stirring for 22 h. The reaction was quenched with water (10 mL) and the products extracted with EtOAc (2×20 mL), washed with water (2×20 mL), brine (2×20 mL), dried (Na₂SO₄) and concentrated under reduced pressure. Purification by flash chromatography was performed initially using an elution gradient of hexane to EtOAc. In cases where more than one regioisomer was observed, the alkylated products were separated by chromatography using DCM as eluent.

### 3-(3′-Ethyl-4′-methoxy-biphenyl-4-yl)-1-methyl-1H-pyrazole 45

Prepared by general procedure 3 using iodomethane. Yield 31%: mp=167-170° C.; ¹H NMR δ (400 MHz, CDCl₃) 1.25 (3H, t, J=7.6 Hz), 2.71 (2H, q, J=7.6 Hz), 3.87 (3H, s), 3.96 (3H, s), 6.56 (1H, d, J=2.0 Hz), 6.91 (1H, d, J=9.0 Hz), 7.38 (1H, d, J=2.3 Hz), 7.43-7.46 (2H, m), 7.60 (2H, d, J=8.2 Hz), 7.84 (2H, d, J=8.2 Hz); ¹³C NMR δ (100 MHz, CDCl₃) 14.2, 23.4, 39.0, 55.4, 102.8, 110.4, 125.1, 125.8, 126.8, 127.6, 131.3, 131.7, 132.9, 133.0, 140.2, 151.3, 157.0; HPLC>99% (Rₜ 2.94, 90% MeCN in H₂O); APCI (M)⁺ 292.57 m/z; FAB-HRMS calcd for C₁₉H₂₁N₂O 293.1648 found (M+H)⁺ 293.1646 m/z.

### 5-(3′-Ethyl-4′-methoxy-biphenyl-4-yl)-1-methyl-1H-pyrazole 46

Prepared by general procedure 3 using iodomethane. Yield 19%: mp=97-100° C.; ¹H NMR δ (400 MHz, CDCl₃) 1.25 (3H, t, J=7.4 Hz), 2.71 (2H, q, J=7.6 Hz), 3.88 (3H, s), 3.93 (3H, s), 6.34 (1H, d, J=2.0 Hz), 6.92 (1H, d, J=9.0 Hz), 7.43-7.47 (4H, m), 7.53 (1H, d, J=2.0 Hz), 7.64 (2H, d, J=8.2 Hz); ¹³C NMR δ (100 MHz, CDCl₃) 14.2, 23.4, 37.6, 55.4, 106.0, 110.4, 125.3, 126.9, 127.7, 128.8, 129.0, 132.4, 133.1, 138.5, 141.2, 143.4, 157.3; HPLC>99% (Rₜ 2.88, 90% MeCN in H₂O); APCI (M)⁺ 292.57 m/z; FAB-HRMS calcd for C₁₉H₂₁N₂O 293.1648 found (M+H)⁺ 293.1648 m/z.

### 3-(3′-Ethyl-4′-methoxy-biphenyl-4-yl)-1-(2-methoxyethyl-1H-pyrazole) 47

Prepared by general procedure 3 using 1-bromo-2-methoxyethane. Yield 42%: mp=102-105° C.; ¹H NMR δ (400 MHz, CDCl₃) 1.25 (3H, t, J=7.6 Hz), 2.71 (2H, q, J=7.6 Hz), 3.35 (3H, s), 3.78-3.81 (2H, m), 3.86 (3H, s), 4.32-4.35 (2H, m), 6.57 (1H, d, J=2.3 Hz), 6.91 (1H, d, J=9.4 Hz), 7.43-7.46 (2H, m), 7.50 (1H, d, J=2.3 Hz), 7.60 (2H, d, J=8.5 Hz) 7.85 (2H, d, J=8.2 Hz); ¹³C NMR (100 MHz, CDCl₃) δ 14.2, 23.4, 52.2, 53.4, 55.4, 59.0, 71.3, 102.7, 110.4, 125.1, 125.8, 126.8, 127.6, 131.4, 131.8, 132.8, 133.0, 140.1, 151.3, 156.9; HPLC>99% (Rₜ 4.24, 90% MeCN in H₂O); APCI (M)⁺336.84 m/z; FAB-HRMS calcd for C₂₁H₂₅N₂O₂ 337.1911 found (M+H)⁺ 337.1908 m/z.

### [3-(3′-Ethyl-4′-methoxy-biphenyl-4-yl)-pyrazol-1-yl]-acetic Acid Methyl Ester 48

Prepared by general procedure 3 using methyl-2-choroacetate. Yield 65%: mp=116-120° C.; ¹H NMR δ (400 MHz, CDCl₃) 1.24 (3H, t, J=7.4 Hz), 2.70 (2H, q, J=7.4 Hz), 3.78 (3H, s), 3.87 (3H, s), 4.98 (2H, s), 6.65 (1H, d, J=2.3 Hz), 6.91 (1H, d, J=9.4 Hz), 7.43-7.45 (2H, m), 7.50 (1H, d, J=2.3 Hz), 7.59 (2H, d, J=8.2 Hz) 7.84 (2H, d, J=8.6 Hz); ¹³C NMR δ (100 MHz, CDCl₃) 14.2, 23.4, 52.7, 53.1, 55.4, 103.9, 110.4, 125.2, 126.0, 126.8, 127.6, 131.4, 132.0, 132.9, 133.0, 140.4, 152.0, 157.0, 168.4; HPLC>92% (Rₜ 3.04, 70% MeCN in H₂O); APCI (M)⁺350.63 m/z; FAB-HRMS calcd for C₂₁H₂₃N₂O₃ 351.1703 found (M+H)⁺ 351.1703 m/z.

### [3-(3′-Ethyl-4′-methoxy-biphenyl-4-yl)-pyrazol-1-yl]-acetic Acid Ethyl Ester 49

To a stirred solution of 3-(3′-ethyl-4′-methoxy-biphenyl-4-yl)-1H-pyrazole 44 (0.440 g, 1.6 mmol) in dry DMF (10 mL), cooled to −10° C., was added NaH (0.96 g of a 60% dispersion, 2.4 mmol) and the mixture was stirred at −10° C. for 20 min. To this was then added ethyl chloroacetate (0.2 mL, 1.9 mmol) and the reaction was allowed to warm slowly to rt with stirring for 17 h. The reaction was quenched with water (50 mL) and the organics extracted with EtOAc (2×50 mL). The organic layers were combined, washed with water (2×50 mL), brine (2×50 mL), dried (Na₂SO₄) and concentrated under reduced pressure. Recrystallisation from EtOAc/hexane gave white powder, 0.316 g, 54%: ¹H NMR δ (270 MHz, CDCl₃) 1.21-1.31 (6H, 2×t), 2.69 (2H, q, J=7.4 Hz), 3.86 (3H, s), 4.24 (2H, q, J=7.1 Hz), 4.96 (2H, s), 6.65 (1H, d, J=2.5 Hz), 6.90 (1H, d, J=9.1 Hz), 7.42-7.45 (2H, m), 7.51 (1H, d, J=2.2 Hz), 7.58 (2H, d, J=8.4 Hz) 7.83 (2H, d, J=8.4 Hz); HPLC>98% (Rₜ 6.08, 90% MeCN in H₂O); APCI (M+H)⁺ 365.57 m/z.

### [3-(3′-Ethyl-4′-methoxy-biphenyl-4-yl)-pyrazol-1-yl]-acetic Acid 50

To a stirred solution of [3-(3′-ethyl-4′-methoxy-biphenyl-4-yl)-pyrazol-1-yl]-acetic acid ethyl ester 49 (0.227 g, 0.62 mmol) in EtOH/THF (1:1, 20 mL) was added aq. NaOH (0.100 g, 2.5 mmol in 4 mL). A white solid precipitated therefore a further 5 mL of THF were added and the reaction was stirred at rt for 16 h. Water (20 mL) was added and the mixture was concentrated under reduced pressure to 20 mL volume before more water was added and the white precipitate collected by filtration and washed with water. ¹H NMR δ (270 MHz, DMSO-d₆) 1.18 (3H, t, J=7.5 Hz), 2.63 (2H, q, J=7.4 Hz), 3.83 (3H, s), 4.40 (2H, s), 6.63 (1H, d, J=2.2 Hz), 7.03 (1H, d, J=8.1 Hz), 7.49-7.53 (2H, m), 7.61-7.64 (3H, m) 7.81 (2H, d, J=8.4 Hz); HPLC>97% (Rₜ 2.23, 90% MeCN in H₂O); ES-ve MS (M−H)⁻ 335.35 m/z.

### 2-[3-(3′-Ethyl-4′-methoxy-biphenyl-4-yl)-pyrazol-1-yl]-N-pyridin-3-ylmethyl-acetamide 51

To a stirred suspension of [3-(3′-ethyl-4′-methoxy-biphenyl-4-yl)-pyrazol-1-yl]-acetic acid 50 (0.120 g, 0.36 mmol) in dry DCM (6 mL) was added DMAP (catalytic) and 3-aminomethylpyridine (72 μL, 0.71 mmol), followed by NEt₃ (50 μL). This mixture was cooled on ice before EDCI (0.136 g, 0.71 mmol) was added and the reaction was allowed to warm to r.t. with stirring overnight. The mixture was diluted with DCM (10 mL) and washed with sat.aq. bicarb. (10 mL) and the product isolated as the first main fraction by flash chromatography using an elution gradient of DCM to 10% MeOH in DCM: ¹H NMR δ (270 MHz, CDCl₃) 1.23 (3H, t, J=7.5 Hz), 2.69 (2H, q, J=7.5 Hz), 3.86 (3H, s), 4.45 (2H, d, J=6.2 Hz), 4.89 (2H, s), 6.65 (1H, d, J=2.5 Hz), 6.91 (1H, d, J=9.1 Hz), 7.00 (1H, bt, J=5.3 Hz), 7.21 (1H, dd, J=7.9, 4.7 Hz), 7.41-7.44 (2H, m), 7.50 (1H, d, J=2.2 Hz), 7.54-7.60 (3H, m) 7.79 (2H, d, J=8.4 Hz), 8.47-8.50 (2H, m); HPLC>98% (Rₜ 4.69, 90% MeCN in H₂O); ES-ve MS (M−H)⁻ 425.51 m/z.

### 2-[3-(3′-Ethyl-4′-hydroxy-biphenyl-4-yl)-pyrazol-1-yl]-N-pyridin-3-ylmethyl-acetamide 52

To a stirred solution of 2-[3-(3′-ethyl-4′-methoxy-biphenyl-4-yl)-pyrazol-1-yl]-N-pyridin-3-ylmethyl-acetamide 51 (0.020 g, 0.05 mmol) in dry DCM (2 mL) under N₂, cooled to −78° C. (dry ice/acetone bath) was added slowly drop-wise BBr₃ (0.25 mL of a 1M solution in DCM, 0.25 mmol) and the reaction was allowed to warm slowly to r.t. with stirring for 20 h. The reaction was quenched with water (10 mL) and the organics extracted into DCM. Flash chromatography using DCM to 10% MeOH in DCM gave the product as the main fraction: beige powder, yield 48%: ¹H NMR δ (270 MHz, CD₃OD) 1.24 (3H, t, J=7.5 Hz), 2.67 (2H, q, J=7.4 Hz), 4.60-4.61 (2H, m), 5.00 (2H, s), 6.72 (1H, d, J=2.2 Hz), 6.81 (1H, d, J=8.1 Hz), 7.30 (1H, dd, J=8.5, 2.3 Hz), 7.37 (1H, d, J=2.3 Hz), 7.59 (2H, d, J=8.7 Hz), 7.73 (1H, d, J=2.4 Hz), 7.81 (2H, d, J=8.7 Hz), 7.90-7.93 (1H, m), 8.42-8.46 (1H, m), 8.67-8.76 (2H, m); LC/MS (AP−) m/z 410.99 (M−H)⁻; HPLC tr=2.01 min (>95%) 70% MeCN in H₂O.

### 3-(3′-Ethyl-4′-hydroxy-biphenyl-4-yl)-1-(2-hydroxyethyl-1H-pyrazole) 53

To a stirred solution of 3-(3′-ethyl-4′-methoxy-biphenyl-4-yl)-1-(2-methoxyethyl-1H-pyrazole) 47 (0.040 g, 0.12 mmol) in dry DCM (2 mL), cooled to −78° C., was added slowly BBr₃ (0.6 mL of a 1M solution, 0.6 mmol) and the reaction was stirred at −78° C. and monitored by tlc until the starting material was virtually completely consumed (2 h). The reaction was quenched with water (5 mL), allowed to warm to rt, and DCM (5 mL) added. The precipitate was collected by filtration and washed with water and was found to be the title compound; the DCM layer was found to contain starting material. Yield 73%: ¹H NMR δ (270 MHz, CD₃OD) 1.23 (3H, t, J=7.4 Hz), 2.68 (2H, q, J=7.4 Hz), 3.93-3.97 (2H, m), 4.36-4.40 (2H, m), 6.81-6.85 (2H, m), 7.33 (1H, dd, J=8.3, 2.4 Hz), 7.39 (1H, d, J=2.2 Hz ), 7.65 (2H, d, J=8.7 Hz) 7.82 (2H, d, J=8.7 Hz) 7.91 (1H, d, J=2.5 Hz); ¹³C NMR δ (100 MHz, CD₃OD) 12.8, 22.5, 53.4, 59.3, 103.2, 114.3, 124.3, 125.8, 125.9, 126.0, 126.8, 130.4, 134.6, 142.2, 149.0, 154.6; HPLC>93% (Rₜ 3.46, 90% MeCN in H₂O); APCI (M+H)⁺ 309.44 m/z; FAB-HRMS calcd for C₁₉H₂₁N₂O₂ 309.1598 found (M+H)⁺ 309.1594 m/z.

### 3-[5-(3′-Ethyl-4′-methoxy-biphenyl-4-yl)-pyrazol-1-yl]-propionitrile 54

To a stirred suspension of 1-(3′-ethyl-4′-methoxy-biphenyl-4-yl)-3-hydroxy-propenone 41 (0.129 g, 0.46 mmol) in EtOH (5 mL) was added 2-cyanoethyl hydrazine (0.047 g, 0.55 mmol) and the reaction was heated to reflux for 2 h. The mixture was allowed to cool to r.t., acidified with AcOH and concentrated under reduced pressure until a precipitate began to form. Water was added and the resulting beige powder was collected by filtration and washed with water. This was subjected to flash chromatography using gradient elution of hexane to 30% EtOAc in hexane to give the title compound: ¹H NMR δ (400 MHz, CDCl₃) 1.24 (3H, t, J=7.5 Hz), 2.70 (2H, q, J=7.5 Hz), 2.96 (2H, t, J=6.9 Hz), 3.88 (3H, s), 4.41 (2H, t, J=6.9 Hz), 6.33 (1H, d, J=1.7 Hz), 6.93 (1H, d, J=8.9 Hz), 7.42-7.46 (4H, m), 7.61 (1H, d, J=1.9 Hz) 7.64-7.68 (2H, m); HPLC>99% (Rₜ 7.87, 90% MeCN in H₂O); APCI (M+H)⁺ 332.49 m/z.

### 3-(3′-Ethyl-4′-hydroxy-biphenyl-4-yl)-1H-pyrazole 55

To a stirred solution of 3-(3′-ethyl-4′-methoxy-biphenyl-4-yl)-1H-pyrazole 44 (0.03 g, 0.11 mmol) in dry DCM (5 mL) under N₂, cooled to −78° C. (dry ice/acetone bath) was added slowly drop-wise BBr₃ (0.55 mL of a 1M solution in DCM, 0.55 mL) and the reaction was allowed to warm slowly to r.t. with stirring overnight. The reaction was quenched with water (5 mL) and diluted with a further 5 mL DCM. The resulting orange precipitate was collected by filtration before the organic layer was separated and concentrated under reduced pressure. ¹H NMR showed the orange precipitate to be product and the organic extracts to contain mainly starting material. Yield of product isolated 7 mg, 24%: ¹H NMR δ (270 MHz, CD₃OD) 1.23 (t, J=7.4 Hz, 3H), 2.68 (q, J=7.5 Hz, 2H), 6.83 (d, J=8.4 Hz, 1H), 6.90 (d, J=2.5 Hz, 1H), 7.31-7.35 (m, 1H), 7.39 (d, J=2.2 Hz, 1H), 7.65-7.69 (2H), 7.79-7.82 (2H), 7.97 (d, J=2.5 Hz, 1H); HPLC>97% (Rₜ 2.98, 80% MeCN in H₂O); APCI (M+H)⁺ 265.12 m/z; FAB-HRMS calcd for C₁₇H₁₇N₂O 265.1341 found (M+H)⁺ 265.1329 m/z.

### 3-(3′-Ethyl-4′-methoxy-biphenyl-4-yl)-3-oxo-propionitrile 56

To a stirred solution of 1-(3′-ethyl-4′-methoxy-biphenyl-4-yl)-ethanone 38 (0.200 g, 0.7 mmol) in toluene (20 mL) and pyridine (0.36 mL) was added O,N-bistrifluoroacetyl hydroxylamine (0.473 g, 2.1 mmol) and the reaction was heated to reflux for 1.5 h. The mixture was cooled, EtOAc (50 mL) was added and the solution was washed with water (60 mL) and brine (50 mL) before being concentrated and dried under reduced pressure. The resulting brown/yellow powder was used without further purification for the preparation of 5-(3′-Ethyl-4′-methoxyoxy-biphenyl-4-yl)-2H-pyrazol-3-ylamine 57.

### 5-(3′-Ethyl-4′-methoxyoxy-biphenyl-4-yl)-2H-pyrazol-3-ylamine 57

To a stirred solution of 3-(3′-ethyl-4′-methoxy-biphenyl-4-yl)-3-oxo-propionitrile 56 (0.7 mmol) in EtOH (15 mL) was added hydrazine monohydrate (1 mL) and the reaction was heated to reflux for 2.5 h before being cooled and concentrated under reduced pressure until a cream coloured powder precipitated. This was collected by filtration and washed with hexane before drying in vacuo. Yield 0.206 g, 0.7 mmol; ¹H NMR, (270 MHz, CDCl₃) 1.17 (t, J=7.4 Hz, 3H), 2.63 (q, J=7.4 Hz, 2H), 3.83 (s, 3H), 4.78 (bs, 2H), 5.78 (bs, 1H), 7.02 (d, J=8.4 Hz, 1H), 7.48-7.51 (2H), 7.62 (d, J=8.4 Hz, 2H), 7.69 (d, J=8.4 Hz, 2H); Purity by LC>96%; APCI (M+H)⁺ 294.18 m/z.

### [5-(3′-Ethyl-4′-methoxyoxy-biphenyl-4-yl)-2H-pyrazol-3-yl]-(3-pyridin-3-yl-propyl)amine 58

To a stirred solution of 3-pyridine propionic acid (0.106 g, 0.7 mmol) in dry DCM (10 mL) and NEt₃ (0.14 mL) was added DMAP (catalytic) and EDCI (0.400 g, 2.1 mmol) and the reaction was stirred at r.t. for 20 min. To this was then added 5-(3′-ethyl-4′-methoxyoxy-biphenyl-4-yl)-2H-pyrazol-3-ylamine 57 (0.205 g, 0.7 mmol) and the reaction was stirred for 21 h. The reaction was quenched with sat. aq. bicarb. and the organic layer was separated and concentrated under reduced pressure. The product was purified by flash chromatography (20 g column, Flashmaster II) using a gradient elution of DCM to 10% MeOH in DCM. Yield 30%: ¹H NMR δ (400 MHz, CDCl₃) 1.29 (t, J=7.4 Hz, 3H), 2.75 (dt, J=7.8, 7.4 Hz, 2H), 3.16 (t, J=7.6 Hz, 2H), 3.56 (t, J=7.6 Hz, 2H), 3.91 (s, 3H), 6.95 (d, J=9.0 Hz, 1H), 7.27-7.30 (m, 1H), 7.46-7.48 (2H), 7.63-7.68 (3H), 7.84-7.87 (2H), 8.52 (dd, J=4.7, 1.6 Hz, 1H), 8.63 (d, J=2.0 Hz, 1H); HPLC>96% (Rₜ 3.26, 90% MeCN in H₂O); APCI (M+H)⁺ 427.26 m/z.

### 5-(3′-Ethyl-4′-hydroxy-biphenyl-4-yl)-2H-pyrazol-3-ylamine 59

To a stirred solution of [5-(3′-Ethyl-4′-methoxyoxy-biphenyl-4-yl)-2H-pyrazol-3-yl]-(3-pyridin-3-yl-propyl)amine 58 in dry DCM (5 mL), cooled to −78° C., was added BBr₃ (1 mL of a 1M solution in DCM, 1 mmol) and the reaction was stirred at −78° C. for 4 h before being quenched with water (10 mL) and a further 10 mL DCM added. The resulting precipitate was collected by filtration and the organic layer was concentrated under reduced pressure. TLC showed both of these to contain the same mixture of compounds therefore they were combined. Purification by flash chromatography using an elution gradient of DCM to 10% MeOH in DCM yielded the title compound as the only pure product isolated. Yield 27%; ¹H NMR δ (270 MHz, CD₃OD) 1.23 (t, J=7.5 Hz, 3H), 2.67 (q, J=7.5 Hz, 2H), 5.94 (bs, 1H), 6.81 (d, J=8.2 Hz, 1H), 7.27-7.31 (m, 1H), 7.36 (d, J=2.5 Hz, 1H), 7.55-7.59 (m, 2H), 7.64-7.67 (m, 2H); HPLC>95% (Rₜ 2.84, 70% MeCN in H₂O); APCI (M+H)⁺ 280.19 m/z; FAB-HRMS calcd for C₁₇H₁₈N₃O 280.1450 found (M+H)⁺ 280.1446 m/z.

### 4-(3′-Ethyl-4′-hydroxy-biphenyl-4-yl)-2-hydroxy-4-oxo-but-2-enoic Acid Ethyl Ester 60

To a stirred solution of 1-(3′-ethyl-4′-methoxy-biphenyl-4-yl)-ethanone 38 (0.127 g, 0.5 mmol) in toluene (10 mL) was added diethyl oxalate (0.15 mL, 1 mmol) followed by KO^{t}Bu (0.079 g, 0.7 mmol) and the reaction was stirred at r.t. overnight (upon addition of base the solution became yellow in colour and a white powder precipitated. The mixture was acidified with glacial AcOH before being concentrated under reduced pressure to give a solution in AcOH. To this was added a small amount of MeOH followed by water to precipitate the product as a yellow powder. This was collected by filtration and washed with water. A further recrystallisation attempt from DCM/hexane also yielded yellow powder which was dried in vacuo and used without further purification for the preparation of 5-(3′-ethyl-4′-methoxy-biphenyl-4-yl)-2H-pyrazole-3-carboxylic acid 61. Yield 0.155 g, 88%: ¹H NMR δ (270 MHz, CDCl₃) 1.24 (t, J=7.5 Hz, 3H), 1.41 (t, J=7.2 Hz, 3H), 2.70 (q, J=7.4 Hz, 2H), 3.86 (s, 3H), 4.40 (q, J=7.2 Hz, 2H), 6.91 (d, J=9.2 Hz, 1H), 7.10 (s, <1H), 7.42-7.47 (2H), 7.59-7.69 (2H), 7.97-8.08 (2H); LCMS (ES−) m/z (M−H)⁻ 353.22.

### 5-(3′-Ethyl-4′-methoxy-biphenyl-4-yl)-2H-pyrazole-3-carboxylic Acid 61

To a stirred suspension of 4-(3′-ethyl-4′-hydroxy-biphenyl-4-yl)-2-hydroxy-4-oxo-but-2-enoic acid ethyl ester 60 (0.155 g, 0.44 mmol) in EtOH (5 mL) was added hydrazine monohydrate (0.03 mL) and the reaction was stirred at r.t. overnight. The resulting white mixture was acidified with glacial acetic acid before being concentrated under reduced pressure to give a solution in AcOH. Water (10 mL) was added to this and the organics were extracted into EtOAc (2×10 mL). The extracts were combined and concentrated under reduced pressure to give a pale yellow powder. This was redissolved in EtOH (20 mL) and pTsOH (catalytic) added before the solution was heated for 10 min to aromatise the pyrazole ring. The solution was concentrated under reduced pressure, EtOAc (20 mL) added and the solution washed with sat. aq. bicarb. to remove any traces of acid. The organic layer was concentrated under reduced pressure to give a pale yellowish powder. This was shown by tlc not to be pure therefore was washed with DCM/hexane. Yield 53%: ¹H NMR δ (270 MHz, DMSO-d₆) 1.18 (t, J=7.5 Hz, 3H), 2.65 (q, J=7.5 Hz, 2H), 3.84 (s, 3H), 7.05 (d, J=8.2 Hz, 1H), 7.53-7.57 (2H), 7.71-7.75 (2H), 7.89-7.95 (2H); LCMS (AP+) m/z 323.23 (M+H)⁺.

### 4-(3′-Ethyl-4′-hydroxy-biphenyl-4-yl)-4-hydroxy-6-oxo-hex-4-enoic Acid 62

To a stirred solution of 1-(3′-ethyl-4′-methoxy-biphenyl-4-yl)-ethanone 38 (0.517 g, 2 mmol) in dry THF (10 mL), cooled to −78° C. (dry ice/acetone bath) was added slowly drop-wise LDA (2.2 mL of a 1.8 M solution, 4 mmol) and the reaction was stirred at −78° C. for a further 1 h. To this was then added slowly a solution of succinic anhydride (0.400 g, 4 mmol) in dry THF (6 mL) before the reaction was allowed to warm slowly to rt with stirring for 22 h. The reaction was quenched with HCl (20 mL of 5%) and the organics extracted into ether (2×20 mL). The extracts were combined and concentrated under reduced pressure before being subjected to flash chromatography (20 g column, Flashmaster II) using an elution gradient of DCM to 5% MeOH in DCM. This gave an impure product which was used without further purification for the next step. HPLC>80% (Rₜ 1.54, 70% MeCN in H₂O).

### 3-[5-(3′-Ethyl-4′-methoxyoxy-biphenyl-4-yl)-2H-pyrazol-3-yl]-propionic Acid 63

To a stirred solution of 62 (0.195 g) in EtOH (25 mL) was added hydrazine monohydrate (0.03 mL) and the reaction was heated to reflux for 2 h. The mixture was cooled to rt, acidified with glacial acetic acid and the resulting yellow powder collected by filtration, washed with water, dissolved in MeOH then concentrated under reduced pressure (×3) before drying in vacuo. Yield 0.170 g, 25% over 2 steps from 38; ¹H NMR δ (270 MHz, DMSO-d₆) 1.18 (t, J=7.7 Hz, 3H), 2.63 (q, J=7.7 Hz, 2H), 2.82-2.87 (2H), 3.83 (s, 3H), 6.51 (s, 1H), 7.03 (d, J=8.6 Hz, 1H), 7.49-7.53 (2H), 7.63-7.67 (m, 2H), 7.76-7.80 (m, 2H); HPLC>95% (Rₜ 1.29, 70% MeCN in H₂O); APCI (M+H)⁺ 351.27 m/z.

### Oxime Resin Bound 3-[5-(3′-ethyl-4′-methoxyoxy-biphenyl-4-yl)-2H-pyrazol-3-yl]-propionic Acid 64

To Oxime resin (0.49 g, 1 mmolg loading), swollen in dry DMF (7 mL) was added 3-[5-(3′-ethyl-4′-methoxyoxy-biphenyl-4-yl)-2H-pyrazol-3-yl]-propionic acid 63 (0.17 g, 0.49 mmol) followed by DIC (0.4 mL) and HOBt (0.337 g, 2.5 mmol) and the reaction was shaken under N₂ for 48 h. The resin was washed with DCM, MeOH (×3) then with DCM (×3) before being dried in vacuo. Weight of dry resin 0.59 g, giving an approximate loading of 0.58 mmo/g.

### 3-[5-(3′-Ethyl-4′-methoxy-biphenyl-4-yl)-2H-pyrazol-3-yl]-N-pyridin-3-ylmethyl-propionamide 65

To the Oxime resin bound 3-[5-(3′-ethyl-4′-methoxy-biphenyl-4-yl)-2H-pyrazol-3-yl]-propionic acid 64 (0.300 g, 1.74 mmol assuming loading of 0.58 mmol/g), swollen in dry DCM (8 mL) under N₂, was added 3-aminomethylpyridine (0.09 mL, 0.87 mmol) and the reaction was heated to 40° C. for 4 d. The mixture was filtered and the resin washed with DCM x3 and the combined filtrates were concentrated under reduced pressure. The product was purified by recrystallisation from DCM/hexane to give a cream coloured powder. Yield of product 0.08 g, 0.182 mmol, giving loading as 0.6 mmol/g: ¹H NMR δ (270 MHz, CDCl₃) 1.23 (t, J=7.5 Hz, 3H), 2.64-2.73 (4H, m), 3.04-3.09 (2H, m), 3.86 (s, 3H), 4.41 (s) & 4.43 (s) (2H), 6.37 (s, 1H), 6.69 (˜bs, 1H), 6.90 (d, J=9.4 Hz, 1H), 7.10-7.14 (m, 1H), 7.40-7.43 (2H), 7.48 (d, J=7.7 Hz, 1H), 7.57 (d, J=8.5 Hz, 2H), 7.69 (d, J=8.5 Hz, 2H), 8.37-8.40 (m, 1H), 8.45 (s, 1H); HPLC>98% (Rₜ 2.22, 80% MeCN in H₂O); APCI (M−H⁺) 439.23 m/z.

### 3-[5-(3′-Ethyl-4′-hydroxy-biphenyl-4-yl)-2H-pyrazol-3-yl]-N-pyridin-3-ylmethyl-propionamide 66

To a stirred solution of 3-[5-(3′-ethyl-4′-methoxy-biphenyl-4-yl)-2H-pyrazol-3-yl]-N-pyridin-3-ylmethyl-propionamide 65 (0.07 g, 0.16 mmol) in dry DCM (5 mL), cooled to −78° C. (dry ice/acetone bath) was added drop-wise BBr₃ (0.8 mL of a 1M solution in DCM, 0.8 mmol) and the reaction was allowed to warm slowly to r.t. with stirring overnight. The reaction was quenched with water (10 mL) and a further 5 mL of DCM were added. The organic layer was separated and the aqueous layer was neutralised with saturated aqueous bicarbonate. The resulting white powder was collected by filtration. Yield 0.053 g, 78%: mp 210-214° C.; ¹H NMR δ (270 MHz, CD₃OD) 1.24 (t, J=7.5 Hz, 3H), 2.614-2.69 (4H), 3.01 (t, J=7.4 Hz, 2H), 4.40 (s, 2H), 6.41 (s, 1H), 6.77 (d, J=8.1 Hz, 1H), 7.24-7.29 (2H), 7.35 (d, J=2.2 Hz, 1H), 7.56-7.67 (5H), 8.34 (dd, J=5.0, 1.5 Hz, 1H), 8.45 (d, J=1.5 Hz, 1H); HPLC>96% (Rₜ 1.76, 90% MeCN in H₂O); APCI (M−H)-425.31 m/z.

### 4-(4-Bromophenyl)-2,2-dimethyl-4-oxo-butyric Acid 67

To a stirred suspension of 2,2-dimethylsuccinic anhydride (0.641 g, 5.0 mmol) in bromobenzene (3.3 mL), cooled to −10° C. (ice/acetone bath) was added aluminium trichloride (1.34 g, 10 mmol) and the reaction was allowed to warm slowly to rt with stirring overnight. The resulting brown solution was poured into cooled (ice bath) aqueous HCl (10 mL, 18%) and stirred for a further 30 min while the solution was allowed to warm to r.t. No precipitate formed therefore DCM (10 mL) was added and the organic layer was separated and concentrated under reduced pressure to give a solution of the product in PhBr. To this was added hexane followed by a small amount of DCM and the resulting white needles were collected by filtration. Yield 51%: ¹H NMR δ (270 MHz, DMSO-d₆) 1.24 (6H, s), 3.29 (2H, s), 7.69 (2H, d, J=8.7 Hz), 7.88 (2H, d, J=8.7 Hz), 7.96 (1H, s); HPLC>93% (Rₜ 2.87, 70% MeCN in H₂O); APCI (M−H)⁻ 283.15, 285.10 m/z.

### 4-(3′-Ethyl-4′-methoxy-biphenyl-4-yl)-2,2-dimethyl-4-oxo-butyric Acid 68

To a stirred solution of 4-(4-bromophenyl)-2,2-dimethyl-4-oxo-butyric acid 67 (0.285 g, 1 mmol) in toluene (9 mL), EtOH (1 mL) and 2M aqueous Na₂CO₃ (1 mL) was added 3-ethyl-4-methoxyphenyl boronic acid 6 (0.198 g, 1.1 mmol) and the mixture was degassed by bubbling N₂ through for 40 min. To this was then added Pd(PPh₃)₄ (catalytic) and the reaction was heated to reflux for 19 h. The reaction was allowed to cool before water (50 mL) was added and the organics were extracted into EtOAc (2×50 mL) followed by DCM (50 mL). The extracts were combined and concentrated under reduced pressure and the product purified by flash chromatography (20 g column, Flashmaster II) using a gradient elution of DCM to 10% MeOH in DCM. Recrystallisation from DCM/hexane gave colourless cubes. Yield 60%: ¹H NMR δ (270 MHz, CDCl₃) 1.29 (t, J=7.4 Hz, 3H), 1.37 (s, 6H), 2.69 (q, J=7.4 Hz, 2H), 3.33 (s, 2H), 3.87 (s, 3H), 6.92 (d, J=8.4 Hz, 1H), 7.42-7.46 (2H), 7.63 (d, J=8.4 Hz, 2H), 7.98 (d, J=8.4 Hz, 2H); HPLC>99% (Rₜ 2.68, 90% MeCN in H₂O); APCI (M−H⁺) 339.17 m/z.

### 5-(3′-Ethyl-4′-methoxy-biphenyl-4-yl)-3,3-dimethyl-4-oxo-3H-furan-2-one 69

From attempted amide coupling: To a stirred solution of 4-(3′-ethyl-4′-methoxy-biphenyl-4-yl)-2,2-dimethyl-4-oxo-butyric acid 68 (0.100 g, 0.3 mmol) in dry DCM (15 mL) was added DMAP (catalytic), EDCI (0.191 g, 1 mmol) and NEt₃ (0.06 mL) and the reaction was stirred at r.t. for 20 min before addition of 3-(2-aminoethyl)pyridine (0.06 mL). The solution was stirred at r.t. for 24 h before the solution was washed with sat. aq. bicarb. and the organic layer was separated and concentrated under reduced pressure. Flash chromatography (20 g column, Flashmaster II) using a gradient elution of DCM to 5% MeOH in DCM yielded the title compound as the first fraction (R_{f} 0.96, 5% MeOH in DCM). Yield 0.026 g, 27%; ¹H NMR δ (270 MHz, CDCl₃) 1.23 (t, J=7.5 Hz, 3H), 1.41 (s, 6H), 2.69 (q, J=7.6 Hz, 2H), 3.87 (s, 3H), 5.81 (s, 1H), 6.91 (d, J=6.9 Hz, 1H), 7.39-7.44 (2H), 7.55-7.65 (4H); HPLC>99% (Rₜ 2.91, 90% MeCN in H₂O); APCI (MH⁺) 323.29 m/z.

From reaction of 4-(3′-ethyl-4′-methoxy-biphenyl-4-yl)-2,2-dimethyl-4-oxo-butyric acid 68 with acetyl chloride: To a stirred solution of 4-(3′-ethyl-4′-methoxy-biphenyl-4-yl)-2, 2-dimethyl-4-oxo-butyric acid 68 (0.055 g, 0.16 mmol) in dry DCM (5 mL) was added acetyl chloride (13 μL) and NEt₃ (30 μL) and the reaction was stirred at r.t. for 48 h. To this was added DCM (5 mL) and water (10 mL) and the organic layer was separated and concentrated under reduced pressure. Purification by flash chromatography (10 g column, Flashmaster II) using an elution gradient of hexane to 10% DCM in hexane yielded 0.029 g of product (56%); ¹H NMR and R_{f} as above.

### 4-(3′-Ethyl-4′-methoxy-biphenyl)-2,2-dimethyl-4-oxo-N-pyridin-3-ylmethyl-butyramide 70

To a stirred solution of 5-(3′-ethyl-4′-methoxy-biphenyl-4-yl)-3,3-dimethyl-4-oxo-3H-furan-2-one 69 (0.0265 g, 0.08 mmol) in dry DCM (5 mL) was added 3-aminomethyl pyridine (0.01 mL) and the mixture was heated to reflux for 22 h before being cooled and concentrated under reduced pressure. The product was purified by flash chromatography using an elution gradient of DCM to 10% MeOH in DCM. Yield 54%: ¹H NMR δ (270 MHz, CDCl₃) 1.20-1.27 (6H), 1.36 (s, 3H), 2.35 (s, 2H), 2.69 (q, J=7.4 Hz, 2H), 3.86 (s, 3H), 3.97 (d, J=15.1 Hz, 1H), 4.68 (d, J=15.1 Hz, 1H), 4.97 (bs, 1H), 6.91 (d, J=9.2 Hz, 1H), 7.06-7.11 (m, 1H), 7.38-7.41 (4H), 7.53-7.61 (3H), 8.12-8.15 (2H); HPLC>99% (Rₜ 2.61, 80% MeCN in H₂O); APCI (MH⁺) 431.42 m/z; FAB-HRMS calcd for C₂₇H₃₁N₂O₃ 431.2335 found (M+H)⁺ 431.2336 m/z.

### 4-(3′-Ethyl-4′-hydroxy-biphenyl)-2,2-dimethyl-4-oxo-N-pyridin-3-ylmethyl-butyramide 71

To a stirred solution of 4-(3′-ethyl-4′-methoxy-biphenyl)-2,2-dimethyl-4-oxo-N-pyridin-3-ylmethyl-butyramide 70 (0.018 g, 0.04 mmol) in dry DCM (1 mL), cooled to −78° C., was added drop-wise BBr₃ (0.2 mL of a 1M solution, 0.2 mmol) and the reaction was allowed to warm slowly with stirring over 21 h. Water (10 mL) was added, followed by DCM (10 mL) and the organic layer was separated. The aqueous layer was neutralised with NaHCO₃ to give the title compound as white powder which was collected by filtration, washed with water and dried in vacuo. Yield 0.012 g, 72%: ¹H NMR δ (400 MHz, CD₃OD) 1.26 (t, J=7.4 Hz, 3H), 1.30 (s, 3H), 1.41 (s, 3H), 2.36-2.45 (m, 2H), 2.70 (q, J=7.6 Hz, 2H), 4.32-4.42 (m, 2H), 6.84 (d, J=8.2 Hz, 1H), 7.27-7.30 (2H), 7.35 (d, J=2.1 Hz, 1H), 7.40 (d, J=8.7 Hz, 2H), 7.50 (d, J=8.5 Hz, 2H), 7.67 (d, J=7.9 Hz, 1H) 8.30 (s, 1H), 8.34 (s, 1H); HPLC>94% (Rₜ 1.98, 70% MeCN in H₂O); FAB-LRMS (M+H)⁺ 417.1 m/z; FAB-HRMS calcd for C₂₆H₂₉N₂O₃ 417.2178 found (M+H)⁺ 417.2176 m/z.

### 4-(3′-Ethyl-4′-methoxy-biphenyl)-2,2-dimethyl-4-oxo-N-pyridin-3-ylethyl-butyramide 72

To a stirred solution of 5-(3′-ethyl-4′-methoxy-biphenyl-4-yl)-3,3-dimethyl-4-oxo-3H-furan-2-one 69 (0.029 g, 0.09 mmol) in dry DCM (5 mL) was added 2-(pyridin-3-yl)ethanamine (0.01 mL) and the mixture was heated to reflux for 46 h before being cooled and concentrated under reduced pressure. The product was purified by flash chromatography using an elution gradient of DCM to 10% MeOH in DCM. Yield (100%): ¹H NMR δ (270 MHz, CDCl₃) 1.19-1.24 (6H), 1.31 (s, 3H), 2.30 (s, 2H), 2.68 (q, J=7.4 Hz, 2H), 2.87-3.08 (3H), 3.54-3.65 (m, 1H), 3.85 (s, 3H), 6.89 (d, J=9.2 Hz, 1H), 7.08-7.13 (m, 1H), 7.36-7.42 (4H), 7.48-7.55 (3H), 8.15 (bs, 2H); HPLC>96% (Rₜ 3.11, 90% MeCN in H₂O); FAB-LRMS (M+H)⁺ 445.2 m/z; FAB-HRMS calcd for C₂₈H₃₃N₂O₃ 445.2491 found (M+H)⁺ 445.2490 m/z.

### 4-(3′-Ethyl-4′-hydroxy-biphenyl)-2,2-dimethyl-4-oxo-N-pyridin-3-ylethyl-butyramide 73

Procedure as for 4-(3′-Ethyl-4′-hydroxy-biphenyl)-2,2-dimethyl-4-oxo-N-pyridin-3-ylmethyl-butyramide 71, from 4-(3′-Ethyl-4′-methoxy-biphenyl)-2,2-dimethyl-4-oxo-N-pyridin-3-ylethyl-butyramide 72. Yield 52%: ¹H NMR δ (270 MHz, CD₃OD) 1.19-1.24 (6H), 1.32 (s, 3H), 2.26-2.36 (m, 2H), 2.66 (q, J=7.4 Hz, 2H), 2.82-2.90 (2H), 3.05-3.16 (m, 1H), 3.42-3.53 (m, 1H), 6.80 (d, J=8.2 Hz, 1H), 7.26-7.30 (m, 2H), 7.35 (d, J=2.2 Hz, 1H), 7.42 (d, J=8.4 Hz, 2H), 7.56-7.62 (3H), 8.28-8.32 (2H); ¹³C NMR δ (100 MHz, CD₃OD) 13.5, 23.1, 25.1, 25.9, 31.5, 39.8, 41.7, 52.2, 90.9, 114.8, 124.9, 126.1, 127.5, 130.9, 131.4, 137.3, 141.3, 141.4, 146.5, 149.0, 154.9, 181.5; HPLC>99% (Rₜ 2.02, 90% MeCN in H₂O); FAB-LRMS (M+H)⁺ 431.2 m/z; FAB-HRMS calcd for C₂₇H₃₁N₂O₃ 431.2335 found (M+H)⁺ 431.2340 m/z.

### 4-(4-Bromo-2-methylphenyl)-4-oxo-butyric Acid 74

To a stirred mixture of succinic anhydride (0.50 g, 5 mmol) in 3-bromotoluene (3.74 mL, 30.85 mmol), cooled to −5° C. (ice/acetone bath) was added in one portion AlCl₃ (1.33 g, 10 mmol). The reaction temperature was maintained at −5° C. for 4 h before being slowly allowed to warm to r.t. overnight. The mixture was poured into cooled (ice bath) stirred aqueous HCl (15 mL, 18%) and stirring was continued for a further 30 min while the solution was allowed to warm to r.t. No precipitate formed therefore DCM (10 mL) was added and the organic layer was separated and concentrated under reduced pressure. To this was added hexane (5 mL) followed by a small amount of DCM (to mix the layers). The white needles obtained were shown by NMR to contain a small amount of the ortho product therefore were recrystallised again from DCM/hexane. Yield 63%: ¹H NMR δ (270 MHz, CDCl₃) 2.46 (s, 3H), 2.75-2.80 (m, 2H), 3.14-3.19 (m, 2H), 7.39-7.42 (2H), 7.55-7.59 (m, 1H); HPLC>99% (Rₜ 1.37, 80% MeCN in H₂O); FAB-LRMS (M+H)⁺ 270.9, 272.9 m/z; FAB-HRMS calcd for C₁₁H₁₂O₃⁷⁹Br 270.9970 found (M+H)⁺ 270.9967, calcd for C₁₁H₁₂O₃ ⁸¹Br 272.9949 found (M+H)⁺ 272.9949 m/z.

### 4-(3′-Ethyl-4′-methoxy-3-methyl-biphenyl-4-yl)-4-oxo-butyric Acid 75

The same procedure was followed as for the formation of 4-(3′-ethyl-4′-methoxy-biphenyl-4-yl)-2,2-dimethyl-4-oxo-butyric acid 68, from 4-(4-bromo-2-methylphenyl)-4-oxo-butyric acid 74. Yield 58%: ¹H NMR δ (270 MHz, CDCl₃) 1.23 (t, J=7.4 Hz, 3H), 2.58 (s, 3H), 2.69 (q, J=7.4 Hz, 2H), 2.78-2.83 (m, 2H), 3.26-3.31 (m, 2H), 3.87 (s, 3H), 6.91 (d, J=8.2 Hz, 1H), 7.41-7.47 (4H), 7.81 (d, J=7.9 Hz, 1H); HPLC>99% (Rₜ 2.23, 50% MeCN in H₂O); FAB-LRMS (M+H)⁺ 327.1 m/z; FAB-HRMS calcd for C₂₀H₂₃O₄ 327.1596 found (M+H)⁺ 327.1585 m/z.

### 4-(3′-Ethyl-4′-methoxy-3-methyl-biphenyl)-4-oxo-N-pyridin-3-ylmethyl-butyramide 76

Prepared by general procedure 1 but using 75 instead of 26. Yield 87%; ¹H NMR δ (270 MHz, CDCl₃) 1.22 (t, J=7.4 Hz, 3H), 2.53 (s, 3H), 2.60-2.72 (4H), 3.29 (t, J=6.4 Hz, 2H), 3.84 (s, 3H), 4.42 (d, J=5.9 Hz, 2H), 6.88 (d, J=9.2 Hz, 1H), 6.93-6.97 (m, 1H, NH), 7.18-7.23 (m, 1H), 7.37-7.43 (4H), 7.61-7.66 (m, 1H), 7.78 (d, J=8.9 Hz, 1H), 8.43-8.45 (m, 1H), 8.49 (d, J=1.7 Hz, 1H); HPLC>98% (Rₜ 2.49, 80% MeCN in H₂O); FAB-LRMS (M+H)⁺ 417.1 m/z; FAB-HRMS calcd for C₂₆H₂₉N₂O₃ 417.2178 found (M+H)⁺ 417.2175 m/z.

### 4-(3′-Ethyl-4′-methoxy-3-methyl-biphenyl)-4-oxo-N-pyridin-3-ylethyl-butyramide 77

Prepared by general procedure 1 but using 75 instead of 26. Yield 80%; ¹H NMR δ (270 MHz, CDCl₃) 1.21 (t, J=7.4 Hz, 3H), 2.53-2.57 (5H), 2.67 (q, J=7.4 Hz, 2H), 2.77-2.83 (m, 2H), 3.23-3.28 (m, 2H), 3.49 (dd, J=13.1, 6.9 Hz, 2H), 3.83 (s, 3H), 6.49-6.53 (m, 1H, NH), 6.87 (d, J=9.2 Hz, 1H), 7.14-7.20 (m, 1H), 7.38-7.44 (4H), 7.50-7.54 (m, 1H), 7.79 (d, J=8.4 Hz, 1H), 8.38-8.43 (2H); HPLC>98% (Rₜ 2.52, 80% MeCN in H₂O); FAB-LRMS (M+H)⁺ 431.2 m/z; FAB-HRMS calcd for C₂₇H₃₁N₂O₃ 431.2335 found (M+H)⁺ 431.2337 m/z.

### 4-(3′-Ethyl-4′-hydroxy-3-methyl-biphenyl)-4-oxo-N-pyridin-3-ylmethyl-butyramide 78

Procedure as for 4-(3′-ethyl-4′-hydroxy-biphenyl)-2,2-dimethyl-4-oxo-N-pyridin-3-ylmethyl-butyramide 33, from 4-(3′-ethyl-4′-methoxy-3-methyl-biphenyl)-4-oxo-N-pyridin-3-ylmethyl-butyramide 76. Yield 93%: ¹H NMR δ (270 MHz, CD₃OD) 1.23 (t, J=7.5 Hz, 3H), 2.50 (s, 3H), 2.63-2.71 (4H), 3.28-3.34 (m, 2H—under MeOH), 4.43 (s, 2H), 6.82 (d, J=8.4 Hz, 1H), 7.30-7.49 (5H), 7.81-7.87 (2H), 8.41 (d, J=4.2 Hz, 2H), 8.52 (s, 1H); ¹³C NMR δ (100 MHz, DMSO-d₆) 14.8, 21.6, 23.4, 29.9, 36.4, 55.4, 115.8, 118.4, 123.6, 123.9, 125.7, 128.1, 129.4, 129.9, 130.1, 131.0, 135.4, 135.7, 138.4, 143.5, 148.5, 129.1, 156.2, 172.1, 202.7; HPLC>97% (Rₜ 1.91, 90% MeCN in H₂O); FAB-LRMS (M+H)⁺ 403.0 m/z; FAB-HRMS calcd for C₂₅H₂₇N₂O₃ 403.20226 found (M+H)⁺ 403.2026 m/z.

### 4-(3′-Ethyl-4′-hydroxy-3-methyl-biphenyl)-4-oxo-N-pyridin-3-ylethyl-butyramide 79

Procedure as for 4-(3′-ethyl-4′-hydroxy-biphenyl)-2,2-dimethyl-4-oxo-N-pyridin-3-ylmethyl-butyramide 33, from 4-(3′-ethyl-4′-methoxy-3-methyl-biphenyl)-4-oxo-N-pyridin-3-ylethyl-butyramide 77. Yield 75%: ¹H NMR δ (400 MHz, CD₃OD) 1.27 (t, J=7.5 Hz, 3H), 2.56-2.57 (5H), 2.71 (q, J=7.5 Hz, 2H), 2.88 (t, J=7.0 Hz, 2H), 3.48 (t, J=7.0 Hz, 2H), 6.84 (d, J=8.3 Hz, 1H), 7.33-7.53 (5H), 7.90 (dt, J=7.8, 1.9 Hz, 1H), 7.87 (d, J=8.0 Hz, 1H), 8.41 (dd, J=4.8, 1.6 Hz, 1H), 8.48 (d, J=1.6 Hz, 1H); ¹³C NMR δ (100 MHz, CD₃OD) 13.5, 20.6, 23.1, 29.5, 32.2, 40.0, 110.0, 115.5, 123.1, 123.8, 125.1, 127.4, 129.1, 129.6, 129.8, 131.3, 134.5, 135.8, 137.5, 138.7, 144.7, 146.6, 149.1, 157.1, 173.80; HPLC>99% (Rₜ 1.91, 90% MeCN in H₂O); FAB-LRMS (M+H)⁺ 417.1 m/z; FAB-HRMS calcd for C₂₆H₂₉N₂O₃ 417.2178 found (M+H)⁺ 417.2179 m/z.

### 1-(4′-Hydroxy-biphenyl-4-yl)-ethanone 80

A mixture of 4′-bromoacetophenone (0.100 g, 0.5 mmol), 4-hydroxyphenylboronic acid (0.103 g, 0.75 mmol), K₂CO₃, 0.172 g, 1.25 mmol, Bu₄NBr (0.161 g, 0.5 mmol) and Pd(OAc)₂ (catalytic) in EtOH (1.5 mL) and water (3.5 mL) was heated at 150° C. in a microwave for 10 min. Water (20 mL) was added and the organics extracted into EtOAc (20 mL). Purification by flash chromatography using an elution gradient of hexane to 30% EtOAc in hexane gave the title compound, 30 mg, 28%: ¹H NMR δ (400 MHz, CD₃OD) 2.59 (3H, s), 6.88 (2H, d, J=8.6 Hz), 7.52 (2H, d, J=8.6 Hz), 7.66 (2H, d, J=8.2 Hz), 7.99 (2H, d, J=8.2 Hz); ¹³C NMR δ (100 MHz, CD₃OD) 24.6, 114.9, 125.4, 127.4, 128.1, 130.2, 134.3, 145.3, 157.2, 198.2; LC/MS (APCI) m/z 211.24 (M−H)⁻; HPLC tᵣ=3.59 min (>94%) 90% MeCN in H₂O.

### 1-(4-Benzo[1,3] dioxol-5-yl-phenyl)-ethanone 81

Procedure and purification as for 80, from 3,4-(methylenedioxy)phenyl boronic acid. Yield 90 mg, 75%: ¹H NMR δ (400 MHz, CDCl₃) 2.59 (3H, s), 5.98 (2H, s), 6.87 (1H, d, J=7.8 Hz), 7.06-7.09 (2H, m), 7.56 (2H, d, J=8.6 Hz), 7.96 (2H, d, J=8.6 Hz); ¹³C NMR δ (100 MHz, CDCl₃) 26.5, 101.3, 107.4, 108.6, 120.9, 126.7, 128.8, 133.9, 135.3, 145.3, 147.8, 148.2, 197.6; LC/MS (APCI) m/z 241.36 (M+H)⁺; HPLC tᵣ=4.34 min (>97%) 90% MeCN in H₂O.

### 1-(4-Benzo[1,3] dioxol-5-yl-phenyl)-ethanol 82

To a stirred solution of 1-(4-benzo[1,3]dioxol-5-yl-phenyl)-ethanone 81 (0.048 g, 0.2 mmol) in THF/EtOH (2:1, 3 mL), cooled on ice, was added NaBH₄ (0.020 g, 0.52 mmol) and the reaction was allowed to warm to r.t. with stirring overnight. To this was then added H₂O (10 mL) and the organics extracted into EtOAc (10 mL). The organic layer was separated and washed with brine (10 mL) and the product was isolated by flash chromatography using an elution gradient of hexane to 30% EtOAc in hexane. Yield 37 mg, 76%; ¹H NMR δ (270 MHz, DMSO-d₆) 1.33 (3H, d, J=6.4 Hz), 4.74 (1H, dd, J=6.4, 4.3 Hz), 5.17 (1H, d, J=4.2 Hz), 6.05 (2H, s), 6.98 (1H, d, J=8.2 Hz), 7.12 (1H, dd, J=8.1, 1.9 Hz), 7.22 (1H, d, J=1.8 Hz), 7.37 (2H, d, J=8.1 Hz), 7.53 (2H, d, J=8.5 Hz); LC/MS (ES+) m/z 225.37 (M−OH)⁺; HPLC tᵣ=4.36 min (>99%) 90% MeCN in H₂O.

### 3-chloro-4-hydroxyphenylboronic Acid 83

To a stirred solution of 4-bromo-2-chlorophenol (5 g, 24 mmol) in dry THF (75 mL), cooled to −78° C., was added slowly drop-wise n-BuLi (12 mL of a 2.44 M solution, 29 mmol) and the reaction was stirred at −78° C. for 2 h. To this was then added trimethyl borate (3.3 mL, 29 mmol) and the reaction was allowed to warm slowly to r.t. with stirring for 19 h. The reaction was quenched with HCl (aq., 2 M) and the organics extracted into EtOAc (2×60 mL). These extracts were combined and concentrated under reduced pressure to give a white precipitate in an oily substance. To this was added hexane and the white powder was collected by filtration and washed with hexane. Yield 15%: ¹H NMR δ (270 MHz, DMSO-d₆) 6.49 (bs), 6.91 (1H, d, J=7.9 Hz), 7.54 (1H, dd, J=8.2, 1.5 Hz), 7.72 (1H, d, J=1.5 Hz), 8.02 (bs), 10.32 (1H, s); HPLC tᵣ=3.36 min (>91%) 90% MeCN in H₂O; LC/MS (APCI) m/z 171.16 (M−H)⁻.

### 1-(3′-Chloro-4′-hydroxy-biphenyl-4-yl)-ethanone 84

To a mixture of 4′-bromoacetophenone (0.100 g, 0.5 mmol), 3-chloro-4-hydroxyphenylboronic acid 83 (0.103 g, 0.6 mmol), K₂CO₃ (0.173 g, 1.25 mmol) and Bu₄NBr (0.161 g, 0.5 mmol) in EtOH (1.2 mL) and water (2.8 mL) was added Pd(OAc)₂ (catalytic) and the reaction was microwaved at 150° C. for 10 min. Water (10 mL) was added and the organics extracted into EtOAc (10 mL). Flash chromatography using an elution gradient of hexane to 30% EtOAc in hexane gave the product in a mixture. This was dissolved in EtOAc and extracted with base. The aqueous layer was acidified and organics extracted into EtOAc then concentrated under reduced pressure. Flash chromatography using DCM as eluent gave the product as the first fraction: ¹H NMR δ (270 MHz, CDCl₃) 2.62 (3H, s), 5.86 (1H, s), 7.11 (1H, d, J=8.4 Hz), 7.44 (1H, dd, J=8.4, 2.2 Hz), 7.57-7.62 (3H, m), 8.00 (2H, d, J=8.7 Hz); HPLC>92% (Rₜ 3.98, 90% MeCN in H₂O); ES-ve MS (M−H)⁻ 245.22 m/z

### 4′-Methoxy-2-methyl-biphenyl 85

A suspension of 4-methyoxyphenyl boronic acid (0.57 g), 2-bromotoluene (0.3 mL), K₂CO₃ (0.86 g) and Bu₄NBr (0.805 g) in EtOH (7.5 mL) and water (17.5 mL) was degassed by bubbling N₂ through for 30 min while heating to 80° C. To the resulting solution was added Pd(OAc)₂ (catalytic) and the reaction was heated at 80° C. for 1 h with vigorous stirring. The reaction mixture was then allowed to cool before EtOAc (50 mL) was added and the mixture was washed with 1M NaOH (aq) (2×50 mL), water (2×50 mL) and brine (2×50 mL) before being concentrated under reduced pressure. Flash chromatography (20 g column, Flashmaster II) using a gradient elution of hexane to 10% EtOAc in hexane yielded the title compound (0.602 g, 3.0 mmol): ¹H NMR δ (270 MHz, CDCl₃) 2.53 (3H, s), 4.01 (3H, s), 7.15-7.20 (2H, m), 7.43-7.51 (6H, m); LC/MS (ES−) m/z 197.28 (M−H)⁻; HPLC tr=2.51 min (>92%) 90% MeCN in H₂O.

### 1-(4′-Methoxy-2-methyl-biphenyl-4-yl)-ethanone 86

To a stirred solution of 4′-methoxy-2-methyl-biphenyl 85 (06.00 g, 3 mmol) in dry DCM (5 mL) was added acetic anhydride (0.34 mL, 3.6 mmol) and the solution was cooled to −10° C. (ice/acetone bath). To this was then added AlCl₃ (0.800 g, 6 mmol) and the reaction was allowed to warm slowly to r.t. with stirring over 65 h. The reaction was quenched with 2M HCl (aq) and the organics extracted into DCM. Purification by flash chromatography using hexane to 10% EtOAc in hexane gave the product as the second fraction in 33% yield. ¹H NMR δ (270 MHz, CDCl₃) 2.27 (3H, s), 2.66 (3H, s), 3.95 (3H, s), 7.02 (1H, d, J=8.7 Hz), 7.19-7.25 (4H, m), 7.44 (1H, dd, J=8.5, 2.5 Hz), 7.73 (1H, d, J=8.5 Hz); LC/MS (ES+) m/z 263.45 (M+Na)⁺; HPLC tr=2.20 min (>99%) 90% MeCN in H₂O.

### 1-(4′-Hydroxy-2-methyl-biphenyl-4-yl)-ethanone 87

To a stirred solution of 1-(4′-methoxy-2-methyl-biphenyl-4-yl)-ethanone 86 (0.205 g, 0.85 mmol) in dry DCM (5 mL), cooled to −78° C., was added slowly drop-wise BBr₃ (2.6 mL of 1M, 2.6 mmol) and the reaction was allowed to warm slowly to r.t. with stirring for 20 h. Water was added and the organics extracted into DCM. Purification by flash chromatography (20 g, Flashmaster) using hexane to 5% then 10% EtOAc gave the title compound as the first major fraction. Yield 0.156 g, 81%: ¹H NMR δ (270 MHz, CDCl₃) 2.31 (3H, s), 2.65 (3H, s), 7.05 (1H, d, J=8.4 Hz), 7.22-7.33 (4H, m), 7.47 (1H, dd, J=8.4, 2.3 Hz), 7.71 (1H, d, J=2.2 Hz), 12.32 (˜1H, s); LC/MS (AP−) m/z 224.99 (M−H)⁻; HPLC tr=2.75 min (>99%) 90% MeCN in H₂O.

### 1-Bromo-4-methoxy-2-methyl-benzene 88

To a stirred solution of 4-bromo-3-methyl phenol (1 g, 5.3 mmol) in 6 mL DMF was added K₂CO₃ (2.21 g, 16 mmol) and the reaction was stirred at r.t. for 5 min before addition of MeI (0.41 mL, 6.6 mmol). Stirring was continued for a further 24 h before water was added and the organics were extracted into EtOAc. The solution was concentrated under reduced pressure to give pale brown liquid, 0.814 g, 75% yield: ¹H NMR δ (270 MHz, CDCl₃) 2.36 (3H, s), 3.76 (3H, s), 6.61 (1H, dd, J=3.0, 8.8 Hz), 6.78 (1H, d, J=3.0 Hz), 7.39 (1H, d, J=8.7 Hz).

### 2-Methyl-4-methoxyphenyl Boronic Acid 89

To a stirred solution of 1-bromo-4-methoxy-2-methyl-benzene 88 (4 mmol) in dry THF (10 mL), cooled to −78° C., was slowly over 10 min added n-BuLi (5 mmol) and the reaction was stirred at −78° C. for 1 h. To this was then added trimethyl borate (2.2 mL, 20 mmol) and the reaction was allowed to warm to r.t. with stirring overnight. The reaction was quenched with HCl (2M aq.) and the organics extracted into EtOAc. Recrystallisation from DCM/hexane followed by washing with Et₂O gave the title compound as white powder: LC/MS (APCI) m/z 164.76 (M−H)⁻; HPLC tᵣ=1.27 min (>99%) 90% MeCN in H₂O.

### 1-(4′-Methoxy-2′-methyl-biphenyl-4-yl)-ethanone 90

A mixture of 2-methyl-4-methoxyphenyl boronic acid 89 (0.083 g, 0.5 mmol), 4-bromoacetophenone (0.10 g, 0.5 mmol), K₂CO₃ (0.138 g, 1 mmol), Bu₄NBr (0.161 g, 0.5 mmol) and Pd(OAc)₂ (catalytic) was heated in the microwave at 150° C. for 10 min. Water (10 mL) was added and the organics extracted into EtOAc (20 mL). The product was isolated as the third fraction by flash chromatography (20 g, Flashmaster) using an elution gradient of hexane to 5% to 10% EtOAc in hexane. Yield 0.087 g, 72%. ¹H NMR δ (270 MHz, CDCl₃) 2.26 (3H, s), 2.63 (3H, s), 3.83 (3H, s), 6.78-6.82 (2H, m), 7.15 (1H, d, J=8.4 Hz), 7.39 (2H, d, J=8.4 Hz), 7.98 2H, d, J=8.4 Hz); LC/MS (AP+) m/z 241.29 (M+H)⁺; HPLC tr=2.42 min (>99%) 90% MeCN in H₂O.

### 1-(4′-Hydroxy-2′-methyl-biphenyl-4-yl)-ethanone 91

To a stirred solution of 1-(4′-methoxy-2′-methyl-biphenyl-4-yl)-ethanone 90 (55 mg, 0.23 mmol) in dry DCM (2 mL), cooled to −78° C., was added slowly BBr₃ (0.7 mL of 1M, 0.7 mmol) and the reaction was allowed to warm to r.t. with stirring over 20 h. Water was added and the organics extracted into DCM. An insoluble precipitate formed which was added to the extracts and the mixture was columned using DCM to 5% MeOH. No separation was achieved therefore the mixture was dissolved in DCM and precipitated using hexane. A further column using hexane to 30% EtOAc in hexane resulted in some separation of the product with 91% purity in 13% yield: ¹H NMR δ (270 MHz, CDCl₃) 2.23 (3H, s), 2.64 (3H, s), 5.03 (1H, bs), 6.72-6.78 (2H, m), 7.10 (1H, d, J=8.1 Hz), 7.39 (2H, d, J=8.4 Hz), 7.98 (2H, d, J=8.4 Hz); LC/MS (AP−) m/z 224.92 (M−H)⁻; HPLC tr=1.72 min (>91%) 90% MeCN in H₂O.

### 3-Ethyl-4′-methoxy-biphenyl 92

To a mixture of 1-bromo-3-ethyl benzene (0.185 g, 1 mmol), 4-methoxyphenyl boronic acid (0.182 g, 1.2 mmol), K₂CO₃ (0.276 g, 2 mmol) and Bu₄NBr (0.322 g, 1 mmol) in EtOH (1.5 mL) and water (3.5 mL) was added Pd(OAc)₂ (catalytic) and the reaction was heated in the microwave at 150° C. for 10 min. Water (10 mL) was added and the organics extracted into EtOAc and concentrated under reduced pressure. The product was purified by flash chromatography (20 g, Flashmaster) using hexane to 20% EtOAc in hexane followed by recrystallisation from DCM/hexane. Yield 0.16 g, 75%. ¹H NMR δ (270 MHz, CDCl₃) 1.36 (3H, t, J=7.7 Hz), 2.78 (2H, q, J=7.7 Hz), 3.89 (3H, s), 7.04 (2H, d, J=8.9 Hz), 7.23 (1H, d, J=5.1 Hz), 7.38-7.47 (3H, m), 7.60 (2H, d, J=8.9 Hz); HPLC tr=2.35 min (>97%) 90% MeCN in H₂O.

### 1-(3-Ethyl-4′-methoxy-biphenyl-4-yl)-ethanone 93

To a stirred solution of 3-ethyl-4′-methoxy-biphenyl 92 (0.16 g, 0.75 mmol) in 1,2-dichloroethane (4 mL) was added Ac₂O (0.2 mL) and the solution was cooled in an ice/acetone bath. To this was then added AlCl₃ (0.48 g) and the reaction was allowed to warm to rt with stirring over 24 h. The reaction was quenched with HCl (2M, aq) and the organics extracted into DCM. Attempts to separate the products by chromatography (hexane to 20% EtOAc) and recrystallisation (Et₂O/hexane) failed therefore the mixture was demethylated to give 94 and 95 as described.

### 1-(3-Ethyl-4′-hydroxy-biphenyl-4-yl)-ethanone 94

To a stirred solution containing 1-(3-ethyl-4′-methoxy-biphenyl-4-yl)-ethanone 93 and 1-(3′-ethyl-4-methoxy-biphenyl-3-yl)-ethanone (0.14 g, 0.55 mmol, mixture) in dry DCM (2 mL), cooled to −78° C., was added BBr₃ (1.5 mL of 1M, 1.5 mmol) and the reaction was allowed to warm to rt with stirring over 20 h. Water was added and the organics extracted into DCM and concentrated under reduced pressure. The mixture was columned using hexane to 20% EtOAc in hexane to give the title compound as the second main fraction; ¹H NMR δ (270 MHz, CDCl₃) 1.25 (3H, t, J=7.4 Hz), 2.62 (3H, s), 2.97 (2H, q, J=7.4 Hz), 6.94 (2H, d, J=8.7 Hz), 7.38-7.45 (2H, m), 7.51 (2H, d, J=8.6 Hz), 7.73 (1H, d, J=7.7 Hz); LC/MS (ES−) m/z 239.03 (M−H)⁻; HPLC tr=1.80 min (>95%) 90% MeCN in H₂O.

The first fraction was found to be:

### 1-(3′-Ethyl-4-hydroxy-biphenyl-3-yl)-ethanone 95

¹H NMR δ (270 MHz, CDCl₃) 1.30 (3H, t, J=7.4 Hz), 2.70 (3H, s), 2.71 (2H, q, J=7.4 Hz), 7.06 (1H, d, J=8.7 Hz), 7.19-7.23 (1H, m), 7.33-7.40 (3H, m), 7.71 (1H, dd, J=8.7, 2.2 Hz), 7.91 (1H, d, J=2.2 Hz), 12.3 (˜1H, s); LC/MS (ES−) m/z 239.03 (M−H); HPLC tr=3.06 min (>99%) 90% MeCN in H₂O.

### 4-(4′-Methoxy-3-ethyl-biphenyl-4-yl)-4-oxo-butyric Acid Methyl Ester 96

To a stirred solution of 3-ethyl-4′-methoxy-biphenyl 92 (0.175 g, 0.82 mmol) and succinic anhydride (1 mmol) in 1,2-dichloroethane (5 mL), cooled to −10° C. (ice/acetone bath), was added AIC₃ (0.40 g, 3 mmol) and the reaction was allowed to warm to rt with stirring over 93 h. The reaction was quenched with HCl (2M, aq) and the organics extracted into DCM. Purification by flash chromatography using DCM to 5% MeOH in DCM gave the title compound as the third fraction. LC/MS (ES+) m/z 349 (M+Na)⁺; HRMS calcd for C₂₀H₂₃O₄ 327.1591 found (M+H)⁺ 327.1600 m/z; HPLC tr=5.59 min (>94%) 90% MeCN in H₂O.

### 4-(4′-Methoxy-3-ethyl-biphenyl-4-yl)-4-oxo-butyric Acid 97

To a stirred solution of 4-(4′-methoxy-3-ethyl-biphenyl-4-yl)-4-oxo-butyric acid methyl ester 96 (0.044 g, 0.13 mmol) in THF (2 mL) was added aqueous NaOH (0.027 g in 1 mL) and the reaction was stirred at r.t. for 19 h. Water (10 mL) was added but no precipitate formed therefore the organics were extracted into DCM and EtOAc, adding salt to aid separation of the layers. The extracts were combined and concentrated under reduced pressure to give white powder: ¹H NMR δ (270 MHz, acetone-d₆) 1.21 (3H, t, J=7.4 Hz), 1.27 (bs, 1H), 2.69-2.74 (2H, m), 2.88 (2H, q, J=7.4 Hz), 3.21-3.26 (2H, m), 3.84 (3H, s), 7.03 (2H, d, J=8.8 Hz), 7.52-7.56 (2H, m), 7.66 (2H, d, J=8.8 Hz), 7.86 (1H, d, J=8.0 Hz); LC/MS (ES−) m/z 311.16 (M−H)⁻; HPLC tr=3.19 min (>99%) 70% MeCN in H₂O.

### 4-(4′-Methoxy-3-ethyl-biphenyl)-4-oxo-N-pyridin-3-ylmethyl-butyramide 98

To a stirred suspension of 4-(4′-methoxy-3-ethyl-biphenyl-4-yl)-4-oxo-butyric acid 97 (0.1 mmol) in dry DCM (5 mL) was added 3-aminomethylpyridine (30 μL) and the solution was cooled (ice/acetone bath). To this was then added EDCI (58 mg, 0.3 mmol) and the reaction was allowed to warn slowly to r.t. overnight. The reaction was quenched with bicarb. and the DCM layer was separated and concentrated under reduced pressure. Purification by flash chromatography (10 g, Flashmaster) using DCM to 5% MeOH in DCM gave the product as the main fraction. Yield 33 mg, 82%: ¹H NMR δ (270 MHz, CDCl₃) 1.20 (3H, t, J=7.4 Hz), 2.61-2.66 (2H, m), 2.86 (2H, q, J=7.4 Hz), 3.28-3.33 (2H, m), 3.83 (3H, s), 4.44 (2H, d, J=5.8 Hz), 6.84 (1H, bs, NH), 6.97 (2H, d, J=8.8 Hz), 7.20-7.25 (1H, m), 7.39-7.43 (2H, m), 7.53 (2H, d, J=8.6 Hz), 7.64 (1H, d, J=8.0 Hz), 7.74 (1H, d, J=8.6 Hz), 8.45-8.49 (2H, m); HPLC tr=2.08 min (>99%) 90% MeCN in H₂O; LC/MS (ES+) m/z 403.12 (M+H)⁺; ES-HRMS calcd for C₂₅H₂₇N₂O₃ 403.2016 found (M+H)⁺ 403.2030 m/z.

### 4-(4′-Hydroxy-3-ethyl-biphenyl)-4-oxo-N-pyridin-3-ylmethyl-butyramide 99

To a stirred solution of 4-(4′-methoxy-3-ethyl-biphenyl)-4-oxo-N-pyridin-3-ylmethyl-butyramide 98 (0.016 g, 0.04 mmol) in dry DCM (2 mL), cooled to −78° C., was added BBr₃ (0.12 mL of 1M, 0.12 mmol) and the reaction was allowed to warm to r.t. with stirring over 19 h. Water was added and the organics extracted into DCM. The mixture was concentrated and columned using DCM 5% MeOH in DCM to give the product in 62% yield: LC/MS (ES−) m/z 387.12 (M−H)⁻; HPLC tr=1.58 min (>95%) 90% MeCN in H₂O.

### Trifluoro-methanesulphonic Acid 5-oxo-5,6,7,8-tetrahydro-naphthalen-2-yl Ester 100

To a solution of 6-hydroxytetralone (2.4 g, 14.8 mmol) in dry DCM (50 mL), cooled to −10° C., was added anhydrous pyridine (1.7 mL, 21 mmol) followed by trifluoro-methanesulphonic anhydride (5.0 g) and the reaction was allowed to warm to r.t. with stirring over 16 h. The reaction was quenched with sat. aq. bicarb. and the organic layer separated, washed with water and concentrated under reduced pressure. Purification by flash chromatography using an elution gradient of hexane to 20% EtOAc in hexane gave the title compound in 80% yield: R_{f} 0.55 (20% EtOAc in hexane); ¹H NMR δ (270 MHz, CDCl₃) 2.11-2.23 (2H, m), 2.64-2.69 (2H, m), 2.98-3.02 (2H, m), 7.16-7.20 (2H, m), 8.11 (1H, d, J=9.3 Hz)

### 6-(4-Methoxy-phenyl)-3,4-dihydro-2H-naphthalen-1-one 101

To a mixture of trifluoro-methanesulphonic acid 5-oxo-5,6,7,8-tetrahydro-naphthalen-2-yl ester 100 (0.147 g, 0.5 mmol), 4-methoxyphenyl boronic acid (0.114 g, 0.75 mmol), K₂CO₃ (0.172 g, 1.25 mmol) and Bu₄NBr (0.161 g, 0.5 mmol) in EtOH (1.5 mL) and water (3.5 mL) was added Pd(OAc)₂ (catalytic) and the reaction was microwaved at 150° C. for 10 min. Water (5 mL) was added and the organics extracted into EtOAc and concentrated under reduced pressure. Purification by flash chromatography using hexane to 20% EtOAc in hexane gave the title compound as the second fraction 73 mg, 58%: ¹H NMR δ (270 MHz, CDCl₃) 2.09-2.19 (2H, m), 2.63-2.68 (2H, m), 2.97-3.01 (2H, m), 3.84 (3H, s), 6.97 (2H, d, J=8.7 Hz), 7.40 (1H, s), 7.48 (1H, dd, J=8.2, 1.7 Hz), 7.55 (2H, d, J=8.7 Hz), 8.06 (1H, d, J=8.2 Hz); LC/MS (ES+) m/z 252.95 (M+H)⁺; HPLC tr=2.38 min (>99%) 90% MeCN in H₂O.

### 6-(4-Hydroxy-phenyl)-3,4-dihydro-2H-naphthalen-1-one 102

To a stirred solution of 6-(4-methoxy-phenyl)-3,4-dihydro-2H-naphthalen-1-one 101 (0,070 g, 0.28 mmol) in dry DCM (5 mL), cooled to −78° C. (dry ice/acetone) was added slowly BBr₃ (0.8 mL of 1M, 0.8 mmol) and the reaction was allowed to warm slowly to r.t. with stirring over 18 h. Water was added and the organic layer separated. Some precipitate formed which was collected and added to the DCM layer before flash chromatography using DCM to 5% MeOH in DCM gave the product not fully separated from other fractions. The mixture was columned again using hexane to 30% EtOAc in hexane to give different fractions with approximately the same R_{f}. The first of these was found to be product; ¹H NMR δ (270 MHz, CDCl₃) 2.03-2.08 (2H, m), 2.57-2.62 (2H, m), 2.97-3.01 (2H, 6.86 (1H, d, J=8.7 Hz), 7.55-7.60 (4H, m), 7.88 (1H, d, J=8.9 Hz); 9.72 (1H, bs); LC/MS (ES−) m/z 236.95 (M−H)⁻; HPLC tr=1.81 min (>95%) 90% MeCN in H₂O; ES-HRMS calcd for C₁₆H₁₅O₂ 239.1067 found (M+H)⁺ 239.1065 m/z.

### Ethyl 2-(1,2,3,4-tetrahydro-6-(4-methoxyphenyl)-1-oxonaphthalen-2-yl)acetate 103

To a stirred solution of 6-(4-methoxy-phenyl)-3,4-dihydro-2H-naphthalen-1-one 101 (0.092 g, 0.36 mmol) in dry THF (10 mL), cooled to −78° C., was added slowly drop-wise LDA (0.4 mL of 1.8 M, 0.7 mmol) and the reaction was stirred at −78° C. for 1 h. To this was then added slowly drop-wise ethyl bromoacetate (1.4 mL, 1.3 mmol) and the reaction was allowed to warm slowly to rt with stirring over 17 h. Water (10 mL) was added and the organics extracted into Et₂O. The extracts were combined and concentrated under reduced pressure to give a liquid substance. Addition of hexane/DCM to this gave beige powder. LC/MS (ES+) m/z 361.17 (M+Na)⁺; HPLC tr=2.80 min (>94%) 90% MeCN in H₂O.

### 2-(1,2,3,4-Tetrahydro-6-(4-methoxyphenyl)-1-oxonaphthalen-2-yl)acetic Acid 104

To a stirred solution of ethyl 2-(1,2,3,4-tetrahydro-6-(4-methoxyphenyl)-1-oxonaphthalen-2-yl)acetate 103 (34 mg, 0.1 mmol) in THF (1 mL) was added NaOH aq. (8 mg, 0.2 mmol, in 1 mL) and the reaction was stirred at rt for 18 h. Water was added and the aqueous mixture was washed with EtOAc. The aqueous layer was then acidified and the remaining organics extracted into EtOAc and concentrated under reduced pressure to give beige powder: HPLC tr=1.51 min (>92%) 90% MeCN in H₂O; ES-HRMS calcd for C₁₉H₁₉O₄ 311.1278 found (M+H)⁺ 311.1273 m/z.

### 6-(3-Ethyl-4-methoxyphenyl)-3,4-dihydro-2H-naphthalen-1-one 105

To a mixture of 3-ethyl-4-methoxyphenylboronic acid 6 (0.146 g, 0.81 mmol), trifluoro-methanesulphonic acid 5-oxo-5,6,7,8-tetrahydro-naphthalen-2-yl ester 100 (0.213 g, 0.72 mmol), K₂CO₃ (0.249 g, 1.8 mmol) and Bu₄NBr (0.232 g, 0.72 mmol) in EtOH (1.5 mL) and water (3.5 mL) was added Pd(OAc)₂ (catalytic) and the reaction was microwaved at 150° C. for 10 min. To the mixture was added water and the organics extracted into EtOAc. The organic layer was concentrated under reduced pressure and the product isolated by flash chromatography using hexane to 20% EtOAc: ¹H NMR δ (270 MHz, CDCl₃) 1.24 (3H, t, J=7.4 Hz), 2.11-2.20 (2H, m), 2.64-2.74 (4H, m), 2.98-3.03 (2H, m), 3.87 (3H, s), 6.91 (1H, d, J=8.9 Hz), 7.42-7.52 (4H, m), 8.07 (1H, d, J=8.2 Hz); LC/MS (ES+) m/z 281.11 (M+H)⁺; HPLC tr=3.40 min (>97%) 90% MeCN in H₂O; ES-HRMS calcd for C₁₉H₂₁O₂ 281.1536 found (M+H)⁺ 281.1535 m/z.

### 6-(3-Ethyl-4-hydroxyphenyl)-3,4-dihydro-2H-naphthalen-1-one 106

Procedure as for 6-(4-hydroxy-phenyl)-3,4-dihydro-2H-naphthalen-1-one 102, using 30 mg of 6-(3-ethyl-4-methoxyphenyl)-3,4-dihydro-2H-naphthalen-1-one 105. Water was added and the organic layer separated. Columned as for 102 then washed with MeOH: ¹H NMR δ (270 MHz, CDCl₃) 1.23 (3H, t, J=7.5 Hz), 2.12-2.19 (2H, m), 2.63-2.71 (4H, m), 3.02-3.06 (2H, m), 6.83 (1H, d, J=8.2 Hz), 7.34-7.37 (1H, m), 7.42 (1H, d, J=1.5 Hz), 7.51-7.54 (4H, m), 7.97 (1H, d, J=8.7 Hz); LC/MS (ES−) m/z 265.36 (M−H)⁻; ES-HRMS calcd for C₁₈H₁₉O₂ 267.1380 found (M+H)⁺ 267.1379 m/z.

### 3-Iodo-biphenyl-4-ol 107

To a stirred solution of 4-phenyl phenol (3.15 g, 18.5 mmol), sodium iodide (2.77 g, 18 5 mmol) and NaOH (0.74 g, 18.5 mmol) in MeOH (50 mL), cooled to <0° C. (ice/acetone) was added slowly drop-wise (over 1 h) sodium hypochlorite (aq., 13.8 g of 10-13% avg. C1) and the reaction was stirred for a further 1.5 h at 0-2° C. To this was then added sodium thiosulphate (20 mL of 10% aq) and the mixture was neutralised with 2M HCl. The resulting precipitate was collected by filtration and washed with water: ¹H NMR δ (270 MHz, acetone-d₆) 7.04 (1H, d, J=8.4 Hz), 7.28-7.34 (1H, m), 7.39-7.45 (2H, m), 7.52 (1H, dd, J=8.4, 2.2 Hz), 7.55-7.60 (2H, m), 7.99 (1H, d, J=2.2 Hz); LC/MS (ES−) m/z 294.97 (M−H)⁻; HPLC tr=2.19 min (>90%) 90% MeCN in H₂O.

### 3-Iodo-4-methoxy-biphenyl 108

To a stirred solution of 3-iodo-biphenyl-4-ol 107 (0.74 g, 2.5 mmol) in dry DMF (5 mL) was added K₂CO₃ (7.5 mmol) and MeI (0.19 mL, 3 mmol) and the reaction was stirred at rt for 90 h. Water was added and the organics extracted into Et₂O. The organic layer was concentrated under reduced pressure and the product recrystallised from DCM/hexane. 25% yield initially: ¹H NMR δ (270 MHz, CDCl₃) 3.91 (3H, s), 6.88 (1H, d, J=8.5 Hz), 7.28-7.34 (1H, m), 7.37-7.44 (2H, m), 7.48-7.55 (3H, m), 8.00 (1H, d, J=2.2 Hz); HPLC tr=3.22 min (>96%) 90% MeCN in H₂O.

### 4-Methoxy-3-phenylethynyl-biphenyl 109

To a stirred solution of 3-iodo-4-methoxy-biphenyl 108 (0.154 g, 0.5 mmol) in THF (5 mL) was added NEt₃ (1.5 mL) and phenylacetylene (66 μL, 0.6 mmol) and the solution was degassed by bubbling N₂ through for 10 min. To this was then added PdCl₂(PPh₃)₂ (catalytic) and CuI (catalytic) and stirring under N₂ was continued for a further 17 h. Water (10 mL) was added and the organics extracted into Et₂O. Purification by flash chromatography using hexane to 10% EtOAc in hexane, follwed by recrystallisation from DCM/hexane gave white needles, 0.046 g, 32% yield: ¹H NMR δ (270 MHz, CDCl₃) 3.95 (3H, s), 6.97 (1H, d, J=8.4 Hz), 7.30-7.63 (1H, m), 7.79 (1H, d, J=2.5 Hz); HPLC tr=3.81 min (>99%) 90% MeCN in H₂O.

### 4-Methoxy-3-phenethyl-biphenyl 110

A solution of 4-methoxy-3-phenylethynyl-biphenyl 109 (46 mg, 0.16 mmol) in THF (3 mL) and MeOH (3 mL) was heated to reflux and cooled under N₂. Pd/C (5% wt., catalytic) was added and the degassing process was repeated before H₂ gas was passed over the reaction. The reaction was stirred under a H₂ blanket for 20 h. The mixture was filtered through celite (washed through with EtOAc) and the filtrate concentrated under reduced pressure: ¹H NMR δ (270 MHz, CDCl₃) 2.93-3.03 (4H, m), 3.89 (3H, s), 6.95 (1H, d, J=8.4 Hz), 7.22-7.35 (7H, m), 7.40-7.47 (3H, m), 7.52-7.56 (2H, m); HPLC tr=4.32 min (>98%) 90% MeCN in H₂O.

### 1-(4′-Methoxy-3′-phenethyl-biphenyl-4-yl)-ethanone 111

To a stirred solution of 4-methoxy-3-phenethyl-biphenyl 110 (0.16 mmol) in dry DCM (1 mL) was added Ac₂O (19 mL, 0.2 mmol) and the mixture was cooled to −5° C. (ice/acetone bath). To this was then added AlCl₃ (43 mg, 0.32 mmol) and the reaction was allowed to warm to rt with stirring over 18 h. The reaction was quenched with HCl (2M, aq) and the organics extracted into DCM. Purification by flash chromatography using hexane to 20% EtOAc in hexane gave the product as the second fraction in 47% yield: ¹H NMR δ (270 MHz, CDCl₃) 2.62 (3H, s), 2.88-3.86 (4H, m), 3.88 (3H, s), 6.94 (1H, d, J=8.4 Hz), 7.18-7.33 (6H, m), 7.46 (1H, dd, J=8.4, 2.5 Hz), 7.58 (2H, d, J=8.4 Hz), 7.99 (2H, d, J=8.4 Hz); LC/MS (ES+) m/z 353.36 (M+Na)⁺; HPLC tr=3.41 min (>97%) 90% MeCN in H₂O.

### 6-(3-Benzyloxy-phenyl)-3,4-dihydro-2H-naphthalen-1-one 112

To a mixture of 3-benzyloxyphenylboronic acid (0.217 g, 0.95 mmol), trifluoro-methanesulphonic acid 5-oxo-5,6,7,8-tetrahydro-naphthalen-2-yl ester 100 (0.187 g, 0.64 mmol), K₂CO₃ (0.22 g, 1.6 mmol) and Bu₄NBr (0.205 g, 0.64 mmol) in EtOH (2 mL) and water (4.5 mL) was added Pd(OAc)₂ (catalytic) and the reaction was microwaved at 150° C. for 20 min. To the mixture was added water and the organics extracted into EtOAc. The organic layer was concentrated under reduced pressure and the product isolated in 15% yield by flash chromatography using hexane to 20% EtOAc: LC/MS (ES+) m/z 328.98 (M+H)⁺; HPLC tr=3.49 min (>90%) 90% MeCN in H₂O.

### 6-(3-Methoxy-phenyl)-3,4-dihydro-2H-naphthalen-1-one 113

From debenzylation of 6-(3-benzyloxy-phenyl)-3,4-dihydro-2H-naphthalen-1-one 112. To a stirred solution of 6-(3-benzyloxy-phenyl)-3,4-dihydro-2H-naphthalen-1-one 112 (0.19 mmol) in dry DCM (3 mL), cooled to −78° C., was added drop-wise BBr₃ (0.4 mL of 1M) and the reaction was allowed to warm slowly to rt over 24 h. The reaction was quenched with water and the organic layer separated and concentrated under reduced pressure. Flash chromatography using DCM to 5% MeOH didn't give good separation therefore recrystallisation was tried from DCM/hexane to give cream coloured powder. ¹H NMR δ (270 MHz, CDCl₃) 2.13-2.51 (2H, m), 2.64-2.69 (2H, m), 2.98-3.03 (2H, m), 3.85 (3H, s), 6.98 (2H, d, J=8.8 Hz), 7.42 (1H, bs/d), 7.49 (1H, dd, J=8.0 Hz), 7.56 (2H, d, J=8.8 Hz), 8.07 (1H, d, J=8.0 Hz); LC/MS (ES+) m/z 275.06 (M+Na)⁺; HPLC tr=2.33 min (>99%) 90% MeCN in H₂O.

### 6-(3-Hydroxy-phenyl)-3,4-dihydro-2H-naphthalen-1-one 114

To a solution of 6-(3-benzyloxy-phenyl)-3,4-dihydro-2H-naphthalen-1-one 112 (0.09 mmol) in THF (1.2 mL) was added conc. HCl (0.6 mL) and the solution was heated to reflux for 17 h. The reaction was allowed to cool, neutralised with bicarb. and the organics extracted into EtOAc. Precipitation from DCM/hexane gave beige powder. LC/MS (ES−) m/z 236.89 (M−H)⁻; HPLC tr=2.26 min (>99%) 70% MeCN in H₂O.

### 1-(4-Methoxyphenoxy)benzene 115

To a stirred solution of 4-phenoxyphenol (3.72 g, 20 mmol) in dry DMF (40 mL) was added K₂CO₃ (8.29 g, 60 mmol) followed by methyl iodide (1.4 mL, 22 mmol) and the reaction was stirred at rt for 17 h. The mixture was diluted with EtOAc (50 mL) and the solution washed with water (3×100 mL). The organic layer was concentrated under reduced pressure to give pale brown oil. Purification by flash chromatography (20 g column, Flashmaster II) using a gradient elution of hexane to 5% and then 10% EtOAc in hexane yielded the title compound (3.75 g, 94%): R_{f}: 0.5 (EtOAc:hexane 1:2); ¹H NMR δ (270 MHz, CDCl₃) 3.82 (3H, s, OCH₃), 6.93 (2H, d, J=9.1 Hz), 6.99-7.11 (5H), 7.31-7.37 (2H); LC/MS (APCI) m/z 200.53 (M⁺); HPLC tᵣ=2.68 min (>98%) 80% MeCN in H₂O.

### 4-(4-(4-Methoxyphenoxy)phenyl)-4-oxobutanoic Acid 116

To a stirred suspension of succinic anhydride (0.175 g, 1 mmol) in 1-(4-methoxyphenoxy)benzene 115 (2.17 g, 10.8 mmol) and DCM (1 mL), cooled to −5° C. (ice/acetone bath) was added aluminium chloride (0.468 g, 3.5 mmol) and the reaction was allowed to warm slowly to r.t. with stirring for 18 h. The mixture was poured into ice cold HCl (18% aq, 5 mL) and the mixture was stirred for a further 30 min while being allowed to warm to r.t. The resulting white powder was collected by filtration and washed with water. Recrystallisation from DCM/hexane yielded the title compound (0.52 g, 99%): ¹H NMR δ (400 MHz, CDCl₃) 2.70-2.73 (2H), 3.28-3.31 (2H), 3.84 (3H, s, OCH₃), 6.96-7.07 (6H, m), 8.01 (2H, d, J=9.2 Hz); ¹³C NMR δ (100 MHz, CDCl₃) 27.6, 32.7, 54.7, 114.8, 115.9, 121.4, 130.1, 130.8, 148.6, 157.0, 163.2, 175.3, 197.7; LC/MS (APCI) m/z 299.03 (M−H)⁻; HPLC tᵣ=2.20 min (>96%) 60% MeCN in H₂O.

### 4-(4-(4-Methoxyphenoxy)phenyl)-4-oxo-N-((pyridin-3-yl)methyl)butanamide 117

To a stirred solution of 4-(4-(4-methoxyphenoxy)phenyl)-4-oxobutanoic acid 116 (0.15 g, 0.5 mmol) in dry DCM (10 mL) was added DMAP (catalytic), EDCI (1.5 mmol) and NEt₃ (0.1 mL) and the reaction was stirred at rt for 10 min before addition of 3-aminomethylpyridine (0.1 mL). Stirring was continued for a further 24 h before the reaction was quenched with sat. aq. bicarb. and the organic layer separated and concentrated under reduced pressure. Purification by flash chromatography (20 g column, Flashmaster II) using a gradient elution of DCM to 10% MeOH in DCM yielded the title compound (0.137 g, 70%): ¹H NMR δ (400 MHz, CDCl₃) 2.68-2.71 (2H), 3.37-3.40 (2H), 3.86 (3H, s, OCH₃), 4.50 (2H, d, J=5.9 Hz), 6.33 (1H, bs, NH), 6.95-6.98 (4H, m), 7.05 (2H, d, J=9.4 Hz), 7.27-7.30 (1H, m), 7.65-7.68 (1H, m), 7.97 (2H, d, J=9.0 Hz), 8.54-8.57 (2H, m); ¹³C NMR δ (100 MHz, CDCl₃) 30.3, 33.8, 41.1, 55.7, 115.1, 116.4, 121.8, 123.6, 130.4, 130.6, 134.0, 135.5, 148.4, 148.9, 149.1, 156.8, 163.2, 172.4, 197.6; LC/MS (APCI) m/z 389.21 (M−H)⁻; HRMS (FAB⁺) calcd. for C₂₃H₂₃N₂O₄ (M+H)⁺ 391.1658, found 391.1662; HPLC tᵣ=3.39 min (>97%) 40% MeCN in H₂O.

### 4-(4-(4-Hydroxyphenoxy)phenyl)-4-oxo-N-((pyridin-3-yl)methyl)butanamide 118

To a stirred solution of 4-(4-(4-methoxyphenoxy)phenyl)-4-oxo-N-((pyridin-3-yl)methyl)butanamide 117 (0.100 g, 0.26 mmol) in dry DCM (5 mL), cooled to −78° C. (dry ice/acetone bath) was added slowly BBr₃ (1.3 mL of a 1M solution in DCM (1.3 mmol) and the reaction was allowed to warm slowly to r.t. with stirring over 6 h. The reaction was quenched with water and the resulting cream coloured powder was colleted by filtration and washed with water. Recrystallisation from MeOH/water yielded the title compound (0.045 g, 46%): mp>191° C. (decomposed); ¹H NMR δ (400 MHz, DMSO-d₆) 2.56-2.59 (2H, m), 3.24-3.27 (2H, m), 4.47 (2H, d, J=5.9 Hz), 6.85 (2H, d, J=9.0 Hz), 6.95-6.99 (4H, m), 7.98 (2H, d, J=9.0 Hz), 8.00-8.04 (1H, m), 8.42 (1H, d, J=8.2 Hz), 8.72-8.74 (1H, m, NH), 8.80 (1H, s), 8.82 (1H, d, J=5.5 Hz); ¹³C NMR δ (100 MHz, DMSO-d₆) 29.6, 33.5, 116.4, 116.9, 122.2, 127.0, 130.8, 131.1, 139.9, 141.6, 141.7, 144.1, 146.9, 155.1, 163.1, 172.7, 197.9; LC/MS (APCI) m/z 375.17 (M−H)⁻; HRMS (FAB⁺) calcd. for C₂₂H₂₁N₂O₄ (M+H)⁺ 377.1501, found 377.1509; HPLC tᵣ=2.05 min (>95%) 60% MeCN in H₂O.

### 4-(4-(4-Methoxyphenoxy)phenyl)-4-oxo-N-(2-(pyridin-3-yl)ethyl)butanamide 119

To a stirred solution of 4-(4-(4-methoxyphenoxy)phenyl)-4-oxobutanoic acid 116 (0.10 g, 0.33 mmol) in dry DCM (2.5 mL) was added DMAP (catalytic) and 3-(2-aminoethyl)pyridine (0.1 mL) before the solution was cooled to 0° C. (ice bath). To this was then added EDCI (0.192 g, 1 mmol) and the reaction was allowed to warm to r.t. with stirring for 18 h. To this was added sat. aq. NaHCO₃ (10 mL) followed by DCM (10 mL) before the organic layer was separated and concentrated under reduced pressure. Purification by flash chromatography using a gradient elution of DCM to 10% MeOH in DCM yielded the title compound (0.078 g, 58%); ¹H NMR δ (400 MHz, CDCl₃) 2.57 (2H, t, J=6.6 Hz), 2.82 (2H, t, J=7.0 Hz), 3.28 (2H, t, J=6.6 Hz), 3.48-3.53 (2H, m), 3.82 (3H, s, OCH₃), 6.52-6.55 (1H, m, NH), 6.91-6.95 (4H, m), 7.01 (2H, d, J=9.4 Hz), 7.18-7.22 (1H, m), 7.52-7.55 (1H, m), 7.92 (2H, d, J=9.0 Hz), 8.41-8.44 (2H, m); ¹³C NMR δ (100 MHz, CDCl₃) 30.2, 32.9, 33.7, 40.5, 55.7, 115.1, 116.4, 121.7, 123.5, 130.3, 130.7, 134.6, 136.4, 147.8, 148.4, 150.1, 156.7, 163.1, 172.4, 197.6; LC/MS (APCI) m/z 403.32 (M−H)⁻; HRMS (FAB⁺) calcd. for C₂₄H₂₅N₂O₄ (M+H)⁺ 405.1814, found 405.1816; HPLC tᵣ=2.11 min (>99%) 80% MeCN in H₂O.

### 4-(4-(4-Hydroxyphenoxy)phenyl)-4-oxo-N-(2-(pyridin-3-yl)ethyl)butanamide 120

To a solution of 4-(4-(4-methoxyphenoxy)phenyl)-4-oxo-N-(2-(pyridin-3-yl)ethyl)butanamide 119 (0.072 g, 0.18 mmol) in dry DCM (3 mL), cooled to −78° C. (dry ice/acetone bath) was added slowly BBr₃ (0.9 mL of 1M solution in DCM, 0.9 mmol) and the reaction was allowed to warm slowly to r.t. with stirring over 22 h. The reaction was quenched with water (10 mL) and the resulting precipitate was collected by filtration and washed with water. This wet powder was dissolved in MeOH then concentrated under reduced pressure and dried under high vacuum to yield the title compound (0.060 g, 85%); ¹H NMR δ (400 MHz, CDCl₃) 2.54-2.57 (2H, m), 2.95-2.99 (2H, m), 3.26-2.29 (2H, m), 3.50-3.53 (2H, m), 6.84-6.89 (2H, m), 6.94-6.98 (4H, m), 7.65-7.68 (1H, m), 7.98-8.01 (2H, m), 8.12-8.15 (1H, m), 8.55 (2H, dd, J=5.5, 1.2 Hz), 8.65 (1H, d, J=1.5 Hz); ¹³C NMR δ (100 MHz, CDCl₃) 29.3, 32.2, 32.9, 39.6, 115.8, 116.1, 121.5, 125.1, 130.1, 130.5, 137.7, 141.6, 143.5, 146.0, 147.4, 154.6, 163.5, 173.8, 197.8; LC/MS (APCI) m/z 391.35 (M+H)⁺; HRMS (FAB⁺) calcd. for C₂₃H₂₃N₂O₄ (M+H)⁺ 391.1658, found 391.1664; HPLC tᵣ=1.98 min (>94%) 80% MeCN in H₂O.

### 4-(4-(4-Methoxyphenoxy)phenyl)-4-oxo-N-(2-(pyridin-2-yl)ethyl)butanamide 121

Preparation as for 4-(4-(4-methoxyphenoxy)phenyl)-4-oxo-N-(2-(pyridin-3-yl)ethyl)butanamide 119 using 2-(2-aminoethyl)pyridine. Yield 0.120 g, 90%; ¹H NMR δ (400 MHz, CDCl₃) 2.56-2.60 (2H, m), 2.96-2.99 (2H, m), 3.26-3.29 (2H, m), 3.63-3.68 (2H, m), 3.80 (3H, s, OCH₃), 6.84-6.87 (1H, m, NH), 6.90-6.93 (4H, m), 7.00 (2H, d, J=9.4 Hz), 7.09-7.13 (1H, m), 7.15 (1H, d, J=7.8 Hz), 7.57 (1H, dt, J=7.8, 2.0 Hz), 7.92 (2H, d, J=9.0 Hz), 8.48-8.50 (1H, m); ¹³C NMR δ (100 MHz, CDCl₃) δ0.3, 33.4, 37.1, 38.9, 55.6, 115.1, 116.3, 121.6, 121.7, 123.5, 130.3, 130.8, 136.6, 148.4, 149.2, 156.7, 159.5, 163.0, 172.1, 197.5; LC/MS (APCI) m/z 403.32 (M−H)⁻; HRMS (FAB⁺) calcd. for C₂₄H₂₅N₂O₄ (M+H)⁺ 405.1814, found 405.1819; HPLC tᵣ=2.03 min (>99%) 80% MeCN in H₂O.

### 4-(4-(4-Hydroxyphenoxy)phenyl)-4-oxo-N-(2-(pyridin-2-yl)ethyl)butanamide 122

To a solution of 4-(4-(4-methoxyphenoxy)phenyl)-4-oxo-N-(2-(pyridin-2-yl)ethyl)butanamide 121 (0.096 g, 0.24 mmol) in dry DCM (3 mL), cooled to −78° C. (dry ice/acetone bath) was added slowly BBr₃ (1.2 mL of 1M solution in DCM, 1.2 mmol) and the reaction was allowed to warm slowly to r.t. with stirring over 22 h. The reaction was quenched with water (10 mL) and the solution washed with DCM (10 mL). The aqueous layer was neutralised with NaOH (10% aq.) and the resulting white powder collected by filtration and washed with water. Purification by flash chromatography (20 g column, Flashmaster II) using a gradient elution of DCM to 10% MeOH in DCM, followed by recrystallisation from MeOH/water yielded the title compound (0.054 g, 57%); ¹H NMR δ (270 MHz, CDCl₃) 2.53 (2H, t, J=6.7 Hz), 2.93-2.98 (2H, m), 3.24 (2H, t, J=6.7 Hz), 3.49-3.54 (2H, m), 6.80-6.84 (2H, m), 6.90-6.94 (4H, m), 7.22-7.27 (1H, m), 7.34 (1H, d, J=7.7 Hz), 7.74 (1H, dt, J=7.7, 1.7 Hz), 7.95 (2H, d, J=8.9 Hz), 8.44 (1H, d, J=4.9 Hz); ¹³C NMR δ (68 MHz, CDCl₃) 30.1, 33.6, 37.1, 39.2, 116.2, 116.4, 121.8, 121.9, 123.9, 130.3, 137.3, 147.4, 148.7, 154.1, 158.9, 163.4, 173.0, 198.0; LC/MS (APCI) m/z 389.46 (M−H)⁻; HPLC tᵣ=3.76 min (>92%) 80% MeCN in H₂O.

### 1-(4-(4-Methoxyphenoxy)phenyl)ethanone 123

To a stirred solution of 4′-fluoroacetophenone (1.38 g, 10 mmol) and 4-methoxyphenol (1.24 g, 10 mmol) in dry DMA (10 mL) was added K₂CO₃ (1.66 g, 12 mmol) and the suspension was refluxed with vigorous stirring for 8 h. The mixture was allowed to cool, before being diluted with water (10 mL) and the products extracted with CHCl₃ (2×10 mL). The organic layers were combined and concentrated under reduced pressure to give a concentrated solution in DMA. To this was added water followed by a small amount of MeOH and the resulting beige powder was collected by filtration and washed with water. This was recrystallised from EtOH to yield the title compound (1.64 g, 68%): ¹H NMR δ (400 MHz, CDCl₃) 2.60 (3H, s), 3.86 (3H, s), 6.95-6.99 (4H, m), 7.05 (2H, d, J=9.0 Hz), 7.95 (2H, d, J=8.6 Hz); ¹³C NMR δ (100 MHz, CDCl₃) 26.5, 55.9, 115.1, 116.4, 121.7, 130.6, 131.4, 148.5, 156.7, 163.0.

### 3-(4-(4-Methoxyphenoxy)phenyl)-3-oxopropanal 124

To a stirred solution of 1-(4-(4-methoxyphenoxy)phenyl)ethanone 123 (0.242 g, 1 mmol) in toluene (5 mL) was added ethyl formate (0.8 mL) followed by t-BuOK (0.135 g, 1.2 mmol) and the reaction was stirred at rt for 22 h. The mixture was acidified with glacial AcOH and concentrated slightly under reduced pressure before water (10 mL) was added and the products extracted with EtOAc (10 mL). The organic layer was separated and concentrated under reduced pressure and the title compound used without further purification for subsequent reactions.

### 3-(4-(4-Methoxyphenoxy)phenyl)-1H-pyrazole 125

To a stirred solution of 3-(4-(4-methoxyphenoxy)phenyl)-3-oxopropanal 124 (crude, assume ˜0.5 mmol) in EtOH (4 mL) was added hydrazine monohydrate (0.03 mL, 0.6 mmol) and the reaction was heated to reflux for 1 h. The mixture was allowed to cool to rt before being acidified with glacial acetic acid, diluted with water and the resulting white powder collected by filtration and washed with water. The presence of product was confirmed by LCMS but HPLC showed the purity to be only 70%. The product was purified further by flash chromatography (10 g column, Flashmaster II) using a gradient elution of DCM to 10% MeOH in DCM. The product was then used without further purification for the next step. Yield ˜36%; LC/MS (APCI) m/z 267.23 (M+H)⁺; HPLC tᵣ=2.46 min (>88%) 80% MeCN in H₂O.

### 4-(4-(1H-Pyrazol-3-yl)phenoxy)phenol 126

To a stirred solution of 3-(4-(4-methoxyphenoxy)phenyl)-1H-pyrazole 125 (0.18 mmol) in dry DCM (6 mL), cooled to −78° C., was added slowly BBr₃ (0.9 mL of a 1M solution in DCM, 0.9 mmol) and the reaction was allowed to warm slowly to r.t. with stirring over 17 h. The reaction was quenched with water and the resulting yellow powder collected by filtration, washed with water, dissolved in MeOH and the solution concentrated under reduced pressure. The product was purified by flash chromatography (10 g column, Flashmaster II) using a gradient elution of DCM to 10% MeOH in DCM to yield the title compound (0.025 g, 55%); ¹H NMR δ (400 MHz, CDCl₃) 6.61 (1H, bs), 6.83 (2H, d, J=9.0 Hz), 6.93 (2H, d, J=9.0 Hz), 6.96 (2H, d, J=8.6 Hz) 7.66-7.72 (3H, m); LC/MS (APCI) m/z 251.11 (M−H)⁻; HPLC tᵣ=2.03 min (>92%) 80% MeCN in H₂O.

### 5-(4-(4-Methoxyphenoxy)phenyl)isoxazole 127

To a stirred solution of 3-(4-(4-methoxyphenoxy)phenyl)-3-oxopropanal 124 (crude, assume ˜0.5 mmol) in EtOH (4 mL) was added hydroxylamine hydrochloride (0.042 g, 0.6 mmol) and the reaction was heated to reflux for 1 h. The mixture was allowed to cool to r.t. before being acidified with glacial acetic acid, diluted with water and the resulting white powder collected by filtration and washed with water. The presence of product was confirmed by LCMS but HPLC showed the purity to be only 55%. The product was purified by flash chromatography (10 g column, Flashmaster II) using a gradient elution of DCM to 10% MeOH in DCM to yield the title compound (0.045 g, 34%); ¹H NMR δ (270 MHz, CDCl₃) 3.74 (3H, s), 6.34 (1H, s, J=1.7 Hz), 6.84 (2H, d, J=9.2 Hz), 6.92 (2H, d, J=8.9 Hz), 6.94 (2H, d, J=9.2 Hz), 7.64 (2H, d, J=8.9 Hz), 8.18 (1H, d, J=2.0 Hz); LC/MS (APCI) m/z 268.17 (M+H)⁺; HPLC tᵣ=2.91 min (>93%) 80% MeCN in H₂O.

### 4-(4-(Isoxazol-5-yl)phenoxy)phenol 128

To a stirred solution of 5-(4-(4-methoxyphenoxy)phenyl)isoxazole 127 (0.17 mmol) in dry DCM (3 mL), cooled to −78° C., was added slowly BBr₃ (0.8 mL of a 1M solution in DCM, 0.8 mmol) and the reaction was allowed to warm slowly to r.t. with stirring over 17 h. The reaction was quenched with water (5 mL), diluted with DCM (5 mL) and the organic layer was separated and concentrated under reduced pressure to give beige powder. Purification by flash chromatography (10 g column, Flashmaster II) using a gradient elution of DCM to 10% MeOH in DCM followed by crystallisation from MeOH/water yielded the title compound: ¹H NMR δ (270 MHz, CDCl₃) 6.66 (1H, s, J=2.0 Hz), 6.82 (2H, d, J=9.1 Hz), 6.92 (2H, d, J=9.1 Hz), 6.99 (2H, d, J=8.9 Hz), 7.78 (2H, d, J=8.9 Hz), 8.38 (1H, d, J=1.7 Hz); LC/MS (APCI) m/z 252.11 (M⁺−H); HPLC tᵣ=2.25 min (>96%) 80% MeCN in H₂O.

### 1-(4-(4-Hydroxyphenoxy)phenyl)ethanone 129

To a stirred solution of 1-(4-(4-methoxyphenoxy)phenyl)ethanone 123 (0.100 g, 0.4 mmol) in dry DCM (5 mL), cooled to −78° C., was added drop-wise BBr₃ (2.0 mL of a 1M solution in DCM, 2.0 mmol) and the reaction was allowed to warm slowly to rt with stirring overnight. The reaction was quenched with water (10 mL), diluted with DCM (5 mL) and the organic layer was separated and concentrated under reduced pressure to give a beige powder. Purification by flash chromatography (20 g column, vacuum box) using DCM followed by 10% MeOH in DCM as eluents yielded the title compound (0.4 mmol, 100%); mp 159-161° C.; ¹H NMR δ (400 MHz, CDCl₃) 2.61 (3H, s), 6.91 (2H, d, J=9.0 Hz), 6.96-7.00 (4H, m), 7.95 (2H, d, J=9.0 Hz); ¹³C NMR δ (100 MHz, CDCl₃) 26.5, 116.3, 116.6, 121.9, 130.7, 131.2, 148.2, 153.2, 163.2, 197.4; LC/MS (APCI) m/z 226.86 (M−H)⁻; HRMS (FAB⁺) calcd. for C₁₄H₁₂O₃ (M⁺) 228.0786, found 228.0786; HPLC tᵣ=2.17 min (>91%) 60% MeCN in H₂O.

### 4-(4-(2-Aminothiazol-4-yl)phenoxy)phenol 130

To a stirred solution of 1-(4-(4-hydroxyphenoxy)phenyl)ethanone 129 (0.075 g, 0.33 mmol) in EtOH (1.3 mL) was added thiourea (0.075 g, 0.99 mmol) and iodine (0.083 g, 0.33 mmol) and the mixture was heated in an open flask at 180° C. for 2 h (EtOH evaporated). The crude residue was washed with diethyl ether (3×5 mL) before recyrstallisation from MeOH/water. Purification by flash chromatography (5 g column, Flashmaster II) using an elution gradient of DCM to 10% MeOH in DCM yielded the title compound (0.026 g, 28%): LC/MS (APCI) m/z 283.16 (M−H)⁻; HPLC tᵣ=1.80 min (>90%) 80% MeCN in H₂O.

### 4-(4-(4-Methoxyphenoxy)phenyl)-2,2-dimethyl-4-oxobutanoic Acid 131

To a stirred suspension of 2,2-dimethylsuccinic anhydride (0.128 g, 1 mmol) in 1-(4-methoxyphenoxy)benzene 115 (1.10 g, 5.5 mmol), cooled to −5° C., was added AlCl₃ (0.266 g, 2 mmol) and the reaction was allowed to warm to rt with stirring overnight. The mixture was poured into a cooled solution of HCl (5 mL of 18% aq.) and was stirred while being allowed to warm to r.t. The white powder was collected by filtration and recrystallised from DCM/hexane: ¹H NMR δ (270 MHz, CDCl₃) 1.35 (6H, s), 3.26 (2H, s), 3.82 (3H, s), 6.89-7.02 (6H, m), 7.90 (2H, d, J=8.9 Hz); HPLC>94% (Rₜ 3.26, 90% MeCN in H₂O); ES-ve MS (M−H)⁻ 327.41 m/z.

### 1-(4-(4-Fluorophenoxy)phenyl)ethanone 132

To a solution of 4-fluorophenol (0.112 g, 1 mmol) and 4′-fluoroacetophenone (0.138 g, 1 mmol) in MeCN (4 mL) was added 18-crown-6 (catalytic) and K₂CO₃ (0.276 g, 2 mmol) and the reaction was microwaved at 150-160° C. for 15 min. Purification by flash chromatography (10 g column, Flashmaster II) using an elution gradient of hexane to 10% EtOAc in hexane, followed by recrystallisation from MeOH/water yielded the title compound (0.046 g, 20%): ¹H NMR δ (270 MHz, DMSO-d₆) 2.51 (3H, s), 6.98-7.03 (2H, m), 7.14-7.19 (2H, m), 7.25-7.31 (2H, m), 7.92-7.98 (2H, m); LC/MS (APCI) m/z 231.09 (M+H)⁺; HRMS (FAB⁺) calcd. for C₁₄H₁₁O₂F (M⁺) 230.0743, found 230.0744; HPLC tᵣ=2.33 min (>95%) 90% MeCN in H₂O.

### 1-(4-(3,4-Difluorophenoxy)phenyl)ethanone 133

To a solution of 3,4-difluorophenol (0.130 g, 1 mmol) and 4′-fluoroacetophenone (0.138 g, 1 mmol) in dry DMA (2 mL) was added K₂CO₃ (0.276 g, 2 mmol) and the reaction was heated at 90° C. for 18 h. The mixture was allowed to cool before water (5 mL) was added and the organics extracted into EtOAc (2×6 mL). The extracts were combined and concentrated under reduced pressure and the title compound isolated by flash chromatography (20 g column, Flashmaster II) using an elution gradient of hexane to 10% EtOAc in hexane (0.03 g, 12%): ¹H NMR δ (270 MHz, CDCl₃) 2.57 (3H, s), 6.76-6.81 (1H, m), 6.86-6.94 (1H, m), 6.99 (2H, d, J=8.9 Hz), 7.12-7.22 (1H, m), 7.95 (2H, d, J=8.9 Hz); LC/MS (APCI) m/z 249.16 (M+H)⁺; HPLC tᵣ=3.71 min (>99%) 80% MeCN in H₂O.

### 1-(4-(4-Fluoro-3-methylphenoxy)phenyl)ethanone 134

To a solution of 4-fluoro-3-methyl phenol (0.12 mL, 1.1 mmol) and 4′-fluoroacetophenone (0.12 mL, 1 mmol) in dry DMA (2 mL) was added K₂CO₃ (0.276 g, 2 mmol) and the reaction was heated at 90° C. for 6 h. The mixture was allowed to cool before water (10 mL) was added and the organics extracted into EtOAc (2×10 mL). The extracts were combined and concentrated under reduced pressure and the title compound isolated by flash chromatography (10 g column, Flashmaster II) using an elution gradient of hexane to 10% EtOAc in hexane (5 mg, 2%): ¹H NMR δ (270 MHz, CDCl₃) 2.26 (3H, d, J=1.7 Hz), 2.56 (3H, s), 6.81-7.04 (5H, m), 7.92 (2H, d, J=8.9 Hz); LC/MS (APCI) m/z 245.00 (M⁺+H); HPLC tᵣ=4.67 min (>99%) 80% MeCN in H₂O.

### 1-(4-(Pyridin-3-yloxy)phenyl)ethanone 135

To a solution of 3-hydroxypyridine (0.095 g, 1 mmol) and 4′-fluoroacetophenone (0.138 g, 1 mmol) in MeCN (4 mL) was added 18-crown-6 (catalytic) and K₂CO₃ (0.276 g, 2 mmol) and the reaction was microwaved at 160° C. for 20 min. Purification by flash chromatography (10 g column, Flashmaster II) using an elution gradient of hexane to EtOAc yielded the title compound (0.106 g, 50%): mp 45-50° C.; ¹H NMR δ (400 MHz, CD₃OD) 2.62 (3H, s), 7.14 (2H, d, J=8.6 Hz), 7.53 (1H, dd, J=8.2, 4.7 Hz), 7.60-7.63 (1H, m), 8.08 (2H, d, J=8.6 Hz), 8.42-8.44 (2H, m); ¹³C NMR (100 MHz, CD₃OD) δ 25.2, 117.5, 125.0, 127.7, 130.7, 132.7, 141.3, 144.8, 153.0, 161.0, 197.6; LC/MS (APCI) m/z 214.09 (M⁺+H); HRMS (FAB⁺) calcd. for C₁₃H₁₂NO₂ (M+H)⁺ 214.0863, found 214.0863; HPLC tᵣ=2.30 min (>95%) 80% MeCN in H₂O.

### 1-(4-(4-Hydroxyphenylthio)phenyl Ethanone 136

To a stirred suspension of t-BuOK (0.22 g, 2.0 mmol) in dry DMSO (2 mL) was added 4-hydroxythiophenol (0.253 g, 2.0 mmol) and the mixture was cooled in an ice/water bath. To this was then added 4′-fluoroactophenone (0.24 mL, 2.0 mmol) and the reaction was allowed to warm slowly to room temperature over 24 h. Water (10 mL) was added and the resulting cream-coloured powder was collected by filtration and washed with water. Recrystallisation from DCM/MeOH/hexane gave a cream coloured powder which still contained a small amount of 4′-fluoroactophenone. Washing this with DCM and MeOH gave the product. ¹H NMR δ (270 MHz, CD₃OD) 2.52 (3H, s), 6.88 (2H, d, J=8.96 Hz), 7.07 (2H, d, J=8.6 Hz), 7.36 (2H, d, J=8.7 Hz), 7.82 (2H, d, J=8.7 Hz); ¹³C NMR δ (68 MHz, CDCl₃) 25.9, 116.7.119.1, 125.2, 128.6, 133.1, 140.0, 147.9, 158.4, 198.4; LC/MS (APCI) m/z 242.99 (M−H)⁻; HRMS (FAB⁺) calcd. for C₁₄H₁₃O₂S (M+H)⁺ 245.0631, found 245.0626; HPLC tᵣ=2.41 min (>98%) 80% MeCN in H₂O.

### 1-[4-(4-Hydroxy-benzenesulphinyl)-phenyl]-ethanone 137

To a stirred mixture of 1-(4-(4-hydroxyphenylthio)phenyl ethanone 136 (0.089 g, 0.36 mmol) in AcOH (3 mL, glacial) was added H₂O₂ (45 μL of 27.5 wt. % solution, 0.4 mmol) and the reaction was stirred for 24 h. To this was added water (10 mL) and the white powder was collected by filtration and washed with water and shown my ¹H NMR to be a mixture of starting material and product. The title compound was isolated from this mixture by flash chromatography using an elution gradient of DCM to 5% MeOH in DCM, yield 0.039 g, 42%; ¹H NMR δ (270 MHz, CD₃OD) 2.60 (3H, s), 6.90 (2H, d, J=8.9 Hz), 7.53 (2H, d, J=8.6 Hz), 7.72 (2H, d, J=8.6 Hz), 8.09 (2H, d, J=8.4 Hz); LC/MS (APCI) m/z 259.31 (M−H)⁻; HPLC tᵣ=3.24 min (>97%) 90% MeCN in H₂O.

### 3-Ethyl-4-methoxybenzaldehyde 138

A solution of 2-ethyl anisole (1.17 g, 8.6 mmol) in dry DMF (0.7 mL, 8.8 mol) was cooled to −5° C. before POCl₃ (1 mL, 10.7 mmol) was added slowly drop-wise. The resulting suspension was allowed to warm to r.t. over 2 h before being refluxed for 21 h. The reaction was allowed to cool before water (20 mL) was added. Addition of excess NaOH (10% aqueous) resulted in the formation of yellow oil which was extracted into diethyl ether (3×20 mL). The combined extracts were washed with water, brine, dried (MgSO₄) and concentrated under reduced pressure to give yellow oil. Purification by flash chromatography yielded the title compound (0.672 g, 47%): ¹H NMR δ (270 MHz, CDCl₃) 1.20 (3H, t, J=7.5 Hz), 2.66 (2H, q, J=7.5 Hz), 3.90 (3H, s), 6.92 (1H, d, J=8.9 Hz), 7.68-7.72 (2H, m), 9.85 (1H, s); HPLC tᵣ=2.59 min (>84%) 90% MeCN in H₂O.

### 3-Ethyl-4-methoxyphenol 139

To a stirred solution of 3-ethyl-4-methoxybenzaldehyde 138 (0.216 g, 1.3 mmol) in dry DCM (7 mL) was added m-CPBA (0.328 g, 1.9 mmol) and the reaction was heated to reflux for 19 h. The solution was allowed to cool before being washed with sat. aq. NaHCO₃ (3×20 mL) and concentrated under reduced pressure to give an oil. This oil (the crude formate) was dissolved in minimum MeOH (2 mL) and aq. KOH (2 mL of 10%) was added under a N₂ atmosphere. To this was then added EtOAc (20 mL), the organic layer was separated and the aqueous layer washed with EtOAc (20 mL). The organic layers were combined, dried (MgSO₄), concentrated under reduced pressure and dried under high vacuum to yield the title compound (0.185 g, 94%): ¹H NMR δ (270 MHz, CD₃OD) 1.12 (3H, t, J=7.5 Hz), 2.53 (2H, q, J=7.5 Hz), 3.67 (3H, s), 4.92 (1H, bs), 6.54-6.68 (3H, m); LC/MS (APCI) m/z 135.85 (M−OH); HPLC tᵣ=2.30 min (>97%) 80% MeCN in H₂O.

### 1-(4-(3-Ethyl-4-methoxyphenoxy)phenyl)ethanone 140

To a solution of 3-ethyl-4-methoxyphenol 139 (0.38 g, 2.5 mmol) and 4′-fluoroacetophenone (0.138 mL, 1.0 mmol) in MeCN (4 mL) was added K₂CO₃ (0.276 g, 2.0 mmol) and 18-crown-6 (catalytic) the reaction was heated in the microwave at 160° C. for 40 min. The mixture was concentrated under reduced pressure and the product isolated by flash chromatography (20 g, Flashmaster II) using an elution gradient of hexane to 5% then 10% EtOAc in hexane. Yield 0.174 g, 64%; mp=53-56° C.; ¹H NMR δ (270 MHz, CDCl₃) 1.16 (3H, t, J=7.5 Hz), 2.54 (3H, s), 2.62 (2H, q, J=7.5 Hz), 3.83 (3H, s), 6.80-6.95 (5H, m), 7.87-7.92 (2H, d, J=8.9 Hz); LC/MS (APCI) m/z 271.19 (M+H)⁺; HPLC tᵣ=5.68 min (>99%) 80% MeCN in H₂O; FAB-HRMS calcd for C₁₇H₁₉O₃ 271.1329 found (M+H)⁺ 271.1329 m/z.

### 1-(4-(3-Ethyl-4-hydroxyphenoxy)phenyl)ethanone 141

To a solution of 1-(4-(3-ethyl-4-methoxyphenoxy)phenyl)ethanone 140 (0.174 g, 0.64 mmol) in dry DCM (5 mL), cooled to −78° C., was added slowly drop-wise BBr₃ (1.2 mL of a 1M solution in DCM, 1.2 mmol) and the reaction was stirred for 2 h before being allowed to warm to r.t. Stirring at r.t. was continued for a further 2 h before tlc showed the absence of starting material and the reaction was quenched with water. The organic layer was separated and concentrated under reduced pressure. Purification by flash chromatography (20 g, Flashmaster II) using a slow elution gradient of DCM to 2% to 5% MeOH yielded the title compound, 0.115 g, 70%: ¹H NMR δ (270 MHz, CDCl₃) 1.21 (3H, t, J=7.5 Hz), 2.58 (3H, s), 2.65 (2H, q, J=7.5 Hz), 6.64 (1H, s), 6.74-7.01 (5H, m), 7.89-7.95 (2H, m); ¹³C NMR δ (68 MHz, CDCl₃) 13.9, 23.2, 26.4, 116.2, 116.4, 118.9. 121.5, 130.9, 132.4, 148.0, 151.2, 163.6, 198.2; LC/MS (APCI) m/z 257.29 (M+H)⁺; HRMS (FAB⁺) calcd. for C₁₆H₁₇O₃ (M+H)⁺ 257.1172, found 257.1174; HPLC tᵣ=2.16 min (>96%) 90% MeCN in H₂O.

### 4-(4-Fluorophenyl)-4-oxobutanoic Acid 142

To a stirred mixture of succinic anhydride (1.00 g, 10 mmol) in fluorobenzene (5.6 mL, 60 mmol), cooled to −5° C., was added aluminium chloride (2.66 g, 20 mmol). The reaction temperature was maintained at −5° C. for 1 h before being allowed to warm slowly to r.t. over 17 h. The mixture was poured into a stirred solution of aqueous HCl (30 mL of 18%) at 0° C. and stirring was continued for a further 30 min while the mixture was allowed to warm to r.t. The resulting white powder was collected by filtration and washed with water. Yield 1.67 g, 85%: ¹H NMR δ (400 MHz, CD₃OD) 2.70-2.74 (2H, m), 3.32-3.35 (2H, m), 6.64 (1H, s), 7.20-7.26 (2H, m), 8.06-8.10 (2H, m); LC/MS (APCI) m/z 195.17 (M−H)⁻; HPLC tᵣ=1.53 min (>99%) 80% MeCN in H₂O.

### tert-Butyl 4-(4-fluorophenyl)-4-oxobutanoate 143

To a vigorously stirred suspension of anhydrous MgSO₄ (0.866 g, 7.2 mmol) in DCM (10 mL) in a sealed tube was added c.H₂SO₄ (0.1 mL, 1.8 mmol) and the mixture was stirred for 15 min. To this was then added 4-(4-fluorophenyl)-4-oxobutanoic acid 142 (0.355 g, 1.8 mmol) followed by t-BuOH (0.86 mL, 9 mmol), the reaction was sealed and stirred for 18 h at r.t. The reaction was quenched with sat. aq. NaHCO₃ (10 mL) and the mixture stirred until the MgSO₄ dissolved. The organic layer was separated, washed with brine, dried (MgSO₄) and concentrated under reduced pressure. Flash chromatography using an elution gradient of hexane to 30% EtOAc in hexane yielded the title compound, 0.162 g, 36%: ¹H NMR δ (400 MHz, CDCl₃) 1.46 (9H, s), 2.69 (2H, t, J=6.6 Hz), 3.23 (2H, t, J=6.6 Hz), 7.11-7.17 (2H, m), 8.00-8.04 (2H, m); LC/MS (APCI) m/z 179.04 (M−O^{t}Bu)⁻; HPLC tᵣ=2.71 min (>98%) 90% MeCN in H₂O.

### tert-Butyl 4-(4-(3-ethyl-4-methoxyphenoxy)phenyl)-4-oxobutanoate 144

To a solution of tert-butyl 4-(4-fluorophenyl)-4-oxobutanoate 143 (0.162 g, 0.64 mmol) and 3-ethyl-4-methoxyphenol 139 (0.152 g, 1.00 mmol) in MeCN (4 mL) was added K₂CO₃ (0.276 g, 2.00 mmol) and 18-crown-6 (catalytic) and the reaction was microwaved at 160° C. for 40 min. The mixture was concentrated under reduced pressure and the product isolated by flash chromatography using an elution gradient of hexane to 10% EtOAc in hexane. Yield 0.088 g, 36%; ¹H NMR δ (270 MHz, CDCl₃) 1.16 (2H, t, J=7.4 Hz), 1.43 (9H, s), 2.60-2.68 (4H, m), 3.17-3.22 (2H, m), 3.83 (3H, s), 6.80-6.94 (5H, m), 7.90-7.97 (2H, m); LC/MS (APCI) m/z 311.40 (M−O^{t}Bu)⁻; HPLC tᵣ=4.92 min (>98%) 90% MeCN in H₂O.

### 4-(4-(3-Ethyl-4-methoxyphenoxy)phenyl)-4-oxobutanoic Acid 145

To a stirred solution of tert-butyl 4-(4-(3-ethyl-4-methoxyphenoxy)phenyl)-4-oxobutanoate 144 (0.064 g, 0.17 mmol) in DCM (6 mL) was added drop-wise TFA (2 mL) and the reaction was stirred for 1 h. The mixture was concentrated under reduced pressure and the product precipitated from MeOH/H₂O, washed with water, dissolved in MeOH, concentrated under reduced pressure and dried under high vacuum. Yield 0.052 g, 93%; ¹H NMR δ (270 MHz, CD₃OD) 1.15 (2H, t, J=7.5 Hz), 2.57-2.70 (4H, m), 3.23-3.29 (2H, m), 3.83 (3H, s), 6.85-6.96 (5H, m), 7.95-7.99 (2H, m); LC/MS (APCI) m/z 327.39 (M−H)⁻.

### 4-(4-(3-Ethyl-4-methoxyphenoxy)phenyl)-4-oxo-N-((pyridin-3-yl)methyl)butanamide 146

To a stirred solution of 4-(4-(3-ethyl-4-methoxyphenoxy)phenyl)-4-oxobutanoic acid 145 (0.046 g, 0.14 mmol) in dry DCM (2 mL) was added NEt₃ (29 μL, 0.21 mmol) and 3-aminomethyl pyridine (21 μL, 0.21 mmol). To this was then added EDCI (0.0805 g, 0.42 mmol) and the reaction was stirred at rt for 19 h. To this was added sat. aq. bicarb. and the organic layer was separated and concentrated under reduced pressure. Purification by flash chromatography using an elution gradient of DCM to 5% MeOH in DCM gave the title compound, 0.056 g, 96%: ¹H NMR δ (270 MHz, CDCl₃) 1.15 (3H, t, J=7.5 Hz), 2.56-2.65 (4H, m), 3.28-3.33 (2H, m), 3.81 (3H, s), 4.41 (2H, d, J=5.9 Hz), 6.74-6.93 (5H, m), 7.18-7.23 (1H, m), 7.58-7.62 (1H, m), 7.86-7.92 (2H, m), 8.42-8.47 (2H, m); LC/MS (APCI) m/z 419.45 (M+H)⁺; HPLC tᵣ=2.59 min (>99%) 70% MeCN in H₂O.

### 4-(4-(3-Ethyl-4-hydroxyphenoxy)phenyl)-4-oxo-N-((pyridin-3-yl)methyl)butanamide 147

To a stirred solution of 4-(4-(3-Ethyl-4-methoxyphenoxy)phenyl)-4-oxo-N-((pyridin-3-yl)methyl)butanamide 146 in dry DCM (2 mL), cooled to −78° C., was added drop-wise BBr₃ (0.26 mL of a 1M solution, 0.26 mmol) and the reaction was allowed to warm slowly to room temperature for 20 h. Water was then added and the organic layer was separated and concentrated under reduced pressure: ¹H NMR δ (270 MHz, CDCl₃) 1.19 (3H, t, J=7.5 Hz), 2.54-2.69 (4H, m), 3.30-3.35 (2H, m), 4.47 (2H, d, J=6.0 Hz), 6.38-6.42 (1H, m, NH), 6.64-6.94 (6H, m), 7.23-7.30 (1H, m), 7.65 (1H, d, J=7.6 Hz), 7.89 (2H, d, J=9.0 Hz), 8.50 (1H, d, J=4.3 Hz), 8.53 (1H, s); LC/MS (APCI) m/z 403.46 (M−H)—; HPLC tᵣ=2.24 min (>91%) 70% MeCN in H₂O.

### 4-Acetyl-N-(4-methoxy-phenyl)-benzenesulphonamide 148

To a stirred solution of p-anisidine (0.152 g, 1.23 mmol) in dry DCM (3 mL) was added 4-acetylbenzenesulphonyl chloride (0.270 g, 1.23 mmol) and dry pyridine (1 mL) and the reaction was stirred for 19 h. To this was then added HCl aq. (20 mL of 1M) followed by DCM (20 mL) and the organic layer was separated and concentrated under reduced pressure. Purification by flash chromatography using an elution gradient of hexane to 50% EtOAc in hexane gave the title compound, 0.36 g, 96%: ¹H NMR δ (270 MHz, CDCl₃) 2.57 (3H, s), 3.68 (3H, s), 6.69 (2H, d, J=8.9 Hz), 6.97 (2H, d, J=9.2 Hz), 7.57 (1H, s), 7.77 (2H, d, J=8.6 Hz), 7.92 (2H, d, J=8.7 Hz); ¹³C NMR δ (100 MHz, CDCl₃) 26.8, 55.4, 114.5, 125.7, 127.6, 128.1, 128.7, 140.0, 142.7, 158.2, 196.9; LC/MS (APCI) m/z 304.47 (M−H)⁻; HPLC tᵣ=3.70 min (>99%) 90% MeCN in H₂O

### 4-Acetyl-N-(4-hydroxy-phenyl)-benzenesulphonamide 149

To a stirred solution of 4-acetyl-N-(4-methoxy-phenyl)-benzenesulphonamide 148 (0.253 g, 0.83 mmol) in dry DCM (10 mL), cooled to −78° C., was added slowly BBr₃ (1.6 mL of a 1M solution, 1.6 mmol) and the reaction was allowed to warm slowly to r.t. with stirring overnight before being quenched with water. The precipitate was collected by filtration and the product isolated from this by flash chromatography using an elution gradient of DCM to 10% MeOH in DCM. The first fraction was found to be product, yield 15 mg: ¹H NMR δ (270 MHz, CD₃OD) 2.60 (3H, s), 6.61 (2H, d, J=8.9 Hz), 6.84 (2H, d, J=8.9 Hz), 7.76 (2H, d, J=8.7 Hz), 8.05 (2H, d, J=8.7 Hz); HPLC tᵣ=7.25 min (>93%) 70% MeCN in H₂O; LC/MS (ES−) m/z 290.25 (M−H)⁻.

### 4-Acetyl-N-(4-methoxy-phenyl)-benzamide 150

To a stirred solution of 4-acetylbenzoic acid (0.164 g, 1 mmol) and p-anisidine (0.148 g, 1.2 mmol) in dry DCM (5 mL) and NEt₃ (0.17 mL) was added DMAP (catalytic) and the solution was cooled on ice. To this was then added EDCI (0.383 g, 2 mmol) and the reaction was allowed to warm to rt with stirring over 22 h. To this was added sat. aq. bicarb. and the mixture was filtered. The white powder collected was shown to be product. To the filtrate was added DCM (10 mL) and the organic layer was separated. Further product was isolated by flash chromatography using an elution gradient of hexane to 1:1 EtOAc:hexane followed by recrystallisation from DCM/hexane. Yield 90%: ¹H NMR δ (400 MHz, DMSO-d₆) 2.64 (3H, s), 3.75 (3H, s), 6.94 (2H, d, J=9.1 Hz), 7.69 (2H, d, J=9.1 Hz), 8.05-8.09 (4H, m), 10.31 (1H, s); ¹³C NMR δ (100 MHz, DMSO-d₆) 27.0, 55.2, 113.8, 122.1, 127.9, 128.2, 132.0, 138.7, 138.8, 155.7, 164.3, 197.7; HPLC tᵣ=3.73 min (>99%) 90% MeCN in H₂O; LC/MS (APCI) m/z 270.43 (M+H)⁺; FAB-HRMS calcd for C₁₆H₁₆NO₃ 270.1125 found (M+H)⁺ 270.1124 m/z.

### 4-Acetyl-N-(4-hydroxy-phenyl)-benzamide 151

Procedure as for 4-Acetyl-N-(4-hydroxy-phenyl)-benzenesulphonamide 149, from 4-acetyl-N-(4-methoxy-phenyl)-benzamide 150. Purified by flash chromatography using an elution gradient of hexane to EtOAc to give the title compound, 13 mg, 7%: ¹H NMR δ (270 MHz, CD₃OD) 2.65 (3H, s), 6.79 (2H, d, J=8.9 Hz), 7.47 (2H, d, J=8.9 Hz), 8.01 (2H, d, J=8.6 Hz), 8.11 (2H, d, J=8.6 Hz); HPLC tᵣ=3.33 min (>93%) 90% MeCN in H₂O; LC/MS (APCI) m/z 256.39 (M+H)⁺.

### 4-Acetyl-N-(4-methoxy-phenyl)-N-methyl-benzamide 152

To a stirred solution of 4-acetylbenzoic acid (0.164 g, 1 mmol) and N-methyl-p-anisidine (0.165 g, 1.2 mmol) in dry DCM (5 mL) and NEt₃ (0.17 mL) was added DMAP (catalytic) and the solution was cooled on ice. To this was then added EDCI (0.383 g, 2 mmol) and the reaction was allowed to warm to rt with stirring over 22 h. To this was added sat. aq. bicarb. and DCM and the organic layer was separated and concentrated under reduced pressure. Purification by flash chromatography using an elution gradient of hexane to 30% EtOAc in hexane gave the title compound, 0.186 g, 66%: ¹H NMR δ (400 MHz, CDCl₃) 2.47 (3H, s), 3.40 (3H, s), 3.66 (3H, s), 6.67 (2H, d, J=8.6 Hz), 6.89 (2H, d, J=8.2 Hz), 7.31 (2H, d, J=7.8 Hz), 7.69 (2H, d, J=7.8 Hz); ¹³C NMR δ (100 MHz, CDCl₃) 26.5, 38.2, 55.2, 114.3, 127.5, 127.9, 128.5, 136.9, 137.0, 140.4, 158.0, 169.5, 197.4; LC/MS (APCI) m/z 304.47 (M−H)⁻; HPLC tᵣ=3.61 min (>99%) 90% MeCN in H₂O.

### General Procedure 4: Preparation of 4-(4-fluoro-n-methylphenyl)-4-oxobutanoic Acids

To a stirred mixture of succinic anhydride (0.500 g, 5 mmol) and fluorotoluene or fluoroxylene (30 mmol), cooled to −5° C., was added AlCl₃ (1.33 g, 10 mmol) and the reaction was allowed to warm slowly to rt with stirring over 64 h. The mixture was cooled on ice before HCl aq. (15 mL of 18%) was added then stirred for a further 30 min while allowing to warm to rt. The white precipitate was collected by filtration and washed with water, dissolved in MeOH, concentrated under reduced pressure and dried in vacuo.

### 4-(4-Fluoro-3-methylphenyl)-4-oxobutanoic Acid 153

Prepared according to general procedure 4 from 2-fluorotoluene. LC/MS (APCI) m/z 209.22 (M−H)⁻; HPLC tᵣ=2.24 min (>99%) 90% MeCN in H₂O.

### 4-(4-Fluoro-2-methylphenyl)-4-oxobutanoic Acid 154

Prepared according to general procedure 4 from 3-fluorotoluene. LC/MS (APCI) m/z 209.22 (M−H)⁻; HPLC tᵣ=3.11 min (>69%) 25 to 95% MeCN in H₂O over 6 min.

### 4-(4-Fluoro-3,5-dimethylphenyl)-4-oxobutanoic Acid 155

Prepared according to general procedure 4 from 2-fluoro-1,3-dimethylbenzene. LC/MS (APCI) m/z 223.27 (M−H)⁻; HPLC tᵣ=2.46 min (>99%) 90% MeCN in H₂O.

### 4-(4-Fluoro-2,6-dimethylphenyl)-4-oxobutanoic Acid 156

Prepared according to general procedure 4 from 1-fluoro-3,5-dimethylbenzene. LC/MS (APCI) m/z 223.27 (M−H)⁻; HPLC tᵣ=2.83 min (>83%) 25 to 95% MeCN in H₂O over 6 min.

### General Procedure 5: Preparation of 4-(4-fluoro-n-methylphenyl)-4-oxo-N-((pyridin-3-yl)methyl)butanamides

To a stirred solution of 4-(4-fluoro-n-methylphenyl)-4-oxobutanoic acid in dry DCM (˜2 mL) was added 3-aminomethylpyridine (2 eq.) and the solution was cooled on ice. To this was then added EDCI (2 eq.) and the reaction was allowed to warm to rt with stirring for 17 h. Sat. aq. bicarb. (5 mL) was added followed by DCM (3 mL) and the organic layer was separated and concentrated under reduced pressure. The amides were purified by flash chromatography using an elution gradient of DCM to 5% MeOH in DCM.

### 4-(4-Fluoro-3-methylphenyl)-4-oxo-N-((pyridin-3-yl)methyl)butanamide 157

Prepared according to general procedure 5 from 4-(4-fluoro-3-methylphenyl)-4-oxobutanoic acid 153: ¹H NMR δ (270 MHz, CDCl₃) 2.21 (3H, d, J=1.7 Hz), 2.57 (2H, t, J=6.4 Hz), 3.23 (2H, t, J=6.4 Hz), 4.34 (2H, d, J=6.0 Hz), 6.93-6.99 (1H, m), 7.11-7.16 (1H, m), 7.32 (1H, bt, J=5.5 Hz), 7.53-7.56 (1H, m), 7.67-7.75 (2H, m), 8.35 (1H, d, J=3.8 Hz), 8.40 (1H, s); LC/MS (ES−) m/z 299.39 (M−H)⁻; HPLC tᵣ=3.78 min (>99%) 90% MeCN in H₂O.

### 4-(4-Fluoro-2-methylphenyl)-4-oxo-N-((pyridin-3-yl)methyl)butanamide 158

Prepared according to general procedure 5 from 4-(4-fluoro-2-methylphenyl)-4-oxobutanoic acid 154: ¹H NMR δ (270 MHz, CDCl₃) 2.36 and 2.45 (3H, 2×s), 2.58-2.62 (2H, m), 3.21-3.26 and 3.29-3.35 (2H, 2×m), 4.42 (2H, d, J=5.9 Hz), 6.62 (1H, bs), 6.88-7.00 (2H, m), 7.18-7.25 (1H, m), 7.61 (1H, d, J=7.6 Hz), 7.70-7.78 (1H, m), 8.45-8.47 (2H, m); LC/MS (ES+) m/z 299.45 (M−H)⁻; HPLC tr=3.58 min (>99%) 90% MeCN in H₂O.

### 4-(4-Fluoro-3,5-dimethylphenyl)-4-oxo-N-((pyridin-3-yl)methyl)butanamide 159

Prepared according to general procedure 5 from 4-(4-fluoro-3,5-dimethylphenyl)-4-oxobutanoic acid 155:1 H NMR δ (270 MHz, CDCl₃) 2.26 (6H, d, J=2.2 Hz), 2.62 (2H, t, J=6.4 Hz), 3.31 (2H, t, J=6.4 Hz), 4.43 (2H, d, J=6.0 Hz), 6.56 (1H, bs), 7.20-7.25 (1H, m), 7.59-7.64 (3H, m), 8.46-8.50 (2H, m); LC/MS (ES+) m/z 313.43 (M−H)⁻; HPLC tᵣ=3.83 min (>97%) 90% MeCN in H₂O.

### 4-(4-Fluoro-2,6-dimethylphenyl)-4-oxo-N-((pyridin-3-yl)methyl)butanamide 160

Prepared according to general procedure 5 from 4-(4-fluoro-2,6-dimethylphenyl)-4-oxobutanoic acid 156: ¹H NMR ((270 MHz, CDCl₃) 2.10, 2.16 and 2.22 (6H, 3×s), 2.51-2.56 (2H, m), 2.96-3.01 and 3.06-3.11 (2H, 2×m), 4.34 (2H, t, J=6.7 Hz), 6.60-6.72 (2H, m), 7.10-7.16 (1H, m), 7.20-7.30 (1H, m), 7.53-7.59 (1H, m), 8.35-8.39 (2H, m); LC/MS (ES+) m/z 313.43 (M−H)⁻; HPLC tr=3.73 min (>99%) 90% MeCN in H₂O.

### 4-(4-(3-Ethyl-4-methoxyphenoxy)-3-methylphenyl)-4-oxo-N-((pyridin-3-yl)methyl)butanamide 161

A mixture of 4-(4-fluoro-3-methylphenyl)-4-oxo-N-((pyridin-3-yl)methyl)butanamide 157 (0.156 g, 0.5 mmol), 3-ethyl-4-methoxyphenol 139 (0.152 g, 1 mmol), K₂CO₃ (1 mmol) and 18-crown-6 (catalytic) in MeCN (4 mL) was microwaved at 160° C. for 40 min. The mixture was then concentrated under reduced pressure, water was added and the organics extracted into EtOAc. The product was isolated as the middle of three fractions by flash chromatography using an elution gradient of DCM to 5% MeOH in DCM. The glassy product was precipitated from DCM/hexane to give white powder. Yield 14%: ¹H NMR δ (270 MHz, CDCl₃) 1.15 (3H, t, J=7.5 Hz), 2.34 (3H, s), 2.56-2.65 (4H, m), 3.31 (2H, t, J=6.4 Hz), 3.81 (3H, s), 4.42 (2H, d, J=5.9 Hz), 6.65 (1H, d, J=8.4 Hz), 6.73 (1H, bt, J=5.8 Hz), 6.79-6.83 (3H, m), 7.18-7.25 (1H, m), 7.58-7.62 (1H, m), 7.67 (1H, dd, J=8.7, 2.2 Hz), 7.83 (1H, d, J=1.4 Hz), 8.44-8.46 (1H, m), 8.48 (1H, d, J=1.5 Hz); HPLC>97% (Rt 2.23, 90% MeCN in H₂O); ES-ve MS (M−H)⁻ 335.35 m/z.

### [2-Ethyl-4-(1-oxo-indan-5-yl)-phenoxy]-acetic Acid Ethyl Ester 162

A solution of 5-(3-ethyl-4-hydroxyphenyl)-indan-1-one 10 (0.080 g, 0.32 mmol) in dry THF (10 mL) under an inert atmosphere was cooled to −10° C. A solution of LDA (1.8 M solution in heptane/THF/ethyl benzene, 0.193 mL, 0.35 mmol) was added over 20 minutes at −10° C. The mixture was cooled to −60° C. before ethyl bromoacetate (0.041 mL, 0.38 mmol) was added drop-wise and the mixture stirred at −60° C. for 3-4 h and allowed to warm to r.t. overnight. The reaction mixture was diluted with EtOAc and quenched with sat. NH₄Cl. The organics were combined, washed with water and concentrated to obtain the title compound: ¹H NMR δ (CDCl₃, 270 MHz) 1.24-1.32 (2×t, overlapped, 2×3H), 2.72-2.78 (m, 4H), 3.15-3.19 (m, 2H), 4.25-4.30 (q, J=7.1 Hz, 2H), 4.66-4.68 (s, 2H), 6.78 (2, J=8.4 Hz, 1H), 7.40 (dd, J=5.9, 2.2 Hz, 1H), 7.45 (appd, 1H), 7.55 (d, J=8.4 Hz, 1H), 7.61 (s, 1H), 7.78 (d, J=7.9 Hz, 1H); APCI-MS (M+H)⁺ 339.1 m/z.

### Biological Data

The following assay to determine 17B-HSD Type I activities was performed

### 17B-HSD Type I Assay

### Standard Operating Procedure

Purpose

To determine regulatory activity of compounds on 17β-hydroxysteroid dehydrogenase activity (type 1) in human breast cancer cells (T-47D)

Safety Notes
Wear gloves and a lab coat when working with human cell lines.
³H-E₁, ³H-E₂, ¹⁴C-E₁, and ¹⁴C-E₂ are radioactive molecules.
Assume E₁, E₂ and inhibitors to be carcinogenic and/or teratogenic.
DMSO promotes absorption through the skin and so when used as a solvent the solute may be easily absorbed—avoid bodily contact. THF can form potentially explosive peroxides upon long standing in air.
Diethyl ether has extremely flammable liquid and vapour. It is harmful when swallowed, inhaled or absorbed through skin. Causes irritation to skin, eyes and respiratory tract and affects the central nervous system.
Solid CO₂— contact with the product may cause frostbite or freeze burns in exposed tissues.
Methanol is highly flammable.
Dichloromethane must be handled with caution—causes respiratory tract, skin and eye irritation and may be a carcinogen.
Ethyl acetate—flammable liquid and vapour which is harmful if swallowed or inhaled. Causes irritation to skin, eyes and respiratory tract and affects the central nervous system.
Ultima gold MV—contact hazard—will irritate skin and eyes.

Procedure

| Materials | Supplier | Cat. No. | Comments |
|---|---|---|---|
| a. [6,7-³H(N)]-E₁ | ARC | ART-819 | 20-60 |
| | | | Ci/mmol |
| b. [4-¹⁴C]-E₂ | ARC | ARC-1324 | 55.00 |
| | | | mCi/mmol |
| c. T-47D human breast cancer cells | | | |
| d. Oestrone | Sigma | E-9750 | |
| e. β-Oestradiol | Sigma | E-8875 | |
| f. DMSO | Sigma | D-2650 | |
| g. Diethyl Ether | Prolabo | | |
| h. Solid CO₂ | | | |
| i. Methanol | Merck | | |
| j. Ethanol | Merck | | |
| k. Dichloromethane ‘AnalaR’ | SDS | | |
| l. Ethyl Acetate ‘AnalaR’ | SDS | | |
| m. Ultima Gold MV | Packard | | |
| n. Test Tubes | Kimble | 125 × 16 mm borosilicate | |
| | | glass | |
| o. Test Tubes | Kimble | 75 × 12 mm borosilicate | |
| | | glass | |
| p. Scintillation Vials | Starstedt | 20 ml, polypropylene | |
| q. Glass vials and caps | Chromacol | | |

Equipment
Wallac 1414 liquid Scintillation counter (Perkin Elmer).
Multi-pulse Vortexer (Glas-Col)
Sample concentrator (Techne)
Fume Hood
Automatic TLC sampler ATS4 (Camag)
TLC Aluminium Sheets Silica Gel 60 F₂₅₄.MERCK.

Calculations

Use Specific Activity of ³H-E₁ to calculate the ³H-E₁ needed to be added to each flask for physiological concentration (5 pmol per flask). Also calculate how much needs to be added to each well for physiological concentration (3 pmol per well) when carrying out the assay in 24-well plate format.

Assay of 17β-hydroxysteroid Dehydrogenase Activity in T-47D Cells (E₁→E₂)
Remove the growth medium from all wells.
Add 1.5 ml of substrate [(assay medium +³H-E₁)+appropriate inhibitor] to each well.
Incubate the plates at 37° C. for 3 hours.
Take 1 ml from each well and add directly to tubes containing ¹⁴C-oestradiol (−20000 dpm).
Extract the steroids from the medium with 4 ml diethyl ether.
Mechanically shake the tubes for 3 minutes to get a good partition.
Separate the ether phase by freezing the aqueous phase in a solid carbon dioxide/methanol mixture and decant the ether into tubes containing 50 μg (20 μl of 10 mM solution) unlabelled product to help visualise the labelled product upon TLC.
Evaporate the ether phase to dryness under an airstream at 40° C.
Dissolve the residue in 50 μl Ethanol.
Spot onto a TLC plate with the automatic TLC sampler containing a fluorescent indicator.
Separate the oestrone and oestradiol by TLC using dichloromethane/ethyl acetate (4:1 v/v) as solvents.
Visualise the spots of product steroid (3H-E₂) under UV light, cut them out, and place them in scintillation vials.
Add 0.5 ml methanol to the vials to elute the pieces of TLC.
Add 0.5 ml assay medium to each vial to correct for volume.
Add 10 ml Ultima Gold MV to each vial.
Measure the total activity of the ³H isotope by counting 0.5 ml substrate solution containing the ³H isotope (3H-E₁), 0.5 ml methanol and 10 ml Ultima Gold MV. Measure the total activity of the 14C isotope by counting ¹⁴C-oestradiol (20000 dpm), 0.5 ml assay medium, 0.5 ml methanol and 10 ml Ultima Gold MV.
Count product and recovery radioactivity in a liquid scintillation counter using a program for dual [³H/¹⁴C] isotopes.

Calculations

Overall, three corrections are applied to the raw data:

### Recovery Correction.

### Blank Correction.

### Dilution Correction.

### Use Microsoft Excel.

B & C represent columns of ³H & ¹⁴C raw data (dpm) respectively:

| | |
|---|---|
| D. C/Mean Total Activity ¹⁴C = | Recovery |
| E. B/D = | Recovery Corrected |
| F. E − Blank (mean) = | Blank Corrected |
| G. F × Constant (see below) = | fmol/flask/0.5 hrs |
| H. cells/flask (millions) | |
| I. G/H = | fmol/3 hrs/million cells |
| J. I/3 = | fmol/hr/million cells |
| K. Mean of each triplicate = | Mean Activity |
| L. Standard Deviation of each triplicate = | Standard Deviation Activity |
| M. [(100/Mean activity of control) × L] = | % Activity |
| N. 100 − O = | % Inhibition |
| O. Mean % Inhibition of each triplicate = | Mean % Inhibition |
| P. SD % Inhibition of each triplicate = | Standard Deviation % |
| | Inhibition |
| Q. Label Treatments and Concentrations = | Inhibitor Codes & |
| | Concentrations |

Constant = [1.5 × (pmol ³H-E1/total dpm ³H-E1/well)] where 1.5 = Dilution (1.5/1)

Graphs and statistics as required.

### Results

Initially compounds were screened at 10 μM. After some optimisation it was found that a substantial proportion of compounds tested were highly active. As a result compounds were then screened at 1 μM.

In the table below.

Compounds that were tested @ 10 μM and showed >80% inhibition are designated A. Compounds that were tested @ 1 μM and showed >30% inhibition are designated B. Compounds that were tested @ 10 μM and showed <80% inhibition and compounds that were tested @ 1 μM and showed <30% inhibition are designated C.

Typically the sem is ±5%.

| | | Inhibition |
|---|---|---|
| Compound | | of |
| number | Structure | 17β-HSD1 |
| 3 | 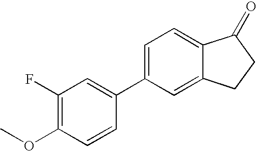 | C |
| 8 | 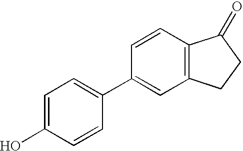 | A |
| 9 | 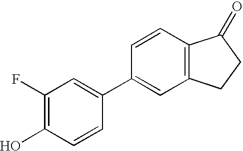 | A |
| 10 | 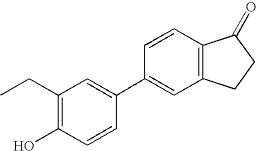 | A |
| 12 | 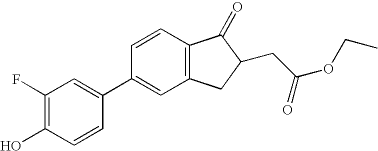 | C |
| 18 | 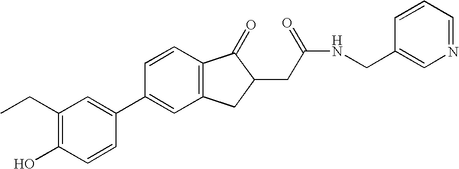 | B |
| 19 | 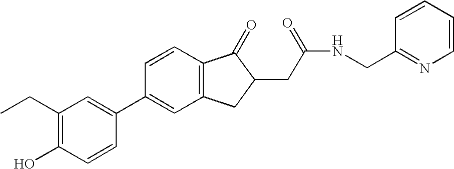 | C |
| 20 | 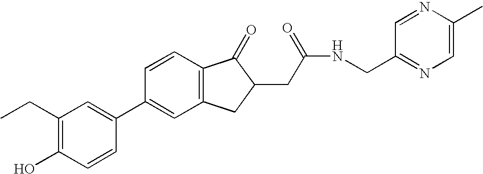 | C |
| 22 | 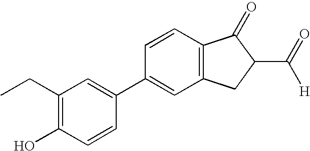 | C |
| 24 | 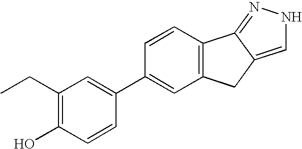 | C |
| 33 | 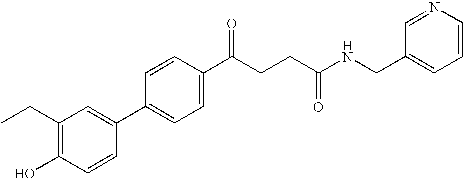 | C |
| 34 | 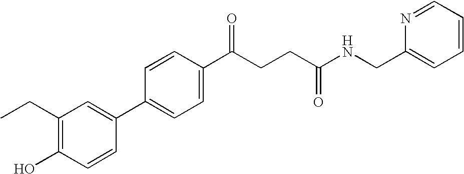 | C |
| 35 | 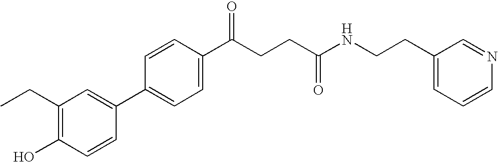 | C |
| 37 | 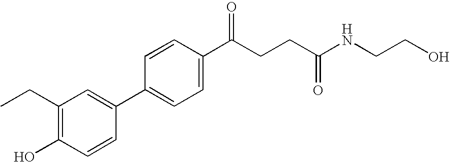 | C |
| 38 | 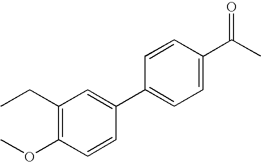 | C |
| 39 | 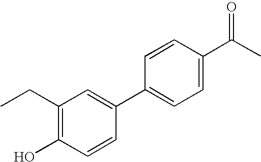 | A |
| 40 | 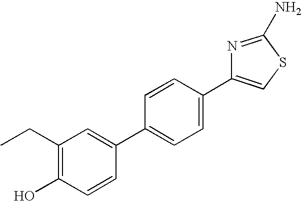 | C |
| 43 | 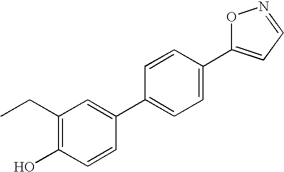 | C |
| 44 | 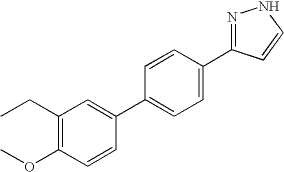 | C |
| 46 | 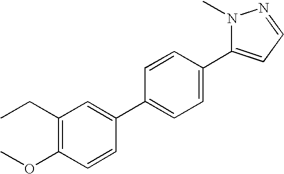 | C |
| 48 | 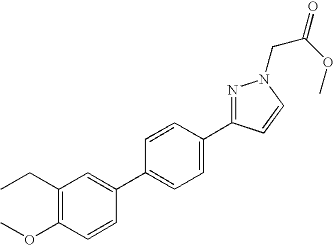 | C |
| 49 | 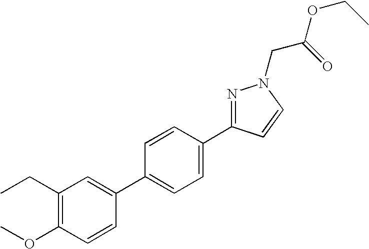 | C |
| 51 | 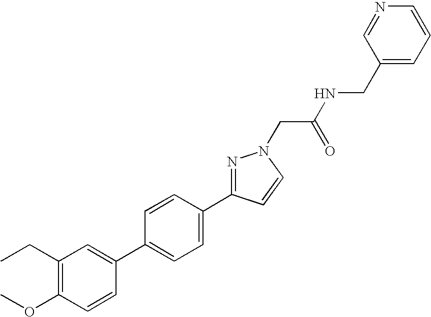 | C |
| 52 | 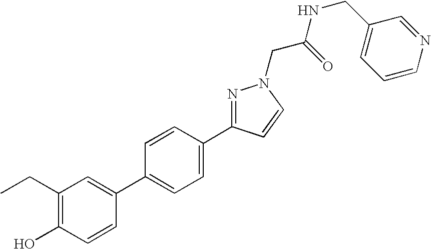 | C |
| 55 | 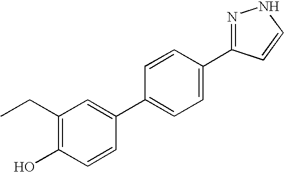 | C |
| 59 | 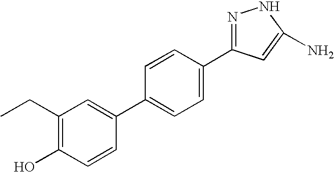 | C |
| 66 | 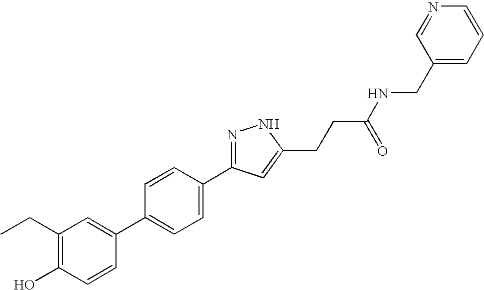 | C |
| 70 | 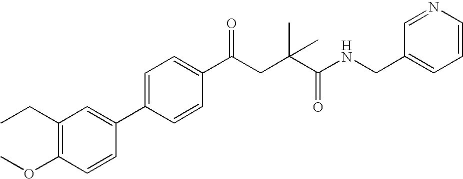 | C |
| 71 | 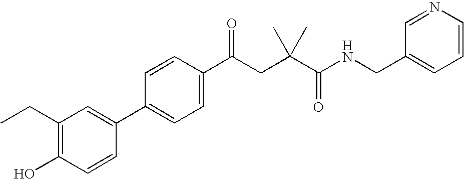 | B |
| 73 | 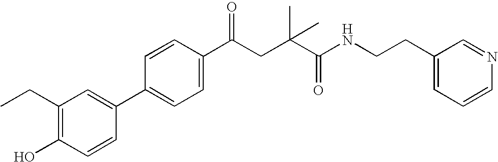 | C |
| 78 | 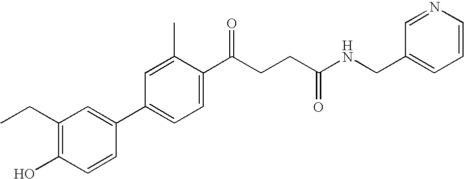 | C |
| 80 | 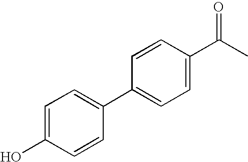 | C |
| 81 | 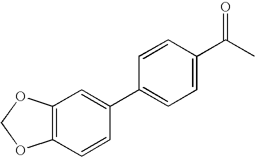 | C |
| 82 | 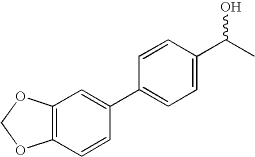 | C |
| 84 | 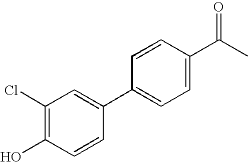 | A |
| 86 | 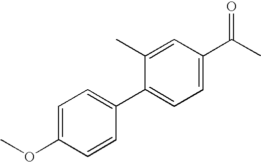 | C |
| 87 | 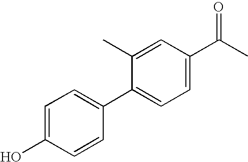 | C |
| 90 | 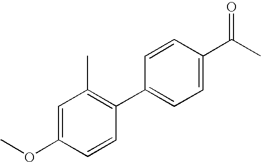 | C |
| 91 | 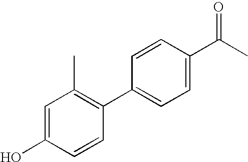 | A |
| 94 | 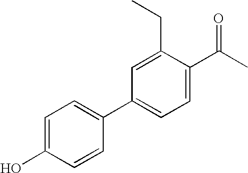 | C |
| 95 | 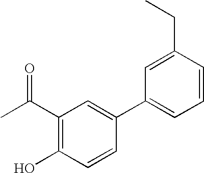 | C |
| 98 | 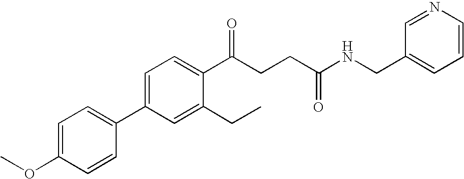 | C |
| 99 | 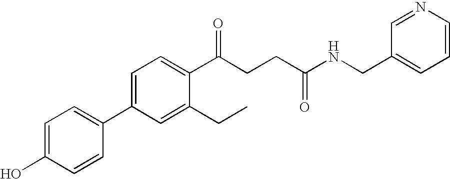 | A |
| 101 | 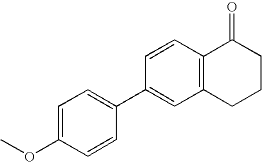 | C |
| 102 | 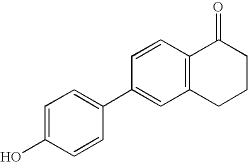 | A |
| 103 | 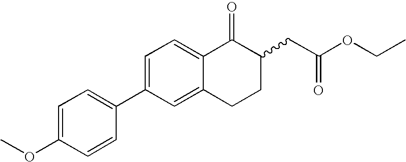 | C |
| 104 | 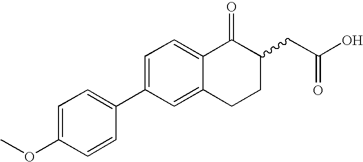 | C |
| 105 | 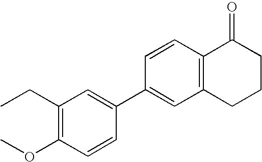 | C |
| 106 | 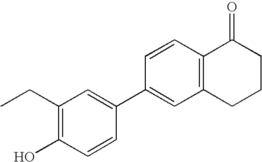 | C |
| 111 | 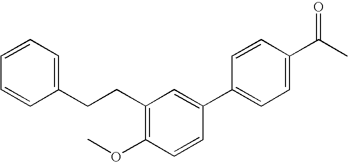 | C |
| 113 | 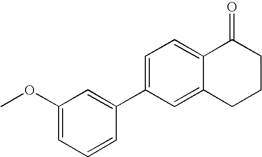 | C |
| 114 | 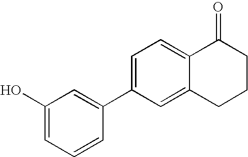 | A |
| 118 | 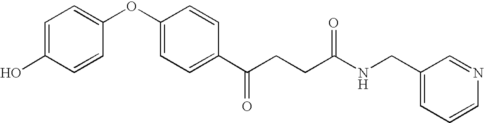 | C |
| 120 | 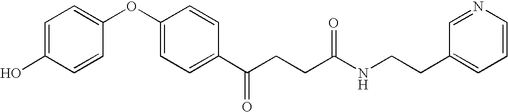 | C |
| 122 | 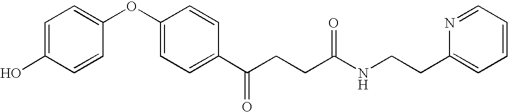 | C |
| 126 | 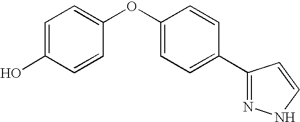 | C |
| 128 | 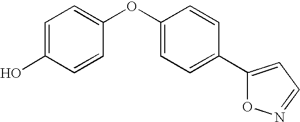 | C |
| 129 | 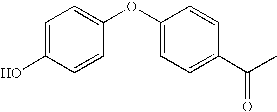 | C |
| 130 | 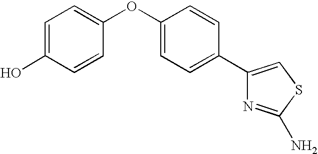 | C |
| 132 | 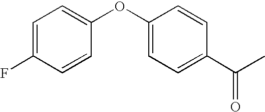 | C |
| 133 | 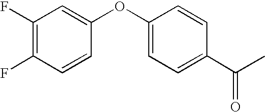 | C |
| 134 | 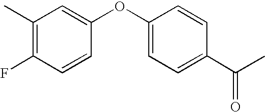 | C |
| 135 | 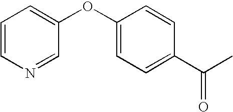 | C |
| 136 | 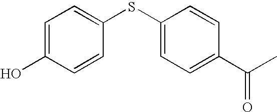 | C |
| 140 | 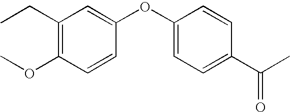 | C |
| 141 | 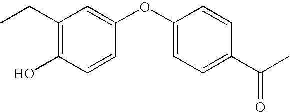 | B |
| 151 | 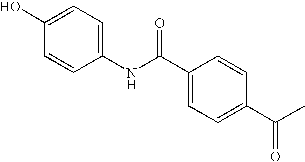 | C |
| 152 | 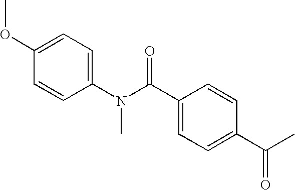 | C |
| 161 | 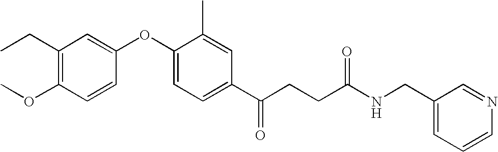 | C |
| 162 | 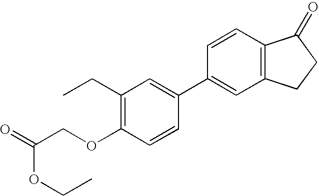 | C |

All publications and patents and patent applications mentioned in the above specification are herein incorporated by reference.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

### REFERENCES

(1) Saunders, C. M.; Baum, M. Management of early breast cancer. Oncol. in Pract. 1994, 3, 4-8
(2) Nicholls P. J. Breast cancer management: science and care together. Pharm. J. 1997, 259, 459-470
(3) Miller, B. A.; Kolonel, L. N.; Bernstein, L.; Young, Jr. J. L.; Swanson, G. M.; West, D.; Key, C. R.; Liff, J. M.; Glover, C. S.; Alexander, G. A.; et al. (eds). Racial/Ethnic patterns of cancer in the united states 1988-1992. National Cancer Institute 1996
(4) (a) Kaae, S, and Johansen, H. Does simple mastectomy followed by irradiation offer the survival comparable to radical procedures? International Journal of Radiation Oncology, Biology, Physics. 1977, 2, 1163-1166 (b) Holli, K.; Saaristo, R.; Isola, J.; Joensuu, H. and Hakama, M. Lumpectomy with or without postoperative radiotherapy for breast cancer with favourable prognostic features: results of a randomised study. Br. J. Cancer 2001, 84(2), 164-169
(5) Early Breast Cancer Trialists' Collaborative Group. Effects of adjuvant Tamoxifen and of cytotoxic therapy on mortality in early breast cancer. N. Eng. J. Med. 1988, 319, 1681-1692
(6) Gorski, J.; Toft, D.; Shyamala, G.; Smith, D.; Notides, A. Hormones receptors: studies on the interaction of estrogens with the uterus. Recent Prog. Horm. Res. 1968, 24, 45-80
(7) Gorski, J. and Gannon F. Current models of steroid hormone action: a critique. Ann. Rev. Physiol. 1976, 38, 425-450
(8) Coulson, C. J. Steroid biosynthesis and action, 2^{nd} edition. Molecular Mechanism of Drug Action. 1994, 95-122
(9) (a) Horwitz, K. B. and McGuire, W. L. Nuclear mechanism of estrogen action: effects of oestradiol and anti-estrogens on estrogens receptors and nuclear receptor processing. J. Biol. Chem. 1978, 253, 8185-8191 (b) Horwitz, K. B.; Koseki, Y. and McGuire, W. L. Oestrogen control of progesterone receptor in human breat cancer: role of oestradiol and antiestrogen. Endocrinology 1978, 103, 1742-1751
(10) (a) Jordan, V. C. The strategic use of antiestrogens to control the development and growth of breast cancer. Cancer. 1992, 70, 977-982 (b) Powles, T. J. Breast cancer prevention Breast Cancer Res. 2000, 2, 10-12
(11) Wakeling, A. E.; Bowler, J. Steroidal pure antiestrogens. J. Endocrinol. 1987, 112, R7-R10
(12) Sexton, M. J.; Gherman, R. B. Selective estrogen receptor modulators: the ideal estrogen replacement? Prim. Care. Update Ob/Gyns 2001, 8(1), 25-30
(13) Agnusdei, D.; Liu-Leage, S.; Augendre-Ferrante, B. Ann. Endocrinol. 1999, 60(3), 242-246
(14) John Smith, H.; Nicholls, P. J.; Simons, C.; Le Lain, R. Inhibitors of steroidogenesis as agents for the treatment of hormone-dependent breast cancer. Exp. Opin. Ther. Patents 2001, 11, 789-824
(15) (a) Castiglione-Gertsch, M. New aromatase inhibitors: more selectivity, less toxicity, unfortunately, the same activity. Eur. J. Cancer 1996, 32A, 393-395 (b) Miller, W. R. Aromatase inhibitors—where are we now? Br. J. Cancer 1996, 73, 415-417
(16) Santner, S. J.; Feil, P. D and Santen, R. J. In situ estrogen production via the oestrone sulphatase pathway in breast tumour: relative importance vs the aromatase pathway. J. Clin. Endocrin. Metab. 1984, 59, 29-33
(17) Purohit, A. Williams, G. J.; Howarth, N. M.; Potter, B. V. L. and Reed, M. J. Inactivation of steroid sulphatase by an active site-directed inhibitor, estrone-3-O-sulfamate. Biochem. 1995, 34, 11508-11514
(18) Purohit, A.; Williams, G. J.; Roberts, C. J.; Potter, B. V. L.; Reed, M. J. In vivo inhibition of oestrone sulphatase and dehydroepiandrosterone sulphatase by estrone-3-O-sulfamate. Int. J. Cancer 1995, 62, 106-111
(19) Woo, L. W. L.; Howarth, N. M.; Purohit, A.; Hejaz, H. A. M.; Reed, M. J. and Potter, B. V. L. Steroidal and nonsteroidal sulfamates as potent inhibitors of steroid sulphatase. J. Med. Chem. 1998, 41, 1068-1083
(20) Purohit, A.; Woo, L. W. L.; Singh, A.; Winterborn, C. J.; Potter, B. V. L. and Reed, M. J. In vivo activity of 4-methylcoumarin-7-O-sulfamate, a non steroidal, non estrogenic steroid sulphatase inhibitor. Cancer Res. 1996, 56, 4950-4955
(21) (a) Woo, L. W. L.; Purohit, A.; Malini, B.; Reed, M. J. and Potter, B. V. L. Potent active site-directed inhibition of steroid sulphatase by tricyclic coumarin-based sulfamates. Chemistry & Biology 2000, 7, 773-791 (b) Malini, B.; Purohit, A.; Ganeshapillai, D.; Woo, L. W. L.; Potter, B. V. L.; Reed, M. J. Inhibition of steroid sulphatase activity by tricyclic coumarin sulfamates. J. Steroid Biochem. Molec. Biol. 2000, 75, 253-25 (c) Purohit, A.; Woo, L. W. L.; Barrow, D.; Hejaz, H. A. M.; Nicholson, R. I.; Potter, B. V. L.; Reed, M. J. Non-steroidal and steroidal sulfamates: new drugs for cancer therapy. Mol. Cell. Endocrinol. 2001, 171, 129-135
(22) Purohit, A.; Woo, L. W. L.; Potter, B. V. L.; Reed, M. J. In vivo inhibition of oestrone sulphatase activity and growth of nitrosomethylurea-induced mammary tumours by 667 COUMATE. Cancer Res. 2000, 60, 3394-3396
(23) Claussner, A.; Nedelec, L.; Nique, F.; Philibert, D.; Teush, G.; Van de Velde, P. 11β-Amidoalkylestradiols as new series of pure anti-estrogens. J. Steroid. Biochem. 1992, 41, 609-614
(24) Li, P-K.; Chu, G-C.; Guo, J. P.; Selcer, K. W. Development of potent non-estrogenic oestrone sulphatase inhibitors. Steroids 1998, 63, 425-432
(25) (a) Jin, J-Z.; Lin, S-X. Human estrogenic 17β-hydroxysteroid dehydrogenase: predominance of oestrone reduction and its induction by NADPH. Biochem. Biophys. Res. 1999, 259, 489-493 (b) Penning, T. M. Molecular endocrinology of hydroxysteroid dehydrogenases. Endocrine Reviews 1997, 18, 281-305
(26) (a) Labrie, F. At the cutting edge. Intracrinology. Mol. Cell. Endocrinol. 1991, 78, C113-C118 (b) Poulin, R.; Labrie, F. Stimulation of cell proliferation and estrogenic response by adrenal C₁₉-?⁵-steroids in the ZR-75-1 Human Breast Cancer Cell Line. Cancer Res. 1986, 46, 4933-4937
(27) (a) Peltoketo, H.; Luu-The, V.; Simard, J.; Adamski, J. 17β-hydroxysteroid dehydrogenase (HSD)/17-ketosteroid reductase (KSR) family; nomenclature and main characteristics of the 17 HSD/KSR enzymes. J. Mol. Endocrinol. 1999, 23, 1-11 (b) Peltoketo, H.; lsomaa, V.; Maentausta, O.; Vihko, R. Complete amino acid sequence of human placental 17β-hydroxysteroid dehydrogenase deduced from cDNA. FEBS Lett. 1988, 239, 73-77 (c) Wu, L.; Einstein, M.; Geissler, W. M.; Chan, H. K.; Elliston, K. O.; Andersson, S. Expression cloning and characterization of human 17β-hydroxysteroid dehydrogenase type 2, a microsomal enzyme possessing 20α-hydroxysteroid dehydrogenase activity. J. Biol. Chem. 1993, 268, 12964-12969 (d) Geissler, W. M.; Davis, D. L.; Wu, L.; Bradshaw, K. D.; Patel, S.; Mendonca, B. B.; Elliston, K. O.; Wilston, J. D.; Russell, D. W.; Andersson, S. Male pseudohermaphroditism caused by mutation of testicular 17β-hydroxysteroid dehydrogenase 3. Nat. Genet. 1994, 7, 34-39 (e) Adamski, J.; Normand, T.; Leenders F.; Monte, D.; Begue, A.; Stehelin, D.; Jungblut, P. W.; de Launoit, Y. Molecular cloning of a novel widely expressed human 80 kDa 17β-hydroxysteroid dehydrogenase IV. Biochem. J. 1995, 311, 437-443 (f) Deyashiki, Y.; Ohshima, K.; Nakanishi, M.; Sato, K.; Matsuura, K.; Hara, A. Molecular cloning and characterization of mouse oestradiol 17β-dehydrogenase (A-specific), a member of the aldoketoreductase family. J. Biol. Chem. 1995, 270, 10461-10467 (f) R. Mindnich, G. Möller, J. Adamski, J. Molec. Cell. Endocrinol. 2004, 218, 7-20. The 17β-HSD type 13 isoform was disclosed at a workshop on 11β- and 17β-hydroxysteroid dehydrogenases: role in human disease, Germany 2005. Here the 17β-HSD type 13 was identified as human retinal shortchain dehyrogenase/reductase 3, PDB code 1YDE.
(28) Tremblay, M. R.; Auger, S. and Poirier, D. Synthesis of 16-(bromoalkyl)-estradiols having inhibitory effect on human placental oestradiol 17β-hydroxysteroid dehydrogenase (17β-HSD type 1). Bioorg. Med. Chem. 1995, 3, 505-523
(29) Tremblay, M. R.; Poirier, D. Overview of a rational approach to design type I 17β-hydroxysteroid dehydrogenase inhibitors without estrogenic activity: chemical synthesis and biological evaluation. J. Steroid. Biochem. 1998, 66, 179-191
(30) Collins, B. M.; Mac Lachlan, J. A.; Arnold, S. F. The estrogenic and anti-oestrogenic activities of phytochemicals with the human estrogen receptor expressed in yeast. Steroids 1997, 62, 365-372
(31) (a) Makela, S.; Poutanen, M.; Kostian, M. L.; Lehtimaki, N.; Strauss, L.; Santti, R.; Vihko, R. Inhibition of 17 beta-hydroxysteroid oxidoreductase by flavonoids in breast and prostate cancer cells. Proc. Soc. Exp. Biol. Med. 1998, 217, 310-316
(32) LeBail, J. C.; Laroche, T.; Marre-Fournier, F.; Habrioux, G. Aromatase and 17β-hydroxysteroid dehydrogenase inhibition by flavonoids. Cancer Lett. 1998, 133, 101-106
(33) Coldham, N. G.; James, V. H. T. A possible mechanism for increased breast cell proliferation by progestins through increased reductive 17β-hydroxysteroid dehydrogenase activity. Int. J. Cancer 1990, 45, 174-178
(34) Purohit, A.; Hejaz, H. A. M.; Walden, J.; MacCarthy-Marrogh, L.; Packam, G.; Potter, B. V. L.; Reed, M. J. The effect of 2-methoxyestrone-3-O-sulphamate on the growth of breast cancer cells and induced mammary tumours. Int. J. Cancer 2000, 85, 584-589
(35) Heer, J.; Miescher, K. Über Steroide. Marrianol-und Doisynolsäure. Über oestrogene carbonsäuren II. Helv. Chim. Acta 1945, 28, 156-165
(36) (a) Matkovics, B.; Taródi, B.; Baláspiri, L. Rearangement of steroids, VII. Schmidt reaction and Beckmann rearrangement of oestrone and its derivatives. Acta Chim. Acad. Scien. Hung. 1974, 80, 79-87 (b) Regan, B. M.; Newton Hayes, F. 17- and 17-Aza-D-homosteroids. J. Am. Chem. Soc. 1956, 78, 639-643
(37) Gupta, R. and Jindal, D. P. Synthesis and biological activity of some D-ring modified oestrone derivatives. Ind. J. Chem. 1999, 38B, 563-571
(38) Love, B. and Dawson, C. R. Alkylphenols related to the poison ivy principle. An improved method of synthesis involving the Na-Butanol cleavage of benzyl ethers. J. Am. Chem. Soc. 1956, 78, 6095-6101
(39) Okada, M.; Iwashita, S.; Koizumi, N. Efficient general method for sulfamoylation of a hydroxyl group. Tet. Lett. 2000, 41, 7047-7051.
(40) C. A. Hioruchi & J. Y. Satoh, Regioselective 2-Iodination of Estradiol, Estriol & Oestrone, J. Chem. Soc., Chem. Commun., 1982, 671-672.
(41) M. Numazawa and Y. Ogura, J. Chem. Soc., Chem. Commun. 1983, 9, 533.
(42) Williams, G. J.; Woo, L. W. L.; Mahon, M. F.; Purohit, A.; Reed, M. J.; Potter, B. V. L. X-ray crystal structure and mechanism of action of oestrone 3-O-sulphamate, a synthetic active site-directed inhibitor of oestrone sulphatase. Pharm. Sci. 1996, 2, 11-16
(43) Ghosh, D.; Pletnev, V. Z.; Zhu, D-W. et al. Structure of the human estrogenic 17 beta-hydroxysteroid dehydrogenase at 2.20 Å resolution. Structure, 1995, 3, 503-513
(44) (a) Lin, S. X.; Han, Q.; Azzi, A.; Zhu, D-W.; Gongloff, A.; Campbell, R. L. 3D structure of human estrogenic 17β-HSD: binding with various steroids. J. Steroid Biochem. Mol. Biol. 1999, 69, 425-429 (b) Puranen, T.; Poutanen, M.; Ghosh, D.; Vihko, R. and Vihko, P. Origin of substrate specificity of human and rat 17β-hydroxysteroid dehydrogenase Type 1, using chimeric enzymes and site-directed substitutions. Endocrinology 1997, 138, 3532-3539
(45) Breton, R.; Housset, D.; Mazza, C.; Fontecilla-Camps, J. C. The structure of a complex of human 17β-hydroxysteroid dehydrogenase with oestradiol and NADP+identifies two principal targets for the design of inhibitors. Structure (Lond) 1996, 4, 905-915
(46) Apel, R.; Berger, G. Über das hydrazidosulfamid Chem. Ber. 1958, 91, 1339-1341
(47) Woo, L. W. W.; Lightowler, M.; Purohit, A.; Reed, M. J.; Potter, B. V. L. Heteroatom-substituted analogues of the active-site directed inhibitor estra-1,3,5 (10)-trien-17-one-3-sulphamate inhibit oestrone sulphatase by different mechanism. J. Steroid Biochem Mol. Biol. 1996, 57, 79-88
(48) Duncan, L.; Purohit, A.; Howarth, N. M.; Potter, B. V. L.; Reed, M. J. Inhibition of oestrone sulphatase activity by estrone-3-methyl-thiophosphonate: a potential therapeutic agent in breast cancer Cancer Res. 1993, 53, 298-303

The invention is further described by the following numbered paragraphs:

1. A compound of Formula I

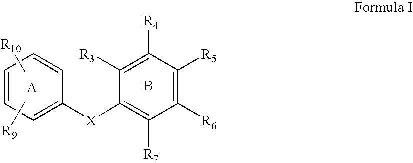

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted
wherein X is a bond or a linker group
wherein
(A) (i) R₉ is selected from alkyl and halogen groups; and
(ii) R₁₀ is selected from —OH, oxyhydrocarbyl and —OSO₂NR₁R₂; wherein R₁ and
   R₂ are independently selected from H and hydrocarbyl,
   wherein when R₉ is a halogen group and R₁₀ is —OH, at least one of R₃, R₄, R₅, R₆ and R₇ are as defined in (B), (C), (D) or (E);
   or
   (B) at least one of R₃, R₄, R₅, R₆ and R₇ is the group —C(═O)—CR₁₁R₁₂-R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
   wherein R₁₁ and R₁₂ are independently selected from H and hydrocarbyl;
   or
   (C) at least one of R₃, R₄, R₅, R₆ and R₇ together with another of R₃, R₄, R₅, R₆ and R₇ forms a ring containing —C(═O)—;
   or
   (D) at least one of R₃, R₄, R₅, R₆ and R₇ is selected from alkylheterocycle group, alkenylheterocycle group, alkylheteroaryl group, alkenylheteroaryl group, and heteroaryl groups
   or
   (E) at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
   wherein R₁₃ and R₁₄ are independently selected from H and hydrocarbyl.

2. Use of a compound in the manufacture of a medicament for use in the therapy of a condition or disease associated with 17β-hydroxysteroid dehydrogenase (17β-HSD),

wherein the compound is of Formula I

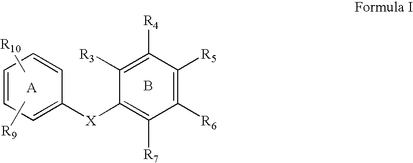

wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted and/or optionally contains one or more hetero atoms (such as N)
wherein X is a bond or a linker group
wherein
(A) (i) R₉ is selected from alkyl and halogen groups; or (ii) R₁₀ is selected from —OH, oxyhydrocarbyl and —OSO₂NR₁R₂; wherein R₁ and R₂ are independently selected from H and hydrocarbyl; or (iii) ring A contains one or more hetero atoms
or
(B) at least one of R₃, R₄, R₅, R₆ and R₇ is the group —C(═O)—CR₁₁R₁₂-R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof wherein R₁₁ and R₁₂ are independently selected from H and hydrocarbyl;
   or
   (C) at least one of R₃, R₄, R₅, R₆ and R₇ together with another of R₃, R₄, R₅, R₆ and R₇ forms a ring containing —C(═O)—;
   or
   (D) at least one of R₃, R₄, R₅, R₆ and R₇ is selected from alkylheterocycle group, alkenylheterocycle group, alkylheteroaryl group, alkenylheteroaryl group, and heteroaryl groups
   or
   (E) at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
   wherein R₁₃ and R₁₄ are independently selected from H and hydrocarbyl.

3. Use according to paragraph 2 wherein (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl

4. Use according to paragraph 2 or 3 wherein ring A contains a nitrogen.

5. Use according to paragraph 4 wherein the compound is of Formula II

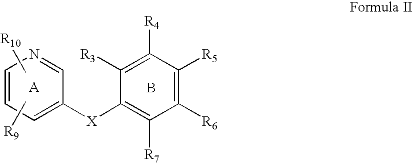

6. Use according to paragraph 5 wherein ring A is unsubstituted (and R₉ and R₁₀ are H).

7. Use according to paragraph 4 wherein the compound is of Formula III

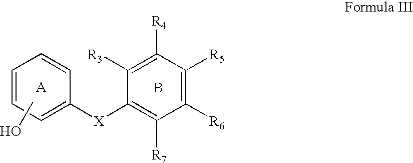

8. Use according to paragraph 4 wherein the compound is of Formula IV

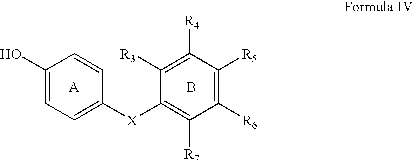

9. Use according to paragraph 4 wherein the compound is of Formula V

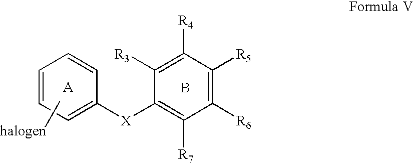

wherein halogen is preferably F.

10. Use according to paragraph 4 wherein the compound is of Formula VI

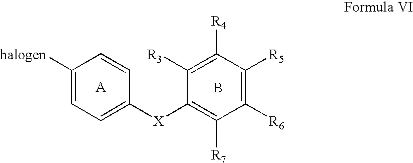

wherein halogen is preferably F.

11. Use according to paragraph 4 wherein the compound is of Formula VII

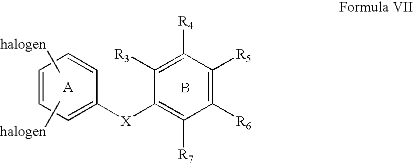

wherein halogen is preferably F.

12. Use according to paragraph 4 wherein the compound is of Formula VIII

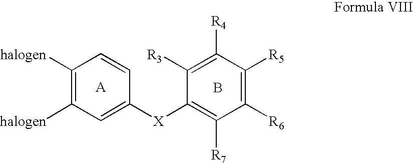

wherein halogen is preferably F.

13. Use of a compound in the manufacture of a medicament for use in the therapy of a condition or disease associated with 17β-hydroxysteroid dehydrogenase (17β-HSD),

wherein the compound is a compound as defined in paragraph 1.

14. The invention of any one of the preceding paragraphs wherein at least one of R₃, R₄, R₅, R₆ and R₇ together with another of R₃, R₄, R₅, R₆ and R₇ forms a ring containing —C(═O)—.

15. The invention of paragraph 14 wherein one of R₃, R₄, R₅, R₆ and R₇ together with another of R₃, R₄, R₅, R₆ and R₇ forms the ring

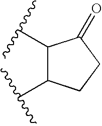

16. The invention of paragraph 14 wherein the compound is of Formula IX

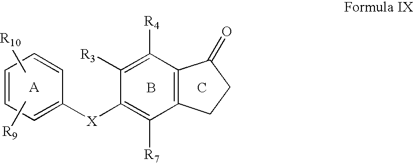

wherein ring C is optionally substituted, preferably wherein ring C is optionally substituted with a group selected from

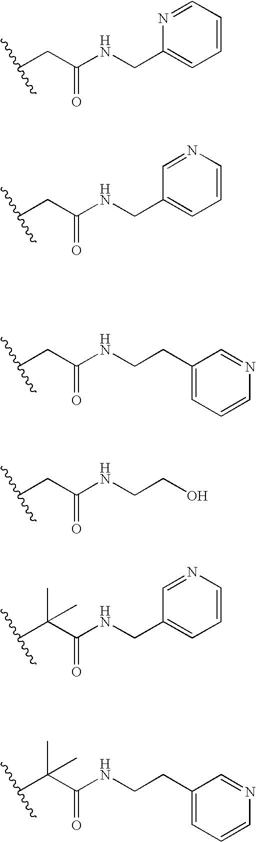

17. The invention of any one of paragraphs 1 to 13 at least one of R₃, R₄, R₅, R₆ and R₇ is the group —C(═O)—CR₁₁R₁₂-R₈

18. The invention of any one of paragraphs 1 to 13 at least one of R₃, R₄, R₅, R₆ and R₇ is the group —C(═O)—CH₂—R₈

19. The invention of any one of paragraphs 1 to 13, 17 or 18 wherein R₈ is selected from

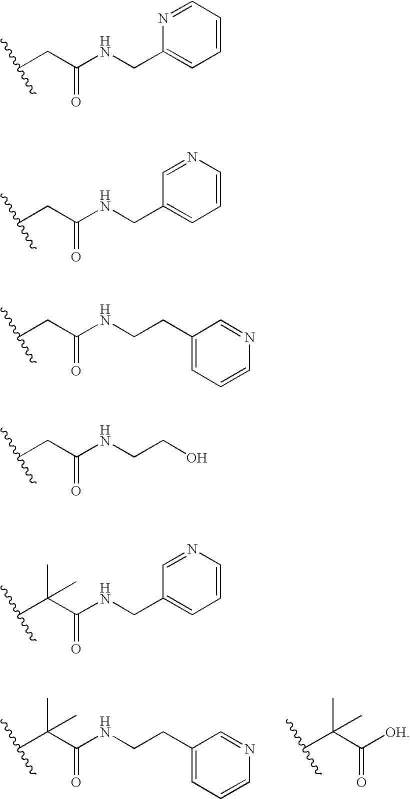

20. The invention of any one of the preceding paragraphs wherein (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl.

21. The invention of any one of the preceding paragraphs wherein at least one of R₃, R₄, R₅, R₆ and R₇ is the group —C(═O)—CR₁₁R₁₂—R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
   wherein R₁₁ and R₁₂ are independently selected from H and hydrocarbyl.

22. The invention of any one of the preceding paragraphs wherein at least one of R₃, R₄, R₅, R₆ and R₇ together with another of R₃, R₄, R₅, R₆ and R₇ forms a ring containing —C(═O)—.

23. The invention of any one of the preceding paragraphs wherein at least one of R₃, R₄, R₅, R₆ and R₇ is selected from alkylheterocycle group, alkenylheterocycle group, alkylheteroaryl group, alkenylheteroaryl group, and heteroaryl groups.

24. The invention of any one of the preceding paragraphs wherein at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;

wherein R₁₃ and R₁₄ are independently selected from H and hydrocarbyl.

25. The invention of any one of the preceding paragraphs wherein the oxyhydrocarbyl group is an alkoxy group.

26. The invention of any one of the preceding paragraphs wherein R₉ is selected from or R₉ of (A) is selected from ethyl and fluoro groups.

27. The invention of any one of the preceding paragraphs wherein R₁₀ is selected from or R₁₀ of (A) is selected from is selected from —OH and methoxy.

28. The invention of any one of the preceding paragraphs wherein X is selected from CH₂, O, S and a bond, preferably X is selected from O, S and a bond.

29. The invention of any one of the preceding paragraphs wherein the compound is of the formula

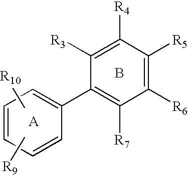

30. A pharmaceutical composition comprising a compound according to any one of paragraphs 1 to 29 optionally admixed with a pharmaceutically acceptable carrier, diluent, excipient or adjuvant.

31. A compound according to any one of paragraphs 1 to 29 for use in medicine.

32. A compound as substantially hereinbefore described with reference to any one of the Examples.

33 A composition as substantially hereinbefore described with reference to any one of the Examples.

34. A use as substantially hereinbefore described with reference to any one of the Examples.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A compound of Formula I 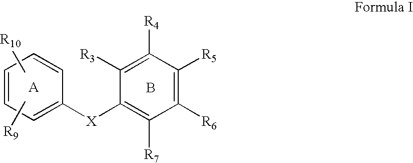
wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted
wherein X is a bond or a linker group
wherein
(A) (i) R₉ is selected from alkyl and halogen groups; and
(ii) R₁₀ is selected from —OH, oxyhydrocarbyl and —OSO₂NR₁R₂; wherein R₁ and
R₂ are independently selected from H and hydrocarbyl,
wherein when R₉ is a halogen group and R₁₀ is —OH, at least one of R₃, R₄, R₅, R₆ and R₇ are as defined in (B), (C), (D) or (E);
or
(B) at least one of R₃, R₄, R₅, R₆ and R₇ is the group —C(═O)—CR₁₁R₁₂—R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
wherein R₁₁ and R₁₂ are independently selected from H and hydrocarbyl;
or
(C) at least one of R₃, R₄, R₅, R₆ and R₇ together with another of R₃, R₄, R₅, R₆ and R₇ forms a ring containing —C(═O)—;
or
(D) at least one of R₃, R₄, R₅, R₆ and R₇ is selected from alkylheterocycle group, alkenylheterocycle group, alkylheteroaryl group, alkenylheteroaryl group, and heteroaryl groups
or
(E) at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
wherein R₁₃ and R₁₄ are independently selected from H and hydrocarbyl.

2. A method of manufacturing a medicament for use in the therapy of a condition or disease associated with 17β-hydroxysteroid dehydrogenase (17β-HSD), comprising a compound of Formula I 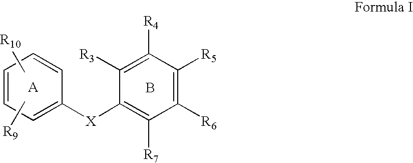
wherein R₃, R₄, R₅, R₆, R₇, R₉, and R₁₀, are independently selected from —H, —OH, hydrocarbyl groups, oxyhydrocarbyl groups, cyano (—CN), nitro (—NO₂), and halogens;
wherein ring A is optionally further substituted and/or optionally contains one or more hetero atoms (such as N)
wherein X is a bond or a linker group
wherein
(A) (i) R₉ is selected from alkyl and halogen groups; or (ii) R₁₀ is selected from —OH, oxyhydrocarbyl and —OSO₂NR₁R₂; wherein R₁ and R₂ are independently selected from H and hydrocarbyl; or (iii) ring A contains one or more hetero atoms
or
(B) at least one of R₃, R₄, R₅, R₆ and R₇ is the group —C(═O)—CR₁₁R₁₂—R₈ wherein R₈ is a selected from
(i) an alkyloxyalkyl group
(ii) a nitrile group,
(iii) alkylaryl group, wherein the aryl group is substituted by other than a C₁₋₁₀ group (iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
wherein R₁₁ and R₁₂ are independently selected from H and hydrocarbyl;
or
(C) at least one of R₃, R₄, R₅, R₆ and R₇ together with another of R₃, R₄, R₅, R₆ and R₇ forms a ring containing —C(═O)—;
or
(D) at least one of R₃, R₄, R₅, R₆ and R₇ is selected from alkylheterocycle group, alkenylheterocycle group, alkylheteroaryl group, alkenylheteroaryl group, and heteroaryl groups
or
(E) at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
wherein R₁₃ and R₁₄ are independently selected from H and hydrocarbyl.

3. A method according to claim 2 wherein (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl

4. A method according to claim 2 wherein ring A contains a nitrogen.

5. A method according to claim 4 wherein the compound is of Formula II

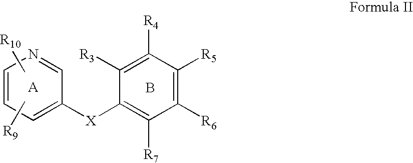

6. A method according to claim 5 wherein ring A is unsubstituted (and R₉ and R₁₀ are H).

7. A method according to claim 4 wherein the compound is of Formula III

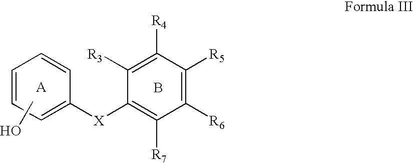

8. A method according to claim 4 wherein the compound is of Formula IV

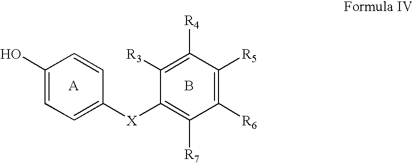

9. A method according to claim 4 wherein the compound is of Formula V 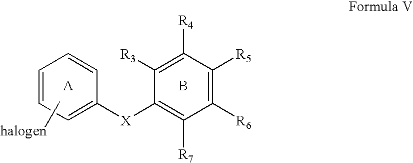
wherein halogen is preferably F.

10. A method according to claim 4 wherein the compound is of Formula VI 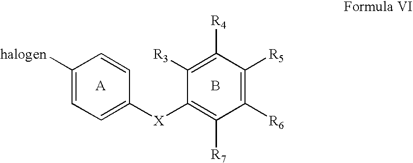
wherein halogen is preferably F.

11. A method according to claim 4 wherein the compound is of Formula VII 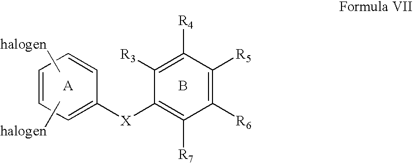
wherein halogen is preferably F.

12. A method according to claim 4 wherein the compound is of Formula VIII 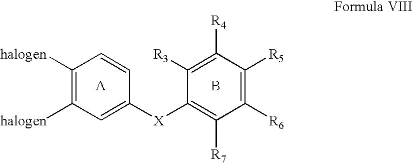
wherein halogen is preferably F.

13. A method of manufacturing a medicament for use in the therapy of a condition or disease associated with 17β-hydroxysteroid dehydrogenase (17β-HSD), comprising a compound as defined in claim 1.

14. A compound according to claim 1 wherein at least one of R₃, R₄, R₅, R₆ and R₇ together with another of R₃, R₄, R₅, R₆ and R₇ forms a ring containing —C(═O)—.

15. A method according to claim 2 wherein at least one of R₃, R₄, R₅, R₆ and R₇ together with another of R₃, R₄, R₅, R₆ and R₇ forms a ring containing —C(═O)—.

16. A compound according to claim 14 wherein one of R₃, R₄, R₅, R₆ and R₇ together with another of R₃, R₄, R₅, R₆ and R₇ forms the ring

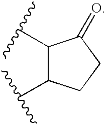

17. A method according to claim 15 wherein one of R₃, R₄, R₅, R₆ and R₇ together with another of R₃, R₄, R₅, R₆ and R₇ forms the ring

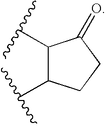

18. A compound according to claim 14 wherein the compound is of Formula IX 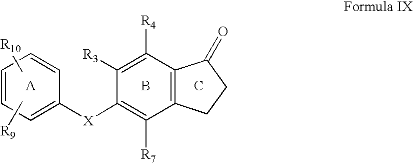
wherein ring C is optionally substituted, preferably wherein ring C is optionally substituted with a group selected from

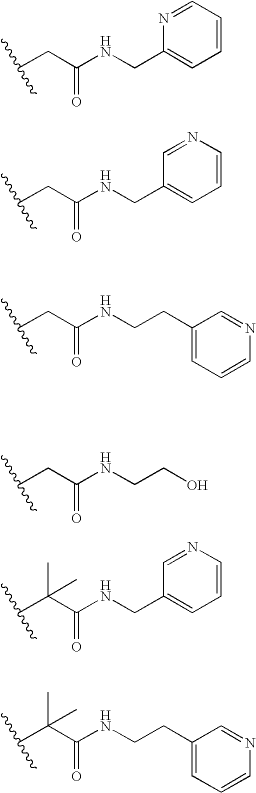

19. A method according to of claim 15 wherein the compound is of Formula IX 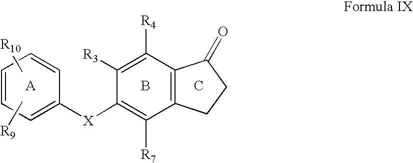
wherein ring C is optionally substituted, preferably wherein ring C is optionally substituted with a group selected from

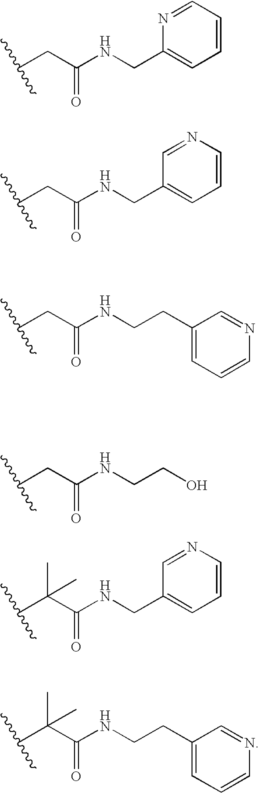

20. A compound according to claim 1 wherein at least one of R₃, R₄, R₅, R₆ and R₇ is the group —C(═O)—CR₁₁R₁₂—R₈.

21. A method according to claim 2 wherein at least one of R₃, R₄, R₅, R₆ and R₇ is the group —C(═O)—CR₁₁R₁₂—R₈.

22. A compound of claim 1 wherein at least one of R₃, R₄, R₅, R₆ and R₇ is the group —C(═O)—CH₂—R₈.

23. A method according to claim 2 wherein at least one of R₃, R₄, R₅, R₆ and R₇ is the group —C(═O)—CH₂—R₈.

24. A compound according to claim 1 wherein R₈ is selected from

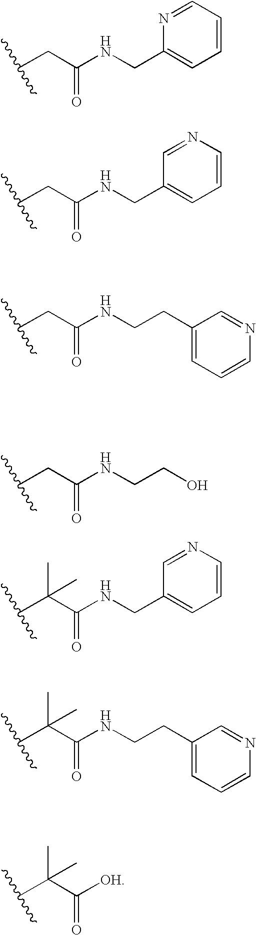

25. A method according to claim 2 wherein R₈ is selected from

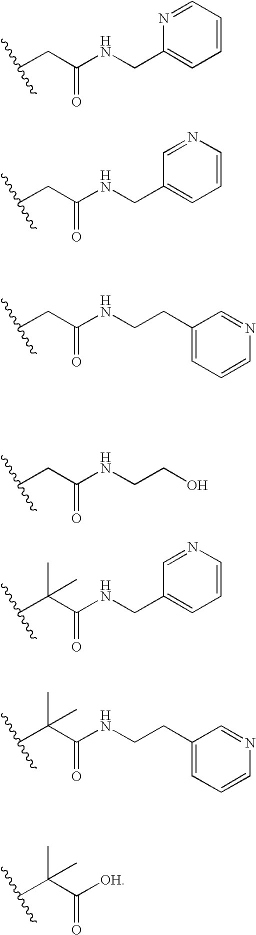

26. A compound according to claim 1 wherein (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl.

27. A method according to claim 2 wherein (i) R₉ is selected from alkyl and halogen groups; and (ii) R₁₀ is selected from —OH, oxyhydrocarbyl and —OSO₂NR₁R₂; wherein R₁ and R₂ are independently selected from H and hydrocarbyl.

28. A compound according to claim 1 wherein at least one of R₃, R₄, R₅, R₆ and R₇ is the group —C(═O)—CR₁₁R₁₂—R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
wherein R₁ and R₁₂ are independently selected from H and hydrocarbyl.

29. A method according to claim 2 wherein at least one of R₃, R₄, R₅, R₆ and R₇ is the group —C(═O)—CR₁₁R₁₂—R₈ wherein R₈ is a selected from
(i) an alkyloxyalkyl group
(ii) a nitrile group,
(iii) alkylaryl group, wherein the aryl group is substituted by other than a C1-10 group
(iv) alkenylaryl group wherein the aryl group is substituted
(v) alkylheteroaryl group, wherein when heteroaryl group comprises only C and N in the ring, the aryl group is substituted by other than a methyl group
(vi) alkenylheteroaryl group
(vii) ═N—O-alkyl or ═N—O—H group
(viii) branched alkenyl
(ix) alkyl-alcohol group or alkenyl-alcohol group
(x) amide or alkylamide wherein (a) the alkyl of the alkylamide is —CH₂— or —CH₂CH₂—, (b) the amide is di-substituted and/or (c) the amide is substituted with at least one of alkylheterocycle group, alkenylheterocycle group, alkylheteroaryl group, alkenylheteroaryl group, heteroaryl group, alkylamine group, alkyloxyalkyl group, alkylaryl group, straight or branched alkyl group,
(xi) —CHO, or together with another of R₃, R₄, R₅, R₆ and R₇ the enol tautomer thereof
wherein R₁₁ and R₁₂ are independently selected from H and hydrocarbyl.

30. A compound according to claim 1 wherein at least one of R₃, R₄, R₅, R₆ and R₇ together with another of R₃, R₄, R₅, R₆ and R₇ forms a ring containing —C(═O)—.

31. A method according to claim 2 wherein at least one of R₃, R₄, R₅, R₆ and R₇ together with another of R₃, R₄, R₅, R₆ and R₇ forms a ring containing —C(═O)—.

32. A compound according to claim 1 wherein at least one of R₃, R₄, R₅, R₆ and R₇ is selected from alkylheterocycle group, alkenylheterocycle group, alkylheteroaryl group, alkenylheteroaryl group, and heteroaryl groups.

33. A method according to claim 2 wherein at least one of R₃, R₄, R₅, R₆ and R₇ is selected from alkylheterocycle group, alkenylheterocycle group, alkylheteroaryl group, alkenylheteroaryl group, and heteroaryl groups.

34. A compound according to claim 1 wherein at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
wherein R₁₃ and R₁₄ are independently selected from H and hydrocarbyl.

35. A method according to claim 2 wherein at least one of R₃, R₄, R₅, R₆ and R₇ is selected from —CN, —C(R₁₃)═N—O-alkyl group, —C(R₁₄)═N—O—H group, optionally substituted pyrazole, optionally substituted thiazole, optionally substituted oxazole, optionally substituted isoxazole, optionally substituted pyridine, and optionally substituted pyrimidine, or together with another of R₃, R₄, R₅, R₆ and R₇ forms a nitrogen containing ring;
wherein R₁₃ and R₁₄ are independently selected from H and hydrocarbyl.

36. A compound according to claim 1 wherein the oxyhydrocarbyl group is an alkoxy group.

37. A method according to claim 2 wherein the oxyhydrocarbyl group is an alkoxy group.

38. A compound according to claim 1 wherein R₉ is selected from or R₉ of (A) is selected from ethyl and fluoro groups.

39. A method according to claim 2 wherein R₉ is selected from or R₉ of (A) is selected from ethyl and fluoro groups.

40. A compound according to claim 1 wherein R₁₀ is selected from or R₁₀ of (A) is selected from is selected from —OH and methoxy.

41. A method according to claim 2 wherein R₁₀ is selected from or R₁₀ of (A) is selected from is selected from —OH and methoxy.

42. A compound according to claim 1 wherein X is selected from CH₂, O, S and a bond, preferably X is selected from O, S and a bond.

43. A method according to claim 2 wherein X is selected from CH₂, O, S and a bond, preferably X is selected from O, S and a bond.

44. A compound according to claim 1 wherein the compound is of the formula

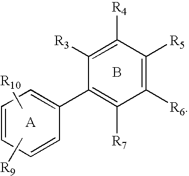

45. A method according to claim 2 wherein the compound is of the formula

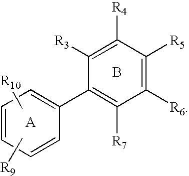

46. A pharmaceutical composition comprising a compound according to claim 1 optionally admixed with a pharmaceutically acceptable carrier, diluent, excipient or adjuvant.

47. A compound according to claim 1 for use in medicine.
